(19) United States
(12) Patent Application Publication
(10) Pub. No.: US 2025/0000891 A1
(43) Pub. Date: Jan. 2, 2025

(54) BIOMARKERS OF MEGAKARYOCYTE-DERIVED EXTRACELLULAR VESICLES

(71) Applicant: STRM.bio Incorporated, Cambridge, MA (US)

(72) Inventors: Jonathan THON, Cambridge, MA (US); Daniel BODE, York (GB); Laura GOLDBERG, Cambridge, MA (US)

(21) Appl. No.: 18/708,872

(22) PCT Filed: Nov. 11, 2022
(86) PCT No.: PCT/US22/49737

§ 371 Date: May 9, 2024

Related U.S. Application Data
(60) Provisional application No. 63/380,576, filed on Oct. 23, 2022.
(60) Provisional application No. 63/335,181, filed on Apr. 26, 2022.
(60) Provisional application No. 63/278,378, filed on Nov. 11, 2021.

(51) Int. Cl. A61K 31/7115; A61K 9/127; A61K 38/16; C12N 5/0735; C12N 15/11

(52) U.S. Cl. CPC A61K 31/7115; A61K 9/127; A61K 38/16; C12N 5/0606; C12N 15/11; C12N 2310/20

(57) ABSTRACT

Disclosed herein are compositions and methods related to megakaryocyte-derived extracellular vesicles derived from human pluripotent stem cells, where the megakaryocyte-derived extracellular vesicles present unique biomarkers.

### CROSS-REFERENCE TO RELATED APPLICATIONS

This application claims the benefit of priority to U.S. Provisional Patent Application Nos. 63/278,378, filed Nov. 11, 2021; 63/335,181, filed Apr. 26, 2022; and 63/380,576, filed Oct. 23, 2022, all of which are incorporated by reference herein in their entireties.

### FIELD

The present disclosure relates to biomarkers of megakaryocyte-derived extracellular vesicles derived and methods of using the same.

### BACKGROUND

In order to avoid drug cytotoxicity and side effects and/or rapid clearance, a variety of nano-vesicles have been developed to administer therapeutic agents.

The largest category of clinically approved nanoparticles is liposomes, which consist of a simple lipid bilayer surrounding an aqueous compartment. However, liposomes can also trigger adverse effects in a patient, including immune reactions and cytotoxicity, in addition to target non-specificity and inefficient unloading of therapeutic agents, because liposomes are foreign, synthetic entities, with limited cell or tissue targeting machinery. Adenovirus, retrovirus, AAV, and lentivirus vectors are currently the most popular viral vectors for gene therapy. Nevertheless, conventional methods of viral vector production using adherent cell lines and transient transfections in the presence of serum are not scalable.

Alternatively nano-vesicles, like extracellular vesicles, would benefit from further understanding of their biological and compositional characteristics.

Accordingly, there is a need for compositions and methods that enhance the effectiveness of cargo packaging and delivery to cells and that are well-characterized, to allow predictable isolation and standardization.

### SUMMARY

Disclosed herein are compositions and methods related to manufactured megakaryocyte-derived extracellular vesicles. Specifically, inter alia, the present megakaryocyte-derived extracellular vesicles demonstrate a unique biomarker profile, which make them well-suited for utilization in therapeutic delivery and treating various diseases or disorders. In various embodiments, the compositions and methods disclosed herein may be utilized for drug delivery and treatment of one or more genetic disorders. In embodiments, the compositions and methods disclosed herein may be utilized for drug delivery and treatment of infectious diseases. In embodiments, the compositions and methods disclosed herein may be utilized for drug delivery and treatment of a disease or disorder of hematopoiesis, e.g. thrombocytopenias/anemias. In embodiments, the compositions and methods disclosed herein may be utilized for drug delivery and treatment of hemoglobinopathies. The methods disclosed herein may be in vivo or ex vivo and may be used in for example, gene replacement therapy and gene-editing.

In one aspect, the present invention relates to a composition comprising: a plurality of substantially purified megakaryocyte-derived extracellular vesicles derived from a human pluripotent stem cell, wherein the composition comprises one or more proteins that are unique to or preferentially present in the megakaryocyte-derived extracellular vesicles as compared to other types of extracellular vesicles.

In one aspect, the present invention relates to a composition comprising: a plurality of substantially purified megakaryocyte-derived extracellular vesicles derived from a human pluripotent stem cell, wherein the composition comprises one or more nucleic acids encoding one or more proteins that are unique to or preferentially present in the megakaryocyte-derived extracellular vesicles as compared to other types of extracellular vesicles.

In one aspect, the present invention relates to a composition comprising: a plurality of substantially purified megakaryocyte-derived extracellular vesicles derived from a human pluripotent stem cell, wherein the composition comprises a lipid bilayer membrane surrounding a lumen and, wherein: the megakaryocyte-derived extracellular vesicle lumen comprises one or more megakaryocyte-derived nucleic acid molecules selected from mRNA, tRNA, rRNA, siRNA, microRNA, regulating RNA, and non-coding and coding RNA and the lipid bilayer membrane comprises one or more proteins associated with or embedded within, wherein the one or more proteins are unique to or preferentially present in the megakaryocyte-derived extracellular vesicles as compared to other types of extracellular vesicles.

In one aspect, the present invention relates to a composition comprising: a plurality of substantially purified megakaryocyte-derived extracellular vesicles derived from a human pluripotent stem cell, wherein the composition comprises a lipid bilayer membrane surrounding a lumen and, wherein: the megakaryocyte-derived extracellular vesicle lumen comprises one or more megakaryocyte-derived nucleic acid molecules selected from mRNA, tRNA, rRNA, siRNA, microRNA, regulating RNA, and non-coding and coding RNA and the lipid bilayer membrane comprises one or more nucleic acids, wherein the one or more nucleic acids encode one or more proteins that are associated with or embedded within the lipid bilayer membrane and are unique to or preferentially present in the megakaryocyte-derived extracellular vesicles as compared to other types of extracellular vesicles.

In one aspect, the present invention relates to a composition comprising: a plurality of substantially purified megakaryocyte-derived extracellular vesicles derived from a human pluripotent stem cell, wherein the composition comprises one or more nucleic acids that are unique to or preferentially present in the megakaryocyte-derived extracellular vesicles as compared to other types of extracellular vesicles.

In one aspect, the present invention relates to a composition comprising: a plurality of substantially purified megakaryocyte-derived extracellular vesicles derived from a human pluripotent stem cell, wherein the composition comprises one or more proteins encoded by one or more nucleic acids that are unique to or preferentially present in the megakaryocyte-derived extracellular vesicles as compared to other types of extracellular vesicles.

In another aspect, the present invention relates to a pharmaceutical composition comprising a composition disclosed herein and a pharmaceutically acceptable excipient or carrier.

In another aspect, the present invention relates to a method for transferring a deliverable therapeutic agent, comprising: (a) obtaining the megakaryocyte-derived extracellular vesicles of a composition disclosed herein; (b) incubating the megakaryocyte-derived extracellular vesicle with a therapeutic agent to allow the therapeutic agent to populate the lumen of the megakaryocyte-derived extracellular vesicle and/or associate with the surface of the megakaryocyte-derived extracellular vesicle and yield a deliverable therapeutic agent; and (c) administering the deliverable therapeutic agent to a patient or contacting the deliverable therapeutic agent with a biological cell in vitro and administering the contacted biological cell to a patient.

In another aspect, the present invention relates to a method of generating the megakaryocyte-derived extracellular vesicles of a composition disclosed herein, comprising: (a) obtaining a human pluripotent stem cell, the human pluripotent stem cell being a primary CD34+ hematopoietic stem cell sourced from peripheral blood or cord blood; (b) differentiating the human pluripotent stem cell to a megakaryocyte in the absence of added erythropoietin and in the presence of added thrombopoietin; and (c) isolating megakaryocyte-derived extracellular vesicles from the megakaryocytes.

In various embodiments, the compositions and methods disclosed herein may be utilized for drug delivery and treatment of one or more genetic disorders.

In another aspect, the present invention relates to a method for treating or preventing an infectious disease, comprising administering an effective amount of a composition disclosed herein.

In another aspect, the present invention relates to a method for treating or preventing an infectious disease, comprising administering an effective amount of a composition comprising a cell, which is contacted with a composition disclosed herein in vitro. In another aspect, the present invention relates to a method for treating a disease or disorder of hematopoiesis. In an aspect, the present invention relates to a method for treating a thrombocytopenia, comprising administering an effective amount of a composition disclosed herein, wherein the composition comprises megakaryocyte-derived extracellular vesicles which comprise a nucleic acid encoding a functional thrombocytopenia-related gene, or a protein product thereof, or a nucleic acid encoding a gene-editing protein capable of creating a functional thrombocytopenia-related gene, or a protein product thereof.

In another aspect, the present invention relates to a method for treating a thrombocytopenia, comprising administering an effective amount of a composition comprising a cell which is contacted with a composition disclosed herein in vitro, wherein the composition comprises megakaryocyte-derived extracellular vesicles which comprise a nucleic acid encoding a functional thrombocytopenia-related gene, or a protein product thereof, or a nucleic acid encoding a gene-editing protein capable of creating a functional thrombocytopenia-related gene, or a protein product thereof.

In another aspect, the present invention relates to a method for treating a hemoglobinopathy, comprising administering an effective amount of a composition disclosed herein, wherein the composition comprises megakaryocyte-derived extracellular vesicles which comprise a nucleic acid encoding a functional hemoglobinopathy-related gene, or a protein product thereof, or a nucleic acid encoding a gene-editing protein capable of creating a functional hemoglobinopathy-related gene, or a protein product thereof.

In another aspect, the present invention relates to a method for treating a hemoglobinopathy, comprising administering an effective amount of a composition comprising a cell which is contacted with a composition disclosed herein in vitro, wherein the composition comprises megakaryocyte-derived extracellular vesicles which comprise a nucleic acid encoding a functional hemoglobinopathy-related gene, or a protein product thereof, or a nucleic acid encoding a gene-editing protein capable of creating a functional hemoglobinopathy-related gene, or a protein product thereof.

### BRIEF DESCRIPTION OF THE DRAWINGS

The summary, as well as the following detailed description, are further understood when read in conjunction with the appended drawings. For the purpose of illustrating the disclosed methods, there are shown in the drawings' exemplary embodiments of the methods however, these should not be limited to the specific embodiments disclosed.

FIGS. 1A-1B are series of pie charts showing the overlap of top 100 exosome marker proteins as identified in the database ExoCarta (Keerthikumar et al. J Mol Biol. 2016; 428 (4): 688-692) (at world wide web at exocarta.org) with the disclosed MkEV proteome. 96 of these proteins were identified in the disclosed MkEV proteome, while 4 were not identified (FIG. 1A). As shown in FIG. 1B, of the top 100 EV marker proteins as defined by the VesiclePedia database (Pathan et al. Nucleic Acids Res. 2019 Jan. 8; 47: D516-D519) (at world wide web at microvesicles.org), 91 of these proteins were identified within the disclosed MkEV proteome, while 9 were not. Collectively, disclosed MkEVs largely contain common extracellular vesicle and exosome markers.

FIGS. 2A-2B are series of Venn diagrams showing the overlap of the disclosed MkEV proteome with the entire VesiclePedia and ExoCarta databases of human EV proteomes, revealing 147 proteins that are unique to disclosed MkEVs (FIG. 2A). Overlap of cell surface proteins on disclosed MkEVs and surface proteins captured by the VesiclePedia database showed that 13 cell surface and membrane proteins are unique to disclosed MKEVs (FIG. 2B). Surface and membrane proteins were defined according to Bausch-Fluck et al. (Bausch-Fluck et al. PLOS One. 10: e0121314) and the Human Protein Atlas (Thul P. J., Akesson L., Science. 2017. 365 (6340)).

FIG. 3 is a histogram showing KEGG Pathway enrichment amongst unique disclosed MkEV proteins. A list of significantly enriched KEGG pathways of unique disclosed MkEV proteins (p-value <0.05) is shown and includes pathways involved in aminoacyl-tRNA biosynthesis and neutrophil extracellular trap formation.

FIG. 4 is a diagram showing the direct protein-protein interaction network of unique MkEV proteins. The network was constructed to include curated direct protein-protein interactions (interaction score >0.4). Edge intensity depicts the associated interaction score (confidence of interaction). Clusters with >3 nodes were highlighted and numbered in descending order. Non-interacting nodes were excluded. Number 1 represents a histone cluster, 2 represents the tRNA aminoacylation for protein translation cluster, 3 represents the cluster of Septin proteins, 4 represents a mitochondrial-related cluster, and 5 represents cellular biosynthesis processes (transcription and translation).

FIG. 5 is a diagram showing the functional and physical protein-protein interaction network of unique MkEV proteins. The protein network was constructed using functional association and direct protein-protein interactions. Experimental and database evidence was included (interaction score >0.4). Clusters with >3 nodes were highlighted and numbered in descending order. Non-interacting nodes were excluded. Number 1 represents a histone cluster, 2 represents the tRNA aminoacylation for protein translation cluster, 3 represents the cluster of Septin proteins, 4 represents cellular biosynthesis processes (transcription and translation), 5 represents a hematopoietic cluster, and 6 represents a mitochondrial-related cluster.

FIG. 6 is a Venn diagram depicting the overlap of protein identifications between two MkEV batches. Out of 2332 proteins, 973 were shared between both batches, 1226 were identified in the first batch and 133 in the second MkEV batch.

FIG. 7 is a heatmap of the protein intensities of 973 overlapping MkEV proteins (sample loading normalized), highlighting strong consistency of protein quantities across MkEV batches.

FIG. 8 is a Venn diagram visualizing the overlap between the MkEV proteome and a comprehensive list of plasma EVs proteins (assembled from four published sources and a newly generated dataset). The comparison revealed 508 proteins that are uniquely identified in MkEVs.

FIG. 9A-9B are series of Venn diagrams to reveal the set of MkEV proteins not previously identified in healthy donor-derived CD61+ EVs. (FIG. 9A). Strong overlap between CD61+ EVs and plasma EVs. (FIG. 2B). A total of 2109 proteins were identified in MkEVs, but not present in CD61+ EVs.

FIG. 10 is a Venn diagram showing protein coding transcripts in the disclosed MkEVs. Overlap between MkEV protein coding genes (RNA-seq) and transcripts identified across the VesiclePedia database reveal 2701 mRNA transcripts that are unique to the disclosed MkEVs.

FIG. 11 is a histogram depicting the KEGG pathways of unique protein coding transcripts in the disclosed MkEVs. KEGG pathway enrichment is shown of all unique MkEV protein coding transcripts (2701 genes in total). A manually curated set of KEGG pathways is displayed.

FIG. 12 is a table listing the genes (RNA-seq) unique to the MkEVs disclosed herein that are associated with selected KEGG pathways.

FIG. 13 shows confocal microscopy images of HSPCs (Lineage depleted CD150+CD48-murine bone marrow cells) cocultured with MkEVs loaded with GFP-tagged Cas9 ribonucleoprotein (RNP). Cells cocultured with the cargo-loaded MkEVs were GFP-positive indicating cellular uptake of the GFP-tagged Cas9 loaded MkEVs. In contrast, control samples including cells alone and cells cocultured with MkEVs plus RNP (prior to co-culture with cells, the MKEVs were mixed with RNP but not loaded by electroporation, (no EP)) showed no GFP positivity. These data indicate successful delivery of RNP cargo-loaded MKEVs into HSPCs.

FIGS. 14A-14C show preferential targeting of MkEVs for hematopoietic stem and progenitor cells ex vivo. MkEVs that were loaded with either a GFP-tagged Cas9 protein (FIG. 14A) or labeled with a lipophilic fluorescent dye, DiD (FIG. 14B) were cocultured with primary whole bone marrow derived from wild type mice. Following 24-hours in co-culture, cells were analyzed by flow cytometry for the % of cells that were GFP+ or DiD+ (i.e., MkEV+). In addition, the percent of Lineage positive (Lin+), Lineage negative (Lin−), and Lineage negative/c-Kit+/Sca-1+ cells were simultaneously determined using fluorescently labeled antibodies against Lineage positive markers, Sca-1, and c-Kit cell surface proteins. The percentage of each subtype of cells in the heterogenous whole bone marrow population is shown in FIG. 14A. For cells cocultured with GFP-tagged Cas9 loaded MkEVs (as shown by the bar graphs in FIG. 14A), despite the vast majority (95%) of the cells in culture being Lin+ cells (differentiated cells), only up to 23% of these cells were positive for MkEVs. In contrast, while <5% were the hematopoietic stem and progenitor cells (Lin-cells), almost 50% of these cells were positive for MKEVs at the 300EVs per cell dose. Finally, for the rarest and most pluripotent hematopoietic stem cells evaluated in these cultures, the LSK cells, making up only 0.25% of the population, almost 40% of this population were positive for MKEVs. These data indicate the preferential ex vivo targeting of bone marrow-derived hematopoietic stem and progenitor cells. Similarly, as shown in FIG. 14B, for whole bone marrow cells cocultured with DiD-labeled MkEVs; only 20% of Lin+ cells were positive for MkEVs. In contrast, 30% of the rarer population of Lin-cells were positive for MkEVs. Finally, for the rarest and most pluripotent hematopoietic stem cells evaluated in these cultures (LSKs), up to 48% % of this population were positive for MKEVs. There were no significant changes in the percentage of total Lin+ and Lin-cells in the whole bone marrow cultures across all the conditions of MkEV co-culture when compared to controls (FIG. 14C).

FIGS. 15A-15K show in vivo biodistribution of fluorescently-labeled MkEVs following in vivo delivery to wild type mice. The experimental design is shown in FIG. 15A (n=3-5 mice/group). Fluorescently-labeled MkEVs were injected intravenously via tail vein into wild type mice, and tissues were harvested and analyzed for fluorescence 16 hours post injection. FIG. 15B shows fluorescent signal detected by IVIS in femurs dissected from mice. N=5 mice/group. Fluorescence in each homogenized tissue, as assayed by plate reader and normalized by tissue weight (FIG. 15C). In FIG. 15C, each data point from left to right for each tissue is saline, dye alone, and MkEVs, respectively. FIGS. 15D and 15E show graphs of experimental data of bone marrow cells stained using antibodies against CD45, Lineage markers, CD150, CD201, and CD48 are analyzed by flow cytometry to determine the % of hematopoietic (CD45+ cells; FIG. 15D) and % of very primitive long-term hematopoietic stem cells (CD45+/Lin−/CD150+/CD201+/CD48-cells; FIG. 15E) that were positive for MkEVs. MkEVs preferentially target the hematopoietic cells within the bone marrow (FIG. 15D) and within that compartment, are targeting the very rare (<0.03% of marrow cells) long-term hematopoietic stem cells (FIG. 15E). There were no changes in the peripheral white blood cell count (FIG. 15F), hemoglobin (FIG. 15G), platelet count (FIG. 15H), WBC differential (FIG. 15I), or % of CD45+ and CD45-cells (FIG. 11J), or % of the long-term hematopoietic stem cells in the marrow (FIG. 15K) 16 hours following MkEV injection indicating lack of hematopoietic toxicity following in vivo injection.

FIG. 16 shows a graph of experimental data demonstrating Cas9 loading into MkEVs. MkEVs were electroporated with Cas9, treated with proteinase K to remove any un-internalized cargo (free cargo and vesicle surface-associated cargo), or with filtration to remove any free cargo, and then analyzed by western blotting for quantification of Cas9. Controls included MkEVs plus Cas9 without electroporation±Proteinase K±filtration. Cas9 was present in electroporated MkEVs, but not in control un-electroporated MkEVs, following filtration indicating the successful vesicle association and/or internalization of protein cargo. Cas9 was present in electroporated MkEVs, but not in control un-electroporated MkEVs following proteinase K digestion indicating successful internalization and protection of loaded protein cargo following electroporation.

FIGS. 17A-17B show in vivo biodistribution of pDNA-loaded EVs was analyzed. A non-limiting schematic of the methods are shown in FIG. 17A. Reporter pDNA (CMV-driven GFP) was loaded into MkEVs by electroporation, labeled with DiD and then injected via tail vein into wild type mice. Tissues were isolated 16 hours post injection and analyzed for EV association by flow cytometry. Vehicle alone injected (Saline Ctrl) served as a negative control. As shown in FIG. 17B, Cargo-loaded EVs targeted bone marrow following intravenous injection and showed preferential uptake in hematopoietic stem and progenitor cells within the bone marrow compartment. Bars represent average percent DiD+ cells±SD; n=1 mouse/group.

FIGS. 18A-18C show that pDNA-loaded MkEVs deliver the pDNA cargo to hematopoietic stem and progenitors (HSPCs) in vivo. A non-limiting schematic of the methods is shown in FIG. 18A. Reporter pDNA (pDNA encoding CMV-driven GFP) was loaded into MkEVs by electroporation, labeled with DiD and then injected via tail vein into wild type mice. HSPCs (Lineage negative/c-Kit+/Sca-1+ cells) were isolated 16 hours post injection and analyzed for pDNA delivery by qPCR. Two representative experiments are shown in FIG. 18B-18C. In FIG. 18B, bars represent relative pDNA levels in HSPCs (fold change over saline controls) in mice injected with vehicle alone (Saline ctrl), 100 ng pDNA alone (pDNA only ctrl), and pDNA-loaded EVs. n=1 mouse/group. As shown, pDNA was successfully delivered to HSPCs following intravenous injection. In FIG. 18C, bars represent relative pDNA levels in HSPCs (fold change over saline controls) in mice injected with vehicle alone (Saline ctrl) and pDNA-loaded EVs. As shown, pDNA was successfully delivered to HSPCs following intravenous injection. Furthermore, pDNA cargo was only recovered from those HSPCs that were positive for EVs (DiD+) following intravenous injection. In contrast, minimal to no pDNA was recovered from the HSPCs that did not take up EVs (DiD−) following intravenous injection. n=1 mouse/group.

### DETAILED DESCRIPTION

The present invention is based, in part on the discovery of compositions and methods for making or using substantially purified megakaryocyte-derived extracellular vesicles that are characterized by particular sets of physical characteristics, such as biomarker composition (e.g. the presence, absence, or amount of protein-biomarker(s) or gene-biomarker(s)) and size, and can carry cargo in the lumen for use in delivering therapeutic agents. In embodiments, the megakaryocyte-derived extracellular vesicles of the disclosure are distinct from the naturally occurring products, which are collected from whole blood (Platelet Free Plasma) or derived from activated platelets (Platelet EVs). Accordingly, in aspects, the present invention provides compositions and methods of obtaining and using megakaryocyte-derived extracellular vesicles that are consistently produced, with desirable properties, and carry specific cargo-making their therapeutic use more likely to be successful.

Megakaryocyte-derived extracellular vesicles, which are relatively immune silent, can be repeatedly dosed; a distinct advantage when compared to immunogenic viral vectors. In some aspects, the megakaryocyte-derived extracellular vesicles are useful for in vivo genomic medicines that do not need conditioning treatments, so people can receive them in an outpatient setting. This platform is an important paradigm shift in gene therapy from ex vivo to in vivo delivery, that will democratize gene therapy by reducing time to treatment and cost.

In one aspect, the present invention relates to a composition comprising: a plurality of substantially purified megakaryocyte-derived extracellular vesicles derived from a human pluripotent stem cell, wherein the composition comprises one or more proteins that are unique to or preferentially present in the megakaryocyte-derived extracellular vesicles as compared to other types of extracellular vesicles.

In one aspect, the present invention relates to a composition comprising: a plurality of substantially purified megakaryocyte-derived extracellular vesicles derived from a human pluripotent stem cell, wherein the composition comprises one or more nucleic acids encoding one or more proteins that are unique to or preferentially present in the megakaryocyte-derived extracellular vesicles as compared to other types of extracellular vesicles. In embodiments, the one or more proteins or the one or more nucleic acids encoding one or more proteins are involved in aminoacyl-tRNA biosynthesis and/or in neutrophil extracellular trap formation.

In one aspect, the present invention relates to a composition comprising: a plurality of substantially purified megakaryocyte-derived extracellular vesicles derived from a human pluripotent stem cell, wherein the composition comprises a lipid bilayer membrane surrounding a lumen and, wherein: the megakaryocyte-derived extracellular vesicle lumen comprises one or more megakaryocyte-derived nucleic acid molecules selected from mRNA, tRNA, rRNA, siRNA, microRNA, regulating RNA, and non-coding and coding RNA and the lipid bilayer membrane comprises one or more proteins associated with or embedded within, wherein the one or more proteins are unique to or preferentially present in the megakaryocyte-derived extracellular vesicles as compared to other types of extracellular vesicles.

In one aspect, the present invention relates to a composition comprising: a plurality of substantially purified megakaryocyte-derived extracellular vesicles derived from a human pluripotent stem cell, wherein the composition comprises a lipid bilayer membrane surrounding a lumen and, wherein: the megakaryocyte-derived extracellular vesicle lumen comprises one or more megakaryocyte-derived nucleic acid molecules selected from mRNA, tRNA, rRNA, siRNA, microRNA, regulating RNA, and non-coding and coding RNA and the lipid bilayer membrane comprises one or more nucleic acids, wherein the one or more nucleic acids encode one or more proteins that are associated with or embedded within the lipid bilayer membrane and are unique to or preferentially present in the megakaryocyte-derived extracellular vesicles as compared to other types of extracellular vesicles. In embodiments, the nucleic acid molecule is exogenously derived.

In one aspect, the present invention relates to a composition comprising: a plurality of substantially purified megakaryocyte-derived extracellular vesicles derived from a human pluripotent stem cell, wherein the composition comprises one or more nucleic acids that are unique to or preferentially present in the megakaryocyte-derived extracellular vesicles as compared to other types of extracellular vesicles.

In one aspect, the present invention relates to a composition comprising: a plurality of substantially purified megakaryocyte-derived extracellular vesicles derived from a human pluripotent stem cell, wherein the composition comprises one or more proteins encoded by one or more nucleic acids that are unique to or preferentially present in the megakaryocyte-derived extracellular vesicles as compared to other types of extracellular vesicles. In embodiments, the one or more nucleic acids, or the one or more proteins encoded by one or more nucleic acids are involved in neutrophil extracellular trap formation, hematopoietic cell lineage, cell adhesion, ATP-binding cassette (ABC) transport, chemokine signaling pathway, phagocytosis, and/or aminoacyl-tRNA biosynthesis.

In embodiments, the megakaryocyte-derived extracellular vesicles are suitable for loading with cargo into the lumen and/or loading with cargo associated with the surface of the megakaryocyte-derived extracellular vesicles. In embodiments, in addition to or as an alternative to the cargo located in the lumen of the megakaryocyte-derived extracellular vesicles, the cargo is loaded into the megakaryocyte for packaging into the extracellular vesicles. In embodiments, in addition to or as an alternative to the cargo located in the lumen of the megakaryocyte-derived extracellular vesicles, the cargo is loaded directly into the megakaryocyte-derived extracellular vesicles. In embodiments, in addition to or as an alternative to the cargo located in the lumen of the megakaryocyte-derived extracellular vesicles, the cargo is loaded into the megakaryocyte for packaging into the extracellular vesicles. In embodiments, in addition to or as an alternative to the cargo located in the lumen of the megakaryocyte-derived extracellular vesicles, the cargo is loaded directly into the megakaryocyte-derived extracellular vesicles.

In embodiments, the cargo is one or more therapeutic agents, including therapeutic agents described herein. In embodiments, the cargo comprises one or more megakaryocyte-derived nucleic acid molecules selected from mRNA, tRNA, rRNA, siRNA, microRNA, regulating RNA, and non-coding and coding RNA. In embodiments, in addition to or as an alternative to the cargo located in the lumen of the megakaryocyte-derived extracellular vesicles, the cargo is loaded into the megakaryocyte for packaging into the extracellular vesicles. In embodiments, in addition to or as an alternative to the cargo located in the lumen of the megakaryocyte-derived extracellular vesicles, the cargo is loaded directly into the megakaryocyte-derived extracellular vesicles. In embodiments, the megakaryocyte-derived extracellular vesicles are suitable for loading with cargo associated with the surface of the megakaryocyte-derived extracellular vesicles.

In another aspect, the present invention relates to a pharmaceutical composition comprising a composition disclosed herein and a pharmaceutically acceptable excipient or carrier.

In another aspect, the present invention relates to a method for transferring a deliverable therapeutic agent, comprising: (a) obtaining the megakaryocyte-derived extracellular vesicles of a composition disclosed herein; (b) incubating the megakaryocyte-derived extracellular vesicle with a therapeutic agent to allow the therapeutic agent to populate the lumen of the megakaryocyte-derived extracellular vesicle and yield a deliverable therapeutic agent; and (c) administering the deliverable therapeutic agent to a patient or contacting the deliverable therapeutic agent with a biological cell in vitro and administering the contacted biological cell to a patient.

In another aspect, the present invention relates to a method for treating various diseases or disorders with the present megakaryocyte-derived extracellular vesicles.

### Biomarker Profile or Fingerprint

In various embodiments, the present megakaryocyte-derived extracellular vesicles are characterized by a unique biomarker profile or fingerprint that distinguishes them from, for instance, naturally-occurring megakaryocyte-derived extracellular vesicles and/or vesicles or extracellular vesicles derived from platelets. In various embodiments, the present megakaryocyte-derived extracellular vesicles are characterized by a such a biomarker profile or fingerprint, which comprises the identity (e.g. the presence or absence) or amount (e.g. substantial presence or substantial absence of a biomarker in a megakaryocyte-derived extracellular vesicle population; or presence on or absence from a majority of megakaryocyte-derived extracellular vesicle in a population; or percentage megakaryocyte-derived extracellular vesicles having a biomarker).

### Protein-Biomarkers

In embodiments, the composition comprises substantially purified megakaryocyte-derived extracellular vesicles comprising a lipid bilayer membrane surrounding a lumen and derived from a human pluripotent stem cell, wherein the lipid bilayer membrane comprises one or more proteins (also referred to as biomarkers) associated with or embedded within.

In embodiments, the composition comprises substantially purified megakaryocyte-derived extracellular vesicles comprising one or more nucleic acids encoding one or more proteins that are unique to or preferentially present in the megakaryocyte-derived extracellular vesicles as compared to other types of extracellular vesicles.

In embodiments, a direct protein-protein interaction exits between the unique MkEV proteins presently disclosed. In embodiments, the network includes curated direct protein-protein interactions (interaction score >0.4), where cluster number 1 represents a histone cluster, cluster number 2 represents the tRNA aminoacylation for protein translation, cluster number 3 represents the cluster of Septin proteins, cluster number 4 represents a mitochondrial-related cluster, and cluster number 5 represents cellular biosynthesis processes (both for transcription and translation).

In embodiments, the one or more proteins (e.g. at least 3, or at least 5, or at least 10, or at least 25, or at least 50, or at least 100, or at least 150, or at least 175) or the one or more nucleic acids (e.g. at least 3, or at least 5, or at least 10, or at least 25, or at least 50, or at least 100, or at least 150, or at least 175) encoding one or more proteins (e.g. at least 3, or at least 5, or at least 10, or at least 25, or at least 50, or at least 100, or at least 150, or at least 175) are involved in aminoacyl-tRNA biosynthesis and are optionally selected from YARS1, AARS1, GARS1, LARS1, EPRS1, TARS1, DARS1, WARS1, VARS1, NARS1, RARS1, SARS1, KARS1, IARS1, CARS1, QARS, and HARS1.

In embodiments, the one or more proteins (e.g. at least 3, or at least 5, or at least 10, or at least 25, or at least 50, or at least 100, or at least 150, or at least 175) or the one or more nucleic acids (e.g. at least 3, or at least 5, or at least 10, or at least 25, or at least 50, or at least 100, or at least 150, or at least 175) encoding one or more proteins (e.g. at least 3, or at least 5, or at least 10, or at least 25, or at least 50, or at least 100, or at least 150, or at least 175) are involved in neutrophil extracellular trap formation and are optionally selected from H4C1, H2AC20, H3C1, H2BC12, H3C15, MACROH2A1, H2AX, H2AZ2, H3-3A, H2AC14, and MACROH2A2.

In embodiments, the one or more proteins (e.g. at least 3, or at least 5, or at least 10, or at least 25, or at least 50, or at least 100, or at least 150, or at least 175) or the one or more nucleic acids (e.g. at least 3, or at least 5, or at least 10, or at least 25, or at least 50, or at least 100, or at least 150, or at least 175) encoding one or more proteins (e.g. at least 3, or at least 5, or at least 10, or at least 25, or at least 50, or at least 100, or at least 150, or at least 175), and are selected from H4C16, H2AC17, H3C13, RPL9P9, PHB1, SETSIP, H2AZ1, XP32, COPS9, SEC61A2, MARS1, MRE11, ATP5F1C, H4C1, H2AC20, H3C1, H2BC12, FCSK, CAVIN2, YARS1, H3C15, MACROH2A1, H2AX, H3-2, SELENOF, MPIG6B, SEPTIN7, AARS1, H1-5, H1-2, H2AZ2, GARS1, LARS1, THPO, RACK1, H3-3A, CCDC191, H2BC19P, SEPTIN2, EPRS1, TARS1, ATP5F1B, ERBIN, DARS1, WARS1, VARS1, NIBAN2, SEPTIN6, NARS1, RARS1, SEPTIN11, SEPTIN5, SARS1, NIBAN1, SNRPGP15, PIP4P2, CYRIB, CARMIL1, KARS1, IARS1, SEPTIN9, H1-4, ARHGAP45, H1-0, CARS1, GCN1, FADS2, TBC1D13, GET3, RO60, LAMTOR5, ELOC, H2AC14, SCARF1, RNF24, GCSAML, NRDC, ECPAS, MACROH2A2, ATP5F1A, DMTN, TANGO2, CSF2RB, WASHC5, KCNA3, QARS1, MINDY1, PTPA, EXOC3L2, PRUNE1, PLPBP, THUMPD1, WASHC4, NECTIN2, GFUS, ADGRE2, AKAP8L, FAM234A, ADSS2, ANKRD13D, KCT2, NT5C3A, PIP4P1, CCDC9, ELOB, HPGDS, MEAK7, TOMM70, SMIM1, HARS1, ATP5PD, OGA, CHSY1, SQLE, SUSD6, IGKV1-27, PEDS1-UBE2V1, CEP44, MYG1, NRROS, IL21R, GRK2, MCEMP1, ELAPOR2, SDAD1, CERT1, UBE2F, CALHM5, H1-10, EIPR1, PBDC1, ARMH3, VIPAS39, MESD, PROSER2, RABL6, FYB1, C17orf49, RMDN3, KYAT3, TTMP, ERO1A, CD244, CZIB, PLPP3, BABAM2, H1-3, NAXE, ENSA, PALS2, GUCY1B1, UMAD1, MIX23, PRXL2B, SNU13, RTRAF, KXD1, VSIR, EPOR, or MARCHF2.

In embodiments, more than about 1%, or more than about 5%, or more than about 10%, or more than about 15%, or more than about 20%, or more than about 25%, or more than about 30%, or more than about 35%, or more than about 40%, or more than about 45%, or more than about 50%, or more than about 55%, or more than about 60%, or more than about 65%, or more than about 70%, or more than about 75%, or more than about 80%, or more than about 85%, or more than about 90%, or more than about 95% of the megakaryocyte-derived extracellular vesicles comprise the one or more proteins, or the one or more nucleic acids encoding one or more proteins.

In embodiments, more than about 1%, more than about 2%, more than about 3%, more than about 4%, more than about 5%, more than about 6%, more than about 7%, more than about 8%, more than about 9%, more than about 10%, more than about 11%, more than about 12%, more than about 13%, more than about 14%, more than about 15%, more than about 16%, more than about 17%, more than about 18%, more than about 19%, more than about 20%, more than about 21%, more than about 22%, more than about 23%, more than about 24%, more than about 25%, more than about 26%, more than about 27%, more than about 28%, more than about 29%, more than about 30%, more than about 31%, more than about 32%, more than about 33%, more than about 34%, more than about 35%, more than about 36%, more than about 37%, more than about 38%, more than about 39%, more than about 40%, more than about 41%, more than about 42%, more than about 43%, more than about 44%, more than about 45%, more than about 46%, more than about 47%, more than about 48%, more than about 49%, more than about 50%, more than about 51%, more than about 52%, more than about 53%, more than about 54%, more than about 55%, more than about 56%, more than about 57%, more than about 58%, more than about 59%, more than about 60%, more than about 61%, more than about 62%, more than about 63%, more than about 64%, more than about 65%, more than about 66%, more than about 67%, more than about 68%, more than about 69%, more than about 70%, more than about 71%, more than about 72%, more than about 73%, more than about 74%, more than about 75%, more than about 76%, more than about 77%, more than about 78%, more than about 79%, more than about 80%, more than about 81%, more than about 82%, more than about 83%, more than about 84%, more than about 85%, more than about 86%, more than about 87%, more than about 88%, more than about 89%, more than about 90%, more than about 91%, more than about 92%, more than about 93%, more than about 94%, more than about 95%, more than about 96%, more than about 97%, more than about 98%, more than about 99%, or more of the megakaryocyte-derived extracellular vesicles comprise the one or more proteins, or the one or more nucleic acids encoding one or more proteins.

In embodiments, less than about 95%, or less than about 90%, or less than about 85%, or less than about 80%, or less than about 75%, or less than about 70%, or less than about 65%, or less than about 60%, or less than about 55%, or less than about 50%, or less than about 45%, or less than about 40%, or less than about 35%, or less than about 30%, or less than about 25%, or less than about 20%, or less than about 15%, or less than about 10%, or less than about 5%, or less than about 1% of the megakaryocyte-derived extracellular vesicles comprise the one or more proteins, or the one or more nucleic acids encoding one or more proteins.

In embodiments, less than about 99%, or less than about 98%, or less than about 97%, or less than about 96%, or less than about 95%, or less than about 94%, or less than about 93%, or less than about 92%, or less than about 91%, or less than about 90%, or less than about 89%, or less than about 88%, or less than about 87%, or less than about 86% or less, less, about 85%, or less than about 84%, or less than about 83%, or less than about 82%, or less than about 81%, or less than about 80%, or less than about 79%, or less than about 78%, or less than about 77%, or less than about 76%, or less than about 75%, or less than about 74%, or less than about 73%, or less than about 72%, or less than about 71%, or less than about 70%, or less than about 69%, or less than about 68%, or less than about 67%, or less than about 66%, or less than about 65%, or less than about 64%, or less than about 63%, or less than about 62%, or less than about 61%, or less than about 60%, or less than about 59%, or less than about 58%, or less than about 57%, or less than about 56%, or less than about 55%, or less than about 54%, or less than about 53%, or less than about 52%, or less than about 51%, or less than about 50%, or less than about 49%, or less than about 48%, or less than about 47%, or less than about 46%, or less than about 45%, or less than about 44%, or less than about 43%, or less than about 42%, or less than about 41%, or less than about 40%, or less than about 39%, or less than about 38%, or less than about 37%, or less than about 36% or less, less, about 35%, or less than about 34%, or less than about 33%, or less than about 32%, or less than about 31%, or less than about 30%, or less than about 29%, or less than about 28%, or less than about 27%, or less than about 26%, or less than about 25%, or less than about 24%, or less than about 23%, or less than about 22%, or less than about 21%, or less than about 20%, or less than about 19%, or less than about 18%, or less than about 17%, or less than about 16%, or less than about 15%, or less than about 14%, or less than about 13%, or less than about 12%, or less than about 11%, or less than about 10%, or less than about 9%, or less than about 8%, or less than about 7%, or less than about 6%, or less than about 5%, or less than about 4%, or less than about 3%, or less than about 2%, or less than about 1% of the megakaryocyte-derived extracellular vesicles comprise the one or more proteins, or the one or more nucleic acids encoding one or more proteins.

In embodiments, the lipid bilayer membrane comprises one or more proteins (e.g. at least 3, or at least 5, or at least 10), or one or more nucleic acids (e.g. at least 3, or at least 5, or at least 10) encoding one or more proteins (e.g. at least 3, or at least 5, or at least 10) selected from CAVIN2, MPIG6B, ERBIN, ELOC, CSF2RB, KCNA3, NECTIN2, IL21R, MCEMP1, PROSER2, FYB1, CD244, and EPOR. In embodiments, more than about 1%, or more than about 5%, or more than about 10%, or more than about 15%, or more than about 20%, or more than about 25%, or more than about 30%, or more than about 35%, or more than about 40%, or more than about 45%, or more than about 50%, or more than about 55%, or more than about 60%, or more than about 65%, or more than about 70%, or more than about 75%, or more than about 80%, or more than about 85%, or more than about 90%, or more than about 95% of the megakaryocyte-derived extracellular vesicles comprise a lipid bilayer membrane comprising the one or more proteins, or the one or more nucleic acids encoding one or more proteins.

In embodiments, more than about 1%, more than about 2%, more than about 3%, more than about 4%, more than about 5%, more than about 6%, more than about 7%, more than about 8%, more than about 9%, more than about 10%, more than about 11%, more than about 12%, more than about 13%, more than about 14%, more than about 15%, more than about 16%, more than about 17%, more than about 18%, more than about 19%, more than about 20%, more than about 21%, more than about 22%, more than about 23%, more than about 24%, more than about 25%, more than about 26%, more than about 27%, more than about 28%, more than about 29%, more than about 30%, more than about 31%, more than about 32%, more than about 33%, more than about 34%, more than about 35%, more than about 36%, more than about 37%, more than about 38%, more than about 39%, more than about 40%, more than about 41%, more than about 42%, more than about 43%, more than about 44%, more than about 45%, more than about 46%, more than about 47%, more than about 48%, more than about 49%, more than about 50%, more than about 51%, more than about 52%, more than about 53%, more than about 54%, more than about 55%, more than about 56%, more than about 57%, more than about 58%, more than about 59%, more than about 60%, more than about 61%, more than about 62%, more than about 63%, more than about 64%, more than about 65%, more than about 66%, more than about 67%, more than about 68%, more than about 69%, more than about 70%, more than about 71%, more than about 72%, more than about 73%, more than about 74%, more than about 75%, more than about 76%, more than about 77%, more than about 78%, more than about 79%, more than about 80%, more than about 81%, more than about 82%, more than about 83%, more than about 84%, more than about 85%, more than about 86%, more than about 87%, more than about 88%, more than about 89%, more than about 90%, more than about 91%, more than about 92%, more than about 93%, more than about 94%, more than about 95%, more than about 96%, more than about 97%, more than about 98%, more than about 99%, or more of the megakaryocyte-derived extracellular vesicles comprise a lipid bilayer membrane comprising the one or more proteins, or the one or more nucleic acids encoding one or more proteins.

In embodiments, less than about 95%, or less than about 90%, or less than about 85%, or less than about 80%, or less than about 75%, or less than about 70%, or less than about 65%, or less than about 60%, or less than about 55%, or less than about 50%, or less than about 45%, or less than about 40%, or less than about 35%, or less than about 30%, or less than about 25%, or less than about 20%, or less than about 15%, or less than about 10%, or less than about 5%, or less than about 1% of the megakaryocyte-derived extracellular vesicles comprise a lipid bilayer membrane comprising the one or more proteins, or the one or more nucleic acids encoding one or more proteins.

In embodiments, less than about 99%, or less than about 98%, or less than about 97%, or less than about 96%, or less than about 95%, or less than about 94%, or less than about 93%, or less than about 92%, or less than about 91%, or less than about 90%, or less than about 89%, or less than about 88%, or less than about 87%, or less than about 86% or less, less, about 85%, or less than about 84%, or less than about 83%, or less than about 82%, or less than about 81%, or less than about 80%, or less than about 79%, or less than about 78%, or less than about 77%, or less than about 76%, or less than about 75%, or less than about 74%, or less than about 73%, or less than about 72%, or less than about 71%, or less than about 70%, or less than about 69%, or less than about 68%, or less than about 67%, or less than about 66%, or less than about 65%, or less than about 64%, or less than about 63%, or less than about 62%, or less than about 61%, or less than about 60%, or less than about 59%, or less than about 58%, or less than about 57%, or less than about 56%, or less than about 55%, or less than about 54%, or less than about 53%, or less than about 52%, or less than about 51%, or less than about 50%, or less than about 49%, or less than about 48%, or less than about 47%, or less than about 46%, or less than about 45%, or less than about 44%, or less than about 43%, or less than about 42%, or less than about 41%, or less than about 40%, or less than about 39%, or less than about 38%, or less than about 37%, or less than about 36% or less, less, about 35%, or less than about 34%, or less than about 33%, or less than about 32%, or less than about 31%, or less than about 30%, or less than about 29%, or less than about 28%, or less than about 27%, or less than about 26%, or less than about 25%, or less than about 24%, or less than about 23%, or less than about 22%, or less than about 21%, or less than about 20%, or less than about 19%, or less than about 18%, or less than about 17%, or less than about 16%, or less than about 15%, or less than about 14%, or less than about 13%, or less than about 12%, or less than about 11%, or less than about 10%, or less than about 9%, or less than about 8%, or less than about 7%, or less than about 6%, or less than about 5%, or less than about 4%, or less than about 3%, or less than about 2%, or less than about 1% of the megakaryocyte-derived extracellular vesicles comprise a lipid bilayer membrane comprising the one or more proteins, or the one or more nucleic acids encoding one or more proteins.

In embodiments, the lipid bilayer membrane comprises one or more proteins (e.g. at least 3, or at least 5, or at least 10), or one or more nucleic acids (e.g. at least 3, or at least 5, or at least 10) encoding one or more proteins (e.g. at least 3, or at least 5, or at least 10) selected from CD54, CD18, CD43, CD11b, CD62P, CD41, CD61, CD21, CD51, phosphatidylserine (PS), CLEC-2, LAMP-1 (CD107a), CD63, CD42b, CD9, CD31, CD47, CD147, CD32a, and GPVI.

In embodiments, the lipid bilayer membrane comprises phosphatidylserine, e.g., without limitation by testing for Annexin V.

In embodiments, the lipid bilayer membrane comprises one or more proteins selected from CD62P, CD41, and CD61.

In embodiments, greater than about 40%, greater than about 50%, greater than about 60%, greater than about 70%, greater than about 80%, greater than about 90%, greater than about 95%, or greater than about 99% of the megakaryocyte-derived extracellular vesicles comprising a lipid bilayer membrane comprising CD41 also comprise CD61 in the lipid bilayer membrane.

In embodiments, the present megakaryocyte-derived extracellular vesicles are characterized by the expression and/or presence of one or more of CD54, CD18, CD43, CD11b, CD62P, CD41, CD61, CD21, CD51, and CLEC-2. In embodiments, the present megakaryocyte-derived extracellular vesicles are characterized by the expression and/or presence of one or more of PS, CD62P, LAMP-1 (CD107a), CD42b, CD9, CD43, CD31, and CD11b. In embodiments, the present megakaryocyte-derived extracellular vesicles are characterized by the expression and/or presence of one or more of PS, CD61, CD62P, LAMP-1 (CD107a), CLEC-2, and CD63. In embodiments, the present megakaryocyte-derived extracellular vesicles are characterized by the expression and/or presence of one or more of PS, CD62P, CLEC-2, CD9, CD31, CD147, CD32a, and GPVI. In embodiments, the present megakaryocyte-derived extracellular vesicles are characterized by the expression and/or presence of one or more of PS, CD62P, LAMP-1 (CD107a), CLEC-2, CD9, and CD31. In embodiments, the present megakaryocyte-derived extracellular vesicles are characterized by the expression and/or presence of one or more of CD62P, CD41, and CD61. In embodiments, the present megakaryocyte-derived extracellular vesicles are characterized by a substantial expression and/or presence of one or more of CD54, CD18, CD43, CD11b, CD62P, CD41, CD61, CD21, CD51, and CLEC-2. In embodiments, the present megakaryocyte-derived extracellular vesicles are characterized by a substantial expression and/or presence of one or more of CD62P, CD41, and CD61. In embodiments, the present megakaryocyte-derived extracellular vesicles are characterized by not expressing and/or comprising a substantial amount of DRAQ5. In embodiments, the present megakaryocyte-derived extracellular vesicles are characterized by being substantially free of DRAQ5.

In embodiments, greater than about 50% of the megakaryocyte-derived extracellular vesicles comprise a lipid bilayer membrane comprising CD62P. In embodiments, greater than about 40% of the megakaryocyte-derived extracellular vesicles comprise a lipid bilayer membrane comprising CD62P. In embodiments, greater than about 60% of the megakaryocyte-derived extracellular vesicles comprise a lipid bilayer membrane comprising CD62P. In embodiments, greater than about 70% of the megakaryocyte-derived extracellular vesicles comprise a lipid bilayer membrane comprising CD62P. In embodiments, greater than about 80% of the megakaryocyte-derived extracellular vesicles comprise a lipid bilayer membrane comprising CD62P. In embodiments, greater than about 90% of the megakaryocyte-derived extracellular vesicles comprise a lipid bilayer membrane comprising CD62P. In embodiments, greater than about 95% of the megakaryocyte-derived extracellular vesicles comprise a lipid bilayer membrane comprising CD62P. In embodiments, greater than about 99% of the megakaryocyte-derived extracellular vesicles comprise a lipid bilayer membrane comprising CD62P.

In embodiments, about 50% or less of the megakaryocyte-derived extracellular vesicles comprise a lipid bilayer membrane comprising CD62P. In embodiments, about 40% or less of the megakaryocyte-derived extracellular vesicles comprise a lipid bilayer membrane comprising CD62P. In embodiments, about 60% or less of the megakaryocyte-derived extracellular vesicles comprise a lipid bilayer membrane comprising CD62P. In embodiments, about 70% or less of the megakaryocyte-derived extracellular vesicles comprise a lipid bilayer membrane comprising CD62P. In embodiments, about 80% or less of the megakaryocyte-derived extracellular vesicles comprise a lipid bilayer membrane comprising CD62P. In embodiments, about 90% or less of the megakaryocyte-derived extracellular vesicles comprise a lipid bilayer membrane comprising CD62P. In embodiments, about 95% or less of the megakaryocyte-derived extracellular vesicles comprise a lipid bilayer membrane comprising CD62P. In embodiments, about 99% or less of the megakaryocyte-derived extracellular vesicles comprise a lipid bilayer membrane comprising CD62P.

In embodiments, less than about 50% of the megakaryocyte-derived extracellular vesicles comprise a lipid bilayer membrane comprising CD62P. In embodiments, less than about 40% of the megakaryocyte-derived extracellular vesicles comprise a lipid bilayer membrane comprising CD62P. In embodiments, less than about 30% of the megakaryocyte-derived extracellular vesicles comprise a lipid bilayer membrane comprising CD62P. In embodiments, less than about 25% of the megakaryocyte-derived extracellular vesicles comprise a lipid bilayer membrane comprising CD62P. In embodiments, less than about 20% of the megakaryocyte-derived extracellular vesicles comprise a lipid bilayer membrane comprising CD62P. In embodiments, less than about 15% of the megakaryocyte-derived extracellular vesicles comprise a lipid bilayer membrane comprising CD62P. In embodiments, less than about 10% of the megakaryocyte-derived extracellular vesicles comprise a lipid bilayer membrane comprising CD62P. In embodiments, less than about 5% of the megakaryocyte-derived extracellular vesicles comprise a lipid bilayer membrane comprising CD62P.

In embodiments, less than about 1% of the megakaryocyte-derived extracellular vesicles comprise a lipid bilayer membrane comprising CD62P.

In embodiments, between about 1% to about 99% of the megakaryocyte-derived extracellular vesicles comprise a lipid bilayer membrane comprising CD62P. In embodiments, between about 1% to about 50% of the megakaryocyte-derived extracellular vesicles comprise a lipid bilayer membrane comprising CD62P. In embodiments, between about 1% to about 25% of the megakaryocyte-derived extracellular vesicles comprise a lipid bilayer membrane comprising CD62P. In embodiments, between about 1% to about 10% of the megakaryocyte-derived extracellular vesicles comprise a lipid bilayer membrane comprising CD62P. In embodiments, between about 1% to about 5% of the megakaryocyte-derived extracellular vesicles comprise a lipid bilayer membrane comprising CD62P. In embodiments, between about 1% to about 2% of the megakaryocyte-derived extracellular vesicles comprise a lipid bilayer membrane comprising CD62P. In embodiments, between about 50% to about 99% of the megakaryocyte-derived extracellular vesicles comprise a lipid bilayer membrane comprising CD62P. In embodiments, between about 75% to about 99% of the megakaryocyte-derived extracellular vesicles comprise a lipid bilayer membrane comprising CD62P. In embodiments, between about 90% to about 99% of the megakaryocyte-derived extracellular vesicles comprise a lipid bilayer membrane comprising CD62P. In embodiments, between about 95% to about 99% of the megakaryocyte-derived extracellular vesicles comprise a lipid bilayer membrane comprising CD62P.

In embodiments, less than about 1%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or greater than about 99% of the megakaryocyte-derived extracellular vesicles comprise a lipid bilayer membrane comprising CD62P.

In embodiments, the megakaryocyte-derived extracellular vesicles are free of, or substantially free of CD62P.

In embodiments, the megakaryocyte-derived extracellular vesicles are characterized by a higher expression and/or presence of CD62P than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles have about a 2-fold, or about a 10-fold, or about a 50-fold, or about a 100-fold, or about a 300-fold, or about a 500-fold, or about a 1000-fold greater amount of CD62P than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles.

In embodiments, the megakaryocyte-derived extracellular vesicles are characterized by a lower expression and/or presence of CD62P than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles have about a 2-fold, or about a 10-fold, or about a 50-fold, or about a 100-fold, or about a 300-fold, or about a 500-fold, or about a 1000-fold lower amount of CD62P than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles.

In embodiments, the megakaryocyte-derived extracellular vesicles have about a 4-fold to about a 32-fold or about an 8-fold to about a 16-fold lower amount of CD62P than platelet free plasma (PFP) MKEVs. In embodiments, the megakaryocyte-derived extracellular vesicles have about a 15-fold or about a 16-fold lower amount of CD62P than platelet free plasma (PFP) MKEVs. In embodiments, the megakaryocyte-derived extracellular vesicles have about a 32-fold to about a 128-fold, about a 50-fold to about a 75-fold, or about a 60-fold to about a 70-fold lower amount of CD62P than platelet derived extracellular vesicles (PLT EVs). In embodiments, the megakaryocyte-derived extracellular vesicles have about a 60-fold, about a 64-fold, or about a 70-fold lower amount of CD62P than platelet derived extracellular vesicles (PLT EVs).

In embodiments, greater than about 50% of the megakaryocyte-derived extracellular vesicles comprise a lipid bilayer membrane comprising CD41. In embodiments, greater than about 40% of the megakaryocyte-derived extracellular vesicles comprise a lipid bilayer membrane comprising CD41. In embodiments, greater than about 60% of the megakaryocyte-derived extracellular vesicles comprise a lipid bilayer membrane comprising CD41. In embodiments, greater than about 70% of the megakaryocyte-derived extracellular vesicles comprise a lipid bilayer membrane comprising CD41. In embodiments, greater than about 80% of the megakaryocyte-derived extracellular vesicles comprise a lipid bilayer membrane comprising CD41. In embodiments, greater than about 90% of the megakaryocyte-derived extracellular vesicles comprise a lipid bilayer membrane comprising CD41. In embodiments, greater than about 95% of the megakaryocyte-derived extracellular vesicles comprise a lipid bilayer membrane comprising CD41. In embodiments, greater than about 99% of the megakaryocyte-derived extracellular vesicles comprise a lipid bilayer membrane comprising CD41.

In embodiments, about 50% or less of the megakaryocyte-derived extracellular vesicles comprise a lipid bilayer membrane comprising CD41. In embodiments, about 40% or less of the megakaryocyte-derived extracellular vesicles comprise a lipid bilayer membrane comprising CD41. In embodiments, about 60% or less of the megakaryocyte-derived extracellular vesicles comprise a lipid bilayer membrane comprising CD41. In embodiments, about 70% or less of the megakaryocyte-derived extracellular vesicles comprise a lipid bilayer membrane comprising CD41. In embodiments, about 80% or less of the megakaryocyte-derived extracellular vesicles comprise a lipid bilayer membrane comprising CD41. In embodiments, about 90% or less of the megakaryocyte-derived extracellular vesicles comprise a lipid bilayer membrane comprising CD41. In embodiments, about 95% or less of the megakaryocyte-derived extracellular vesicles comprise a lipid bilayer membrane comprising CD41. In embodiments, about 99% or less of the megakaryocyte-derived extracellular vesicles comprise a lipid bilayer membrane comprising CD41.

In embodiments, less than about 50% of the megakaryocyte-derived extracellular vesicles comprise a lipid bilayer membrane comprising CD41. In embodiments, less than about 40% of the megakaryocyte-derived extracellular vesicles comprise a lipid bilayer membrane comprising CD41. In embodiments, less than about 30% of the megakaryocyte-derived extracellular vesicles comprise a lipid bilayer membrane comprising CD41. In embodiments, less than about 25% of the megakaryocyte-derived extracellular vesicles comprise a lipid bilayer membrane comprising CD41. In embodiments, less than about 20% of the megakaryocyte-derived extracellular vesicles comprise a lipid bilayer membrane comprising CD41. In embodiments, less than about 15% of the megakaryocyte-derived extracellular vesicles comprise a lipid bilayer membrane comprising CD41. In embodiments, less than about 10% of the megakaryocyte-derived extracellular vesicles comprise a lipid bilayer membrane comprising CD41. In embodiments, less than about 5% of the megakaryocyte-derived extracellular vesicles comprise a lipid bilayer membrane comprising CD41. In embodiments, less than about 1% of the megakaryocyte-derived extracellular vesicles comprise a lipid bilayer membrane comprising CD41.

In embodiments, between about 1% to about 99% of the megakaryocyte-derived extracellular vesicles comprise a lipid bilayer membrane comprising CD41. In embodiments, between about 1% to about 50% of the megakaryocyte-derived extracellular vesicles comprise a lipid bilayer membrane comprising CD41. In embodiments, between about 1% to about 25% of the megakaryocyte-derived extracellular vesicles comprise a lipid bilayer membrane comprising CD41. In embodiments, between about 1% to about 10% of the megakaryocyte-derived extracellular vesicles comprise a lipid bilayer membrane comprising CD41. In embodiments, between about 1% to about 5% of the megakaryocyte-derived extracellular vesicles comprise a lipid bilayer membrane comprising CD41. In embodiments, between about 1% to about 2% of the megakaryocyte-derived extracellular vesicles comprise a lipid bilayer membrane comprising CD41. In embodiments, between about 50% to about 99% of the megakaryocyte-derived extracellular vesicles comprise a lipid bilayer membrane comprising CD41. In embodiments, between about 75% to about 99% of the megakaryocyte-derived extracellular vesicles comprise a lipid bilayer membrane comprising CD41. In embodiments, between about 90% to about 99% of the megakaryocyte-derived extracellular vesicles comprise a lipid bilayer membrane comprising CD41. In embodiments, between about 95% to about 99% of the megakaryocyte-derived extracellular vesicles comprise a lipid bilayer membrane comprising CD41.

In embodiments, less than about 1%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or greater than about 99% of the megakaryocyte-derived extracellular vesicles comprise a lipid bilayer membrane comprising CD41.

30) In embodiments, the megakaryocyte-derived extracellular vesicles comprise CD41. In embodiments, the megakaryocyte-derived extracellular vesicles are characterized by a higher expression and/or presence or CD41 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles have about a 2-fold, or about a 10-fold, or about a 50-fold, or about a 100-fold, or about a 300-fold, or about a 500-fold, or about a 1000-fold greater amount of CD41 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles.

In embodiments, the megakaryocyte-derived extracellular vesicles are characterized by a lower expression and/or presence or CD41 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles have about a 2-fold, or about a 10-fold, or about a 50-fold, or about a 100-fold, or about a 300-fold, or about a 500-fold, or about a 1000-fold lower amount of CD41 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles.

In embodiments, the megakaryocyte-derived extracellular vesicles have about a 2-fold to about an 8-fold or about a 2-fold to about a 4-fold greater amount of CD41/CD61 than platelet free plasma (PFP) MKEVs. In embodiments, the megakaryocyte-derived extracellular vesicles have about a 2-fold, about a 3-fold, or about a 4-fold greater amount of CD41/CD61 than platelet free plasma (PFP) MKEVs. In embodiments, the megakaryocyte-derived extracellular vesicles have about a 1.1-fold to about a 2-fold greater amount of CD41/CD61 than platelet derived extracellular vesicles (PLT EVs). In embodiments, the megakaryocyte-derived extracellular vesicles have about a 1.1-fold or about a 1.2-fold greater amount of CD41/CD61 than platelet derived extracellular vesicles (PLT EVs). In embodiments, the megakaryocyte-derived extracellular vesicles have an amount of CD41/CD61 that is substantially the same as platelet derived extracellular vesicles (PLT EVs).

In embodiments, greater than about 50% of the megakaryocyte-derived extracellular vesicles comprise a lipid bilayer membrane comprising CD61. In embodiments, greater than about 40% of the megakaryocyte-derived extracellular vesicles comprise a lipid bilayer membrane comprising CD61. In embodiments, greater than about 60% of the megakaryocyte-derived extracellular vesicles comprise a lipid bilayer membrane comprising CD61. In embodiments, greater than about 70% of the megakaryocyte-derived extracellular vesicles comprise a lipid bilayer membrane comprising CD61. In embodiments, greater than about 80% of the megakaryocyte-derived extracellular vesicles comprise a lipid bilayer membrane comprising CD61. In embodiments, greater than about 90% of the megakaryocyte-derived extracellular vesicles comprise a lipid bilayer membrane comprising CD61. In embodiments, greater than about 95% of the megakaryocyte-derived extracellular vesicles comprise a lipid bilayer membrane comprising CD61. In embodiments, greater than about 99% of the megakaryocyte-derived extracellular vesicles comprise a lipid bilayer membrane comprising CD61.

In embodiments, about 50% or less of the megakaryocyte-derived extracellular vesicles comprise a lipid bilayer membrane comprising CD61. In embodiments, less than about 40% of the megakaryocyte-derived extracellular vesicles comprise a lipid bilayer membrane comprising CD61. In embodiments, about 60% or less of the megakaryocyte-derived extracellular vesicles comprise a lipid bilayer membrane comprising CD61. In embodiments, about 70% or less of the megakaryocyte-derived extracellular vesicles comprise a lipid bilayer membrane comprising CD61. In embodiments, about 80% or less of the megakaryocyte-derived extracellular vesicles comprise a lipid bilayer membrane comprising CD61. In embodiments, less than about 90% of the megakaryocyte-derived extracellular vesicles comprise a lipid bilayer membrane comprising CD61. In embodiments, about 95% or less of the megakaryocyte-derived extracellular vesicles comprise a lipid bilayer membrane comprising CD61. In embodiments, about 99% or less of the megakaryocyte-derived extracellular vesicles comprise a lipid bilayer membrane comprising CD61.

In embodiments, less than about 50% of the megakaryocyte-derived extracellular vesicles comprise a lipid bilayer membrane comprising CD61. In embodiments, less than about 40% of the megakaryocyte-derived extracellular vesicles comprise a lipid bilayer membrane comprising CD61. In embodiments, less than about 30% of the megakaryocyte-derived extracellular vesicles comprise a lipid bilayer membrane comprising CD61. In embodiments, less than about 25% of the megakaryocyte-derived extracellular vesicles comprise a lipid bilayer membrane comprising CD61. In embodiments, less than about 20% of the megakaryocyte-derived extracellular vesicles comprise a lipid bilayer membrane comprising CD61. In embodiments, less than about 15% of the megakaryocyte-derived extracellular vesicles comprise a lipid bilayer membrane comprising CD61. In embodiments, less than about 10% of the megakaryocyte-derived extracellular vesicles comprise a lipid bilayer membrane comprising CD61. In embodiments, less than about 5% of the megakaryocyte-derived extracellular vesicles comprise a lipid bilayer membrane comprising CD61. In embodiments, less than about 1% of the megakaryocyte-derived extracellular vesicles comprise a lipid bilayer membrane comprising CD61.

In embodiments, between about 1% to about 99% of the megakaryocyte-derived extracellular vesicles comprise a lipid bilayer membrane comprising CD61. In embodiments, between about 1% to about 50% of the megakaryocyte-derived extracellular vesicles comprise a lipid bilayer membrane comprising CD61. In embodiments, between about 1% to about 25% of the megakaryocyte-derived extracellular vesicles comprise a lipid bilayer membrane comprising CD61. In embodiments, between about 1% to about 10% of the megakaryocyte-derived extracellular vesicles comprise a lipid bilayer membrane comprising CD61. In embodiments, between about 1% to about 5% of the megakaryocyte-derived extracellular vesicles comprise a lipid bilayer membrane comprising CD61. In embodiments, between about 1% to about 2% of the megakaryocyte-derived extracellular vesicles comprise a lipid bilayer membrane comprising CD61. In embodiments, between about 50% to about 99% of the megakaryocyte-derived extracellular vesicles comprise a lipid bilayer membrane comprising CD61. In embodiments, between about 75% to about 99% of the megakaryocyte-derived extracellular vesicles comprise a lipid bilayer membrane comprising CD61. In embodiments, between about 90% to about 99% of the megakaryocyte-derived extracellular vesicles comprise a lipid bilayer membrane comprising CD61. In embodiments, between about 95% to about 99% of the megakaryocyte-derived extracellular vesicles comprise a lipid bilayer membrane comprising CD61.

In embodiments, less than about 1%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or greater than about 99% of the megakaryocyte-derived extracellular vesicles comprise a lipid bilayer membrane comprising CD61.

In embodiments, the megakaryocyte-derived extracellular vesicles are characterized by a higher expression and/or presence of CD61 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles have about a 2-fold, or about a 10-fold, or about a 50-fold, or about a 100-fold, or about a 300-fold, or about a 500-fold, or about a 1000-fold greater amount of CD61 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles.

In embodiments, the megakaryocyte-derived extracellular vesicles are characterized by a lower expression and/or presence of CD61 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles have about a 2-fold, or about a 10-fold, or about a 50-fold, or about a 100-fold, or about a 300-fold, or about a 500-fold, or about a 1000-fold lower amount of CD61 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles.

In embodiments, the megakaryocyte-derived extracellular vesicles have about a 2-fold to about an 8-fold or about a 2-fold to about a 4-fold greater amount of CD61 than platelet free plasma (PFP) MkEVs. In embodiments, the megakaryocyte-derived extracellular vesicles have about a 2-fold, about a 3-fold, or about a 4-fold greater amount of CD61 than platelet free plasma (PFP) MKEVs. In embodiments, the megakaryocyte-derived extracellular vesicles have about a 1.1-fold to about a 2-fold lower amount of CD61 than platelet derived extracellular vesicles (PLT EVs). In embodiments, the megakaryocyte-derived extracellular vesicles have about a 1.1-fold or about a 1.2-fold lower amount of CD61 than platelet derived extracellular vesicles (PLT EVs). In embodiments, the megakaryocyte-derived extracellular vesicles have an amount of CD61 that is substantially the same as platelet derived extracellular vesicles (PLT EVs).

In embodiments, greater than about 50% of the megakaryocyte-derived extracellular vesicles comprise a lipid bilayer membrane comprising CD54. In embodiments, greater than about 40% of the megakaryocyte-derived extracellular vesicles comprise a lipid bilayer membrane comprising CD54. In embodiments, greater than about 60% of the megakaryocyte-derived extracellular vesicles comprise a lipid bilayer membrane comprising CD54. In embodiments, greater than about 70% of the megakaryocyte-derived extracellular vesicles comprise a lipid bilayer membrane comprising CD4. In embodiments, greater than about 80% of the megakaryocyte-derived extracellular vesicles comprise a lipid bilayer membrane comprising CD54. In embodiments, greater than about 90% of the megakaryocyte-derived extracellular vesicles comprise a lipid bilayer membrane comprising CD54. In embodiments, greater than about 95% of the megakaryocyte-derived extracellular vesicles comprise a lipid bilayer membrane comprising CD54. In embodiments, greater than about 99% of the megakaryocyte-derived extracellular vesicles comprise a lipid bilayer membrane comprising CD54.

In embodiments, about 50% or less of the megakaryocyte-derived extracellular vesicles comprise a lipid bilayer membrane comprising CD54. In embodiments, about 40% or less of the megakaryocyte-derived extracellular vesicles comprise a lipid bilayer membrane comprising CD54. In embodiments, about 60% or less of the megakaryocyte-derived extracellular vesicles comprise a lipid bilayer membrane comprising CD54. In embodiments, about 70% or less of the megakaryocyte-derived extracellular vesicles comprise a lipid bilayer membrane comprising CD54. In embodiments, about 80% or less of the megakaryocyte-derived extracellular vesicles comprise a lipid bilayer membrane comprising CD54. In embodiments, about 90% or less of the megakaryocyte-derived extracellular vesicles comprise a lipid bilayer membrane comprising CD54. In embodiments, about 95% or less of the megakaryocyte-derived extracellular vesicles comprise a lipid bilayer membrane comprising CD54. In embodiments, about 99% or less of the megakaryocyte-derived extracellular vesicles comprise a lipid bilayer membrane comprising CD54.

In embodiments, less than about 50% of the megakaryocyte-derived extracellular vesicles comprise a lipid bilayer membrane comprising CD54. In embodiments, less than about 40% of the megakaryocyte-derived extracellular vesicles comprise a lipid bilayer membrane comprising CD54. In embodiments, less than about 30% of the megakaryocyte-derived extracellular vesicles comprise a lipid bilayer membrane comprising CD54. In embodiments, less than about 25% of the megakaryocyte-derived extracellular vesicles comprise a lipid bilayer membrane comprising CD54. In embodiments, less than about 20% of the megakaryocyte-derived extracellular vesicles comprise a lipid bilayer membrane comprising CD54. In embodiments, less than about 15% of the megakaryocyte-derived extracellular vesicles comprise a lipid bilayer membrane comprising CD54. In embodiments, less than about 10% of the megakaryocyte-derived extracellular vesicles comprise a lipid bilayer membrane comprising CD54. In embodiments, less than about 5% of the megakaryocyte-derived extracellular vesicles comprise a lipid bilayer membrane comprising CD54. In embodiments, less than about 1% of the megakaryocyte-derived extracellular vesicles comprise a lipid bilayer membrane comprising CD54.

In embodiments, between about 1% to about 99% of the megakaryocyte-derived extracellular vesicles comprise a lipid bilayer membrane comprising CD54. In embodiments, between about 1% to about 50% of the megakaryocyte-derived extracellular vesicles comprise a lipid bilayer membrane comprising CD54. In embodiments, between about 1% to about 25% of the megakaryocyte-derived extracellular vesicles comprise a lipid bilayer membrane comprising CD54. In embodiments, between about 1% to about 10% of the megakaryocyte-derived extracellular vesicles comprise a lipid bilayer membrane comprising CD54. In embodiments, between about 1% to about 5% of the megakaryocyte-derived extracellular vesicles comprise a lipid bilayer membrane comprising CD54. In embodiments, between about 1% to about 2% of the megakaryocyte-derived extracellular vesicles comprise a lipid bilayer membrane comprising CD54. In embodiments, between about 50% to about 99% of the megakaryocyte-derived extracellular vesicles comprise a lipid bilayer membrane comprising CD54. In embodiments, between about 75% to about 99% of the megakaryocyte-derived extracellular vesicles comprise a lipid bilayer membrane comprising CD54. In embodiments, between about 90% to about 99% of the megakaryocyte-derived extracellular vesicles comprise a lipid bilayer membrane comprising CD54. In embodiments, between about 95% to about 99% of the megakaryocyte-derived extracellular vesicles comprise a lipid bilayer membrane comprising CD54.

In embodiments, less than about 1%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or greater than about 99% of the megakaryocyte-derived extracellular vesicles comprise a lipid bilayer membrane comprising CD54.

In embodiments, the megakaryocyte-derived extracellular vesicles are characterized by a higher expression and/or presence of CD54 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles have about a 2-fold, or about a 10-fold, or about a 50-fold, or about a 100-fold, or about a 300-fold, or about a 500-fold, or about a 1000-fold greater amount of CD54 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles.

In embodiments, the megakaryocyte-derived extracellular vesicles have about a 2-fold to about a 10-fold or about a 2-fold to about a 4-fold greater amount of CD54 than platelet free plasma (PFP) MKEVs. In embodiments, the megakaryocyte-derived extracellular vesicles have about a 3-fold greater amount of CD54 than platelet free plasma (PFP) MkEVs. In embodiments, the megakaryocyte-derived extracellular vesicles have about a 1.1-fold to about a 4-fold or about a 1.1-fold to about a 2-fold greater amount of CD54 than platelet derived extracellular vesicles (PLT EVs). In embodiments, the megakaryocyte-derived extracellular vesicles have about a 1.5-fold greater amount of CD54 than platelet derived extracellular vesicles (PLT EVs).

In embodiments, the megakaryocyte-derived extracellular vesicles are characterized by a lower expression and/or presence of CD54 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles have about a 2-fold, or about a 10-fold, or about a 50-fold, or about a 100-fold, or about a 300-fold, or about a 500-fold, or about a 1000-fold lower amount of CD54 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles.

In embodiments, greater than about 50% of the megakaryocyte-derived extracellular vesicles comprise a lipid bilayer membrane comprising CD18. In embodiments, greater than about 40% of the megakaryocyte-derived extracellular vesicles comprise a lipid bilayer membrane comprising CD18. In embodiments, greater than about 60% of the megakaryocyte-derived extracellular vesicles comprise a lipid bilayer membrane comprising CD18. In embodiments, greater than about 70% of the megakaryocyte-derived extracellular vesicles comprise a lipid bilayer membrane comprising CD18. In embodiments, greater than about 80% of the megakaryocyte-derived extracellular vesicles comprise a lipid bilayer membrane comprising CD18. In embodiments, greater than about 90% of the megakaryocyte-derived extracellular vesicles comprise a lipid bilayer membrane comprising CD18. In embodiments, greater than about 95% of the megakaryocyte-derived extracellular vesicles comprise a lipid bilayer membrane comprising CD18. In embodiments, greater than about 99% of the megakaryocyte-derived extracellular vesicles comprise a lipid bilayer membrane comprising CD18.

In embodiments, about 50% or less of the megakaryocyte-derived extracellular vesicles comprise a lipid bilayer membrane comprising CD18. In embodiments, about 40% or less of the megakaryocyte-derived extracellular vesicles comprise a lipid bilayer membrane comprising CD18. In embodiments, about 60% or less of the megakaryocyte-derived extracellular vesicles comprise a lipid bilayer membrane comprising CD18. In embodiments, about 70% or less of the megakaryocyte-derived extracellular vesicles comprise a lipid bilayer membrane comprising CD18. In embodiments, about 80% or less of the megakaryocyte-derived extracellular vesicles comprise a lipid bilayer membrane comprising CD18. In embodiments, about 90% or less of the megakaryocyte-derived extracellular vesicles comprise a lipid bilayer membrane comprising CD18. In embodiments, about 95% or less of the megakaryocyte-derived extracellular vesicles comprise a lipid bilayer membrane comprising CD18. In embodiments, about 99% or less of the megakaryocyte-derived extracellular vesicles comprise a lipid bilayer membrane comprising CD18.

In embodiments, less than about 50% of the megakaryocyte-derived extracellular vesicles comprise a lipid bilayer membrane comprising CD18. In embodiments, less than about 40% of the megakaryocyte-derived extracellular vesicles comprise a lipid bilayer membrane comprising CD18. In embodiments, less than about 30% of the megakaryocyte-derived extracellular vesicles comprise a lipid bilayer membrane comprising CD18. In embodiments, less than about 25% of the megakaryocyte-derived extracellular vesicles comprise a lipid bilayer membrane comprising CD18. In embodiments, less than about 20% of the megakaryocyte-derived extracellular vesicles comprise a lipid bilayer membrane comprising CD18. In embodiments, less than about 15% of the megakaryocyte-derived extracellular vesicles comprise a lipid bilayer membrane comprising CD18. In embodiments, less than about 10% of the megakaryocyte-derived extracellular vesicles comprise a lipid bilayer membrane comprising CD18. In embodiments, less than about 5% of the megakaryocyte-derived extracellular vesicles comprise a lipid bilayer membrane comprising CD18. In embodiments, less than about 1% of the megakaryocyte-derived extracellular vesicles comprise a lipid bilayer membrane comprising CD18.

In embodiments, between about 1% to about 99% of the megakaryocyte-derived extracellular vesicles comprise a lipid bilayer membrane comprising CD18. In embodiments, between about 1% to about 50% of the megakaryocyte-derived extracellular vesicles comprise a lipid bilayer membrane comprising CD18. In embodiments, between about 1% to about 25% of the megakaryocyte-derived extracellular vesicles comprise a lipid bilayer membrane comprising CD18. In embodiments, between about 1% to about 10% of the megakaryocyte-derived extracellular vesicles comprise a lipid bilayer membrane comprising CD18. In embodiments, between about 1% to about 5% of the megakaryocyte-derived extracellular vesicles comprise a lipid bilayer membrane comprising CD18. In embodiments, between about 1% to about 2% of the megakaryocyte-derived extracellular vesicles comprise a lipid bilayer membrane comprising CD18. In embodiments, between about 50% to about 99% of the megakaryocyte-derived extracellular vesicles comprise a lipid bilayer membrane comprising CD18. In embodiments, between about 75% to about 99% of the megakaryocyte-derived extracellular vesicles comprise a lipid bilayer membrane comprising CD18. In embodiments, between about 90% to about 99% of the megakaryocyte-derived extracellular vesicles comprise a lipid bilayer membrane comprising CD18. In embodiments, between about 95% to about 99% of the megakaryocyte-derived extracellular vesicles comprise a lipid bilayer membrane comprising CD18.

In embodiments, less than about 1%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or greater than about 99% of the megakaryocyte-derived extracellular vesicles comprise a lipid bilayer membrane comprising CD18.

In embodiments, the megakaryocyte-derived extracellular vesicles are characterized by a higher expression and/or presence of CD18 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles have about a 2-fold, or about a 10-fold, or about a 50-fold, or about a 100-fold, or about a 300-fold, or about a 500-fold, or about a 1000-fold greater amount of CD18 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles.

In embodiments, the megakaryocyte-derived extracellular vesicles have about a 2-fold to about a 10-fold, an 8-fold to about a 64-fold, or about a 16-fold to about a 32-fold, or about a 16-fold to about a 24-fold greater amount of CD18 than platelet free plasma (PFP) MkEVs. In embodiments, the megakaryocyte-derived extracellular vesicles have about a 20-fold greater amount of CD18 than platelet free plasma (PFP) MKEVs. In embodiments, the megakaryocyte-derived extracellular vesicles have about a 1.1-fold to about a 4-fold or about a 1.1-fold to about a 2-fold greater amount of CD18 than platelet derived extracellular vesicles (PLT EVs). In embodiments, the megakaryocyte-derived extracellular vesicles have about a 1.5-fold greater amount of CD18 than platelet derived extracellular vesicles (PLT EVs).

In embodiments, the megakaryocyte-derived extracellular vesicles are characterized by a lower expression and/or presence of CD18 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles have about a 2-fold, or about a 10-fold, or about a 50-fold, or about a 100-fold, or about a 300-fold, or about a 500-fold, or about a 1000-fold lower amount of CD18 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles.

In embodiments, greater than about 50% of the megakaryocyte-derived extracellular vesicles comprise a lipid bilayer membrane comprising CD43. In embodiments, greater than about 40% of the megakaryocyte-derived extracellular vesicles comprise a lipid bilayer membrane comprising CD43. In embodiments, greater than about 60% of the megakaryocyte-derived extracellular vesicles comprise a lipid bilayer membrane comprising CD43. In embodiments, greater than about 70% of the megakaryocyte-derived extracellular vesicles comprise a lipid bilayer membrane comprising CD43. In embodiments, greater than about 80% of the megakaryocyte-derived extracellular vesicles comprise a lipid bilayer membrane comprising CD43. In embodiments, greater than about 90% of the megakaryocyte-derived extracellular vesicles comprise a lipid bilayer membrane comprising CD43. In embodiments, greater than about 95% of the megakaryocyte-derived extracellular vesicles comprise a lipid bilayer membrane comprising CD43. In embodiments, greater than about 99% of the megakaryocyte-derived extracellular vesicles comprise a lipid bilayer membrane comprising CD43.

In embodiments, about 50% or less of the megakaryocyte-derived extracellular vesicles comprise a lipid bilayer membrane comprising CD43. In embodiments, about 40% or less of the megakaryocyte-derived extracellular vesicles comprise a lipid bilayer membrane comprising CD43. In embodiments, about 60% or less of the megakaryocyte-derived extracellular vesicles comprise a lipid bilayer membrane comprising CD43. In embodiments, about 70% or less of the megakaryocyte-derived extracellular vesicles comprise a lipid bilayer membrane comprising CD43. In embodiments, about 80% or less of the megakaryocyte-derived extracellular vesicles comprise a lipid bilayer membrane comprising CD43. In embodiments, about 90% or less of the megakaryocyte-derived extracellular vesicles comprise a lipid bilayer membrane comprising CD43. In embodiments, about 95% or less of the megakaryocyte-derived extracellular vesicles comprise a lipid bilayer membrane comprising CD43. In embodiments, about 99% or less of the megakaryocyte-derived extracellular vesicles comprise a lipid bilayer membrane comprising CD43.

In embodiments, less than about 50% of the megakaryocyte-derived extracellular vesicles comprise a lipid bilayer membrane comprising CD43. In embodiments, less than about 40% of the megakaryocyte-derived extracellular vesicles comprise a lipid bilayer membrane comprising CD43. In embodiments, less than about 30% of the megakaryocyte-derived extracellular vesicles comprise a lipid bilayer membrane comprising CD43. In embodiments, less than about 25% of the megakaryocyte-derived extracellular vesicles comprise a lipid bilayer membrane comprising CD43. In embodiments, less than about 20% of the megakaryocyte-derived extracellular vesicles comprise a lipid bilayer membrane comprising CD43. In embodiments, less than about 15% of the megakaryocyte-derived extracellular vesicles comprise a lipid bilayer membrane comprising CD43. In embodiments, less than about 10% of the megakaryocyte-derived extracellular vesicles comprise a lipid bilayer membrane comprising CD43. In embodiments, less than about 5% of the megakaryocyte-derived extracellular vesicles comprise a lipid bilayer membrane comprising CD43. In embodiments, less than about 1% of the megakaryocyte-derived extracellular vesicles comprise a lipid bilayer membrane comprising CD43.

In embodiments, between about 1% to about 99% of the megakaryocyte-derived extracellular vesicles comprise a lipid bilayer membrane comprising CD43. In embodiments, between about 1% to about 50% of the megakaryocyte-derived extracellular vesicles comprise a lipid bilayer membrane comprising CD43. In embodiments, between about 1% to about 25% of the megakaryocyte-derived extracellular vesicles comprise a lipid bilayer membrane comprising CD43. In embodiments, between about 1% to about 10% of the megakaryocyte-derived extracellular vesicles comprise a lipid bilayer membrane comprising CD43. In embodiments, between about 1% to about 5% of the megakaryocyte-derived extracellular vesicles comprise a lipid bilayer membrane comprising CD43. In embodiments, between about 1% to about 2% of the megakaryocyte-derived extracellular vesicles comprise a lipid bilayer membrane comprising CD43. In embodiments, between about 50% to about 99% of the megakaryocyte-derived extracellular vesicles comprise a lipid bilayer membrane comprising CD43. In embodiments, between about 75% to about 99% of the megakaryocyte-derived extracellular vesicles comprise a lipid bilayer membrane comprising CD43. In embodiments, between about 90% to about 99% of the megakaryocyte-derived extracellular vesicles comprise a lipid bilayer membrane comprising CD43. In embodiments, between about 95% to about 99% of the megakaryocyte-derived extracellular vesicles comprise a lipid bilayer membrane comprising CD43.

In embodiments, less than about 1%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%,) about 97%, about 98%, about 99%, or greater than about 99% of the megakaryocyte-derived extracellular vesicles comprise a lipid bilayer membrane comprising CD43.

In embodiments, the megakaryocyte-derived extracellular vesicles are characterized by a higher expression and/or presence of CD43 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles have about a 2-fold, or about a 10-fold, or about a 50-fold, or about a 100-fold, or about a 300-fold, or about a 500-fold, or about a 1000-fold greater amount of CD43 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles.

In embodiments, the megakaryocyte-derived extracellular vesicles have about an 4-fold to about a 64-fold, or about a 8-fold to about a 32-fold, or about a 8-fold to about a 16-fold greater amount of CD43 than platelet free plasma (PFP) MKEVs. In embodiments, the megakaryocyte-derived extracellular vesicles have about a 10-fold or about a 12-fold greater amount of CD43 than platelet free plasma (PFP) MkEVs. In embodiments, the megakaryocyte-derived extracellular vesicles have about a 1.5-fold to about an 8-fold or about a 2-fold to about a 4-fold greater amount of CD43 than platelet derived extracellular vesicles (PLT EVs). In embodiments, the megakaryocyte-derived extracellular vesicles have about a 3-fold or about a 4-fold greater amount of CD43 than platelet derived extracellular vesicles (PLT EVs).

In embodiments, the megakaryocyte-derived extracellular vesicles are characterized by a lower expression and/or presence of CD43 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles have about a 2-fold, or about a 10-fold, or about a 50-fold, or about a 100-fold, or about a 300-fold, or about a 500-fold, or about a 1000-fold lower amount of CD43 than naturally-occurring megakaryocyte-derived extracellular vesicles, vesicles or extracellular vesicles) derived from platelets such as platelet derived extracellular vesicles (PLT EVs), and/or platelet-free plasma (PPF) megakaryocyte-derived extracellular vesicles.

In embodiments, greater than about 50% of the megakaryocyte-derived extracellular vesicles comprise a lipid bilayer membrane comprising CD11b. In embodiments, greater than about 40% of the megakaryocyte-derived extracellular vesicles comprise a lipid bilayer membrane comprising CD11b. In embodiments, greater than about 60% of the megakaryocyte-derived extracellular vesicles comprise a lipid bilayer membrane comprising CD11b. In embodiments, greater than about 70% of the megakaryocyte-derived extracellular vesicles comprise a lipid bilayer membrane comprising CD11b. In embodiments, greater than about 80% of the megakaryocyte-derived extracellular) vesicles comprise a lipid bilayer membrane comprising CD11b. In embodiments, greater than about 90% of the megakaryocyte-derived extracellular vesicles comprise a lipid bilayer membrane comprising CD11b. In embodiments, greater than about 95% of the megakaryocyte-derived extracellular vesicles comprise a lipid bilayer membrane comprising CD11b. In embodiments, greater than about 99% of the megakaryocyte-derived extracellular vesicles comprise a lipid bilayer membrane comprising CD11b.

In embodiments, about 50% or less of the megakaryocyte-derived extracellular vesicles comprise a lipid bilayer membrane comprising CD11b. In embodiments, about 40% or less of the megakaryocyte-derived extracellular vesicles comprise a lipid bilayer membrane comprising CD11b. In embodiments, about 60% or less of the megakaryocyte-derived extracellular vesicles comprise a lipid bilayer membrane comprising CD11b. In embodiments, about 70% or less of the megakaryocyte-derived extracellular vesicles comprise a lipid bilayer membrane comprising CD11b. In embodiments, about 80% or less of the megakaryocyte-derived extracellular vesicles comprise a lipid bilayer membrane comprising CD11b. In embodiments, about 90% or less of the megakaryocyte-derived extracellular vesicles comprise a lipid bilayer membrane comprising CD11b. In embodiments, about 95% or less of the megakaryocyte-derived extracellular vesicles comprise a lipid bilayer membrane comprising CD11b. In embodiments, about 99% or less of the megakaryocyte-derived extracellular vesicles comprise a lipid bilayer membrane comprising CD11b.

In embodiments, less than about 50% of the megakaryocyte-derived extracellular vesicles comprise a lipid bilayer membrane comprising CD11b. In embodiments, less than about 40% of the megakaryocyte-derived extracellular vesicles comprise a lipid) bilayer membrane comprising CD11b. In embodiments, less than about 30% of the megakaryocyte-derived extracellular vesicles comprise a lipid bilayer membrane comprising CD11b. In embodiments, less than about 25% of the megakaryocyte-derived extracellular vesicles comprise a lipid bilayer membrane comprising CD11b.

In embodiments, less than about 20% of the megakaryocyte-derived extracellular vesicles comprise a lipid bilayer membrane comprising CD11b. In embodiments, less than about 15% of the megakaryocyte-derived extracellular vesicles comprise a lipid bilayer membrane comprising CD11b. In embodiments, less than about 10% of the megakaryocyte-derived extracellular vesicles comprise a lipid bilayer membrane comprising CD11b. In embodiments, less than about 5% of the megakaryocyte-) derived extracellular vesicles comprise a lipid bilayer membrane comprising CD11b. In embodiments, less than about 1% of the megakaryocyte-derived extracellular vesicles comprise a lipid bilayer membrane comprising CD11b.

In embodiments, between about 1% to about 99% of the megakaryocyte-derived extracellular vesicles comprise a lipid bilayer membrane comprising CD11b. In embodiments, between about 1% to about 50% of the megakaryocyte-derived extracellular vesicles comprise a lipid bilayer membrane comprising CD11b. In embodiments, between about 1% to about 25% of the megakaryocyte-derived extracellular vesicles comprise a lipid bilayer membrane comprising CD11b. In embodiments, between about 1% to about 10% of the megakaryocyte-derived extracellular vesicles comprise a lipid bilayer membrane comprising CD11b. In embodiments, between about 1% to about 5% of the megakaryocyte-derived) extracellular vesicles comprise a lipid bilayer membrane comprising CD11b. In embodiments, between about 1% to about 2% of the megakaryocyte-derived extracellular vesicles comprise a lipid bilayer membrane comprising CD11b. In embodiments, between about 50% to about 99% of the megakaryocyte-derived extracellular vesicles comprise a lipid bilayer membrane comprising CD11b. In embodiments, between about 75% to about 99% of the megakaryocyte-derived extracellular vesicles comprise a lipid bilayer membrane comprising CD11b. In embodiments, between about 90% to about 99% of the megakaryocyte-derived extracellular vesicles comprise a lipid bilayer membrane comprising CD11b. In embodiments, between about 95% to about 99% of the megakaryocyte-derived extracellular vesicles comprise a lipid bilayer membrane comprising CD11b. 20 In embodiments, less than about 1%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%,) about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or greater than about 99% of the megakaryocyte-derived extracellular vesicles comprise a lipid bilayer membrane comprising CD11b.

In embodiments, the megakaryocyte-derived extracellular vesicles are characterized by a higher expression and/or presence of CD11b than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles have about a 2-fold, or about a 10-fold, or about a 50-fold, or about a 100-fold, or about a 300-fold, or about a 500-fold, or about a 1000-fold greater amount of CD11b than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles.

In embodiments, the megakaryocyte-derived extracellular vesicles have about a 2-fold to about an 8-fold, or about a 2-fold to about a 4-fold greater amount of CD11b than platelet free plasma (PFP) MkEVs. In embodiments, the megakaryocyte-derived extracellular vesicles have about a 3-fold greater amount of CD11b than platelet free plasma (PFP) MkEVs. In embodiments, the megakaryocyte-derived extracellular vesicles have about a 1.1-fold to about a 4-fold, or about a 1.1-fold to about a 2-fold greater amount of CD11b than platelet derived extracellular vesicles (PLT EVs). In embodiments, the megakaryocyte-derived extracellular vesicles have about a 1.5-fold greater amount of CD11b than platelet derived extracellular vesicles (PLT EVs).

In embodiments, the megakaryocyte-derived extracellular vesicles are characterized by a higher expression and/or presence of CD11b than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles have about a 2-fold, or about a 10-fold, or about a 50-fold, or about a 100-fold, or about a 300-fold, or about a 500-fold, or about a 1000-fold greater amount of CD11b than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles.

In embodiments, the megakaryocyte-derived extracellular vesicles are characterized by a lower expression and/or presence of CD11b than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles have about a 2-fold, or about a 10-fold, or about a 50-fold, or about a 100-fold, or about a 300-fold, or about a 500-fold, or about a 1000-fold lower amount of CD11b than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles.

In embodiments, greater than about 50% of the megakaryocyte-derived extracellular vesicles comprise a lipid bilayer membrane comprising CD21. In embodiments, greater than about 40% of the megakaryocyte-derived extracellular vesicles comprise a lipid bilayer membrane comprising CD21. In embodiments, greater than about 60% of the megakaryocyte-derived extracellular vesicles comprise a lipid bilayer membrane comprising CD21. In embodiments, greater than about 70% of the megakaryocyte-derived extracellular vesicles comprise a lipid bilayer membrane comprising CD21. In embodiments, greater than about 80% of the megakaryocyte-derived extracellular vesicles comprise a lipid bilayer membrane comprising CD21. In embodiments, greater than about 90% of the megakaryocyte-derived extracellular vesicles comprise a lipid bilayer membrane comprising CD21. In embodiments, greater than about 95% of the megakaryocyte-derived extracellular vesicles comprise a lipid bilayer membrane comprising CD21. In embodiments, greater than about 99% of the megakaryocyte-derived extracellular vesicles comprise a lipid bilayer membrane comprising CD21.

In embodiments, about 50% or less of the megakaryocyte-derived extracellular vesicles comprise a lipid bilayer membrane comprising CD21. In embodiments, about) 40% or less of the megakaryocyte-derived extracellular vesicles comprise a lipid bilayer membrane comprising CD21. In embodiments, about 60% or less of the megakaryocyte-derived extracellular vesicles comprise a lipid bilayer membrane comprising CD21. In embodiments, about 70% or less of the megakaryocyte-derived extracellular vesicles comprise a lipid bilayer membrane comprising CD21. In embodiments, about 80% or less of the megakaryocyte-derived extracellular vesicles comprise a lipid bilayer membrane comprising CD21. In embodiments, about 90% or less of the megakaryocyte-derived extracellular vesicles comprise a lipid bilayer membrane comprising CD21. In embodiments, about 95% or less of the megakaryocyte-derived extracellular vesicles comprise a lipid bilayer membrane comprising CD21. In embodiments, about 99% or less of the megakaryocyte-derived extracellular vesicles comprise a lipid bilayer membrane comprising CD21.

In embodiments, less than about 50% of the megakaryocyte-derived extracellular vesicles comprise a lipid bilayer membrane comprising CD21. In embodiments, less than about 40% of the megakaryocyte-derived extracellular vesicles comprise a lipid bilayer membrane comprising CD21. In embodiments, less than about 30% of the megakaryocyte-derived extracellular vesicles comprise a lipid bilayer membrane comprising CD21. In embodiments, less than about 25% of the megakaryocyte-derived extracellular vesicles comprise a lipid bilayer membrane comprising CD21. In embodiments, less than about 20% of the megakaryocyte-derived extracellular vesicles comprise a lipid bilayer membrane comprising CD21. In embodiments, less than about 15% of the megakaryocyte-derived extracellular vesicles comprise a lipid bilayer membrane comprising CD21. In embodiments, less than about 10% of the megakaryocyte-derived extracellular vesicles comprise a lipid bilayer membrane comprising CD21. In embodiments, less than about 5% of the megakaryocyte-derived extracellular vesicles comprise a lipid bilayer membrane comprising CD21. In embodiments, less than about 1% of the megakaryocyte-derived extracellular vesicles comprise a lipid bilayer membrane comprising CD21.

In embodiments, between about 1% to about 99% of the megakaryocyte-derived extracellular vesicles comprise a lipid bilayer membrane comprising CD21. In embodiments, between about 1% to about 50% of the megakaryocyte-derived extracellular vesicles comprise a lipid bilayer membrane comprising CD21. In embodiments, between about 1% to about 25% of the megakaryocyte-derived extracellular vesicles comprise a lipid bilayer membrane comprising CD21. In embodiments, between about 1% to about 10% of the megakaryocyte-derived extracellular vesicles comprise a lipid bilayer membrane comprising CD21. In embodiments, between about 1% to about 5% of the megakaryocyte-derived extracellular vesicles comprise a lipid bilayer membrane comprising CD21. In embodiments, between about 1% to about 2% of the megakaryocyte-derived extracellular vesicles comprise a lipid bilayer membrane comprising CD21. In embodiments, between about 50% to about 99% of the megakaryocyte-derived extracellular vesicles comprise a lipid bilayer membrane comprising CD21. In embodiments, between about 75% to about 99% of the megakaryocyte-derived extracellular vesicles comprise a lipid bilayer membrane comprising CD21. In embodiments, between about 90% to about 99% of the megakaryocyte-derived extracellular vesicles comprise a lipid bilayer membrane comprising CD21. In embodiments, between about 95% to about 99% of the megakaryocyte-derived extracellular vesicles comprise a lipid bilayer membrane comprising CD21.

In embodiments, less than about 1%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%,) about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or greater than about 99% of the megakaryocyte-derived extracellular vesicles comprise a lipid bilayer membrane comprising CD21.

In embodiments, the megakaryocyte-derived extracellular vesicles are characterized by a higher expression and/or presence of CD21 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles have about a 2-fold, or about a 10-fold, or about a 50-fold, or about a 100-fold, or about a 300-fold, or about a 500-fold, or about a 1000-fold greater amount of than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles.

In embodiments, the megakaryocyte-derived extracellular vesicles have about a 2-fold to about a 64-fold, about a 4-fold to about a 32-fold, or about an 8-fold to about a 16-fold greater amount of CD21 than platelet free plasma (PFP) MKEVs. In embodiments, the megakaryocyte-derived extracellular vesicles have about a 10-fold or about a 12-fold greater amount of CD21 than platelet free plasma (PFP) MKEVs. In embodiments, the megakaryocyte-derived extracellular vesicles have about a 2-fold to about an 8-fold, or about a 4-fold to about an 8-fold greater amount of CD21 than platelet derived extracellular vesicles (PLT EVs). In embodiments, the megakaryocyte-derived extracellular vesicles have about a 4-fold or about a 5-fold greater amount of CD21 than platelet derived extracellular vesicles (PLT EVs).

In embodiments, the megakaryocyte-derived extracellular vesicles are characterized by a lower expression and/or presence of CD21 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles have about a 2-fold, or about a 10-fold, or about a 50-fold, or about a 100-fold, or about a 300-fold, or about a 500-fold, or about a 1000-fold lower amount of CD21 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles.

In embodiments, greater than about 50% of the megakaryocyte-derived extracellular vesicles comprise a lipid bilayer membrane comprising CD51. In embodiments, greater than about 40% of the megakaryocyte-derived extracellular vesicles comprise) a lipid bilayer membrane comprising CD51. In embodiments, greater than about 60% of the megakaryocyte-derived extracellular vesicles comprise a lipid bilayer membrane comprising CD51. In embodiments, greater than about 70% of the megakaryocyte-derived extracellular vesicles comprise a lipid bilayer membrane comprising CD51. In embodiments, greater than about 80% of the megakaryocyte-derived extracellular vesicles comprise a lipid bilayer membrane comprising CD51. In embodiments, greater than about 90% of the megakaryocyte-derived extracellular vesicles comprise a lipid bilayer membrane comprising CD51. In embodiments, greater than about 95% of the megakaryocyte-derived extracellular vesicles comprise a lipid bilayer membrane comprising CD51. In embodiments, greater than about 99% of the megakaryocyte-derived extracellular vesicles comprise a lipid bilayer membrane comprising CD51.

In embodiments, about 50% or less of the megakaryocyte-derived extracellular vesicles comprise a lipid bilayer membrane comprising CD51. In embodiments, about 40% or less of the megakaryocyte-derived extracellular vesicles comprise a lipid bilayer membrane comprising CD51. In embodiments, about 60% or less of the megakaryocyte-derived extracellular vesicles comprise a lipid bilayer membrane comprising CD51. In embodiments, about 70% or less of the megakaryocyte-derived extracellular vesicles comprise a lipid bilayer membrane comprising CD51. In embodiments, about 80% or less of the megakaryocyte-derived extracellular vesicles comprise a lipid bilayer membrane comprising CD51. In embodiments, about 90% or less of the megakaryocyte-derived extracellular vesicles comprise a lipid bilayer membrane comprising CD51. In embodiments, about 95% or less of the megakaryocyte-derived extracellular vesicles comprise a lipid bilayer membrane) comprising CD51. In embodiments, about 99% or less of the megakaryocyte-derived extracellular vesicles comprise a lipid bilayer membrane comprising CD51.

In embodiments, less than about 50% of the megakaryocyte-derived extracellular vesicles comprise a lipid bilayer membrane comprising CD51. In embodiments, less than about 40% of the megakaryocyte-derived extracellular vesicles comprise a lipid bilayer membrane comprising CD51. In embodiments, less than about 30% of the megakaryocyte-derived extracellular vesicles comprise a lipid bilayer membrane comprising CD51. In embodiments, less than about 25% of the megakaryocyte-derived extracellular vesicles comprise a lipid bilayer membrane comprising CD51. In embodiments, less than about 20% of the megakaryocyte-derived extracellular) vesicles comprise a lipid bilayer membrane comprising CD51. In embodiments, less than about 15% of the megakaryocyte-derived extracellular vesicles comprise a lipid bilayer membrane comprising CD51. In embodiments, less than about 10% of the megakaryocyte-derived extracellular vesicles comprise a lipid bilayer membrane comprising CD51. In embodiments, less than about 5% of the megakaryocyte-derived extracellular vesicles comprise a lipid bilayer membrane comprising CD51. In embodiments, less than about 1% of the megakaryocyte-derived extracellular vesicles comprise a lipid bilayer membrane comprising CD51.

In embodiments, between about 1% to about 99% of the megakaryocyte-derived extracellular vesicles comprise a lipid bilayer membrane comprising CD51. In embodiments, between about 1% to about 50% of the megakaryocyte-derived extracellular vesicles comprise a lipid bilayer membrane comprising CD51. In embodiments, between about 1% to about 25% of the megakaryocyte-derived extracellular vesicles comprise a lipid bilayer membrane comprising CD51. In embodiments, between about 1% to about 10% of the megakaryocyte-derived extracellular vesicles comprise a lipid bilayer membrane comprising CD51. In embodiments, between about 1% to about 5% of the megakaryocyte-derived extracellular vesicles comprise a lipid bilayer membrane comprising CD51. In embodiments, between about 1% to about 2% of the megakaryocyte-derived extracellular vesicles comprise a lipid bilayer membrane comprising CD51. In embodiments, between about 50% to about 99% of the megakaryocyte-derived extracellular vesicles comprise a lipid bilayer membrane comprising CD51. In embodiments, between about 75% to about 99% of the megakaryocyte-derived extracellular vesicles comprise a lipid bilayer membrane comprising CD51. In embodiments, between about 90% to about 99% of the megakaryocyte-derived extracellular vesicles comprise a lipid bilayer membrane comprising CD51. In embodiments, between about 95% to about 99% of the megakaryocyte-derived extracellular vesicles comprise a lipid bilayer membrane comprising CD51.

In embodiments, less than about 1%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or greater than about 99% of the megakaryocyte-derived extracellular vesicles comprise a lipid bilayer membrane comprising CD51.

In embodiments, the megakaryocyte-derived extracellular vesicles are characterized by a higher expression and/or presence of CD51 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles have about a 2-fold, or about a 10-fold, or about a 50-fold, or about a 100-fold, or about a 300-fold, or about a 500-fold, or about a 1000-fold greater amount of CD51 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles.

In embodiments, the megakaryocyte-derived extracellular vesicles are characterized by a lower expression and/or presence of CD51 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles have about a 2-fold, or about a 10-fold, or about a 50-fold, or about a 100-fold, or about a 300-fold, or about a 500-fold, or about a 1000-fold lower amount of CD51 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles.

In embodiments, the megakaryocyte-derived extracellular vesicles have about a 1.1-fold to about a 4-fold, or about a 1.1-fold to about a 2-fold lower amount of CD51 than platelet free plasma (PFP) MkEVs. In embodiments, the megakaryocyte-derived extracellular vesicles have about a 1.5-fold lower amount of CD51 than platelet free plasma (PFP) MkEVs. In embodiments, the megakaryocyte-derived extracellular vesicles have about a 1.1-fold to about a 4-fold, or about a 1.1-fold to about a 2-fold lower amount of CD51 than platelet derived extracellular vesicles (PLT EVs). In embodiments, the megakaryocyte-derived extracellular vesicles have about a 1.5-fold lower amount of CD51 than platelet derived extracellular vesicles (PLT EVs).

In embodiments, greater than about 50% of the megakaryocyte-derived extracellular vesicles comprise a lipid bilayer membrane comprising CLEC-2. In embodiments, greater than about 40% of the megakaryocyte-derived extracellular vesicles comprise a lipid bilayer membrane comprising CLEC-2. In embodiments, greater than about 60% of the megakaryocyte-derived extracellular vesicles comprise a lipid bilayer membrane comprising CLEC-2. In embodiments, greater than about 70% of the megakaryocyte-derived extracellular vesicles comprise a lipid bilayer membrane comprising CLEC-2. In embodiments, greater than about 80% of the megakaryocyte-derived extracellular vesicles comprise a lipid bilayer membrane comprising CLEC-2.

In embodiments, greater than about 90% of the megakaryocyte-derived extracellular vesicles comprise a lipid bilayer membrane comprising CLEC-2. In embodiments, greater than about 95% of the megakaryocyte-derived extracellular vesicles comprise a lipid bilayer membrane comprising CLEC-2. In embodiments, greater than about 99% of the megakaryocyte-derived extracellular vesicles comprise a lipid bilayer membrane comprising CLEC-2.

In embodiments, about 50% or less of the megakaryocyte-derived extracellular vesicles comprise a lipid bilayer membrane comprising CLEC-2. In embodiments, about 40% or less of the megakaryocyte-derived extracellular vesicles comprise a lipid bilayer membrane comprising CLEC-2. In embodiments, about 60% or less of the megakaryocyte-derived extracellular vesicles comprise a lipid bilayer membrane comprising CLEC-2. In embodiments, about 70% or less of the megakaryocyte-derived extracellular vesicles comprise a lipid bilayer membrane comprising CLEC-2. In embodiments, about 80% or less of the megakaryocyte-derived extracellular vesicles comprise a lipid bilayer membrane comprising CLEC-2. In embodiments, about 90% or less of the megakaryocyte-derived extracellular vesicles comprise a lipid bilayer membrane comprising CLEC-2. In embodiments, about 95% or less of the megakaryocyte-derived extracellular vesicles comprise a lipid bilayer membrane comprising CLEC-2. In embodiments, about 99% or less of the megakaryocyte-derived extracellular vesicles comprise a lipid bilayer membrane comprising CLEC-2.

In embodiments, less than about 50% of the megakaryocyte-derived extracellular vesicles comprise a lipid bilayer membrane comprising CLEC-2. In embodiments, less than about 40% of the megakaryocyte-derived extracellular vesicles comprise a lipid bilayer membrane comprising CLEC-2. In embodiments, less than about 30% of the megakaryocyte-derived extracellular vesicles comprise a lipid bilayer membrane comprising CLEC-2. In embodiments, less than about 25% of the megakaryocyte-derived extracellular vesicles comprise a lipid bilayer membrane comprising CLEC-2.

In embodiments, less than about 20% of the megakaryocyte-derived extracellular vesicles comprise a lipid bilayer membrane comprising CLEC-2. In embodiments, less than about 15% of the megakaryocyte-derived extracellular vesicles comprise a lipid bilayer membrane comprising CLEC-2. In embodiments, less than about 10% of the megakaryocyte-derived extracellular vesicles comprise a lipid bilayer membrane comprising CLEC-2. In embodiments, less than about 5% of the megakaryocyte-derived extracellular vesicles comprise a lipid bilayer membrane comprising CLEC-2. In embodiments, less than about 1% of the megakaryocyte-derived extracellular vesicles comprise a lipid bilayer membrane comprising CLEC-2.

In embodiments, between about 1% to about 99% of the megakaryocyte-derived extracellular vesicles comprise a lipid bilayer membrane comprising CLEC-2. In embodiments, between about 1% to about 50% of the megakaryocyte-derived extracellular vesicles comprise a lipid bilayer membrane comprising CLEC-2. In embodiments, between about 1% to about 25% of the megakaryocyte-derived extracellular vesicles comprise a lipid bilayer membrane comprising CLEC-2. In embodiments, between about 1% to about 10% of the megakaryocyte-derived extracellular vesicles comprise a lipid bilayer membrane comprising CLEC-2. In embodiments, between about 1% to about 5% of the megakaryocyte-derived extracellular vesicles comprise a lipid bilayer membrane comprising CLEC-2. In embodiments, between about 1% to about 2% of the megakaryocyte-derived extracellular vesicles comprise a lipid bilayer membrane comprising CLEC-2. In embodiments, between about 50% to about 99% of the megakaryocyte-derived extracellular vesicles comprise a lipid bilayer membrane comprising CLEC-2. In embodiments, between about 75% to about 99% of the megakaryocyte-derived extracellular vesicles comprise a lipid bilayer membrane comprising CLEC-2. In embodiments, between about 90% to about 99% of the megakaryocyte-derived extracellular vesicles comprise a lipid bilayer membrane comprising CLEC-2. In embodiments, between about 95% to about 99% of the megakaryocyte-derived extracellular vesicles comprise a lipid bilayer membrane comprising CLEC-2.

In embodiments, less than about 1%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%,) about 97%, about 98%, about 99%, or greater than about 99% of the megakaryocyte-derived extracellular vesicles comprise a lipid bilayer membrane comprising CLEC-2.

In embodiments, the megakaryocyte-derived extracellular vesicles are characterized by a higher expression and/or presence of CLEC-2 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles have about a 2-fold, or about a 10-fold, or about a 50-fold, or about a 100-fold, or about a 300-fold, or about a 500-fold, or about a 1000-fold greater amount of CLEC-2 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles.

In embodiments, the megakaryocyte-derived extracellular vesicles are characterized by a lower expression and/or presence of CLEC-2 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles have about a 2-fold, or about a 10-fold, or about a 50-fold, or about a 100-fold, or about a 300-fold, or about a 500-fold, or about a 1000-fold lower amount of CLEC-2 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles.

In embodiments, the megakaryocyte-derived extracellular vesicles have about a 2-fold to about a 16-fold, or about a 4-fold to about an 8-fold lower amount of CLEC-2 than platelet free plasma (PFP) MkEVs. In embodiments, the megakaryocyte-derived extracellular vesicles have about a 4-fold or about a 5-fold lower amount of CLEC-2 than platelet free plasma (PFP) MKEVs. In embodiments, the megakaryocyte-derived extracellular vesicles have about a 4-fold to about a 32-fold, or about an 8-fold to about a 16-fold lower amount of CLEC-2 than platelet derived extracellular vesicles (PLT EVs). In embodiments, the megakaryocyte-derived extracellular vesicles have about) a 10-fold or about a 12-fold lower amount of CLEC-2 than platelet derived extracellular vesicles (PLT EVs).

In embodiments, greater than about 50% of the megakaryocyte-derived extracellular vesicles comprise a lipid bilayer membrane comprising LAMP-1 (CD107A). In embodiments, greater than about 40% of the megakaryocyte-derived extracellular vesicles comprise a lipid bilayer membrane comprising LAMP-1 (CD107A). In embodiments, greater than about 60% of the megakaryocyte-derived extracellular vesicles comprise a lipid bilayer membrane comprising LAMP-1 (CD107A). In embodiments, greater than about 70% of the megakaryocyte-derived extracellular vesicles comprise a lipid bilayer membrane comprising LAMP-1 (CD107A). In embodiments, greater than about 80% of the megakaryocyte-derived extracellular vesicles comprise a lipid bilayer membrane comprising LAMP-1 (CD107A). In embodiments, greater than about 90% of the megakaryocyte-derived extracellular vesicles comprise a lipid bilayer membrane comprising LAMP-1 (CD107A). In embodiments, greater than about 95% of the megakaryocyte-derived extracellular vesicles comprise a lipid bilayer membrane comprising LAMP-1 (CD107A). In embodiments, greater than about 99% of the megakaryocyte-derived extracellular vesicles comprise a lipid bilayer membrane comprising LAMP-1 (CD107A).

In embodiments, about 50% or less of the megakaryocyte-derived extracellular vesicles comprise a lipid bilayer membrane comprising LAMP-1 (CD107A). In embodiments, about 40% or less of the megakaryocyte-derived extracellular vesicles comprise a lipid bilayer membrane comprising LAMP-1 (CD107A). In embodiments, about 60% or less of the megakaryocyte-derived extracellular vesicles comprise a lipid bilayer membrane comprising LAMP-1 (CD107A). In embodiments, about 70% or less of the megakaryocyte-derived extracellular vesicles comprise a lipid bilayer membrane comprising LAMP-1 (CD107A). In embodiments, about 80% or less of the megakaryocyte-derived extracellular vesicles comprise a lipid bilayer membrane comprising LAMP-1 (CD107A). In embodiments, about 90% or less of the megakaryocyte-derived extracellular vesicles comprise a lipid bilayer membrane comprising LAMP-1 (CD107A). In embodiments, about 95% or less of the megakaryocyte-derived extracellular vesicles comprise a lipid bilayer membrane comprising LAMP-1 (CD107A). In embodiments, about 99% or less of the megakaryocyte-derived extracellular vesicles comprise a lipid bilayer membrane comprising LAMP-1 (CD107A).

In embodiments, less than about 50% of the megakaryocyte-derived extracellular vesicles comprise a lipid bilayer membrane comprising LAMP-1 (CD107A). In embodiments, less than about 40% of the megakaryocyte-derived extracellular vesicles comprise a lipid bilayer membrane comprising LAMP-1 (CD107A). In embodiments, less than about 30% of the megakaryocyte-derived extracellular vesicles comprise a lipid bilayer membrane comprising LAMP-1 (CD107A). In embodiments, less than about 25% of the megakaryocyte-derived extracellular vesicles comprise a lipid bilayer membrane comprising LAMP-1 (CD107A). In embodiments, less than about 20% of the megakaryocyte-derived extracellular vesicles comprise a lipid bilayer membrane comprising LAMP-1 (CD107A). In embodiments, less than about 15% of the megakaryocyte-derived extracellular vesicles comprise a lipid bilayer membrane comprising LAMP-1 (CD107A). In embodiments, less than about 10% of the megakaryocyte-derived extracellular vesicles comprise a lipid bilayer membrane comprising LAMP-1 (CD107A). In embodiments, less than about 5% of the megakaryocyte-derived extracellular vesicles comprise a lipid bilayer membrane comprising LAMP-1 (CD107A). In embodiments, less than about 1% of the megakaryocyte-derived extracellular vesicles comprise a lipid bilayer membrane comprising LAMP-1 (CD107A).

In embodiments, between about 1% to about 99% of the megakaryocyte-derived extracellular vesicles comprise a lipid bilayer membrane comprising LAMP-1 (CD107A). In embodiments, between about 1% to about 50% of the megakaryocyte-derived extracellular vesicles comprise a lipid bilayer membrane comprising LAMP-1 (CD107A). In embodiments, between about 1% to about 25% of the megakaryocyte-derived extracellular vesicles comprise a lipid bilayer membrane comprising LAMP-1 (CD107A). In embodiments, between about 1% to about 10% of the megakaryocyte-derived extracellular vesicles comprise a lipid bilayer membrane comprising LAMP-1 (CD107A). In embodiments, between about 1% to about 5% of the megakaryocyte-derived extracellular vesicles comprise a lipid bilayer membrane comprising LAMP-1 (CD107A). In embodiments, between about 1% to about 2% of the megakaryocyte-derived extracellular vesicles comprise a lipid bilayer membrane comprising LAMP-1 (CD107A). In embodiments, between about 50% to about 99% of the megakaryocyte-derived extracellular vesicles comprise a lipid bilayer membrane comprising LAMP-1 (CD107A). In embodiments, between about 75% to about 99% of the megakaryocyte-derived extracellular vesicles comprise a lipid bilayer membrane comprising LAMP-1 (CD107A). In embodiments, between about 90% to about 99% of the megakaryocyte-derived extracellular vesicles comprise a lipid bilayer membrane comprising LAMP-1 (CD107A). In embodiments, between about 95% to about 99% of the megakaryocyte-derived extracellular vesicles comprise a lipid bilayer membrane comprising LAMP-1 (CD107A).

In embodiments, less than about 1%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or greater than about 99% of the megakaryocyte-derived extracellular vesicles comprise a lipid bilayer membrane comprising LAMP-1 (CD107A).

In embodiments, the megakaryocyte-derived extracellular vesicles are free of, or substantially free of LAMP-1 (CD107A).

In embodiments, the megakaryocyte-derived extracellular vesicles are characterized by a higher expression and/or presence of LAMP-1 (CD107A) than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles have about a 2-fold, or about a 10-fold, or about a 50-fold, or about a 100-fold, or about a 300-fold, or about a 500-fold, or about a 1000-fold greater amount of LAMP-1 (CD107A) than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles.

In embodiments, the megakaryocyte-derived extracellular vesicles are characterized by a lower expression and/or presence of LAMP-1 (CD107A) than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles have about a 2-fold, or about a 10-fold, or about a 50-fold, or about a 100-fold, or about a 300-fold, or about a 500-fold, or about a 1000-fold lower amount of LAMP-1 (CD107A) than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles.

In embodiments, the megakaryocyte-derived extracellular vesicles have about a 1-fold to about a 2-fold, lower amount of LAMP-1 (CD107A) than platelet free plasma (PFP) MkEVs. In embodiments, the megakaryocyte-derived extracellular vesicles have an amount of LAMP-1 (CD107A) that is substantially the same as platelet free plasma (PFP) MkEVs. In embodiments, the megakaryocyte-derived extracellular vesicles have about a 2-fold to about a 8-fold, or about a 2-fold to about a 4-fold lower amount of LAMP-1 (CD107A) than platelet derived extracellular vesicles (PLT EVs). In embodiments, the megakaryocyte-derived extracellular vesicles have about a 3-fold or about a 4-fold lower amount of LAMP-1 (CD107A) than platelet derived extracellular vesicles (PLT EVs).

In embodiments, greater than about 50% of the megakaryocyte-derived extracellular vesicles comprise a lipid bilayer membrane comprising CD63. In embodiments, greater than about 40% of the megakaryocyte-derived extracellular vesicles comprise a lipid bilayer membrane comprising CD63. In embodiments, greater than about 60% of the megakaryocyte-derived extracellular vesicles comprise a lipid bilayer membrane comprising CD63. In embodiments, greater than about 70% of the megakaryocyte-derived extracellular vesicles comprise a lipid bilayer membrane comprising CD63. In embodiments, greater than about 80% of the megakaryocyte-derived extracellular vesicles comprise a lipid bilayer membrane comprising CD63. In embodiments, greater than about 90% of the megakaryocyte-derived extracellular vesicles comprise a lipid bilayer membrane comprising CD63. In embodiments, greater than about 95% of the megakaryocyte-derived extracellular vesicles comprise a lipid bilayer membrane comprising CD63. In embodiments, greater than about 99% of the megakaryocyte-derived extracellular vesicles comprise a lipid bilayer membrane comprising CD63.

In embodiments, about 50% or less of the megakaryocyte-derived extracellular vesicles comprise a lipid bilayer membrane comprising CD63. In embodiments, about 40% or less of the megakaryocyte-derived extracellular vesicles comprise a lipid) bilayer membrane comprising CD63. In embodiments, about 60% or less of the megakaryocyte-derived extracellular vesicles comprise a lipid bilayer membrane comprising CD63. In embodiments, about 70% or less of the megakaryocyte-derived extracellular vesicles comprise a lipid bilayer membrane comprising CD63. In embodiments, about 80% or less of the megakaryocyte-derived extracellular vesicles comprise a lipid bilayer membrane comprising CD63. In embodiments, about 90% or less of the megakaryocyte-derived extracellular vesicles comprise a lipid bilayer membrane comprising CD63. In embodiments, about 95% or less of the megakaryocyte-derived extracellular vesicles comprise a lipid bilayer membrane comprising CD63. In embodiments, about 99% or less of the megakaryocyte-derived extracellular vesicles comprise a lipid bilayer membrane comprising CD63.

In embodiments, less than about 50% of the megakaryocyte-derived extracellular vesicles comprise a lipid bilayer membrane comprising CD63. In embodiments, less than about 40% of the megakaryocyte-derived extracellular vesicles comprise a lipid bilayer membrane comprising CD63. In embodiments, less than about 30% of the megakaryocyte-derived extracellular vesicles comprise a lipid bilayer membrane comprising CD63. In embodiments, less than about 25% of the megakaryocyte-derived extracellular vesicles comprise a lipid bilayer membrane comprising CD63. In embodiments, less than about 20% of the megakaryocyte-derived extracellular vesicles comprise a lipid bilayer membrane comprising CD63. In embodiments, less than about 15% of the megakaryocyte-derived extracellular vesicles comprise a lipid bilayer membrane comprising CD63. In embodiments, less than about 10% of the megakaryocyte-derived extracellular vesicles comprise a lipid bilayer membrane) comprising CD63. In embodiments, less than about 5% of the megakaryocyte-derived extracellular vesicles comprise a lipid bilayer membrane comprising CD63. In embodiments, less than about 1% of the megakaryocyte-derived extracellular vesicles comprise a lipid bilayer membrane comprising CD63.

In embodiments, between about 1% to about 99% of the megakaryocyte-derived extracellular vesicles comprise a lipid bilayer membrane comprising CD63. In embodiments, between about 1% to about 50% of the megakaryocyte-derived extracellular vesicles comprise a lipid bilayer membrane comprising CD63. In embodiments, between about 1% to about 25% of the megakaryocyte-derived extracellular vesicles comprise a lipid bilayer membrane comprising CD63. In embodiments, between about 1% to about 10% of the megakaryocyte-derived extracellular vesicles comprise a lipid bilayer membrane comprising CD63. In embodiments, between about 1% to about 5% of the megakaryocyte-derived extracellular vesicles comprise a lipid bilayer membrane comprising CD63. In embodiments, between about 1% to about 2% of the megakaryocyte-derived extracellular vesicles comprise a lipid bilayer membrane comprising CD63. In embodiments, between about 50% to about 99% of the megakaryocyte-derived extracellular vesicles comprise a lipid bilayer membrane comprising CD63. In embodiments, between about 75% to about 99% of the megakaryocyte-derived extracellular vesicles comprise a lipid bilayer membrane comprising CD63. In embodiments, between about 90% to about 99% of the megakaryocyte-derived extracellular vesicles comprise a lipid bilayer membrane comprising CD63. In embodiments, between about 95% to about 99% of the megakaryocyte-derived extracellular vesicles comprise a lipid bilayer membrane comprising CD63.

In embodiments, less than about 1%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%,) about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or greater than about 99% of the megakaryocyte-derived extracellular vesicles comprise a lipid bilayer membrane comprising CD63.

In embodiments, between about 1% to about 30% of the megakaryocyte-derived extracellular vesicles comprise a lipid bilayer membrane comprising CD63. In embodiments, between about 5% to about 25% of the megakaryocyte-derived extracellular vesicles comprise a lipid bilayer membrane comprising CD63. In embodiments, between about 10% to about 20% of the megakaryocyte-derived extracellular vesicles comprise a lipid bilayer membrane comprising CD63. In embodiments, between about 13% to about 19% of the megakaryocyte-derived extracellular vesicles comprise a lipid bilayer membrane comprising CD63.

In embodiments, the megakaryocyte-derived extracellular vesicles are characterized by a higher expression and/or presence of CD63 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles have about a 2-fold, or about a 10-fold, or about a 50-fold, or about a 100-fold, or about a 300-fold, or about a 500-fold, or about a 1000-fold greater amount of CD63 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles.

In embodiments, the megakaryocyte-derived extracellular vesicles are characterized by a lower expression and/or presence of CD63 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles have about a 2-fold, or about a 10-fold, or about a 50-fold, or about a 100-fold, or about a 300-fold, or about a 500-fold, or about a 1000-fold lower amount of CD63 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles.

In embodiments, the megakaryocyte-derived extracellular vesicles have about a 2-fold to about an 8-fold, or about a 2-fold to about a 4-fold greater amount of CD63 than platelet free plasma (PFP) MKEVs. In embodiments, the megakaryocyte-derived extracellular vesicles have about a 2-fold or about a 3-fold greater amount of CD63 than platelet free plasma (PFP) MKEVs. In embodiments, the megakaryocyte-derived) extracellular vesicles have about a 1.1-fold to about a 2-fold lower amount of CD63 than platelet derived extracellular vesicles (PLT EVs). In embodiments, the megakaryocyte-derived extracellular vesicles have about a 1.1-fold or about a 1.2-fold lower amount of CD63 than platelet derived extracellular vesicles (PLT EVs). In embodiments, the megakaryocyte-derived extracellular vesicles have an amount of CD63 that is substantially the same as platelet derived extracellular vesicles (PLT EVs).

In embodiments, greater than about 50% of the megakaryocyte-derived extracellular vesicles comprise a lipid bilayer membrane comprising CD42b. In embodiments, greater than about 40% of the megakaryocyte-derived extracellular vesicles comprise a lipid bilayer membrane comprising CD42b. In embodiments, greater than about 60% of the megakaryocyte-derived extracellular vesicles comprise a lipid bilayer membrane comprising CD42b. In embodiments, greater than about 70% of the megakaryocyte-derived extracellular vesicles comprise a lipid bilayer membrane comprising CD42b.

In embodiments, greater than about 80% of the megakaryocyte-derived extracellular vesicles comprise a lipid bilayer membrane comprising CD42b. In embodiments, greater than about 90% of the megakaryocyte-derived extracellular vesicles comprise a lipid bilayer membrane comprising CD42b. In embodiments, greater than about 95% of the megakaryocyte-derived extracellular vesicles comprise a lipid bilayer membrane comprising CD42b. In embodiments, greater than about 99% of the megakaryocyte-derived extracellular vesicles comprise a lipid bilayer membrane comprising CD42b.

In embodiments, about 50% or less of the megakaryocyte-derived extracellular vesicles comprise a lipid bilayer membrane comprising CD42b. In embodiments, about 40% or less of the megakaryocyte-derived extracellular vesicles comprise a lipid bilayer membrane comprising CD42b. In embodiments, about 60% or less of the megakaryocyte-derived extracellular vesicles comprise a lipid bilayer membrane comprising CD42b. In embodiments, about 70% or less of the megakaryocyte-derived extracellular vesicles comprise a lipid bilayer membrane comprising CD42b. In embodiments, about 80% or less of the megakaryocyte-derived extracellular vesicles comprise a lipid bilayer membrane comprising CD42b. In embodiments, about 90% or less of the megakaryocyte-derived extracellular vesicles comprise a lipid bilayer membrane comprising CD42b. In embodiments, about 95% or less of the megakaryocyte-derived extracellular vesicles comprise a lipid bilayer membrane comprising CD42b. In embodiments, about 99% or less of the megakaryocyte-derived extracellular vesicles comprise a lipid bilayer membrane comprising CD42b.

In embodiments, less than about 50% of the megakaryocyte-derived extracellular vesicles comprise a lipid bilayer membrane comprising CD42b. In embodiments, less than about 40% of the megakaryocyte-derived extracellular vesicles comprise a lipid bilayer membrane comprising CD42b. In embodiments, less than about 30% of the megakaryocyte-derived extracellular vesicles comprise a lipid bilayer membrane comprising CD42b. In embodiments, less than about 25% of the megakaryocyte-derived extracellular vesicles comprise a lipid bilayer membrane comprising CD42b. In embodiments, less than about 20% of the megakaryocyte-derived extracellular vesicles comprise a lipid bilayer membrane comprising CD42b. In embodiments, less than about 15% of the megakaryocyte-derived extracellular vesicles comprise a lipid bilayer membrane comprising CD42b. In embodiments, less than about 10% of the megakaryocyte-derived extracellular vesicles comprise a lipid bilayer membrane comprising CD42b. In embodiments, less than about 5% of the megakaryocyte-derived extracellular vesicles comprise a lipid bilayer membrane comprising CD42b. In embodiments, less than about 1% of the megakaryocyte-derived extracellular vesicles comprise a lipid bilayer membrane comprising CD42b.

In embodiments, between about 1% to about 99% of the megakaryocyte-derived extracellular vesicles comprise a lipid bilayer membrane comprising CD42b. In embodiments, between about 1% to about 50% of the megakaryocyte-derived extracellular vesicles comprise a lipid bilayer membrane comprising CD42b. In embodiments, between about 1% to about 25% of the megakaryocyte-derived extracellular vesicles comprise a lipid bilayer membrane comprising CD42b. In 20) embodiments, between about 1% to about 10% of the megakaryocyte-derived extracellular vesicles comprise a lipid bilayer membrane comprising CD42b. In embodiments, between about 1% to about 5% of the megakaryocyte-derived extracellular vesicles comprise a lipid bilayer membrane comprising CD42b. In embodiments, between about 1% to about 2% of the megakaryocyte-derived extracellular vesicles comprise a lipid bilayer membrane comprising CD42b. In embodiments, between about 50% to about 99% of the megakaryocyte-derived extracellular vesicles comprise a lipid bilayer membrane comprising CD42b. In embodiments, between about 75% to about 99% of the megakaryocyte-derived) extracellular vesicles comprise a lipid bilayer membrane comprising CD42b. In embodiments, between about 90% to about 99% of the megakaryocyte-derived extracellular vesicles comprise a lipid bilayer membrane comprising CD42b. In embodiments, between about 95% to about 99% of the megakaryocyte-derived extracellular vesicles comprise a lipid bilayer membrane comprising CD42b.

In embodiments, less than about 1%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or greater than about 99% of the megakaryocyte-derived extracellular vesicles comprise a lipid bilayer membrane comprising CD42b.

In embodiments, the megakaryocyte-derived extracellular vesicles are characterized by a higher expression and/or presence of CD42b than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles have about a 2-fold, or about a 10-fold, or about a 50-fold, or about a 100-fold, or about a 300-fold, or about a 500-fold, or about a 1000-fold greater amount of CD42b than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles.

In embodiments, the megakaryocyte-derived extracellular vesicles are characterized by a lower expression and/or presence of CD42b than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles have about a 2-fold, or about a 10-fold, or about a 50-fold, or about a 100-fold, or about a 300-fold, or about a 500-fold, or about a 1000-fold lower amount of CD42b than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles.

In embodiments, the megakaryocyte-derived extracellular vesicles have about an 8-fold to about a 32-fold, or about a 10-fold to about a 20-fold lower amount of CD42b than platelet free plasma (PFP) MKEVs. In embodiments, the megakaryocyte-derived extracellular vesicles have about a 16-fold or about a 20-fold lower amount of CD42b than platelet free plasma (PFP) MKEVs. In embodiments, the megakaryocyte-derived extracellular vesicles have about a 64-fold to about a 128-fold, or about a 50-fold to about a 75-fold lower amount of CD42b than platelet derived extracellular vesicles (PLT EVs). In embodiments, the megakaryocyte-derived extracellular vesicles have about a 64-fold or about a 70-fold lower amount of CD42b than platelet derived extracellular vesicles (PLT EVs).

In embodiments, greater than about 50% of the megakaryocyte-derived extracellular vesicles comprise a lipid bilayer membrane comprising CD9. In embodiments, greater) than about 40% of the megakaryocyte-derived extracellular vesicles comprise a lipid bilayer membrane comprising CD9. In embodiments, greater than about 60% of the megakaryocyte-derived extracellular vesicles comprise a lipid bilayer membrane comprising CD9. In embodiments, greater than about 70% of the megakaryocyte-derived extracellular vesicles comprise a lipid bilayer membrane comprising CD9. In embodiments, greater than about 80% of the megakaryocyte-derived extracellular vesicles comprise a lipid bilayer membrane comprising CD9. In embodiments, greater than about 90% of the megakaryocyte-derived extracellular vesicles comprise a lipid bilayer membrane comprising CD9. In embodiments, greater than about 95% of the megakaryocyte-derived extracellular vesicles comprise a lipid bilayer membrane comprising CD9. In embodiments, greater than about 99% of the megakaryocyte-derived extracellular vesicles comprise a lipid bilayer membrane comprising CD9.

In embodiments, about 50% or less of the megakaryocyte-derived extracellular vesicles comprise a lipid bilayer membrane comprising CD9. In embodiments, about 40% or less of the megakaryocyte-derived extracellular vesicles comprise a lipid bilayer membrane comprising CD9. In embodiments, about 60% or less of the megakaryocyte-derived extracellular vesicles comprise a lipid bilayer membrane comprising CD9. In embodiments, about 70% or less of the megakaryocyte-derived extracellular vesicles comprise a lipid bilayer membrane comprising CD9. In embodiments, about 80% or less of the megakaryocyte-derived extracellular vesicles comprise a lipid bilayer membrane comprising CD9. In embodiments, about 90% or less of the megakaryocyte-derived extracellular vesicles comprise a lipid bilayer membrane comprising CD9. In embodiments, about 95% or less of the megakaryocyte-derived extracellular vesicles comprise a lipid bilayer membrane comprising CD9. In embodiments, about 99% or less of the megakaryocyte-derived extracellular vesicles comprise a lipid bilayer membrane comprising CD9.

In embodiments, less than about 50% of the megakaryocyte-derived extracellular vesicles comprise a lipid bilayer membrane comprising CD9. In embodiments, less than about 40% of the megakaryocyte-derived extracellular vesicles comprise a lipid bilayer membrane comprising CD9. In embodiments, less than about 30% of the megakaryocyte-derived extracellular vesicles comprise a lipid bilayer membrane comprising CD9. In embodiments, less than about 25% of the megakaryocyte-derived extracellular vesicles comprise a lipid bilayer membrane comprising CD9. In) embodiments, less than about 20% of the megakaryocyte-derived extracellular vesicles comprise a lipid bilayer membrane comprising CD9. In embodiments, less than about 15% of the megakaryocyte-derived extracellular vesicles comprise a lipid bilayer membrane comprising CD9. In embodiments, less than about 10% of the megakaryocyte-derived extracellular vesicles comprise a lipid bilayer membrane comprising CD9. In embodiments, less than about 5% of the megakaryocyte-derived extracellular vesicles comprise a lipid bilayer membrane comprising CD9. In embodiments, less than about 1% of the megakaryocyte-derived extracellular vesicles comprise a lipid bilayer membrane comprising CD9.

In embodiments, between about 1% to about 99% of the megakaryocyte-derived extracellular vesicles comprise a lipid bilayer membrane comprising CD9. In embodiments, between about 1% to about 50% of the megakaryocyte-derived extracellular vesicles comprise a lipid bilayer membrane comprising CD9. In embodiments, between about 1% to about 25% of the megakaryocyte-derived extracellular vesicles comprise a lipid bilayer membrane comprising CD9. In embodiments, between about 1% to about 10% of the megakaryocyte-derived extracellular vesicles comprise a lipid bilayer membrane comprising CD9. In embodiments, between about 1% to about 5% of the megakaryocyte-derived extracellular vesicles comprise a lipid bilayer membrane comprising CD9. In embodiments, between about 1% to about 2% of the megakaryocyte-derived extracellular vesicles comprise a lipid bilayer membrane comprising CD9. In embodiments, between about 50% to about 99% of the megakaryocyte-derived extracellular vesicles comprise a lipid bilayer membrane comprising CD9. In embodiments, between about 75% to about 99% of the megakaryocyte-derived extracellular vesicles comprise a lipid bilayer membrane comprising CD9. In embodiments, between about 90% to about 99% of the megakaryocyte-derived extracellular vesicles comprise a lipid bilayer membrane comprising CD9. In embodiments, between about 95% to about 99% of the megakaryocyte-derived extracellular vesicles comprise a lipid bilayer membrane comprising CD9.

In embodiments, less than about 1%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%,) about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or greater than about 99% of the megakaryocyte-derived extracellular vesicles comprise a lipid bilayer membrane comprising CD9.

In embodiments, between about 50% to about 70% of the megakaryocyte-derived extracellular vesicles comprise a lipid bilayer membrane comprising CD9. In embodiments, between about 60% to about 70% of the megakaryocyte-derived extracellular vesicles comprise a lipid bilayer membrane comprising CD9. In embodiments, between about 62% to about 68% of the megakaryocyte-derived extracellular vesicles comprise a lipid bilayer membrane comprising CD9. In embodiments, between about 65% to about 66% of the megakaryocyte-derived extracellular vesicles comprise a lipid bilayer membrane comprising CD9.

In embodiments, between about 50% to about 99% of the megakaryocyte-derived extracellular vesicles comprise a lipid bilayer membrane comprising CD9.

In embodiments, the megakaryocyte-derived extracellular vesicles are characterized by a higher expression and/or presence of CD9 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles have about a 2-fold, or about a 10-fold, or about a 50-fold, or about a 100-fold, or about a 300-fold, or about a 500-fold, or about a 1000-fold greater amount of CD9 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles.

20) In embodiments, the megakaryocyte-derived extracellular vesicles are characterized by a lower expression and/or presence of CD9 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles have about a 2-fold, 25 or about a 10-fold, or about a 50-fold, or about a 100-fold, or about a 300-fold, or about a 500-fold, or about a 1000-fold lower amount of CD9 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. 30) In embodiments, the megakaryocyte-derived extracellular vesicles have about a 1.5-fold to about a 4-fold, or about a 2-fold to about a 4-fold greater amount of CD9 than platelet free plasma (PFP) MkEVs. In embodiments, the megakaryocyte-derived extracellular vesicles have about a 2-fold greater amount of CD9 than platelet free plasma (PFP) MkEVs. In embodiments, the megakaryocyte-derived extracellular vesicles have about a 1.1-fold to about a 2-fold lower amount of CD9 than platelet derived extracellular vesicles (PLT EVs). In embodiments, the megakaryocyte-derived extracellular vesicles have about a 1.1-fold or about a 1.2-fold lower amount of CD9 than platelet derived extracellular vesicles (PLT EVs). In embodiments, the megakaryocyte-derived extracellular vesicles have an amount of CD9 that is substantially the same as platelet derived extracellular vesicles (PLT EVs).

In embodiments, greater than about 50% of the megakaryocyte-derived extracellular vesicles comprise a lipid bilayer membrane comprising CD31. In embodiments, greater than about 40% of the megakaryocyte-derived extracellular vesicles comprise a lipid bilayer membrane comprising CD31. In embodiments, greater than about 60% of the megakaryocyte-derived extracellular vesicles comprise a lipid bilayer membrane comprising CD31. In embodiments, greater than about 70% of the megakaryocyte-derived extracellular vesicles comprise a lipid bilayer membrane comprising CD31. In embodiments, greater than about 80% of the megakaryocyte-derived extracellular vesicles comprise a lipid bilayer membrane comprising CD31. In embodiments, greater than about 90% of the megakaryocyte-derived extracellular vesicles comprise a lipid bilayer membrane comprising CD31. In embodiments, greater than about 95% of the megakaryocyte-derived extracellular vesicles comprise a lipid bilayer membrane comprising CD31. In embodiments, greater than about 99% of the megakaryocyte-derived extracellular vesicles comprise a lipid bilayer membrane comprising CD31.

In embodiments, about 50% or less of the megakaryocyte-derived extracellular vesicles comprise a lipid bilayer membrane comprising CD31. In embodiments, about 40% or less of the megakaryocyte-derived extracellular vesicles comprise a lipid bilayer membrane comprising CD31. In embodiments, about 60% or less of the megakaryocyte-derived extracellular vesicles comprise a lipid bilayer membrane comprising CD31. In embodiments, about 70% or less of the megakaryocyte-derived extracellular vesicles comprise a lipid bilayer membrane comprising CD31. In embodiments, about 80% or less of the megakaryocyte-derived extracellular vesicles comprise a lipid bilayer membrane comprising CD31. In embodiments, about 90% or less of the megakaryocyte-derived extracellular vesicles comprise a lipid bilayer membrane comprising CD31. In embodiments, about 95% or less of the megakaryocyte-derived extracellular vesicles comprise a lipid bilayer membrane comprising CD31. In embodiments, about 99% or less of the megakaryocyte-derived extracellular vesicles comprise a lipid bilayer membrane comprising CD31.

In embodiments, less than about 50% of the megakaryocyte-derived extracellular vesicles comprise a lipid bilayer membrane comprising CD31. In embodiments, less than about 40% of the megakaryocyte-derived extracellular vesicles comprise a lipid bilayer membrane comprising CD31. In embodiments, less than about 30% of the megakaryocyte-derived extracellular vesicles comprise a lipid bilayer membrane comprising CD31. In embodiments, less than about 25% of the megakaryocyte-derived extracellular vesicles comprise a lipid bilayer membrane comprising CD31. In embodiments, less than about 20% of the megakaryocyte-derived extracellular vesicles comprise a lipid bilayer membrane comprising CD31. In embodiments, less than about 15% of the megakaryocyte-derived extracellular vesicles comprise a lipid bilayer membrane comprising CD31. In embodiments, less than about 10% of the megakaryocyte-derived extracellular vesicles comprise a lipid bilayer membrane comprising CD31. In embodiments, less than about 5% of the megakaryocyte-derived extracellular vesicles comprise a lipid bilayer membrane comprising CD31. In embodiments, less than about 1% of the megakaryocyte-derived extracellular vesicles comprise a lipid bilayer membrane comprising CD31.

In embodiments, between about 1% to about 99% of the megakaryocyte-derived extracellular vesicles comprise a lipid bilayer membrane comprising CD31. In embodiments, between about 1% to about 50% of the megakaryocyte-derived extracellular vesicles comprise a lipid bilayer membrane comprising CD31. In embodiments, between about 1% to about 25% of the megakaryocyte-derived extracellular vesicles comprise a lipid bilayer membrane comprising CD31. In embodiments, between about 1% to about 10% of the megakaryocyte-derived extracellular vesicles comprise a lipid bilayer membrane comprising CD31. In embodiments, between about 1% to about 5% of the megakaryocyte-derived extracellular vesicles comprise a lipid bilayer membrane comprising CD31. In embodiments, between about 1% to about 2% of the megakaryocyte-derived extracellular vesicles comprise a lipid bilayer membrane comprising CD31. In embodiments, between about 50% to about 99% of the megakaryocyte-derived extracellular vesicles comprise a lipid bilayer membrane comprising CD31. In embodiments, between about 75% to about 99% of the megakaryocyte-derived extracellular vesicles comprise a lipid bilayer membrane comprising CD31. In embodiments, between about 90% to about 99% of the megakaryocyte-derived extracellular vesicles comprise a lipid bilayer membrane comprising CD31. In embodiments, between about 95% to about 99% of the megakaryocyte-derived extracellular vesicles comprise a lipid bilayer membrane comprising CD31.

In embodiments, less than about 1%, about 1%, about 2%, about 3%, about 4%, about 5%, about 6%, about 7%, about 8%, about 9%, about 10%, about 11%, about 12%,) about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%,) about 83%, about 84%, about 85%, about 86%, about 87%, about 88%, about 89%, about 90%, about 91%, about 92%, about 93%, about 94%, about 95%, about 96%, about 97%, about 98%, about 99%, or greater than about 99% of the megakaryocyte-derived extracellular vesicles comprise a lipid bilayer membrane comprising CD31.

In embodiments, between about 5% to about 50% of the megakaryocyte-derived extracellular vesicles comprise a lipid bilayer membrane comprising CD31. In embodiments, between about 10% to about 40% of the megakaryocyte-derived extracellular vesicles comprise a lipid bilayer membrane comprising CD31. In embodiments, between about 10% to about 35% of the megakaryocyte-derived extracellular vesicles comprise a lipid bilayer membrane comprising CD31. In embodiments, between about 13% to about 31% of the megakaryocyte-derived extracellular vesicles comprise a lipid bilayer membrane comprising CD31.

In embodiments, the megakaryocyte-derived extracellular vesicles are characterized by a higher expression and/or presence of CD31 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles have about a 2-fold, or about a 10-fold, or about a 50-fold, or about a 100-fold, or about a 300-fold, or about a 500-fold, or about a 1000-fold greater amount of CD31 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles.

In embodiments, the megakaryocyte-derived extracellular vesicles are characterized by a lower expression and/or presence of CD31 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles have about a 2-fold, or about a 10-fold, or about a 50-fold, or about a 100-fold, or about a 300-fold, or about a 500-fold, or about a 1000-fold lower amount of CD31 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. 20) In embodiments, the megakaryocyte-derived extracellular vesicles have about a 1.1-fold to about a 4-fold, or about a 1.1-fold to about a 2-fold lower amount of CD31 than platelet free plasma (PFP) MkEVs. In embodiments, the megakaryocyte-derived extracellular vesicles have about a 1.5-fold lower amount of CD31 than platelet free plasma (PFP) MkEVs. In embodiments, the megakaryocyte-derived extracellular vesicles have about a 2-fold to about a 4-fold lower amount of CD31 than platelet derived extracellular vesicles (PLT EVs). In embodiments, the megakaryocyte-derived extracellular vesicles have about a 2-fold or about a 3-fold lower amount of CD31 than platelet derived extracellular vesicles (PLT EVs).

In embodiments, greater than about 50% of the megakaryocyte-derived extracellular vesicles comprise a lipid bilayer membrane comprising CD47. In embodiments, greater than about 40% of the megakaryocyte-derived extracellular vesicles comprise a lipid bilayer membrane comprising CD47. In embodiments, greater than about 60% of the megakaryocyte-derived extracellular vesicles comprise a lipid bilayer membrane comprising CD47. In embodiments, greater than about 70% of the megakaryocyte-derived extracellular vesicles comprise a lipid bilayer membrane comprising CD47. In embodiments, greater than about 80% of the megakaryocyte-derived extracellular vesicles comprise a lipid bilayer membrane comprising CD47. In embodiments, greater than about 90% of the megakaryocyte-derived extracellular vesicles comprise a lipid bilayer membrane comprising CD47. In embodiments, greater than about 95% of the megakaryocyte-derived extracellular vesicles comprise a lipid bilayer membrane comprising CD47. In embodiments, greater than about 99% of the megakaryocyte-derived extracellular vesicles comprise a lipid bilayer membrane comprising CD47.

In embodiments, about 50% or less of the megakaryocyte-derived extracellular vesicles comprise a lipid bilayer membrane comprising CD47. In embodiments, about 40% or less of the megakaryocyte-derived extracellular vesicles comprise a lipid bilayer membrane comprising CD47. In embodiments, about 60% or less of the megakaryocyte-derived extracellular vesicles comprise a lipid bilayer membrane comprising CD47. In embodiments, about 70% or less of the megakaryocyte-derived extracellular vesicles comprise a lipid bilayer membrane comprising CD47. In embodiments, about 80% or less of the megakaryocyte-derived extracellular vesicles comprise a lipid bilayer membrane comprising CD47. In embodiments, about 90% or less of the megakaryocyte-derived extracellular vesicles comprise a lipid bilayer membrane comprising CD47. In embodiments, about 95% or less of the megakaryocyte-derived extracellular vesicles comprise a lipid bilayer membrane comprising CD47. In embodiments, about 99% or less of the megakaryocyte-derived extracellular vesicles comprise a lipid bilayer membrane comprising CD47.

In embodiments, less than about 50% of the megakaryocyte-derived extracellular vesicles comprise a lipid bilayer membrane comprising CD47. In embodiments, less than about 40% of the megakaryocyte-derived extracellular vesicles comprise a lipid bilayer membrane comprising CD47. In embodiments, less than about 30% of the megakaryocyte-derived extracellular vesicles comprise a lipid bilayer membrane comprising CD47. In embodiments, less than about 25% of the megakaryocyte-derived extracellular vesicles comprise a lipid bilayer membrane comprising CD47. In embodiments, less than about 20% of the megakaryocyte-derived extracellular vesicles comprise a lipid bilayer membrane comprising CD47. In embodiments, less than about 15% of the megakaryocyte-derived extracellular vesicles comprise a lipid bilayer membrane comprising CD47. In embodiments, less than about 10% of the megakaryocyte-derived extracellular vesicles comprise a lipid bilayer membrane comprising CD47. In embodiments, less than about 5% of the megakaryocyte-derived extracellular vesicles comprise a lipid bilayer membrane comprising CD47. In embodiments, less than about 1% of the megakaryocyte-derived extracellular vesicles comprise a lipid bilayer membrane comprising CD47.

In embodiments, between about 1% to about 99% of the megakaryocyte-derived extracellular vesicles comprise a lipid bilayer membrane comprising CD47. In embodiments, between about 1% to about 50% of the megakaryocyte-derived extracellular vesicles comprise a lipid bilayer membrane comprising CD47. In embodiments, between about 1% to about 25% of the megakaryocyte-derived extracellular vesicles comprise a lipid bilayer membrane comprising CD47. In embodiments, between about 1% to about 10% of the megakaryocyte-derived extracellular vesicles comprise a lipid bilayer membrane comprising CD47. In embodiments, between about 1% to about 5% of the megakaryocyte-derived extracellular vesicles comprise a lipid bilayer membrane comprising CD47. In embodiments, between about 1% to about 2% of the megakaryocyte-derived extracellular vesicles comprise a lipid bilayer membrane comprising CD47. In embodiments, between about 50% to about 99% of the megakaryocyte-derived extracellular vesicles comprise a lipid bilayer membrane comprising CD47. In embodiments, between about 75% to about 99% of the megakaryocyte-derived extracellular vesicles comprise a lipid bilayer membrane comprising CD47. In embodiments, between about 90% to about 99% of the megakaryocyte-derived extracellular vesicles comprise a lipid bilayer membrane comprising CD47. In embodiments, between about 95% to about 99% of the megakaryocyte-derived extracellular vesicles comprise a lipid bilayer membrane comprising CD47.

In embodiments, less than about 1%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or greater than about 99% of the megakaryocyte-derived extracellular vesicles comprise a lipid bilayer membrane comprising CD47.

In embodiments, between about 1% to about 50% of the megakaryocyte-derived extracellular vesicles comprise a lipid bilayer membrane comprising CD47. In embodiments, between about 10% to about 40% of the megakaryocyte-derived extracellular vesicles comprise a lipid bilayer membrane comprising CD47. In embodiments, between about 20% to about 40% of the megakaryocyte-derived extracellular vesicles comprise a lipid bilayer membrane comprising CD47. In embodiments, between about 25% to about 35% of the megakaryocyte-derived extracellular vesicles comprise a lipid bilayer membrane comprising CD47.

In embodiments, the megakaryocyte-derived extracellular vesicles are characterized by a higher expression and/or presence of CD47 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles have about a 2-fold, or about a 10-fold, or about a 50-fold, or about a 100-fold, or about a 300-fold, or about a 500-fold, or about a 1000-fold greater amount of CD47 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles.

In embodiments, the megakaryocyte-derived extracellular vesicles are characterized by a lower expression and/or presence of CD47 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles have about a 2-fold, or about a 10-fold, or about a 50-fold, or about a 100-fold, or about a 300-fold, or about a 500-fold, or about a 1000-fold lower amount of CD47 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles.

In embodiments, the megakaryocyte-derived extracellular vesicles have about a 128-fold to about a 512-fold, or about a 256-fold to about a 512-fold, or about a 250-fold to about a 300-fold greater amount of CD47 than platelet free plasma (PFP) MkEVs. In embodiments, the megakaryocyte-derived extracellular vesicles have about a 256-fold or about a 300-fold greater amount of CD47 than platelet free plasma (PFP) MkEVs. In embodiments, the megakaryocyte-derived extracellular vesicles have about a 1.1-fold to about a 2-fold lower amount of CD47 than platelet derived extracellular vesicles (PLT EVs). In embodiments, the megakaryocyte-derived extracellular vesicles have about a 1.1-fold or about a 1.5-fold lower amount of CD47 than platelet derived extracellular vesicles (PLT EVs). In embodiments, the megakaryocyte-derived extracellular vesicles have an amount of CD47 that is substantially the same as platelet derived extracellular vesicles (PLT EVs).

20) In embodiments, greater than about 50% of the megakaryocyte-derived extracellular vesicles comprise a lipid bilayer membrane comprising CD147. In embodiments, greater than about 40% of the megakaryocyte-derived extracellular vesicles comprise a lipid bilayer membrane comprising CD147. In embodiments, greater than about 60% of the megakaryocyte-derived extracellular vesicles comprise a lipid bilayer membrane comprising CD147. In embodiments, greater than about 70% of the megakaryocyte-derived extracellular vesicles comprise a lipid bilayer membrane comprising CD147. In embodiments, greater than about 80% of the megakaryocyte-derived extracellular vesicles comprise a lipid bilayer membrane comprising CD147. In embodiments, greater than about 90% of the megakaryocyte-derived extracellular vesicles comprise a lipid bilayer membrane comprising CD147. In embodiments, greater than about 95% of the megakaryocyte-derived extracellular vesicles comprise a lipid bilayer membrane comprising CD147. In embodiments, greater than about 99% of the megakaryocyte-derived extracellular vesicles comprise a lipid bilayer membrane comprising CD147.

In embodiments, about 50% or less of the megakaryocyte-derived extracellular vesicles comprise a lipid bilayer membrane comprising CD147. In embodiments, about 40% or less of the megakaryocyte-derived extracellular vesicles comprise a lipid bilayer membrane comprising CD147. In embodiments, about 60% or less of the megakaryocyte-derived extracellular vesicles comprise a lipid bilayer membrane comprising CD147. In embodiments, about 70% or less of the megakaryocyte-derived extracellular vesicles comprise a lipid bilayer membrane comprising CD147. In embodiments, about 80% or less of the megakaryocyte-derived extracellular vesicles comprise a lipid bilayer membrane comprising CD147. In embodiments, about 90% or less of the megakaryocyte-derived extracellular vesicles comprise a lipid bilayer membrane comprising CD147. In embodiments, about 95% or less of the megakaryocyte-derived extracellular vesicles comprise a lipid bilayer membrane comprising CD147. In embodiments, about 99% or less of the megakaryocyte-derived extracellular vesicles comprise a lipid bilayer membrane comprising CD147.

In embodiments, less than about 50% of the megakaryocyte-derived extracellular vesicles comprise a lipid bilayer membrane comprising CD147. In embodiments, less than about 40% of the megakaryocyte-derived extracellular vesicles comprise a lipid bilayer membrane comprising CD147. In embodiments, less than about 30% of the megakaryocyte-derived extracellular vesicles comprise a lipid bilayer membrane) comprising CD147. In embodiments, less than about 25% of the megakaryocyte-derived extracellular vesicles comprise a lipid bilayer membrane comprising CD147.

In embodiments, less than about 20% of the megakaryocyte-derived extracellular vesicles comprise a lipid bilayer membrane comprising CD147. In embodiments, less than about 15% of the megakaryocyte-derived extracellular vesicles comprise a lipid bilayer membrane comprising CD147. In embodiments, less than about 10% of the megakaryocyte-derived extracellular vesicles comprise a lipid bilayer membrane comprising CD147. In embodiments, less than about 5% of the megakaryocyte-derived extracellular vesicles comprise a lipid bilayer membrane comprising CD147. In embodiments, less than about 1% of the megakaryocyte-derived extracellular vesicles comprise a lipid bilayer membrane comprising CD147.

In embodiments, between about 1% to about 99% of the megakaryocyte-derived extracellular vesicles comprise a lipid bilayer membrane comprising CD147. In embodiments, between about 1% to about 50% of the megakaryocyte-derived extracellular vesicles comprise a lipid bilayer membrane comprising CD147. In embodiments, between about 1% to about 25% of the megakaryocyte-derived extracellular vesicles comprise a lipid bilayer membrane comprising CD147. In embodiments, between about 1% to about 10% of the megakaryocyte-derived extracellular vesicles comprise a lipid bilayer membrane comprising CD147. In embodiments, between about 1% to about 5% of the megakaryocyte-derived extracellular vesicles comprise a lipid bilayer membrane comprising CD147. In embodiments, between about 1% to about 2% of the megakaryocyte-derived extracellular vesicles comprise a lipid bilayer membrane comprising CD147. In embodiments, between about 50% to about 99% of the megakaryocyte-derived extracellular vesicles comprise a lipid bilayer membrane comprising CD147. In embodiments, between about 75% to about 99% of the megakaryocyte-derived extracellular vesicles comprise a lipid bilayer membrane comprising CD147. In embodiments, between about 90% to about 99% of the megakaryocyte-derived extracellular vesicles comprise a lipid bilayer membrane comprising CD147. In embodiments, between about 95% to about 99% of the megakaryocyte-derived extracellular vesicles comprise a lipid bilayer membrane comprising CD147.

In embodiments, less than about 1%, about 1%, about 2%, about 3%, about 4%, about 5%, about 6%, about 7%, about 8%, about 9%, about 10%, about 11%, about 12%,) about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or greater than about 99% of the megakaryocyte-derived extracellular vesicles comprise a lipid bilayer membrane comprising CD147.

In embodiments, between about 1% to about 15% of the megakaryocyte-derived extracellular vesicles comprise a lipid bilayer membrane comprising CD147. In embodiments, between about 1% to about 10% of the megakaryocyte-derived extracellular vesicles comprise a lipid bilayer membrane comprising CD147. In embodiments, between about 3% to about 8% of the megakaryocyte-derived extracellular vesicles comprise a lipid bilayer membrane comprising CD147. In embodiments, between about 4% to about 7% of the megakaryocyte-derived extracellular vesicles comprise a lipid bilayer membrane comprising CD147.

In embodiments, the megakaryocyte-derived extracellular vesicles are characterized by a higher expression and/or presence of CD147 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles have about a 2-fold, or about a 10-fold, or about a 50-fold, or about a 100-fold, or about a 300-fold, or about a 500-fold, or about a 1000-fold greater amount of CD147 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles.

In embodiments, the megakaryocyte-derived extracellular vesicles are characterized by a lower expression and/or presence of CD147 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles have about a 2-fold, or about a 10-fold, or about a 50-fold, or about a 100-fold, or about a 300-fold, or about a 500-fold, or about a 1000-fold lower amount of CD147 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles.

In embodiments, the megakaryocyte-derived extracellular vesicles have about a 2-fold to about an 8-fold, or about a 2-fold to about a 4-fold lower amount of CD147 than platelet free plasma (PFP) MkEVs. In embodiments, the megakaryocyte-derived extracellular vesicles have about a 2-fold or about a 3-fold lower amount of CD147 than platelet free plasma (PFP) MKEVs. In embodiments, the megakaryocyte-derived extracellular vesicles have about a 1.1-fold to about a 2-fold lower amount of CD147 than platelet derived extracellular vesicles (PLT EVs). In embodiments, the megakaryocyte-derived extracellular vesicles have about a 1.1-fold or about a 1.2-fold lower amount of CD147 than platelet derived extracellular vesicles (PLT EVs). In embodiments, the megakaryocyte-derived extracellular vesicles have an amount of CD147 that is substantially the same as platelet derived extracellular vesicles (PLT EVs).

In embodiments, greater than about 50% of the megakaryocyte-derived extracellular vesicles comprise a lipid bilayer membrane comprising CD32a. In embodiments, greater than about 40% of the megakaryocyte-derived extracellular vesicles comprise a lipid bilayer membrane comprising CD32a. In embodiments, greater than about 60% of the megakaryocyte-derived extracellular vesicles comprise a lipid bilayer membrane comprising CD32a. In embodiments, greater than about 70% of the megakaryocyte-derived extracellular vesicles comprise a lipid bilayer membrane comprising CD32a.

In embodiments, greater than about 80% of the megakaryocyte-derived extracellular vesicles comprise a lipid bilayer membrane comprising CD32a. In embodiments, greater than about 90% of the megakaryocyte-derived extracellular vesicles comprise) a lipid bilayer membrane comprising CD32a. In embodiments, greater than about 95% of the megakaryocyte-derived extracellular vesicles comprise a lipid bilayer membrane comprising CD32a. In embodiments, greater than about 99% of the megakaryocyte-derived extracellular vesicles comprise a lipid bilayer membrane comprising CD32a.

In embodiments, about 50% or less of the megakaryocyte-derived extracellular vesicles comprise a lipid bilayer membrane comprising CD32a. In embodiments, about 40% or less of the megakaryocyte-derived extracellular vesicles comprise a lipid bilayer membrane comprising CD32a. In embodiments, about 60% or less of the megakaryocyte-derived extracellular vesicles comprise a lipid bilayer membrane comprising CD32a. In embodiments, about 70% or less of the megakaryocyte-derived) extracellular vesicles comprise a lipid bilayer membrane comprising CD32a. In embodiments, about 80% or less of the megakaryocyte-derived extracellular vesicles comprise a lipid bilayer membrane comprising CD32a. In embodiments, about 90% or less of the megakaryocyte-derived extracellular vesicles comprise a lipid bilayer membrane comprising CD32a. In embodiments, about 95% or less of the megakaryocyte-derived extracellular vesicles comprise a lipid bilayer membrane comprising CD32a. In embodiments, about 99% or less of the megakaryocyte-derived extracellular vesicles comprise a lipid bilayer membrane comprising CD32a.

In embodiments, less than about 50% of the megakaryocyte-derived extracellular vesicles comprise a lipid bilayer membrane comprising CD32a. In embodiments, less than about 40% of the megakaryocyte-derived extracellular vesicles comprise a lipid bilayer membrane comprising CD32a. In embodiments, less than about 30% of the megakaryocyte-derived extracellular vesicles comprise a lipid bilayer membrane comprising CD32a. In embodiments, less than about 25% of the megakaryocyte-derived extracellular vesicles comprise a lipid bilayer membrane comprising CD32a. In embodiments, less than about 20% of the megakaryocyte-derived extracellular vesicles comprise a lipid bilayer membrane comprising CD32a. In embodiments, less than about 15% of the megakaryocyte-derived extracellular vesicles comprise a lipid bilayer membrane comprising CD32a. In embodiments, less than about 10% of the megakaryocyte-derived extracellular vesicles comprise a lipid bilayer membrane comprising CD32a. In embodiments, less than about 5% of the megakaryocyte-derived extracellular vesicles comprise a lipid bilayer membrane comprising CD32a.

In embodiments, less than about 1% of the megakaryocyte-derived extracellular vesicles comprise a lipid bilayer membrane comprising CD32a. 20) In embodiments, between about 1% to about 99% of the megakaryocyte-derived extracellular vesicles comprise a lipid bilayer membrane comprising CD32a. In embodiments, between about 1% to about 50% of the megakaryocyte-derived extracellular vesicles comprise a lipid bilayer membrane comprising CD32a. In embodiments, between about 1% to about 25% of the megakaryocyte-derived 25 extracellular vesicles comprise a lipid bilayer membrane comprising CD32a. In embodiments, between about 1% to about 10% of the megakaryocyte-derived extracellular vesicles comprise a lipid bilayer membrane comprising CD32a. In embodiments, between about 1% to about 5% of the megakaryocyte-derived extracellular vesicles comprise a lipid bilayer membrane comprising CD32a. In embodiments, between about 1% to about 2% of the megakaryocyte-derived extracellular vesicles comprise a lipid bilayer membrane comprising CD32a. In embodiments, between about 50% to about 99% of the megakaryocyte-derived extracellular vesicles comprise a lipid bilayer membrane comprising CD32a. In embodiments, between about 75% to about 99% of the megakaryocyte-derived extracellular vesicles comprise a lipid bilayer membrane comprising CD32a. In embodiments, between about 90% to about 99% of the megakaryocyte-derived extracellular vesicles comprise a lipid bilayer membrane comprising CD32a. In embodiments, between about 95% to about 99% of the megakaryocyte-derived extracellular vesicles comprise a lipid bilayer membrane comprising CD32a.

In embodiments, less than about 1%, about 1%, about 2%, about 3%, about 4%, about 5%, about 6%, about 7%, about 8%, about 9%, about 10%, about 11%, about 12%,) about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%,) about 83%, about 84%, about 85%, about 86%, about 87%, about 88%, about 89%, about 90%, about 91%, about 92%, about 93%, about 94%, about 95%, about 96%, about 97%, about 98%, about 99%, or greater than about 99% of the megakaryocyte-derived extracellular vesicles comprise a lipid bilayer membrane comprising CD32a.

In embodiments, the megakaryocyte-derived extracellular vesicles are free of, or substantially free of CD32a.

In embodiments, the megakaryocyte-derived extracellular vesicles are characterized by a higher expression and/or presence of CD32a than naturally-occurring megakaryocyte-derived extracellular vesicles, vesicles or extracellular vesicles derived from platelets such as platelet derived extracellular vesicles (PLT EVs), and/or) platelet-free plasma (PPF) megakaryocyte-derived extracellular vesicles. In embodiments, the megakaryocyte-derived extracellular vesicles have about a 2-fold, or about a 10-fold, or about a 50-fold, or about a 100-fold, or about a 300-fold, or about a 500-fold, or about a 1000-fold greater amount of CD32a than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles.

In embodiments, the megakaryocyte-derived extracellular vesicles are characterized by a lower expression and/or presence of CD32a than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles have about a 2-fold, or about a 10-fold, or about a 50-fold, or about a 100-fold, or about a 300-fold, or about a 500-fold, or about a 1000-fold lower amount of CD32a than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles.

In embodiments, the megakaryocyte-derived extracellular vesicles have about a 50-fold to about 100-fold, 128-fold to about a 512-fold, or about a 256-fold to about a 512-fold, or about a 250-fold to about a 300-fold lower amount of CD32a than platelet free plasma (PFP) MkEVs. In embodiments, the megakaryocyte-derived extracellular vesicles have about a 250-fold or about a 256-fold lower amount of CD32a than platelet free plasma (PFP) MkEVs. In embodiments, the megakaryocyte-derived extracellular vesicles have about a 250-fold to about a 400-fold, or a 256-fold to about a 512-fold lower amount of CD32a than platelet derived extracellular vesicles (PLT EVs). In embodiments, the megakaryocyte-derived extracellular vesicles have about a 256-fold or about a 300-fold lower amount of CD32a than platelet derived extracellular vesicles (PLT EVs).

In embodiments, greater than about 50% of the megakaryocyte-derived extracellular vesicles comprise a lipid bilayer membrane comprising GPVI. In embodiments, greater than about 40% of the megakaryocyte-derived extracellular vesicles comprise a lipid bilayer membrane comprising GPVI. In embodiments, greater than about 60% of the megakaryocyte-derived extracellular vesicles comprise a lipid bilayer membrane comprising GPVI. In embodiments, greater than about 70% of the megakaryocyte-derived extracellular vesicles comprise a lipid bilayer membrane comprising GPVI a. In embodiments, greater than about 80% of the megakaryocyte-derived extracellular vesicles comprise a lipid bilayer membrane comprising GPVI. In embodiments, greater than about 90% of the megakaryocyte-derived extracellular vesicles comprise a lipid bilayer membrane comprising GPVI. In embodiments, greater than about 95% of the megakaryocyte-derived extracellular vesicles comprise a lipid bilayer membrane comprising GVPI. In embodiments, greater than about 99% of the megakaryocyte-derived extracellular vesicles comprise a lipid bilayer membrane comprising GVPI.

In embodiments, about 50% or less of the megakaryocyte-derived extracellular vesicles comprise a lipid bilayer membrane comprising GVPI. In embodiments, about 40% or less of the megakaryocyte-derived extracellular vesicles comprise a lipid bilayer membrane comprising GVPI. In embodiments, about 60% or less of the megakaryocyte-derived extracellular vesicles comprise a lipid bilayer membrane comprising GVPI. In embodiments, about 70% or less of the megakaryocyte-derived extracellular vesicles comprise a lipid bilayer membrane comprising GVPI. In embodiments, about 80% or less of the megakaryocyte-derived extracellular vesicles comprise a lipid bilayer membrane comprising GVPI. In embodiments, about 90% or less of the megakaryocyte-derived extracellular vesicles comprise a lipid bilayer membrane comprising GVPI. In embodiments, about 95% or less of the megakaryocyte-derived extracellular vesicles comprise a lipid bilayer membrane comprising GVPI. In embodiments, about 99% or less of the megakaryocyte-derived extracellular vesicles comprise a lipid bilayer membrane comprising GVPI.

In embodiments, less than about 50% of the megakaryocyte-derived extracellular vesicles comprise a lipid bilayer membrane comprising GVPI. In embodiments, less than about 40% of the megakaryocyte-derived extracellular vesicles comprise a lipid bilayer membrane comprising GVPI. In embodiments, less than about 30% of the megakaryocyte-derived extracellular vesicles comprise a lipid bilayer membrane comprising GVPI. In embodiments, less than about 25% of the megakaryocyte-derived extracellular vesicles comprise a lipid bilayer membrane comprising GVPI. In embodiments, less than about 20% of the megakaryocyte-derived extracellular vesicles comprise a lipid bilayer membrane comprising GVPI. In embodiments, less than about 15% of the megakaryocyte-derived extracellular vesicles comprise a lipid bilayer membrane comprising GVPI. In embodiments, less than about 10% of the megakaryocyte-derived extracellular vesicles comprise a lipid bilayer membrane comprising GVPI. In embodiments, less than about 5% of the megakaryocyte-derived extracellular vesicles comprise a lipid bilayer membrane comprising GVPI. In embodiments, less than about 1% of the megakaryocyte-derived extracellular vesicles comprise a lipid bilayer membrane comprising GVPI.

In embodiments, between about 1% to about 99% of the megakaryocyte-derived extracellular vesicles comprise a lipid bilayer membrane comprising GVPI. In embodiments, between about 1% to about 50% of the megakaryocyte-derived extracellular vesicles comprise a lipid bilayer membrane comprising GVPI. In embodiments, between about 1% to about 25% of the megakaryocyte-derived extracellular vesicles comprise a lipid bilayer membrane comprising GVPI. In embodiments, between about 1% to about 10% of the megakaryocyte-derived extracellular vesicles comprise a lipid bilayer membrane comprising GVPI. In embodiments, between about 1% to about 5% of the megakaryocyte-derived extracellular vesicles comprise a lipid bilayer membrane comprising GVPI. In embodiments, between about 1% to about 2% of the megakaryocyte-derived extracellular vesicles comprise a lipid bilayer membrane comprising GVPI. In embodiments, between about 50% to about 99% of the megakaryocyte-derived extracellular vesicles comprise a lipid bilayer membrane comprising GVPI. In embodiments, between about 75% to about 99% of the megakaryocyte-derived extracellular vesicles comprise a lipid bilayer membrane comprising GVPI. In embodiments, between about 90% to about 99% of the megakaryocyte-derived extracellular vesicles comprise a lipid bilayer membrane comprising GVPI. In embodiments, between about 95% to about 99% of the megakaryocyte-derived extracellular vesicles comprise a lipid bilayer membrane comprising GVPI.

In embodiments, less than about 1%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or greater than about 99% of the megakaryocyte-derived extracellular vesicles comprise a lipid bilayer membrane comprising GVPI.

In embodiments, the megakaryocyte-derived extracellular vesicles are characterized by a higher expression and/or presence of GPVI than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles have about a 2-fold, or about a 10-fold, or about a 50-fold, or about a 100-fold, or about a 300-fold, or about a 500-fold, or about a 1000-fold greater amount of GPVI than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles.

In embodiments, the megakaryocyte-derived extracellular vesicles are characterized by a lower expression and/or presence of GPVI than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles have about a 2-fold, or about a 10-fold, or about a 50-fold, or about a 100-fold, or about a 300-fold, or about a 500-fold, or about a 1000-fold lower amount of GPVI than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles.

In embodiments, the megakaryocyte-derived extracellular vesicles have about an 8-fold to about a 64-fold, or about a 16-fold to about a 32-fold greater amount of GPVI than platelet free plasma (PFP) MkEVs. In embodiments, the megakaryocyte-derived extracellular vesicles have about a 30-fold or about a 32-fold greater amount of GPVI than platelet free plasma (PFP) MkEVs. In embodiments, the megakaryocyte-derived extracellular vesicles have about a 2-fold to about a 16-fold, or about a 4-fold to about an 8-fold lower amount of GPVI than platelet derived extracellular vesicles (PLT EVs). In embodiments, the megakaryocyte-derived extracellular vesicles have about a 4-fold or about a 5-fold lower amount of GPVI than platelet derived extracellular vesicles (PLT EVs).

In embodiments, the megakaryocyte-derived extracellular vesicles are free of, or substantially free of LAMP-1 (CD107A). In embodiments, the megakaryocyte-derived extracellular vesicles have less LAMP-1 (CD107A) than naturally-occurring megakaryocyte-derived extracellular vesicles and/or vesicles or extracellular vesicles derived from platelets.

In embodiments, less than about 20%, or less than about 15%, or less than about 10%, or less than about 5% of the megakaryocyte-derived extracellular vesicles comprise a lipid bilayer membrane comprising LAMP-1 (CD107A).

In embodiments, the megakaryocyte-derived extracellular vesicles are characterized by having CD62P and being free of, or substantially free of LAMP-1 (CD107A).

In embodiments, the megakaryocyte-derived extracellular vesicles are characterized by a population of megakaryocyte-derived extracellular vesicles wherein less than about 20%, or less than about 15%, or less than about 10%, or less than about 5% of the megakaryocyte-derived extracellular vesicles comprise a lipid bilayer membrane comprising LAMP-1 (CD107A) and greater than about 40%, greater than about 50%, greater than about 60%, greater than about 70%, greater than about 80%, greater than about 90%, greater than about 95%, or greater than about 99% comprises a lipid bilayer membrane comprising CD62P.

In embodiments, less than about 70%, or less than about 60%, or less than about 50%, less than about 40%, less than about 30%, less than about 20%, less than about 15%, less than about 10%, less than about 5%, or less than about 1% of the megakaryocyte-derived extracellular vesicles comprise a lipid bilayer membrane comprising phosphatidylserine (PS).

In embodiments, the megakaryocyte-derived extracellular vesicles are characterized by a lower expression and/or presence of phosphatidylserine (PS) than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles have about a 2-fold, or about a 10-fold, or about a 50-fold, or about a 100-fold, or about a 300-fold, or about a 500-fold, or about a 1000-fold lower amount of phosphatidylserine (PS) than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles.

In embodiments, the megakaryocyte-derived extracellular vesicles are characterized by being free of, or substantially free of phosphatidylserine (PS).

In embodiments, the megakaryocyte-derived extracellular vesicles are characterized by a population of megakaryocyte-derived extracellular vesicles wherein less than about 50%, less than about 40%, less than about 30%, less than about 20%, less than about 15%, less than about 10%, less than about 5%, or less than about 1% of the megakaryocyte-derived extracellular vesicles comprise a lipid bilayer membrane comprising phosphatidylserine (PS), and greater than about 40%, greater than about 50%, greater than about 60%, greater than about 70%, greater than about 80%, greater than about 90%, greater than about 95%, or greater than about 99% of the megakaryocyte-derived extracellular vesicles comprise a lipid bilayer membrane comprising CD47.

In embodiments, the megakaryocyte-derived extracellular vesicles are characterized by a higher expression and/or presence or CD41 than naturally-occurring megakaryocyte-derived extracellular vesicles and/or vesicles or extracellular vesicles derived from platelets. In embodiments, the megakaryocyte-derived extracellular vesicles have about a 2-fold, or about a 10-fold, or about a 50-fold, or about a 100-fold, or about a 300-fold, or about a 500-fold, or about a 1000-fold greater amount of CD41 than naturally-occurring megakaryocyte-derived extracellular vesicles and/or vesicles or extracellular vesicles derived from platelets.

In embodiments, the megakaryocyte-derived extracellular vesicles are characterized by a lower expression and/or presence or CD41 than naturally-occurring megakaryocyte-derived extracellular vesicles and/or vesicles or extracellular vesicles derived from platelets. In embodiments, the megakaryocyte-derived extracellular vesicles have about a 2-fold, or about a 10-fold, or about a 50-fold, or about a 100-fold, or about a 300-fold, or about a 500-fold, or about a 1000-fold lower amount of CD41 than naturally-occurring megakaryocyte-derived extracellular vesicles and/or vesicles or extracellular vesicles derived from platelets.

In embodiments, the megakaryocyte-derived extracellular vesicles contain full-length filamin A.

In embodiments, the megakaryocyte-derived extracellular vesicles comprise a lipid bilayer membrane that comprises phosphatidylserine. In embodiments, the megakaryocyte-derived extracellular vesicles are characterized by a population of megakaryocyte-derived extracellular vesicles of which greater than about 40%, greater than about 50%, greater than about 60%, greater than about 70%, greater than about 80%, greater than about 90%, greater than about 95%, or greater than about 99% comprises a lipid bilayer membrane that comprises phosphatidylserine.

In embodiments, the megakaryocyte-derived extracellular vesicles comprise a lipid bilayer membrane positive for Annexin V. For instance, Annexin V, which interacts with phosphatidylserine (PS), can be used as a surrogate for phosphatidylserine expression and/or presence or absence. In embodiments, the megakaryocyte-derived extracellular vesicles are characterized by a population of megakaryocyte-derived extracellular vesicles of which greater than about 40%, greater than about 50%, greater than about 60%, greater than about 70%, greater than about 80%, greater than about 90%, or greater than about 95% are positive for PS.

In embodiments, substantially all of the megakaryocyte-derived extracellular vesicles in the population comprise 2, 3, 4, 5, 6, 7, or 8 of Phosphatidylserine (PS), CD62P, LAMP-1 (CD107a), CD42b, CD9, CD43, CD31, and CD11b. In embodiments, substantially all of the megakaryocyte-derived extracellular vesicles in the population comprise 2, 3, or 4 of PS, CD62P, CD9, and CD11b. In embodiments, the megakaryocyte-derived extracellular vesicles have about a 2-fold, or about a 10-fold, or about a 50-fold, or about a 100-fold, or about a 300-fold, or about a 500-fold, or about a 1000-fold greater amount of one or more of Phosphatidylserine (PS), CD62P, LAMP-1 (CD107a), CD42b, CD9, CD43, CD31, and CD11b than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles are characterized by not expressing a substantial amount of DRAQ5. In embodiments, the megakaryocyte-derived extracellular vesicles are characterized by being substantially free of DRAQ5.

In embodiments, substantially all of the megakaryocyte-derived extracellular vesicles in the population comprise 2, 3, 4, 5, or 6 of Phosphatidylserine (PS), CD61, CD62P, LAMP-1 (CD107a), CLEC-2, and CD63. In embodiments, substantially all of the megakaryocyte-derived extracellular vesicles in the population comprise 2 or 3 of PS, CD61, and CD63. In embodiments, substantially all of the megakaryocyte-derived extracellular vesicles in the population comprise Phosphatidylserine (PS) and CD61. In embodiments, the megakaryocyte-derived extracellular vesicles have about a 2-fold, or about a 10-fold, or about a 50-fold, or about a 100-fold, or about a 300-fold, or about a 500-fold, or about a 1000-fold greater amount of one or more of Phosphatidylserine (PS), CD61, CD62P, LAMP-1 (CD107a), CLEC-2, and CD63 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles are characterized by not expressing a substantial amount of DRAQ5. In embodiments, the megakaryocyte-derived extracellular vesicles are characterized by being substantially free of DRAQ5.

In embodiments, substantially all of the megakaryocyte-derived extracellular vesicles in the population comprise 2, 3, 4, 5, 6, 7, or 8 of Phosphatidylserine (PS), CD62P, CLEC-2, CD9, CD31, CD147, CD32a, and GPVI. In embodiments, substantially all of the megakaryocyte-derived extracellular vesicles in the population comprise 2, 3, or 4 of Phosphatidylserine (PS), CD9, CD31, and CD147. In embodiments, the megakaryocyte-derived extracellular vesicles have about a 2-fold, or about a 10-fold, or about a 50-fold, or about a 100-fold, or about a 300-fold, or about a 500-fold, or about a 1000-fold greater amount of one or more of Phosphatidylserine (PS), CD62P, CLEC-2, CD9, CD31, CD147, CD32a, and GPVI than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles are characterized by not expressing a substantial amount of DRAQ5. In embodiments, the megakaryocyte-derived extracellular vesicles are characterized by being substantially free of DRAQ5.

In embodiments, substantially all of the megakaryocyte-derived extracellular vesicles in the population comprise 2, 3, 4, 5, of 6 of Phosphatidylserine (PS), CD62P, LAMP-1 (CD107a), CLEC-2, CD9, and CD31. In embodiments, substantially all of the megakaryocyte-derived extracellular vesicles in the population comprise 2 or 3 of Phosphatidylserine (PS), CD62P, and CD9. In embodiments, substantially all of the megakaryocyte-derived extracellular vesicles in the population comprise PS and CD9. In embodiments, the megakaryocyte-derived extracellular vesicles have about a 2-fold, or about a 10-fold, or about a 50-fold, or about a 100-fold, or about a 300-fold, or about a 500-fold, or about a 1000-fold greater amount of one or more of Phosphatidylserine (PS), CD62P, LAMP-1 (CD107a), CLEC-2, CD9, and CD31 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles are characterized by not expressing a substantial amount of DRAQ5. In embodiments, the megakaryocyte-derived extracellular vesicles are characterized by being substantially free of DRAQ5.

In embodiments, the megakaryocyte-derived extracellular vesicles and/or plurality of megakaryocyte-derived extracellular vesicles and/or population of megakaryocyte-derived extracellular vesicles comprise a lipid bilayer membrane, wherein
less than about 50%, or less than about 40%, or less than about 30%, or less than about 20%, or less than about 10%, or less than about 5% or less than about 1% of the megakaryocyte-derived extracellular vesicles comprise a lipid bilayer membrane comprising CD54, and/or
less than about 50%, or less than about 40%, or less than about 30%, or less than about 20%, or less than about 10%, or less than about 5% or less than about 1% of the megakaryocyte-derived extracellular vesicles comprise a lipid bilayer membrane comprising CD18 and/or
less than about 50%, or less than about 40%, or less than about 30%, or less than about 20%, or less than about 10%, or less than about 5% or less than about 1% of the megakaryocyte-derived extracellular vesicles comprise a lipid bilayer membrane comprising CD43 and/or
less than about 50%, or less than about 40%, or less than about 30%, or less than about 20%, or less than about 10%, or less than about 5% or less than about 1% of the megakaryocyte-derived extracellular vesicles comprise a lipid bilayer membrane comprising CD11b and/or
less than about 50%, or less than about 40%, or less than about 30%, or less than about 20%, or less than about 10%, or less than about 5% or less than about 1% of the megakaryocyte-derived extracellular vesicles comprise a lipid bilayer membrane comprising CD62P and/or
greater than about 40%, or greater than about 50%, or greater than about 60%, or greater than about 70%, or greater than about 80%, or greater than about 90%, greater than about 95%, or greater than about 99% of the megakaryocyte-derived extracellular vesicles comprise a lipid bilayer membrane comprising CD41 and/or
less than about 50%, or less than about 40%, or less than about 30%, or less than about 20%, or less than about 10%, or less than about 5% or less than about 1% of the megakaryocyte-derived extracellular vesicles comprise a lipid bilayer membrane comprising CD21 and/or
less than about 50%, or less than about 40%, or less than about 30%, or less than about 20%, or less than about 10%, or less than about 5% or less than about 1% of the megakaryocyte-derived extracellular vesicles comprise a lipid bilayer membrane comprising CD51 and/or
greater than about 40%, or greater than about 50%, or greater than about 60%, or greater than about 70%, or greater than about 80%, or greater than about 90%, greater than about 95%, or greater than about 99% of the megakaryocyte-derived extracellular vesicles comprise a lipid bilayer membrane comprising CD61 and/or
less than about 50%, or less than about 40%, or less than about 30%, or less than about 20%, or less than about 10%, or less than about 5% or less than about 1% of the megakaryocyte-derived extracellular vesicles comprise a lipid bilayer membrane comprising CD147 and/or
less than about 50%, or less than about 40%, or less than about 30%, or less than about 20%, or less than about 10%, or less than about 5% or less than about 1% of the megakaryocyte-derived extracellular vesicles comprise a lipid bilayer membrane comprising CD31 and/or
less than about 50%, or less than about 40%, or less than about 30%, or less than about 20%, or less than about 10%, or less than about 5% or less than about 1% of the megakaryocyte-derived extracellular vesicles comprise a lipid bilayer membrane comprising CD47 and/or
less than about 50%, or less than about 40%, or less than about 30%, or less than about 20%, or less than about 10%, or less than about 5% or less than about 1% of the megakaryocyte-derived extracellular vesicles comprise a lipid bilayer membrane comprising CD32a and/or
greater than about 40%, or greater than about 50%, or greater than about 60%, or greater than about 70%, or greater than about 80%, or greater than about 90%, greater than about 95%, or greater than about 99% of the megakaryocyte-derived extracellular vesicles comprise a lipid bilayer membrane comprising CD9 and/or
less than about 50%, or less than about 40%, or less than about 30%, or less than about 20%, or less than about 10%, or less than about 5% or less than about 1% of the megakaryocyte-derived extracellular vesicles comprise a lipid bilayer membrane comprising CLEC-2 and/or
less than about 50%, or less than about 40%, or less than about 30%, or less than about 20%, or less than about 10%, or less than about 5% or less than about 1% of the megakaryocyte-derived extracellular vesicles comprise a lipid bilayer membrane comprising LAMP-1 (CD107a) and/or
less than about 50%, or less than about 40%, or less than about 30%, or less than about 20%, or less than about 10%, or less than about 5% or less than about 1% of the megakaryocyte-derived extracellular vesicles comprise a lipid bilayer membrane comprising CD24b and/or
less than about 50%, or less than about 40%, or less than about 30%, or less than about 20%, or less than about 10%, or less than about 5% or less than about 1% of the megakaryocyte-derived extracellular vesicles comprise a lipid bilayer membrane comprising GVPI and/or
less than about 50%, or less than about 40%, or less than about 30%, or less than about 20%, or less than about 10%, or less than about 5% or less than about 1% of the megakaryocyte-derived extracellular vesicles comprise a lipid bilayer membrane comprising CD63, and/or
less than about 50%, or less than about 40%, or less than about 30%, or less than about 20%, or less than about 10%, or less than about 5% or less than about 1% of the megakaryocyte-derived extracellular vesicles comprise a lipid bilayer membrane comprising phosphatidylserine (PS). In embodiments, greater than about 40%, greater than about 50%, or greater than about 60%, or greater than about 70%, or greater than about 80%, or greater than about 90%, greater than about 95%, or greater than about 99% of the megakaryocyte-derived extracellular vesicles and/or plurality of megakaryocyte-derived extracellular vesicles and/or population of megakaryocyte-derived extracellular vesicles comprise a lipid bilayer membrane comprising CD41.

### Nucleic Acid-Biomarkers

In embodiments, the composition comprises substantially purified megakaryocyte-derived extracellular vesicles comprising one or more nucleic acids (e.g. at least 3, or at least 5, or at least 10, or at least 25, or at least 50, or at least 100, or at least 150, or at least 175, or at least 500, or at least 1000, or at least 1500, or at least 2000, or at least 2500), or one or more proteins (e.g. at least 3, or at least 5, or at least 10, or at least 25, or at least 50, or at least 100, or at least 150, or at least 175, or at least 500, or at least 1000, or at least 1500, or at least 2000, or at least 2500) encoded by one or more nucleic acids (e.g. at least 3, or at least 5, or at least 10, or at least 25, or at least 50, or at least 100, or at least 150, or at least 175, or at least 500, or at least 1000, or at least 1500, or at least 2000, or at least 2500) that are unique to or preferentially present in the megakaryocyte-derived extracellular vesicles as compared to other types of extracellular vesicles, wherein the one or more nucleic acids are listed in Table 6 below.

TABLE 6
Unique MkEV genes (RNA-seq)
Unique MkEV genes (RNA-seq)

STPG1, CFTR, RAD52, TMEM176A, CASP10, CAMKK1, VPS50, SLC4A1, MCUB, REXO5,
PRKAR2B, MPO, ITGAL, ABCC8, KDM7A, FARP2, MARK4, DNAH9, GAS7, MATK, CRAMP1,
CDKL5, PLEKHG6, PGLYRP1, MMP25, IL32, IYD, TRAF3IP3, PHF7, STAB1, FUZ, SLC6A13,
BTK, HFE, FMO1, MRC2, MKS1, ANOS1, MBTD1, PPP5C, CD22, LTF, ALOX5, EHD3, NUB1,
RGPD5, CNTN1, VSIG2, MARCO, PRDM11, MRE11, CYP3A43, CPS1, RTF2, NLRP2,
SLC45A4, GRAMD1B, TYMP, BTN3A1, POU2F2, ANK1, SARS1, LRRC7, SLC4A7, DAPK2,
ADSS2, STAP1, SLC18A1, CASR, MSR1, MTREX, RIPOR1, SPDL1, SLC66A1, RIPOR3,
ADAM28, JADE2, LCP2, GPM6B, XK, ARHGAP6, HDAC9, CELF2, ADAMTS6, PER3,
TNFRSF9, LTBP1, FOXP3, MCUR1, PTGER3, TNIP3, SIKE1, FNIP2, FSTL4, METTL24,
MCF2L2, KCNQ1, LY75, CHRDL2, FAM168A, KMT2C, ITIH1, TRAF1, TRAF3IP2, SERPINB3,
CDK14, NDC1, UNKL, GATB, PSD, YBX3, GNA15, QSER1, MPC1, BCAT1, GUCY1B1, DGAT2,
APPBP2, SEZ6, SLC6A16, BICRA, INTS13, TMEM161A, SNCAIP, FAR2, CHI3L2, CALCRL,
TAF11, ANKRD44, KARS1, ZC3H15, PACC1, SNAP91, COL17A1, TBC1D1, TIE1, KDM4A,
SLC9A3, CD84, SPI1, MPPED2, ACSM2B, ZFAT, DDX3Y, CACNB1, ZFY, NAV3, ATP11A,
ABCC9, PLA2G10, HDHD5, UFD1, LRP6, SPTB, DAPP1, TMEM260, FRMPD1, CFAP20,
RAD18, ATP2B1, HLTF, ADGRL1, SPP2, LNX1, UBE2D1, RHOBTB1, FCGR2B, EVC, SIDT1,
IRAG1, MOV10L1, SELENOO, CLCN4, DHRS9, PTGS2, FRY, ATP2A3, HACD3, MYDGF, TXK,
SEMA3C, VPS9D1, SEC31B, ARHGAP15, BAZ2A, SLC46A1, MBNL3, CTTNBP2, RARB,
PPP1R12B, APBB1IP, JADE1, UBE2A, N4BP2, ITM2A, NRDC, PKD2L2, CDH17, FKBP7, LXN,
OPHN1, CARMIL1, PTPRH, CRYBG3, CPB2, CFHR2, MAGI3, CXCL2, MEF2C, PTPRC, DELE1,
SLC13A1, FYB1, MPP4, EPB41L3, ERC1, TRPM3, DOP1A, TUT7, ZNF264, HAL, FAM234B,
SLCO1A2, WBP11, NKAIN1, AK6, PILRA, WNT11, USP40, CEACAM6, ERO1B, HSD17B14,
PGS1, CETP, LPCAT2, TXNDC16, RTRAF, NID2, CASS4, AAMDC, GP6, TMEM40, KIF9,
ARHGAP28, SIGLEC1, KIZ, GCN1, BRAP, HEPH, CDIP1, RCOR1, IRAK3, LYZ, CERS4, AARS1,
PITPNM2, RBM27, SLC26A4, MAP3K20, MYO15A, CCDC80, CMA1, JPH4, TRPM7, CAPN3,
AGO1, ADA2, VNN3, OR1I1, GABRP, FMO2, SUCO, PTGS1, SH2D3C, TPSD1, SMIM24,
CRISP3, PALMD, WASHC2A, STX1B, HSD3B7, ATP5F1D, MARCHF2, CECR2, GGT5, CYTH4,
ESS2, GRK3, PIK3IP1, PATZ1, CRYBB1, SNU13, SEPTIN3, DMC1, RTCB, HMGXB4, TOM1,
NEFH, CABP7, GRAP2, NCF4, CSF2RB, CHADL, ZC3H7B, CDKL1, DDHD1, CNIH1, TMED8,
RIN3, SUSD6, SLC10A1, SLC8A3, GSKIP, PPP4R3A, PRORP, GINS1, CD40, SGK2, SLA2,
RAB5IF, NFATC2, NELFCD, PRELID3B, MRGBP, GID8, ANGPT4, SNPH, SIRPB1, PLCB4, HCK,
TLDC2, MROH8, RPRD1B, BPI, EPPIN, MAP1LC3A, TNNC2, APMAP, CELF4, POLI, TLR8,
SYP, FMR1, EEA1, JADE3, PCYT1B, CD40LG, ZDHHC15, TAF7L, PBDC1, RUBCNL, HTR2A,
CDADC1, FLT1, VWA8, OLFM4, MT4, TSNAXIP1, CCL22, USB1, SMPD3, ESRP2, WFDC1,
SEC14L5, ABCC1, CIAO3, CAPN15, EARS2, ELOB, XYLT1, VPS35L, KNOP1, AQP9, IQCH,
ACSBG1, CD276, ZNF106, BMF, DMXL2, SLC30A4, EYA1, TRPA1, CPQ, BPNT2, TRPS1,
ANXA13, RETN, TLE6, CCDC61, RASAL3, SLC1A6, CLC, EBI3, YJU2, APLP1, DMAC2,
CEACAM4, ICAM4, NOP53, NKG7, ICAM5, CD33, CRX, CCDC114, RAB3D, CAPS, FAM83E,
KLF1, MAST1, KCNN1, UPK1A, ETV2, HPN, SCN1B, CFAP69, DNAJC2, PIK3CG, ITGB8,
GSDME, TFEC, H2AZ2, RNF32, CPED1, MINDY4, PTPRZ1, HYAL4, USP42, ZNF862, SFRP4,
POU6F2, TMEM176B, MYL7, GALNTL5, CNTNAP3, PRUNE2, TRIM14, OGN, ENG, RIC1,
KCNT1, MPDZ, GLIS3, ABHD17B, GATA3, DNMBP, CXCL12, VSIR, CCSER2, STN1, TASOR2,
SHOC2, DNAJC12, ASPA, SEPTIN4, SLC6A4, MED31, C17orf75, CCL8, PEX12, ABI3,
PTGES3L-AARSD1, ALOX12, TMEM33, SLAIN2, CRACD, PF4V1, GAB1, GALNT7, CLNK,
NSD2, BST1, RAPGEF2, LRP2BP, KLF3, ZBTB16, ZPR1, TECTA, SC5D, JHY, VWA5A, KMT5B,
MS4A6A, HPX, FOLR3, SLC15A3, C11orf21, SOX6, POU2AF1, CD69, CLEC2B, BIN2, TNS2,
SLC6A12, GPN3, AKAP3, GSG1, OGFOD2, CLEC4A, C2CD5, COL12A1, FRK, MAK, SMIM8,
RIPOR2, OPRM1, RHAG, PHACTR1, CILK1, MDN1, FBXL4, VNN1, ADGRG6, BICRAL,
DNPH1, IMPG1, TENT5A, ERBIN, TENT4A, CERT1, PDE8B, HAVCR1, ITK, GABRG2,
POLR3G, CDH6, NPR3, TARS1, ST8SIA4, FGF1, NR3C1, TRAPPC13, C9, SEC24A, RARS1,
MACROH2A1, SELENOK, GNAT1, HHLA2, MORC1, CFAP92, NEK11, PLSCR4, HEMK1,
PEX5L, PLCH1, NEK4, MOB1A, PIKFYVE, SF3B6, CYTIP, CLIP4, PAPOLG, IL1R2, IL1RL1,
RMDN2, DARS1, CEP104, OPRD1, EDEM3, ASH1L, C1orf21, NCF2, PRDM2, RO60, NR5A2,
ADPRS, WARS2, NID1, MYCL, SLAMF1, CD48, PADI2, GBP3, RAB29, P3H1, PTCH2, FASLG,
TNFSF4, RSRP1, KMT2A, TREH, MMP8, KLF7, TTR, CA14, IRAG2, ADGB, ALDH8A1, TCF21,
SLC16A7, RXYLT1, RARRES1, EEF2KMT, LDAH, FGF23, ABITRAM, PTPA, NR4A3, CSF3R,
YLPM1, BBOF1, NEK9, ABCD4, NRDE2, SUPT7L, ATAD2B, FKBP1B, CNRIP1, SLF2,
DENND10, KCNIP2, CFAP58, C10orf95, TEK, INSL6, CD274, PLEKHG1, MYCT1, PLXDC2,
MTERF2, CHRNA2, EPX, TMEM131L, TENT4B, IAPP, ZNF549, ZSCAN18, RNF2, DPPA4,
NAA50, KIF18A, GJB6, ADGRB2, FABP3, CCRL2, ZNF639, ZSCAN20, CSMD2, LRIF1, POLK,
CD244, LY9, HS3ST2, SHLD2, PRXL2A, LRRC39, SLC66A2, SEPTIN7, NT5C3A, SRGN,
HVCN1, SPRYD7, ITIH5, NCKAP1L, NR4A1, EEF1AKMT3, USP45, TNFAIP6, GOS2, C4BPB,
RAB38, AGO2, FAM124B, ENKD1, CTCFL, SEMG2, ATP5F1E, CDH26, ZBP1, IL9R, POF1B,
CEACAM8, GRM4, H1-3, H1-1, H2BC11, TREM1, KCNK17, RREB1, CXCL6, EREG, SSUH2,
HROB, SEPTIN6, AMELX, DMAC2L, GRK4, FNDC11, IL1B, CD93, PAX1, MGME1, OTOR,
SIRPD, AGO3, PRRG2, SCAF1, PRR12, PCNX4, PZP, ARMCX1, TMEM35A, ZC4H2, INTS11,
RGS13, MASP1, TSPAN8, AUNIP, ADGRE2, OR7C1, F2RL3, METTL25, GNGT1, FGL2,
STEAP4, KDR, IGLL1, C22orf23, POT1, LSM8, STRIP2, IFT22, LRRC4, IRF5, LRRC17,
PDE11A, ARHGAP22, WDFY4, TMOD2, TTBK2, CCDC32, ICE2, ALDH1A2, VPS13C, KCNC1,
PIMREG, ATP1B2, AP1M2, SIGLEC9, RIPK3, HEATR5A, QRICH2, ARHGEF6, SGO1, PGAP6,
ABHD17A, LBP, CBFA2T3, ERMARD, GALNT8, NECTIN2, XPO7, COLGALT1, QRSL1,
MTRES1, AFDN, IQCN, SULT4A1, SAG, HELZ2, LSP1, TNNT3, HBZ, METTL26, ZC3H4,
ATP5IF1, THEMIS2, PPIL4, ULBP3, LATS1, AAR2, ZNF141, GSE1, ADGRE3, RBSN, GALNT15,
KIF3A, G6PC, CA6, ZNF304, SELENOS, PODNL1, TRIM22, TIMM10B, CLUH, DDC, H3-3B,
RIDA, VPS13B, MATN2, MTSS2, KHDC4, TESMIN, ZSWIM3, OSER1, DMGDH, PATJ,
FBXO44, CPLANE2, PDE6A, PIK3C2B, SPART, GPALPP1, STARD13, SLF1, CEP20, SLC2A11,
C1QTNF6, ATP13A3, CA1, LYVE1, SARAF, ERG28, DGLUCY, ADAMDEC1, KATNBL1,
GNAT2, VAV3, CHIA, PTPN22, LAMTOR5, CMPK2, IAH1, FST, NARS1, RELCH, FBH1,
ECHDC3, HRH4, RERG, KLRD1, SPX, LRP4, RTL8C, RAB33A, STK26, TUT4, C5AR2, PDGFRA,
GGACT, SLC37A2, APC, CEMIP2, HAVCR2, OASL, CCDC146, CD36, UGT2B28, MDFIC,
ANKRD6, LCA5, AMHR2, TESPA1, AGAP2, FAIM2, AFG1L, TEC, MDM2, PCNX2, RGS8,
RNASEL, NIBAN1, RC3H1, PAX3, DOCK10, SLC19A3, ANKRD36, PLXNC1, WASHC4, LRCH1,
EDNRB, RCBTB2, LCP1, RAPGEF5, NUP42, AOAH, HUS1, CIDEB, ZFHX2, TM6SF1,
MYCBPAP, LIMD2, MARCHF7, SCN7A, EPRS1, HLX, GAD2, ECPAS, NIBAN2, TLR4, ALDOB,
GARNL3, GABBR2, HEMGN, NR5A1, TRMO, ENPP2, IL33, DMAC1, FOXP4, ZSCAN9,
CAPN11, TLR2, ANKRD42, LRRC32, TMPRSS4, TRPC6, C11orf1, TMPRSS13, CASP1, SQOR,
KNL1, RMDN3, DUOX1, SLC28A2, SEMA6D, SPTBN5, IFI44L, SELENOI, SLC3A1, ATRAID,
CYP2C9, MFSD13A, ENTPD1, PLCE1, FAM149B1, CARF, SLC49A4, APH1B, PPCDC,
SEMA7A, FAM13A, HERC3, ZGRF1, RASGEF1B, MMRN1, SEPTIN11, CNOT6L, EGF, FBN2,
GUCD1, ALG10, PRICKLE1, AMIGO2, COL2A1, PPFIA2, MARCHF9, TPH2, TMEM19, RITA1,
NUP58, CCDC65, RDH16, GPR84, N4BP2L1, BRCA2, MYG1, SRRM4, ABHD13, TTC6, ESR2,
STON2, EFCAB11, GPR65, PTGR2, WARS1, SLC12A6, ZSCAN29, FGF7, TLNRD1, CYP1A1,
ZNF710, CRTC3, EFL1, RUSF1, IGSF6, MYLK3, CPNE2, MEAK7, RPL3L, ZSCAN32, GID4,
ZNF287, KSR1, UTP4, VPS53, ABCA8, C17orf64, BCAS3, ADCYAP1, RMC1, CCDC40, TTYH2,
SECTM1, CEP131, ZNF750, WDR45B, RNF165, ELAC1, MBD1, TNFRSF11A, P3H4,
CACNA1A, MISP3, TPGS1, VAV1, MVB12A, TMEM91, ZFP14, SIRT3, TRPM2, DOP1B,
ADAMTS10, MYO1F, ERVK3-1, NLRP12, TIMM29, SIGLEC10, SLC2A5, EVA1B, STRIP1,
CD53, MPC2, TBX19, XCL1, NR1I3, HMCN1, ZNF687, ANP32E, MINDY1, ANXA9, SEMA6C,
KCNH1, S100A8, CEP170, SDE2, REN, HNRNPLL, EXOC6B, ST6GAL2, LNPK, TMEFF2,
UNC80, KANSL1L, RETREG2, CNOT9, CNTN4, GADL1, ITGA9, STAC, NXPE3, RUBCN, AMT,
CENPC, SLC10A4, PLAC8, SNCA, PYURF, SPATA5, FABP2, MARCHF1, RNF175, PDGFC,
ROPN1L, MARCHF6, ADAMTS16, OTULINL, HAPLN1, BHMT, PPIP5K2, TNFAIP8, LECT2,
GABRB2, PLA2G7, MMUT, DOK3, ABT1, SCML4, ARMT1, CREB5, NIPSNAP2, TMEM140,
RAB19, LANCL3, DIPK2B, ZNF182, KRBOX4, GPR174, ZNF711, NXF3, RADX, IGSF1,
CSGALNACT1, DOK2, PLPBP, SNTG1, RGS20, PLPP5, NSD3, ADHFE1, ATP6V0D2,
MFSD14B, AOPEP, NR6A1, WDR31, GBGT1, PAXX, DPH7, USP6NL, MINDY3, ST8SIA6,
FAM13C, NRBF2, CDHR1, RGR, LRMDA, LRRC27, CNNM2, ADAM12, SERPING1, HIKESHI,
CCDC82, ZC3H12C, DRD2, SLX4IP, GRIK4, TKFC, TMC2, INCENP, MS4A3, CPSF7, MS4A2,
CNBD2, LSM14B, FERMT3, VPS51, TLCD3B, CNKSR2, CLEC1A, MPP7, FCGR1A, CDH8,
TMEM218, MIA2, ADRA2A, GPM6A, WDR17, CD226, RGS18, ATPSCKMT, NKAPD1,
ABCB9, RILPL2, NOCT, ENKUR, GPR158, TMEM263, IPMK, PLBD2, SLC2A13, DIP2C,
MIPOL1, VIPAS39, QTRT2, AKR1C2, ADAM8, KIN, KCNJ1, CENPU, ZNF385D, GABRA2,
TMEM267, KCNE4, AP1S3, GPATCH11, OBI1, ARL14EP, EPG5, ATP5F1A, PDK1, GUCY1A2,
XRCC4, ZNF776, OLAH, PLEKHH2, SPEF2, NADK2, WDR78, HNRNPDL, ZNF117, PLOD2,
NRSN1, CPB1, ACMSD, PTPRR, CD96, ADGRF1, CYRIB, FAM81B, UBALD1, SPACA7,
TMPRSS11D, SPHKAP, ZNF599, LGI4, GRAP, CABYR, IMPACT, ANKH, TOMM70, ABI3BP,
ANGPT1, ABCA9, ABCA10, ABCA5, MIA3, DIPK1A, ATP5MC3, ELOC, LY96, PSMA8,
TMSB4Y, JAM2, ATP5PF, SLFN13, PIEZO2, SEPTIN14, AK9, PIP4P2, HSPA13, SAMSN1,
GRAMD2B, ZCCHC10, NIFK, LRGUK, MIER3, TTN, VSIG4, FLACC1, C2CD6, ELMO1, SAXO1,
PXYLP1, SLA, CLIC2, AFF2, KIF5A, CARNMT1, MIDEAS, C8orf37, CFAP161, ADAMTSL3,
ART3, ANKRD9, ATP5MPL, MTERF3, PPP2R2B, SAMD8, UNC5D, GLYATL2, TBC1D31,
NTAQ1, FRRS1, MFSD14A, SASS6, B3GNT7, MPV17L, BRPF1, ATP2B2, SLC6A1, RBPMS,
KLHL40, NRG1, ODR4, FCSK, CACNA1D, KIT, ZNF19, KCNJ15, TSPAN18, LCA5L,
PALM2AKAP2, DGKI, TMEM268, SVOPL, SLC34A2, CABP1, PRXL2B, MEGF11, ANKRD61,
BABAM2, CFAP251, UBXN11, GALNT14, PRXL2C, TENT5B, H2BC5, CATIP, CNOT11, CD1C,
NCF1, DYNC1I1, ALAS2, PPP1R15B, PKD1L1, SLAMF8, ITLN2, DMTN, TOMM40L, CCAR2,
CFAP298, PAXBP1, C1QC, ATP5MC1, CIART, GOLGA6A, M1AP, DRC7, ZDHHC1, ZFYVE28,
CARMIL2, PSKH1, ZNF221, CCDC28B, UBASH3A, GATD3A, CFAP410, ZNF66, SPATC1L,
S100B, ZNF208, JAML, S100A1, ZBTB7B, IL6R, MYL3, LRRC71, PWWP3A, CELF5, NPHS1,
ZNF382, SIGLEC11, MPP3, FAM171A2, FMNL3, BCL6B, ASGR2, TNFSF13, CCDC42,
TBC1D24, NEU3, CYB561A3, PPP1R32, LBHD1, PDZK1IP1, TAL1, COA7, CZIB, PLPP3,
MATN1, TSSK3, MYSM1, ADGRL4, LRRIQ3, WDR63, KCNT2, NLRP3, SLAMF6, FCGR3B,
AXDND1, KIF26B, KLHDC8A, REL, MEMO1, TYW5, MAIP1, SLC66A3, KCNJ3, DUSP19,
CFAP36, WDCP, H3-3A, COQ8A, XIRP2, CTSS, BNIPL, TNFAIP8L2, TMOD4, DHX57,
S100A12, ABRAXAS1, GPR155, PBXIP1, INAVA, NAXE, SLC22A15, SLC15A2, PROK2,
TMEM169, NEK10, CIP2A, FBLN2, SGO2, SERPINI1, SPTA1, MNDA, PPM1L, NEPRO,
SPICE1, CCDC191, CADPS, WDFY3, COX18, PRICKLE2, ADAMTS9, SMIM14, CFAP20DC,
CXCL5, PF4, CPA3, CCR1, ELP6, FBXO40, LIPH, RPL39L, SLC51A, RNF168, EXO5, EMCN,
SLC9B1, CAMP, INTU, ABHD18, DUSP7, ETNPPL, MAP9, GUCY1A1, CEP44, HPGD,
C4orf45, TMEM144, NPY1R, ICE1, HHIP, TMEM184C, PRMT9, TMEM161B, ZNF474,
LMBRD2, ADGRV1, F2RL2, SPINK1, CDC20B, ARSK, TIGD6, PRIMPOL, ERAP1, CFAP97,
SEPTIN8, CGAS, SAMD3, IL31RA, ANKRD55, RAET1E, BMT2, KIF6, ELAPOR2, IQUB,
TAGAP, ADCY1, PHKG1, KCNV1, CSMD3, SPIDR, DNAAF5, GPR146, UNCX, VIRMA,
TMEM67, MYORG, CCDC171, ABCA1, C8orf34, KDM1B, HGSNAT, RASEF, CYBB,
METTL27, CARD19, ATP7A, ZNF367, AQP7, INTS6L, MARCHF8, DDIAS, EML5, AKR1E2,
FAAH2, ATP5F1C, FAM204A, CLEC1B, ENTR1, AK8, SPACA9, DIPK1B, JCAD, PIP4P1,
NSMF, C12orf50, CCDC186, TRUB1, CYP2C19, C10orf82, HSPA12A, TTC7B, IFI27, CLMN,
CACNB2, HACD1, SMCO4, CEP295, AMOTL1, LIPC, TCP11L2, GPR176, CMTM5, GLB1L3,
AVPR1A, C16orf71, BORCS7, ATP9B, GARRE1, TRIM66, DENND2B, CDYL2, PRTG,
C16orf46, HDGFL3, CCDC68, CLEC4E, CLEC4D, CIAO2B, CFAP52, BEX3, GOLM2, BMERB1,
MARF1, LDHAL6A, PCLAF, LDHD, CHP2, NEMP1, C15orf48, MS4A7, MS4A14, EPB42,
MEI1, B4GALNT2, SAMD14, PIP5KL1, SLC27A4, LOXHD1, DPEP2, DUS2, ENGASE,
RBFOX3, ATP5MG, OR51I1, FN3K, GNG8, LPO, JSRP1, NIBAN3, SPATA33, KMT2D,
ZNF528, PROSER3, LAIR1, TMIGD2, HDGFL2, SLC43A2, KRT1, CD300C, MRPL58,
TMEM88, NLRC3, BEST1, SPINDOC, ATG16L2, TRANK1, ENTPD3, NAALADL1, CCDC88B,
PTGDR, COA6, H1-4, DEGS2, SCN11A, SEPTIN2, ING5, RHOH, CAVIN2, MTCL1, CHRM1,
GFRA2, ADAM9, IL7R, LRP1B, TET2, PPIP5K1, ZNF507, NSG1, SIGLEC7, GSG1L, MN1,
NPIPB12, GCSAML, NPIPB3, CXCL11, ADRB2, SHE, P2RY12, OR5AU1, RNASE2, RSPH10B2,
PTAFR, CXCL8, HTRA4, GPR183, ZEB2, DTWD2, RAMAC, PROKR1, RGPD8, HEXD, CENPX,
TMEM266, CSGALNACT2, ROBO1, P2RY1, AHSP, ITGAM, ZNF764, TMEM154, SIMC1,
GYPA, ADPRM, FAXDC2, ELP5, TCAF2, ZNF804A, RIOX1, PYM1, MZB1, SLC23A1, EMB,
DLGAP1, ATF7, POLH, HTRA3, CEL, RNF139, TANC2, OR1M1, S1PR1, PKIA, FPR2, FPR1,
OR2M4, LRG1, SOSTDC1, FAM98B, ZNF440, DSEL, COL24A1, LPAR3, PTGER4, OR2AT4,
NMUR1, PTCRA, PAH, GATM, IFNB1, MRM3, FBXW10, DRC3, ZNF554, USP19, SLFN12,
FRMD3, AZU1, TPSAB1, CLEC7A, NEGR1, MACROD2, COPRS, OR5A1, CLEC12A, OR5A2,
CFAP53, PRSS27, RSPH9, COPS9, TRIM40, CTSW, PDE3A, RASGRP1, KLHL6, RND1,
AGAP5, WASHC2C, FUT10, OR4D9, ZNF596, EFCAB12, PSME3IP1, CBWD1, RAB37,
SNX32, RARG, CES3, PHF8, ADH6, ARPP21, TADA2B, GRK2, HECTD4, HPSE, MRPL57,
CYSLTR1, PARP15, ABLIM3, MFSD4B, SYT12, ZBTB21, PPP1R3B, OR10K1, NDNF, IHO1,
MICOS10, EHBP1L1, THAP2, ZNRD2, TNFRSF10C, CEP83, SULT1B1, UGT2A1, TRAPPC3L,
APOBEC4, BCLAF3, CD7, ATP5MD, MARCHF3, NRROS, FBXO45, C9orf131, CD34, TLR6,
TRMT10C, SELP, SNX31, ZNF80, SLC16A13, STARD6, CNTNAP2, GCSAM, MYO1H, MSL2,
ZNF497, OR7D4, B4GAT1, FAM241A, WDR49, PCP2, ADGRE1, DENND6A, CATSPERD,
P2RY14, GPR171, FBXW8, CA5A, TVP23C, NPPA, CENPS, PHYKPL, APOF, ARL10, PCSK1,
LPL, LRRC25, TSGA10IP, LIPT2, KCNE3, ERCC4, MTLN, EIF4E1B, ZNF169, GAPT, CALCB,
ZDHHC21, TSPEAR, ENTHD1, LRRTM4, SMIM19, RTTN, OR4K17, ZNF135, SYNE3, PLAAT3,
USF3, RMDN1, OR4F17, RBIS, NCKAP5, LRRC37A3, ANKLE2, ACER2, PHETA2, FAM9B,
NUDT4B, OR2T12, GATD1, TRIM72, KCNA3, BEND2, SAMD9L, MIEF2, NAP1L5, OR2T8,
CAVIN1, RABEP2, SAMD12, CD163, C18orf32, HASPIN, PGBD5, CRACR2B, NECTIN3,
TMEM94, SOX12, ZNF619, AP3S1, DPY19L2, ADAMTSL1, CALHM5, ZNF518B, ZNF366,
POGLUT3, SPAG11A, GEN1, ZNF354B, KCNG2, KLHL11, CD28, DYNAP, THBD, GP5,
EFCAB13, MSC, DPY19L3, ZNF852, CYBC1, ZNF552, SELENOW, SNX18, EXOC3L1, IGSF22,
PER1, TCAIM, PXT1, MKRN3, ZBTB18, CIITA, OR6C4, FCER1A, PCED1B, AKAP5, GIPC3,
ZNF154, NRXN1, ZNF771, C3orf80, WFDC11, SEPTIN1, TDRP, MYLPF, TIGD2, HCLS1,
MTURN, ARHGAP45, ZNF571, MIGA1, KCNE1, H2BC4, TMEM64, CXCR2, CMTR2, ST20,
OR52E4, BBS12, ZFP82, LSMEM1, SLC25A42, EHMT1, F2R, H2AW, TMEM102, NME9,
OGFOD3, WASHC1, ZNF678, P2RY13, ZFP41, ZNF875, DIPK2A, MACIR, OR5AS1, SLC9A9,
RELL1, COA4, TMEM259, GET1, ERCC6L2, P2RY8, NLRP10, SPACA6, ZSCAN22, KIAA2012,
PGBD4, FES, SATB1, EPGN, NDN, PPP1R27, BRICD5, RGS6, SLC35D3, PAPPA, ADGRG3,
SRPRA, ZNF320, SPNS2, MCEMP1, CSMD1, PTGDR2, GABRR3, MICA, CCDC60, PLGLB1,
CCBE1, SELENOF, ZNF623, C19orf71, GPR132, MX2, TENT5C, PRR14L, H3C13, HMCES,
RP1L1, ZNF530, RFLNB, NOG, MACC1, ACSM2A, CHEK2, TUBA8, CCR4, BTBD9, ANKRD45,
UTY, SDR42E2, KMT5A, CLDN5, OR1D2, GOLGA6L4, CMSS1, PCDH9, H2AC20, H2AC21,
SRPK3, H1-5, CSF1, MAML2, BPIFC, PROS1, STING1, PDE4B, KRBA2, SEPTIN9, H2BC21,
SEPTIN5, APOBR, SMIM10, SATL1, H1-10, RBM43, CHRM5, TMEM106A, NELFA,
SLC24A3, EFCAB10, ADSS1, H2BC13, PRSS57, ZNF445, TCEAL9, RAB11B, PRPF39, ZNF74,
UBALD2, KLHL33, ZBTB37, NUPR2, IL3RA, ARL15, PRKN, TEDC1, SP140L, TARS3,
METTL7A, ZSWIM9, GPRIN3, STAC3, PRKG1, DLK1, TLCD2, LSAMP, INKA1, PCGF3, LMLN,
SHC4, SYN3, MOSMO, ADARB2, C11orf87, IFIT1, IKZF1, DNAH14, EVI2B, NPIPB4, ZNF829,
TMPRSS11B, POMK, C16orf54, KLHDC8B, ZNF267, ZNF566, CD300LF, AGBL4, ARGFX,
LRRC70, ZBTB6, KIF18B, ZNRF1, BPIFB3, MTARC1, BLOC1S4, ZNF749, MRTFB, ZNF555,
TMEM212, GOLGA8R, CCDC30, ZNF501, GNG2, SEPTIN10, SMYD4, SMIM29, PDE2A,
PRG2, CYP27C1, OR10H1, SPIN2B, ZSCAN30, TRABD2A, PPARA, KRTAP13-4, EMID1, RHD,
ENTPD5, AL162231.1, TSPYL4, SESTD1, FAM9C, MAGI2, H1-6, GJA4, IFT140, AGMO,
TLR5, TET3, VMAC, DNAJB13, GABRD, FANCA, FANCM, ZNF70, TMEM220, H1-2, OR6C75,
CCDC157, CLEC17A, PLA2G2C, OR7D2, TPRG1, ZNF490, CLCN1, TMPPE, ZC3H6,
LAMTOR4, POTEE, ZNF793, AGAP4, PEAK3, GPR21, TCTEX1D4, SELL, BLOC1S5, H2AX,
CCDC9B, RTL6, DPYD, RHCE, C2orf80, SMIM15, FSIP2, SNTN, CALHM6, TMC3, FBF1,
MSL1, INSYN2A, HACD4, UTS2B, NELFB, ZNF292, ADAT2, APOD, H1-0, BLOC1S3,
FAM47E, JPT1, NUGGC, GJB5, SLC35E2B, NEMP2, IL1RAP, MYBPC1, ZNF676, ZNF699,
CCDC189, OR8A1, KIF19, SRGAP3, SULT1C3, OR2C3, ZNF107, ZNF471, IARS1, SRGAP2B,
PRTN3, CRYBA4, NPIPB15, ZNF605, C20orf96, ESRRG, BORCS6, MME, CACNA1H, HBG2,
LAMA2, MRTFA, SLC22A25, SDHAF3, RABL6, HSH2D, NF1, DAPK1, H2AC13, OR52K1,
H2AC11, CHRNG, MVB12B, ILRUN, POTEI, H2AC7, H2BU1, WDR5B, EOLA2, H4C3,
ZSCAN26, ZNF772, PCNX3, ADAM32, LRRC8B, ABCB8, H3C12, NOL4L, FITM2, FBXL22,
DACT3, H3C4, VEPH1, GYPE, OR6Y1, ELANE, ZNF624, KCNMB2, CPLANE1, ZNF841,
EOLA1, SERPINB2, DPP4, DNAH10, KRTAP26-1, ZNF460, CFAP43, LHFPL5, TXNRD3,
STPG3, MAP1LC3C, EME2, ZNF81, ZNF780A, SGTB, ZNF790, H2BC12, ZNF677, ERO1A,
ZNF347, FCHSD1, ZNF71, KEL, ZNF667, MARCHF5, NPIPB13, ZBTB14, ADH4, OR2L3,
RPS6KL1, GET3, OGA, TCAF1, OR14L1P, ZNF568, ZNF43, C20orf203, CTNND1, RYR2, TAT,
TAFA2, TUSC1, EIF1AY, MT-ND6, MT-CO2, TOGARAM1, MT-CYB, LDB1, F5, PLXNB3, MT-
ND2, SYCP1, RCSD1, MT-ND5, BRINP2, MT-CO1, LRRC10, SUCNR1, SELENOM, MT-ND3,
SELENOT, TOX, MT-ND4, MT-ND1, CIPC, MT-ATP6, SPOUT1, MT-CO3, SGMS1, C6orf47,
OR2L2, CHML, STUM, FCGR3A, H3C14, H2AC18, H3C15, HSD3B1, SAMD13, NEU4, CHIC1,
NHSL2, AGAP9, MACO1, PTPN20, OXLD1, NBDY, PJVK, SMIM5, NELFE, LY6G6F, AIF1,
ZNF805, KRTAP5-11, ASPDH, OR14J1, SPDYC, MALRD1, MROH6, IGFL4, SPINK9, UQCC3,
FBXO48, CD177, ZNF783, PKHD1L1, TMEM231, ACCSL, TRIQK, ZBTB10, TMEM170B,
ADGRG1, SAMD9, EXOC3L4, TP53TG3D, TMEM256, DENND6B, MFSD2B, HTN3, DPF3,
ETFRF1, CYS1, PLPP6, OLIG2, NYNRIN, GOLGA8O, ANKUB1, B3GALT4, HLA-DRA, SNHG32,
RAB12, CD200R1L, LNP1, COLQ, SELENOH, RNF208, MT-ND4L, DENND1B, CEP43,
TRIM59, OR2F1, GSTM2, ERV3-1, GBP7, RBMXL1, STIMATE, TREX1, UGT2B11, ZNF888,
LTB4R, HBG1, ITGA1, NUDT19, ZNF726, GANC, ZNF891, SMIM30, C19orf38, BBIP1,
FAM187A, PLIN5, SEC14L6, NUTM2D, B3GNT10, ZSWIM8, TOMM6, MS4A4E, TBC1D26,
UBXN2B, GOLGA8B, RPL17-C18orf32, SLC35E2A, IGSF23, CDC14C, CEBPZOS, LINGO3,
PLXNA4, ZNF880, BTBD19, TRIM49D1, ZNF844, TINCR, CPTP, HLA-DPA1, PRR29,
SMIM13, FAM236D, Z82206.1, MCRIP1, HLA-DQA1, LY6G6E, TMEM185B, C13orf42,
AKR1B15, MRLN, OR2M3, MT-ATP8, ORM2, FAM174C, NUTM2E, FNDC10, ARL17B,
PGA4, ORM1, PATL2, PET100, IQCF3, OR5H6, ANKRD18B, HLA-DPB1, STMND1,
CFAP97D1, TSPAN19, PRH1, SMIM26, AJM1, LYRM9, NPIPA9, USP17L11, NPIPB7,
AC092835.1, H2BC15, ZNF879, PINLYP, NPIPB2, IQCM, BRD3OS, SMIM1, HGH1,
SMIM27, ANKRD34C, KIAA0040, SLFN14, C3orf84, C4orf51, ZNF737, LRRC37A2, OR2W3,
DEFA3, TEX35, PLCXD2, TDGF1, AC244197.3, UGT1A1, HYPK, ARPIN, SERPINB10,
CCDC169, STRC, NAA80, ZNF487, LEFTY1, TMEM35B, UGT1A7, CCDC13, H2AJ, INSL3,
APELA, PCP4L1, USP17L22, LGALS16, TERB1, ARPIN-AP3S2, TRMT9B, ZNF718, GYPB,
AC068580.4, SELENOP, ZNF345, ALG1L2, MSANTD3-TMEFF1, C8orf88, CCDC71L,
TMEM200B, SIGLEC14, AP002495.1, SIGLEC12, FPGT, IGLL5, HTD2, NOX5, HCAR3,
AC005833.1, MTRNR2L8, AL117348.2, COA8, TRAPPC2B, SMIM3, ZNF225, AP002748.5,
AP003419.1, SMIM10L1, MTRNR2L1, HP, MGAM, RPL36A-HNRNPH2, INAFM1, TEN1,
CLEC5A, AP001781.2, TRIM34, AC007040.2, AL355916.3, SMIM6, INAFM2, ST20-MTHFS,
AC090527.3, FRRS1L, AL049634.2, AC026464.4, AC140504.1, CLEC19A, ZNF865,
PECAM1, GOLGA6L7, BOLA2-SMG1P6, GFY, LINC00514, AC233723.1, CCNQ, MYZAP,
DPEP2NB, LINC00672, RHEX, GJA5, AC134669.1, ANXA8, FXYD1, AC006538.2, PTGES3L,
AC012309.1, UPK3BL1, FDX2, EDDM13, AC004076.1, SMIM17, AC018755.2,
AC006486.1, AC010522.1, MTRNR2L12, ZNF587B, SLC25A53, NCBP2AS2, HSPE1-MOB4,
RPS10-NUDT3, H4C14, NBPF19, CCL5, MILR1, H2AC19, KMT2B, CD24, GRIN2B, NBPF26,
H4C12, H2BC14, FCAR, SMN2, H2BC8, AC244489.1, USP27X, H3C8, LILRA2, CASTOR2,
LILRA5, MYOM2, NATD1, CCL3, H2BC6, TBC1D3L, LILRB2, RCC1L, H4C6, H2BC17, H3C6,
BDP1, H2AC12, PPP4R3B, H4C13, H2AC15, CCL4L2, H3C11, FCGBP, MUC20, FP565260.1,
H2BC9, H3C1, AC091959.3, TMEM251, AC139677.1, SPATA31A7, MAPT, H4C9, PIK3R6,
AC011043.1, H2AC14, H2BC3, AC245049.1, H2AC16, H4C5, H2AC8, MMEL1, H4C4,
H2BC7, H3C7, ZNF8, CARS1, LENG9, H2AC4, H2BC10, NAIP, C11orf98, MRM1, H4C1,
H2AC17, IQCA1L, BX004987.1, H4C2, H3C10, TSTD3, OR2L8, AC013394.1, OR51F1,
GATD3B, FP565260.6, CCDC163, PAGR1, ZNF546, AC243733.1, HNRNPCL4, NSMCE3,
AL157392.5, AC058822.1, LBHD2, C13orf46, AC001226.2, TEX54, FAM236C, MYOCOS,
VSIG10L2, AC007326.4, AC270107.1, AC270107.2, PLCL2, AC270107.3, AC270107.5,
AC270107.6, AC270107.7, AC025287.4, AC015802.6, AL022318.4, SCYGR4, EEF1AKMT4,
CCDC39, AP000812.5, AC270301.3, AC104389.6, BX255925.3, UPK3BL2, AC093512.2,
AC022506.1, DEFA1B, AC026316.4, AC090517.4, ABCF2-H2BE1, FO393400.1,
AC093827.5, USP17L7, AC097636.1, AC024598.1, AC072022.2, AL135905.2,
NOTCH2NLB, SPEGNB, AC008770.4, DERPC, NOTCH2NLC, AP001453.4, H3C2, H3C3,
AL445524.2, AC275546.1, MG910336.1, AC118282.7, AC270106.1, AL035699.1,
ELOA3D, AC106873.8, AL049569.3, and AC084756.2

In embodiments, the one or more nucleic acids (e.g. at least 3, or at least 5, or at least 10, or at least 25, or at least 50, or at least 100, or at least 150, or at least 175), or the one or more proteins (e.g. at least 3, or at least 5, or at least 10, or at least 25, or at least 50, or at least 100, or at least 150, or at least 175) encoded by one or more nucleic acids (e.g. at least 3, or at least 5, or at least 10, or at least 25, or at least 50, or at least 100, or at least 150, or at least 175) that are unique to or preferentially present in the disclosed megakaryocyte-derived extracellular vesicles as compared to other types of extracellular vesicles, are involved in neutrophil extracellular trap formation, and are optionally selected from H3-3A, H4C12, H2AC18, H2AC13, H2AC19, H3-3B, H2BC12, H4C3, H3C10, H2AC14, H2BC4, H2AC20, H3C13, H4C14, H3C2, H2AC8, H2BC3, H3C7, H2AC17, H3C4, H2BC11, SELP, H3C11, H4C2, H2AC15, H2AC12, H3C1, H4C4, H2AC4, H4C13, H2BC5, H2AC7, H2AC16, H2BC17, H2AJ, H3C3, H2BC10, H2AC21, H3C14, H3C15, H2AC11, H3C12, FPR1, H2BC13, H4C1, MACROH2A1, H2BC14, H2AX, H2BC6, NCF4, H4C6, H2BC15, H2BC21, H2BC7, H2BC8, CYBB, FCGR1A, H2AZ2, H4C5, AQP9, H2BC9, CLCN4, FPR2, H4C9, HDAC9, ITGAL, FCGR3B, ITGAM, H3C8, TLR2, NCF1, NCF2, H3C6, CAMP, MPO, ELANE, FCGR3A, AZU1, H2AW, SIGLEC9, CLEC7A, PLCB4, CASP1, H2BU1, TLR4, and TLR8.

In embodiments, the one or more nucleic acids (e.g. at least 3, or at least 5, or at least 10, or at least 25, or at least 50, or at least 100, or at least 150, or at least 175), or the one or more proteins (e.g. at least 3, or at least 5, or at least 10, or at least 25, or at least 50, or at least 100, or at least 150, or at least 175) encoded by one or more nucleic acids (e.g. at least 3, or at least 5, or at least 10, or at least 25, or at least 50, or at least 100, or at least 150, or at least 175) that are unique to or preferentially present in the disclosed megakaryocyte-derived extracellular vesicles as compared to other types of extracellular vesicles, are involved in neutrophil extracellular trap formation, hematopoietic cell lineage, cell adhesion, ATP-binding cassette (ABC) transport, chemokine signaling pathway, phagocytosis, and/or aminoacyl-tRNA biosynthesis.

In embodiments, the one or more nucleic acids (e.g. at least 3, or at least 5, or at least 10, or at least 25, or at least 50, or at least 100, or at least 150, or at least 175), or the one or more proteins (e.g. at least 3, or at least 5, or at least 10, or at least 25, or at least 50, or at least 100, or at least 150, or at least 175) encoded by one or more nucleic acids (e.g. at least 3, or at least 5, or at least 10, or at least 25, or at least 50, or at least 100, or at least 150, or at least 175) are involved in neutrophil extracellular trap formation, and are optionally selected from H3-3A, H4C12, H2AC18, H2AC13, H2AC19, H3-3B, H2BC12, H4C3, H3C10, H2AC14, H2BC4, H2AC20, H3C13, H4C14, H3C2, H2AC8, H2BC3, H3C7, H2AC17, H3C4, H2BC11, SELP, H3C11, H4C2, H2AC15, H2AC12, H3C1, H4C4, H2AC4, H4C13, H2BC5, H2AC7, H2AC16, H2BC17, H2AJ, H3C3, H2BC10, H2AC21, H3C14, H3C15, H2AC11, H3C12, FPR1, H2BC13, H4C1, MACROH2A1, H2BC14, H2AX, H2BC6, NCF4, H4C6, H2BC15, H2BC21, H2BC7, H2BC8, CYBB, FCGR1A, H2AZ2, H4C5, AQP9, H2BC9, CLCN4, FPR2, H4C9, HDAC9, ITGAL, FCGR3B, ITGAM, H3C8, TLR2, NCF1, NCF2, H3C6, CAMP, MPO, ELANE, FCGR3A, AZU1, H2AW, SIGLEC9, CLEC7A, PLCB4, CASP1, H2BU1, TLR4, and TLR8.

In embodiments, the one or more nucleic acids (e.g. at least 3, or at least 5, or at least, or at least 25, or at least 50, or at least 100, or at least 150, or at least 175), or the one or more proteins (e.g. at least 3, or at least 5, or at least 10, or at least 25, or at least 50, or at least 100, or at least 150, or at least 175) encoded by one or more nucleic acids (e.g. at least 3, or at least 5, or at least 10, or at least 25, or at least 50, or at least 100, or at least 150, or at least 175) are involved in hematopoietic cell lineage, and are optionally selected from CD36, IL9R, FCGR1A, CSF3R, KIT, GP5, CSF1, ITGAM, CD33, IL1B, IL7R, IL6R, HLA-DPB1, CD24, CD22, GYPA, MME, ITGA1, CD1C, IL3RA, HLA-DPA1, IL1R2, CD34, HLA-DRA, HLA-DQA1, and CD7.

In embodiments, the one or more nucleic acids (e.g. at least 3, or at least 5, or at least 10, or at least 25, or at least 50, or at least 100, or at least 150, or at least 175), or the one or more (e.g. at least 3, or at least 5, or at least 10, or at least 25, or at least 50, or at least 100, or at least 150, or at least 175) proteins encoded by one or more nucleic acids (e.g. at least 3, or at least 5, or at least 10, or at least 25, or at least 50, or at least 100, or at least 150, or at least 175) that are involved in cell adhesion, and are optionally selected from CD226, SELP, PECAM1, VSIR, PTPRC, NRXN1, NECTIN2, ITGAL, ITGAM, SELL, CD28, HLA-DPB1, CNTNAP2, CLDN5, JAM2, CD40LG, LRRC4, CD274, CD40, CD276, CD22, ITGA9, NECTIN3, NEGR1, HLA-DPA1, CNTN1, ITGB8, CD34, HLA-DRA, SIGLEC1, and HLA-DQA1.

In embodiments, the one or more nucleic acids (e.g. at least 3, or at least 5, or at least 10, or at least 25, or at least 50, or at least 100, or at least 150, or at least 175), or the one or more proteins (e.g. at least 3, or at least 5, or at least 10, or at least 25, or at least 50, or at least 100, or at least 150, or at least 175) encoded by one or more nucleic acids (e.g. at least 3, or at least 5, or at least 10, or at least 25, or at least 50, or at least 100, or at least 150, or at least 175) are involved in ATP-binding cassette (ABC) transport, and are optionally selected from CFTR, ABCD4, ABCA5, ABCA9, ABCC1, ABCB8, ABCA8, ABCB9, ABCC9, ABCA1, ABCA10, and ABCC8.

In embodiments, the one or more nucleic acids (e.g. at least 3, or at least 5, or at least, or at least 25, or at least 50, or at least 100, or at least 150, or at least 175), or the one or more proteins (e.g. at least 3, or at least 5, or at least 10, or at least 25, or at least 50, or at least 100, or at least 150, or at least 175) encoded by one or more nucleic acids (e.g. at least 3, or at least 5, or at least 10, or at least 25, or at least 50, or at least 100, or at least 150, or at least 175) are involved in Rap1 signaling pathway, and are optionally selected from F2R, LCP2, FYB1, FPR1, F2RL3, P2RY1, RAPGEF2, KIT, EGF, CSF1, ANGPT1, PDGFC, ITGAL, VAV1, VAV3, PDGFRA, ITGAM, RAPGEF5, MAGI2, FARP2, CTNND1, FGF23, FLT1, FGF1, DRD2, AFDN, PLCB4, APBB1IP, PLCE1, MAGI3, KDR, LPAR3, GRIN2B, ADCY1, FGF7, ANGPT4, and TEK.

In embodiments, the one or more nucleic acids (e.g. at least 3, or at least 5, or at least 10, or at least 25, or at least 50, or at least 100, or at least 150, or at least 175), or the one or more proteins (e.g. at least 3, or at least 5, or at least 10, or at least 25, or at least 50, or at least 100, or at least 150, or at least 175) encoded by one or more nucleic acids (e.g. at least 3, or at least 5, or at least 10, or at least 25, or at least 50, or at least 100, or at least 150, or at least 175) are involved in chemokine signaling pathway, and are optionally selected from PF4, CCL5, CXCL2, CXCL8, ELMO1, GRK2, GNG8, CCR4, PF4V1, VAV1, VAV3, PIK3R6, PIK3CG, SHC4, GRK3, NCF1, HCK, GRK4, ITK, GNG2, CXCR2, CCR1, CXCL5, CCL3, PLCB4, CXCL6, CCL22, CCL8, CCL4L2, CXCL12, GNGT1, CXCL11, ADCY1, and XCL1.

In embodiments, the one or more nucleic acids (e.g. at least 3, or at least 5, or at least 10, or at least 25, or at least 50, or at least 100, or at least 150, or at least 175), or the one or more proteins (e.g. at least 3, or at least 5, or at least 10, or at least 25, or at least 50, or at least 100, or at least 150, or at least 175) encoded by one or more nucleic acids (e.g. at least 3, or at least 5, or at least 10, or at least 25, or at least 50, or at least 100, or at least 150, or at least 175) that are involved in phagocytosis, and are optionally selected from TUBA8, NCF4, CD36, CYBB, FCGR1A, CTSS, FCGR3B, PIKFYVE, ITGAM, TLR2, NCF1, NCF2, HLA-DPB1, MPO, EEA1, MSR1, FCGR3A, FCAR, DYNC111, CLEC7A, FCGR2B, HLA-DPA1, ATP6VOD2, MRC2, TLR4, TLR6, HLA-DRA, MARCO, and HLA-DQA1.

In embodiments, the one or more nucleic acids (e.g. at least 3, or at least 5, or at least 10, or at least 25, or at least 50, or at least 100, or at least 150, or at least 175), or the one or more proteins (e.g. at least 3, or at least 5, or at least 10, or at least 25, or at least 50, or at least 100, or at least 150, or at least 175) encoded by one or more nucleic acids (e.g. at least 3, or at least 5, or at least 10, or at least 25, or at least 50, or at least 100, or at least 150, or at least 175) are involved in various pathways, and are optionally selected from H3-3A, H4C12, H2AC18, H2AC13, H2AC19, H3-3B, H2BC12, H4C3, H3C10, H2AC14, H2BC4, H2AC20, H3C13, H4C14, H3C2, H2AC8, H2BC3, H3C7, H2AC17, H3C4, H2BC11, SELP, H3C11, H4C2, H2AC15, H2AC12, H3C1, H4C4, H2AC4, H4C13, H2BC5, H2AC7, H2AC16, H2BC17, H2AJ, H3C3, H2BC10, H2AC21, H3C14, H3C15, H2AC11, H3C12, FPR1, H2BC13, H4C1, MACROH2A1, H2BC14, H2AX, H2BC6, NCF4, H4C6, H2BC15, H2BC21, H2BC7, H2BC8, CYBB, FCGR1A, H2AZ2, H4C5, AQP9, H2BC9, CLCN4, FPR2, H4C9, HDAC9, ITGAL, FCGR3B, ITGAM, H3C8, TLR2, NCF1, NCF2, H3C6, CAMP, MPO, ELANE, FCGR3A, AZU1, H2AW, SIGLEC9, CLEC7A, PLCB4, CASP1, H2BU1, TLR4, TLR8, CD36, IL9R, FCGR1A, CSF3R, KIT, GP5, CSF1, ITGAM, CD33, IL1B, IL7R, IL6R, HLA-DPB1, CD24, CD22, GYPA, MME, ITGA1, CD1C, IL3RA, HLA-DPA1, IL1R2, CD34, HLA-DRA, HLA-DQA1, CD7, CD226, SELP, PECAM1, VSIR, PTPRC, NRXN1, NECTIN2, ITGAL, ITGAM, SELL, CD28, HLA-DPB1, CNTNAP2, CLDN5, JAM2, CD40LG, LRRC4, CD274, CD40, CD276, CD22, ITGA9, NECTIN3, NEGR1, HLA-DPA1, CNTN1, ITGB8, CD34, HLA-DRA, SIGLEC1, HLA-DQA1, CFTR, ABCD4, ABCA5, ABCA9, ABCC1, ABCB8, ABCA8, ABCB9, ABCC9, ABCA1, ABCA10, ABCC8, F2R, LCP2, FYB1, FPR1, F2RL3, P2RY1, RAPGEF2, KIT, EGF, CSF1, ANGPT1, PDGFC, ITGAL, VAV1, VAV3, PDGFRA, ITGAM, RAPGEF5, MAGI2, FARP2, CTNND1, FGF23, FLT1, FGF1, DRD2, AFDN, PLCB4, APBB1IP, PLCE1, MAGI3, KDR, LPAR3, GRIN2B, ADCY1, FGF7, ANGPT4, TEK, PF4, CCL5, CXCL2, CXCL8, ELMO1, GRK2, GNG8, CCR4, PF4V1, VAV1, VAV3, PIK3R6, PIK3CG, SHC4, GRK3, NCF1, HCK, GRK4, ITK, GNG2, CXCR2, CCR1, CXCL5, CCL3, PLCB4, CXCL6, CCL22, CCL8, CCL4L2, CXCL12, GNGT1, CXCL11, ADCY1, XCL1, TUBA8, NCF4, CD36, CYBB, FCGR1A, CTSS, FCGR3B, PIKFYVE, ITGAM, TLR2, NCF1, NCF2, HLA-DPB1, MPO, EEA1, MSR1, FCGR3A, FCAR, DYNC111, CLEC7A, FCGR2B, HLA-DPA1, ATP6VOD2, MRC2, TLR4, TLR6, HLA-DRA, MARCO, HLA-DQA1, RARS1, DARS1, KARS1, SARS1, AARS1, TARS1, NARS1, WARS1, GATB, CARS1, EPRS1, TARS3, EARS2, IARS1, QRSL1, and WARS2.

In embodiments, the one or more nucleic acids (e.g. at least 3, or at least 5, or at least 10, or at least 25, or at least 50, or at least 100, or at least 150, or at least 175), or the one or more proteins (e.g. at least 3, or at least 5, or at least 10, or at least 25, or at least 50, or at least 100, or at least 150, or at least 175) encoded by one or more nucleic acids (e.g. at least 3, or at least 5, or at least 10, or at least 25, or at least 50, or at least 100, or at least 150, or at least 175) that are involved in aminoacyl-tRNA biosynthesis, and are optionally selected from RARS1, DARS1, KARS1, SARS1, AARS1, TARS1, NARS1, WARS1, GATB, CARS1, EPRS1, TARS3, EARS2, IARS1, QRSL1, and WARS2.

In embodiments, more than about 1%, or more than about 5%, or more than about 10%, or more than about 15%, or more than about 20%, or more than about 25%, or more than about 30%, or more than about 35%, or more than about 40%, or more than about 45%, or more than about 50%, or more than about 55%, or more than about 60%, or more than about 65%, or more than about 70%, or more than about 75%, or more than about 80%, or more than about 85%, or more than about 90%, or more than about 95% of the megakaryocyte-derived extracellular vesicles comprise the one or more nucleic acids or the one or more proteins encoded by the one or more nucleic acids.

In embodiments, more than about 1%, more than about 2%, more than about 3%, more than about 4%, more than about 5%, more than about 6%, more than about 7%, more than about 8%, more than about 9%, more than about 10%, more than about 11%, more than about 12%, more than about 13%, more than about 14%, more than about 15%, more than about 16%, more than about 17%, more than about 18%, more than about 19%, more than about 20%, more than about 21%, more than about 22%, more than about 23%, more than about 24%, more than about 25%, more than about 26%, more than about 27%, more than about 28%, more than about 29%, more than about 30%, more than about 31%, more than about 32%, more than about 33%, more than about 34%, more than about 35%, more than about 36%, more than about 37%, more than about 38%, more than about 39%, more than about 40%, more than about 41%, more than about 42%, more than about 43%, more than about 44%, more than about 45%, more than about 46%, more than about 47%, more than about 48%, more than about 49%, more than about 50%, more than about 51%, more than about 52%, more than about 53%, more than about 54%, more than about 55%, more than about 56%, more than about 57%, more than about 58%, more than about 59%, more than about 60%, more than about 61%, more than about 62%, more than about 63%, more than about 64%, more than about 65%, more than about 66%, more than about 67%, more than about 68%, more than about 69%, more than about 70%, more than about 71%, more than about 72%, more than about 73%, more than about 74%, more than about 75%, more than about 76%, more than about 77%, more than about 78%, more than about 79%, more than about 80%, more than about 81%, more than about 82%, more than about 83%, more than about 84%, more than about 85%, more than about 86%, more than about 87%, more than about 88%, more than about 89%, more than about 90%, more than about 91%, more than about 92%, more than about 93%, more than about 94%, more than about 95%, more than about 96%, more than about 97%, more than about 98%, more than about 99%, or more of the megakaryocyte-derived extracellular vesicles comprise the one or more nucleic acids or the one or more proteins encoded by the one or more nucleic acids.

In embodiments, less than about 95%, or less than about 90%, or less than about 85%, or less than about 80%, or less than about 75%, or less than about 70%, or less than about 65%, or less than about 60%, or less than about 55%, or less than about 50%, or less than about 45%, or less than about 40%, or less than about 35%, or less than about 30%, or less than about 25%, or less than about 20%, or less than about 15%, or less than about 10%, or less than about 5%, or less than about 1% of the megakaryocyte-derived extracellular vesicles comprise the one or more nucleic acids or the one or more proteins encoded by the one or more nucleic acids.

In embodiments, less than about 99%, or less than about 98%, or less than about 97%, or less than about 96%, or less than about 95%, or less than about 94%, or less than about 93%, or less than about 92%, or less than about 91%, or less than about 90%, or less than about 89%, or less than about 88%, or less than about 87%, or less than about 86% or less, less, about 85%, or less than about 84%, or less than about 83%, or less than about 82%, or less than about 81%, or less than about 80%, or less than about 79%, or less than about 78%, or less than about 77%, or less than about 76%, or less than about 75%, or less than about 74%, or less than about 73%, or less than about 72%, or less than about 71%, or less than about 70%, or less than about 69%, or less than about 68%, or less than about 67%, or less than about 66%, or less than about 65%, or less than about 64%, or less than about 63%, or less than about 62%, or less than about 61%, or less than about 60%, or less than about 59%, or less than about 58%, or less than about 57%, or less than about 56%, or less than about 55%, or less than about 54%, or less than about 53%, or less than about 52%, or less than about 51%, or less than about 50%, or less than about 49%, or less than about 48%, or less than about 47%, or less than about 46%, or less than about 45%, or less than about 44%, or less than about 43%, or less than about 42%, or less than about 41%, or less than about 40%, or less than about 39%, or less than about 38%, or less than about 37%, or less than about 36% or less, less, about 35%, or less than about 34%, or less than about 33%, or less than about 32%, or less than about 31%, or less than about 30%, or less than about 29%, or less than about 28%, or less than about 27%, or less than about 26%, or less than about 25%, or less than about 24%, or less than about 23%, or less than about 22%, or less than about 21%, or less than about 20%, or less than about 19%, or less than about 18%, or less than about 17%, or less than about 16%, or less than about 15%, or less than about 14%, or less than about 13%, or less than about 12%, or less than about 11%, or less than about 10%, or less than about 9%, or less than about 8%, or less than about 7%, or less than about 6%, or less than about 5%, or less than about 4%, or less than about 3%, or less than about 2%, or less than about 1% of the megakaryocyte-derived extracellular vesicles comprise the one or more nucleic acids or the one or more proteins encoded by the one or more nucleic acids.

### Size Profile

In various embodiments, the present megakaryocyte-derived extracellular vesicles are characterized by a unique size (e.g. vesicle diameter) profile that distinguishes them from, for instance, naturally-occurring megakaryocyte-derived extracellular vesicles and/or vesicles or extracellular vesicles derived from platelets. In various embodiments, the present megakaryocyte-derived extracellular vesicles are characterized by a such a size profile, which favors larger particles, e.g. as compared to naturally-occurring megakaryocyte-derived extracellular vesicles and/or vesicles or extracellular vesicles derived from platelets, that are desirable for, e.g., their higher carrying capacity.

In various embodiments, the present megakaryocyte-derived extracellular vesicles are characterized by a bias for particles of about 30 nm to about 100 nm.

In various embodiments, the present megakaryocyte-derived extracellular vesicles are characterized by a bias for particles of about 30 nm to about 400 nm.

In various embodiments, the present megakaryocyte-derived extracellular vesicles are characterized by a bias for particles of about 100 nm to about 200 nm.

In various embodiments, the present megakaryocyte-derived extracellular vesicles are characterized by a bias for particles of about 100 nm to about 300 nm.

In various embodiments, the present megakaryocyte-derived extracellular vesicles are characterized by a bias for particles of about 100 nm to about 500 nm.

In various embodiments, the present megakaryocyte-derived extracellular vesicles are characterized by a bias for particles of about 100 nm to about 600 nm.

In various embodiments, the present megakaryocyte-derived extracellular vesicles are characterized by a bias for particles of about 200 nm in diameter, on average.

In various embodiments, the present megakaryocyte-derived extracellular vesicles are characterized by a bias for particles of about 250 nm in diameter, on average.

In embodiments, the megakaryocyte-derived extracellular vesicles are substantially of a diameter of less than about 100 nm. In embodiments, the megakaryocyte-derived extracellular vesicles are substantially of a diameter in the range between about 30 nm to about 300 nm. In embodiments, the megakaryocyte-derived extracellular vesicles are substantially of a diameter in the range between about 30 nm to about 400 nm. In embodiments, the megakaryocyte-derived extracellular vesicles are substantially of a diameter in the range between about 100 nm to about 300 nm. In embodiments, the megakaryocyte-derived extracellular vesicles are substantially of a diameter in the range between about 200 nm to about 300 nm. In embodiments, the megakaryocyte-derived extracellular vesicles are substantially of a diameter in the range between about 300 nm to about 400 nm. In embodiments, the megakaryocyte-derived extracellular vesicles are substantially of a diameter in the range between about 400 nm to about 500 nm. In embodiments, the megakaryocyte-derived extracellular vesicles are substantially of a diameter in the range between about 500 nm to about 600 nm. In embodiments, the megakaryocyte-derived extracellular vesicles are substantially of a diameter in the range between about 600 nm to about 700 nm. In embodiments, the megakaryocyte-derived extracellular vesicles are substantially of a diameter in the range between about 700 nm to about 800 nm. In embodiments, the megakaryocyte-derived extracellular vesicles are substantially of a diameter in the range between about 800 nm to about 900 nm. In embodiments, the megakaryocyte-derived extracellular vesicles are substantially of a diameter in the range between about 900 nm to about 1000 nm. In embodiments, the megakaryocyte-derived extracellular vesicles are substantially of a diameter in the range between about 500 nm to about 1000 nm. In embodiments, the megakaryocyte-derived extracellular vesicles are substantially of a diameter in the range between about 600 nm to about 1000 nm. In embodiments, the megakaryocyte-derived extracellular vesicles are substantially of a diameter in the range between about 100 nm to about 500 nm. In embodiments, the megakaryocyte-derived extracellular vesicles are substantially of a diameter in the range between about 100 nm to about 600 nm. In embodiments, the megakaryocyte-derived extracellular vesicles are substantially of a diameter in the range between about 150 nm to about 500 nm. In embodiments, the megakaryocyte-derived extracellular vesicles are substantially of a diameter in the range between about 100 nm to about 200 nm. In embodiments, the megakaryocyte-derived extracellular vesicles are substantially of a diameter in the range between about 100 nm to about 200 nm. In embodiments, the megakaryocyte-derived extracellular vesicles are substantially of a diameter in the range between about 200 nm to about 600 nm. In embodiments, the megakaryocyte-derived extracellular vesicles are substantially of a diameter in the range between about 30 nm to 100 nm, or between about 30 nm to 400 nm, or between about 100 nm to about 200 nm, or between about 100 nm to about 500 nm, or between about 200 nm to about 350 nm, or between about 400 nm to about 600 nm.

In embodiments, about 90% or more, or about 95% or more, or about 97% or more, or about 99% or more of the megakaryocyte-derived extracellular vesicles are of a diameter of between about 30 to 100 nm.

In embodiments, about 90% or more, or about 95% or more, or about 97% or more, or about 99% or more of the megakaryocyte-derived extracellular vesicles are of a diameter of between about 30 to 400 nm.

In embodiments, about 90% or more, or about 95% or more, or about 97% or more, or about 99% or more of the megakaryocyte-derived extracellular vesicles are of a diameter of between about 100 nm to about 200 nm.

In embodiments, about 90% or more, or about 95% or more, or about 97% or more, or about 99% or more of the megakaryocyte-derived extracellular vesicles are of a diameter of between about 100 nm to about 300 nm.

In embodiments, about 90% or more, or about 95% or more, or about 97% or more, or about 99% or more of the megakaryocyte-derived extracellular vesicles are of a diameter of between about 200 nm to about 350 nm.

In embodiments, about 90% or more, or about 95% or more, or about 97% or more, or about 99% or more of the megakaryocyte-derived extracellular vesicles are of a diameter of between about 100 nm to about 600 nm.

In embodiments, about 90% or more, or about 95% or more, or about 97% or more, or about 99% or more of the megakaryocyte-derived extracellular vesicles are of a diameter of between about 400 nm to about 600 nm.

In embodiments, about 90% or more, or about 95% or more, or about 97% or more, or about 99% or more of the megakaryocyte-derived extracellular vesicles are of a diameter of between about 200 nm to about 600 nm.

In embodiments, about 90% or more, or about 95% or more, or about 97% or more, or about 99% or more of the megakaryocyte-derived extracellular vesicles are of a diameter of between about 30 to about 100 nm and/or about 30 to about 400 nm and/or about 100 nm to about 200 nm and/or about 100 nm to about 300 nm and/or between about 200 nm to about 350 nm and/or between about 400 nm to about 600 nm.

In embodiments, the present compositions comprise various subpopulations of vesicles of different diameter. For example, in embodiments, present compositions comprise one or more of (e.g. one, or two, or three, or four of): a subpopulation of about 50 nm in diameter, a subpopulation of about 150 nm in diameter, a subpopulation of about 200 nm in diameter, a subpopulation of about 250 nm in diameter, a subpopulation of about 300 nm in diameter, a subpopulation of about 400 nm in diameter, a subpopulation of about 500 nm in diameter and a subpopulation of about 600 nm in diameter. In embodiments, present compositions comprise one or more of (e.g. one, or two, or three, or four of): a subpopulation of about 45 nm in diameter, a subpopulation of about 135 nm in diameter, a subpopulation of about 285 nm in diameter, and a subpopulation of about 525 nm in diameter.

In embodiments, about 90% or more, or about 95% or more, or about 97% or more, or about 99% or more of the megakaryocyte-derived extracellular vesicles are of about 50 nm in diameter and/or about 150 nm in diameter and/or about 300 nm in diameter and/or about 500 nm in diameter.

In embodiments, the population of megakaryocyte-derived extracellular vesicles exhibits the following characteristics:
a) about 80% or more, about 85% or more, about 90% or more, about 95% or more, about 97% or more, or about 99% or more of the megakaryocyte-derived extracellular vesicles in the population are substantially free of nuclei;
b) about 90% or more, or about 95% or more, or about 97% or more, or about 99% or more of the megakaryocyte-derived extracellular vesicles are of a diameter of between about 100 nm to about 600 nm.;
c) about 40% or more, about 50% or more, about 60% or more, about 70% or more, about 80% or more, or about 90% or more of the megakaryocyte-derived extracellular vesicles in the population comprise CD41; and
d) the population comprises about 1×10⁷ or more, about 1.5×10⁷ or more, about 5×10⁷ or more, about 1×10⁸ or more, about 1.5×10⁸ or more, about 5×10⁸ or more, about 1×10⁹ or more, about 5×10⁹ or more, about 1×10¹⁰ or more, or about 1×10¹⁰ or more megakaryocyte-derived extracellular vesicles.

In embodiments, the population of megakaryocyte-derived extracellular vesicles exhibits the following characteristics:
a) about 80% or more, about 85% or more, about 90% or more, about 95% or more, about 97% or more, or about 99% or more of the megakaryocyte-derived extracellular vesicles in the population are substantially free of nuclei;
b) about 90% or more, or about 95% or more, or about 97% or more, or about 99% or more of the megakaryocyte-derived extracellular vesicles are of a diameter of between about 100 nm to about 600 nm.;
c) about 40% or more, about 50% or more, about 60% or more, about 70% or more, about 80% or more, or about 90% or more of the megakaryocyte-derived extracellular vesicles in the population comprise CD61; and
d) the population comprises about 1×10⁷ or more, about 1.5×10⁷ or more, about 5×10⁷ or more, about 1×10⁸ or more, about 1.5×10⁸ or more, about 5×10⁸ or more, about 1×10⁹ or more, about 5×10⁹ or more, about 1×10¹⁰ or more, or about 1×10¹⁰ or more megakaryocyte-derived extracellular vesicles.

Any method for determining the amount of nuclei in the population of megakaryocyte-derived extracellular vesicles is contemplated by the present disclosure. Non-limiting examples of methods include staining the megakaryocyte-derived extracellular vesicles with a nuclear stain such as DRAQ5, wherein a lack of staining indicates that the megakaryocyte-derived extracellular vesicles are substantially free of nuclei.

### Sources and Characterization of Megakaryocyte-Derived Extracellular Vesicles

Megakaryocytes are large, polyploid cells derived from hematopoietic stem and progenitor cells, contained within the CD34⁺-cell compartment. In embodiments, the megakaryocyte is characterized by the expression and/or presence of one or more of CD41, CD62P, GPVI, CLEC-2, CD42b and CD61. In embodiments, the megakaryocyte is one or more of CD42b+, CD61+, and DNA+. One morphological characteristic of mature megakaryocytes is the development of a large, multi-lobed nucleus. Mature megakaryocytes can stop proliferating, but continue to increase their DNA content through endomitosis, with a parallel increase in cell size.

In embodiments, in addition to extracellular vesicles, megakaryocytes can shed pre- and proplatelets and platelet-like particles. These shed moieties can mature into platelets. In embodiments, the pre- and proplatelets and platelet-like particles are all different products, which can be differentiated by size, morphology, biomarker expression and/or presence, and function.

Megakaryocytes are derived from pluripotent hematopoietic stem cell (HSC) precursors. HSCs are produced primarily by the liver, kidney, spleen, and bone marrow and are capable of producing a variety of blood cells depending on the signals they receive.

Thrombopoietin (TPO) is a primary signal for inducing an HSC to differentiate into a megakaryocyte. Other molecular signals for inducing megakaryocyte differentiation include granulocyte-macrophage colony-stimulating factor (GM-CSF), Interleukin-3 (IL-3), IL-6, IL-11, SCF, fms-like tyrosine kinase 3 ligand (FLT3L), interleukin 9 (IL-9), and the like. Production details are also described elsewhere herein.

In embodiments, the composition comprises substantially purified megakaryocyte-derived extracellular vesicles derived from a human pluripotent stem cell.

In embodiments, the human pluripotent stem cell is a primary CD34+ hematopoietic stem cell. In embodiments, the primary CD34+ hematopoietic stem cell is sourced from peripheral blood or cord blood. In embodiments, the peripheral blood is granulocyte colony-stimulating factor-mobilized adult peripheral blood (mPB). In embodiments, the human pluripotent stem cell is an HSC produced by the liver, kidney, spleen, or bone marrow. In embodiments, the HSC is produced by the liver. In embodiments, the HSC is produced by the kidney. In embodiments, the HSC is produced by the spleen. In embodiments, the HSC is produced by the bone marrow. In embodiments, the HSC is induced to differentiate into a megakaryocyte by receiving a molecular signal selected from one or more of TPO, GM-CSF, IL-3, IL-6, IL-11, SCF, FIt3L, IL-9, and the like. In embodiments, the molecular signal is TPO. In embodiments, the molecular signal is GM-CSF. In embodiments, the molecular signal is IL-3. In embodiments, the molecular signal is IL-6. In embodiments, the molecular signal is IL-11. In embodiments, the molecular signal is IL-6. In embodiments, the molecular signal is SCF. In embodiments, the molecular signal is IL-6. In embodiments, the molecular signal is FIt3L. In embodiments, the molecular signal is IL-6. In embodiments, the molecular signal is IL-9.

In embodiments, the molecular signal is a chemokine.

In embodiments, the molecular signal promotes cell fate decision toward megakaryopoiesis.

In embodiments, the molecular signal is devoid of erythropoietin (EPO).

In embodiments, the human pluripotent stem cell is an embryonic stem cell (ESC).

ESCs have the capacity to form cells from all three germ layers of the body, regardless of the method by which the ESCs are derived. ESCs are functionally stem cells that can have one or more of the following characteristics: (a) be capable of inducing teratomas when transplanted in immunodeficient mice; (b) be capable of differentiating to cell types of all three germ layers (i.e. ectodermal, mesodermal, and endodermal cell types); and (c) express one or more markers of embryonic stem cells (e.g., October 4, alkaline phosphatase. SSEA-3 surface antigen, SSEA-4 surface antigen, SSEA-5 surface antigen, Nanog, TRA-I-60, TRA-1-81, SOX2, REX1, and the like).

In embodiments, the human pluripotent stem cell is an induced pluripotent stem cell (iPCs). Mature differentiated cells can be reprogrammed and dedifferentiated into embryonic-like cells, with embryonic stem cell-like properties. iPSCs can be generated using fetal, postnatal, newborn, juvenile, or adult somatic cells. Fibroblast cells can be reversed into pluripotency via, for example, retroviral transduction of certain transcription factors, resulting in iPSs. In embodiments, iPSs are generated from various tissues, including fibroblasts, keratinocytes, melanocyte blood cells, bone marrow cells, adipose cells, and tissue-resident progenitor cells. In embodiments, iPSCs are generated via one or more reprogramming or Yamanaka factors, e.g. Oct3/4, Sox2, Klf4, and c-Myc. In certain embodiments, at least two, three, or four reprogramming factors are expressed in a somatic cell to reprogram the somatic cell.

Once a pluripotent cell has completed differentiation and become a mature megakaryocyte, it begins the process of producing platelets, which do not contain a nucleus and may be about 1-3 um in diameter. Megakaryocytes also produce extracellular vesicles.

In embodiments, the present megakaryocytes are induced to favor production of megakaryocyte-derived extracellular vesicles over platelets. That is, in embodiments, the present megakaryocytes produce substantially more megakaryocyte-derived extracellular vesicles than platelets. In embodiments, the present compositions are substantially free of platelets. In embodiments, the present compositions contain less than about 10%, or less than about 7%, or less than about 5%, or less than about 3%, or less than about 2%, or less than about 1% platelets.

In embodiments, the present compositions are substantially free of extracellular vesicles derived from platelets. In embodiments, the present compositions contain less than about 10%, or less than about 7%, or less than about 5%, or less than about 3%, or less than about 2%, or less than about 1% of extracellular vesicles derived from platelets.

In embodiments, the megakaryocyte-derived extracellular vesicles of the disclosure are substantially free of organelles. Non-limiting examples of contaminating organelles include, but are not limited to, mitochondria, and nuclei. In embodiments, the megakaryocyte-derived extracellular vesicles of the disclosure are substantially free of mitochondria. In embodiments, the preparation comprising the megakaryocyte-derived extracellular vesicles of the disclosure is substantially free of exosomes. In embodiments, megakaryocyte-derived extracellular vesicles of the disclosure comprise organelles.

In embodiments, the megakaryocyte-derived extracellular vesicles of the disclosure are substantially free of nuclei. In embodiments, about 80% to about 100%, about 85% to about 100%, about 90% to about 100%, or about 95% to about 100% of the megakaryocyte-derived extracellular vesicles in the population are substantially free of nuclei. In embodiments, greater than about 80%, greater than about 85%, greater than about 90%, greater than about 95%, greater than about 99%, or about 100% of the megakaryocyte-derived extracellular vesicles in the population are substantially free of nuclei.

### Targeting

Megakaryocyte-derived extracellular vesicles can home to a range of target cells. When megakaryocyte-derived extracellular vesicles bind to a target cell, they can release their cargo via various mechanisms of megakaryocyte-derived extracellular vesicle internalization by the target cell.

In embodiments, the megakaryocyte-derived extracellular vesicles are suitable for homing to bone marrow in vivo. In embodiments, the megakaryocyte-derived extracellular vesicles are suitable for homing to bone marrow in vitro. In embodiments, the megakaryocyte-derived extracellular vesicles home in vivo to bone marrow with about a 2-fold, or about a 3-fold, or about a 4-fold, or about a 5-fold, or about a 6-fold, or about a 7-fold, or about a 8-fold, or about a 9-fold, or about a 10-fold greater specificity than to another cell type, or to another organ, or to all other cell types combined.

In embodiments, the megakaryocyte-derived extracellular vesicles home in vivo to one or more myelopoeitic cells in bone marrow. In embodiments, the one or more myelopoeitic cells are selected from myeloblasts, promyelocytes, neutrophilic myelocytes, eosinophilic myelocytes, neutrophilic metamyelocytes, eosinophilic metamyelocytes, neutrophilic band cells, eosinophilic band cells, segmented neutrophils, segmented eosinophils, segmented basophils, and mast cells. In embodiments, the megakaryocyte-derived extracellular vesicles home in vivo to one or more erythropoietic cells in bone marrow. In embodiments, the one or more erythropoietic cells are selected from pronormoblasts, basophilic normoblasts, polychromatic normoblasts, and orthochromatic normoblasts. In embodiments, the megakaryocyte-derived extracellular vesicles home in vivo to one or more of plasma cells, reticular cells, lymphocytes, monocytes, and megakaryocytes.

In embodiments, the megakaryocyte-derived extracellular vesicles home in vivo to one or more hematopoietic cells in bone marrow. In embodiments, the megakaryocyte-derived extracellular vesicles home in vivo to one or more hematopoietic cells in bone marrow, e.g. thrombopoietic cells.

In embodiments, the megakaryocyte-derived extracellular vesicles home in vivo to one or more hematopoietic stem cells in bone marrow.

In embodiments, the megakaryocyte-derived extracellular vesicles home ex vivo to one or more hematopoietic stem cells in bone marrow. In embodiments, the one or more hematopoietic stem cells comprise and/or consist of CD45+ HSCs. In embodiments, the megakaryocyte-derived extracellular vesicles home ex vivo to one or more hematopoietic stem cells in bone marrow, wherein the hematopoietic stem cells comprise and/or consist of long-term hematopoietic stem cells. In embodiments, the long-term hematopoietic stem cells comprise and/or consist of CD45+/Lin−/CD150+/CD201+/CD48− HSCs. In embodiments, the megakaryocyte-derived extracellular vesicles home ex vivo to one or more hematopoietic stem cells in bone marrow, wherein the hematopoietic stem cells comprise and/or consist of pluripotent hematopoietic stem cells. In embodiments, the pluripotent hematopoietic stem cells comprise and/or consist of Lineage negative/c-Kit+/Sca-1+ (LSK) HSCs.

In embodiments, the megakaryocyte-derived extracellular vesicles are suitable for homing to an HSC in vivo. In embodiments, the megakaryocyte-derived extracellular vesicles are suitable for homing to an HSC in vitro. In embodiments, the megakaryocyte-derived extracellular vesicles are suitable for homing to an HSC ex vivo. In embodiments, the megakaryocyte-derived extracellular vesicles home in vivo to an HSC with about a 2-fold greater specificity than to another cell type, or than to another organ, or than to all other cell types combined. In embodiments, the megakaryocyte-derived extracellular vesicles home in vivo to an HSC with about a 3-fold greater specificity than to another cell type, or than to another organ, or than to all other cell types combined. In embodiments, the megakaryocyte-derived extracellular vesicles home in vivo to an HSC with about a 4-fold greater specificity than to another cell type, or to another organ, or to all other cell types combined. In embodiments, the megakaryocyte-derived extracellular vesicles home in vivo to an HSC with about a 5-fold greater specificity than to another cell type, or to another organ, or to all other cell types combined. In embodiments, the megakaryocyte-derived extracellular vesicles home in vivo to an HSC with about a 6-fold greater specificity than to another cell type, or to another organ, or to all other cell types combined. In embodiments, the megakaryocyte-derived extracellular vesicles home in vivo to an HSC with about a 7-fold greater specificity than to another cell type, or to another organ, or to all other cell types combined. In embodiments, the megakaryocyte-derived extracellular vesicles home in vivo to an HSC with about a 8-fold greater specificity than to another cell type, or to another organ, or to all other cell types combined. In embodiments, the megakaryocyte-derived extracellular vesicles home in vivo to an HSC with about a 9-fold greater specificity than to another cell type, or to another organ, or to all other cell types combined. In embodiments, the megakaryocyte-derived extracellular vesicles home in vivo to an HSC with about a 10-fold greater specificity than to another cell type, or to another organ, or to all other cell types combined.

In embodiments, the megakaryocyte-derived extracellular vesicles are suitable for homing to an HSC ex vivo. In embodiments, the megakaryocyte-derived extracellular vesicles are suitable for homing to an HSC ex vivo. In embodiments, the megakaryocyte-derived extracellular vesicles are suitable for homing to an HSC ex vivo. In embodiments, the megakaryocyte-derived extracellular vesicles home ex vivo to an HSC with about a 2-fold greater specificity than to another cell type, or than to another organ, or than to all other cell types combined. In embodiments, the megakaryocyte-derived extracellular vesicles home ex vivo to an HSC with about a 3-fold greater specificity than to another cell type, or than to another organ, or than to all other cell types combined. In embodiments, the megakaryocyte-derived extracellular vesicles home ex vivo to an HSC with about a 4-fold greater specificity than to another cell type, or to another organ, or to all other cell types combined. In embodiments, the megakaryocyte-derived extracellular vesicles home ex vivo to an HSC with about a 5-fold greater specificity than to another cell type, or to another organ, or to all other cell types combined. In embodiments, the megakaryocyte-derived extracellular vesicles home ex vivo to an HSC with about a 6-fold greater specificity than to another cell type, or to another organ, or to all other cell types combined. In embodiments, the megakaryocyte-derived extracellular vesicles home ex vivo to an HSC with about a 7-fold greater specificity than to another cell type, or to another organ, or to all other cell types combined. In embodiments, the megakaryocyte-derived extracellular vesicles home ex vivo an HSC with about a 8-fold greater specificity than to another cell type, or to another organ, or to all other cell types combined. In embodiments, the megakaryocyte-derived extracellular vesicles home ex vivo to an HSC with about a 9-fold greater specificity than to another cell type, or to another organ, or to all other cell types combined. In embodiments, the megakaryocyte-derived extracellular vesicles home ex vivo to an HSC with about a 10-fold greater specificity than to another cell type, or to another organ, or to all other cell types combined.

In embodiments, the megakaryocyte-derived extracellular vesicles home ex vivo to one or more hematopoietic stem cells and/or a population of HSCs and/or a plurality of HSCs in and/or derived from bone marrow. In embodiments, the megakaryocyte-derived extracellular vesicles are suitable for homing to one or more hematopoietic stem cells and/or a population of HSCs and/or a plurality of HSCs in vivo. In embodiments, the megakaryocyte-derived extracellular vesicles are suitable for homing to one or more hematopoietic stem cells and/or a population of HSCs and/or a plurality of HSCs in vitro. In embodiments, in a population of HSCs and/or a plurality of HSCs, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, or greater than about 99% of the HSCs comprise MkEVs of the disclosure, including but not limited to cargo-loaded MkEVs, and/or cargo from cargo-loaded MkEVs. In embodiments, the HSCs are Lineage negative (Lin−) HSCs. In embodiments, the HSCs are pluriopotent HSCs. A non-limiting embodiment of pluripotent HSCs include Lineage negative/c-Kit+/Sca-1+ (LSK) HSCs. In embodiments, the HSCs are long-term HSCs. In embodiments, the HSCs are CD45+ HSCs. In embodiments, the HSCs are CD45+/Lin−/CD150+/CD201+/CD48-HSCs.

In embodiments, the megakaryocyte-derived extracellular vesicles home ex vivo to one or more hematopoietic stem cells and/or a population of HSCs and/or a plurality of HSCs in and/or derived from bone marrow. In embodiments, about 50% of the population of HSCs and/or the plurality of HSCs comprise MkEVs of the disclosure, including but not limited to cargo-loaded MkEVs, and/or cargo from cargo-loaded MkEVs. In embodiments, about 50% or less of the population of HSCs and/or the plurality of HSCs comprise MkEVs of the disclosure, including but not limited to cargo-loaded MkEVs, and/or cargo from cargo-loaded MkEVs. In embodiments, about 30% of the population of HSCs and/or the plurality of HSCs comprise MkEVs of the disclosure, including but not limited to cargo-loaded MkEVs, and/or cargo from cargo-loaded MkEVs. In embodiments, about 30% or less of the population of HSCs and/or the plurality of HSCs comprise MkEVs of the disclosure, including but not limited to cargo-loaded MkEVs, and/or cargo from cargo-loaded MkEVs. In embodiments, the HSCs comprise and/or consist of Lineage negative (Lin−) HSCs.

In embodiments, the megakaryocyte-derived extracellular vesicles home ex vivo to one or more hematopoietic stem cells and/or a population of HSCs and/or a plurality of HSCs in and/or derived from bone marrow. In embodiments, about 30% of the population of HSCs and/or the plurality of HSCs comprise MkEVs of the disclosure, including but not limited to cargo-loaded MkEVs, and/or cargo from cargo-loaded MkEVs. In embodiments, about 30% or less of the population of HSCs and/or the plurality of HSCs comprise MkEVs of the disclosure, including but not limited to cargo-loaded MkEVs, and/or cargo from cargo-loaded MkEVs. In embodiments, about 50% of the population of HSCs and/or the plurality of HSCs comprise MkEVs of the disclosure, including but not limited to cargo-loaded MkEVs, and/or cargo from cargo-loaded MkEVs. In embodiments, about 50% or less of the population of HSCs and/or the plurality of HSCs comprise MkEVs of the disclosure, including but not limited to cargo-loaded MkEVs, and/or cargo from cargo-loaded MkEVs. In embodiments, the HSCs comprise and/or consist of Lineage negative/c-Kit+/Sca-1+ (LSK) HSCs.

In embodiments, the megakaryocyte-derived extracellular vesicles are suitable for homing to a lymphatic cell in vivo. In embodiments, the megakaryocyte-derived extracellular vesicles are suitable for homing to a lymphatic cell in vitro. In embodiments, the megakaryocyte-derived extracellular vesicles home in vivo to a lymphatic cell with about a 2-fold greater specificity than to another cell type, or to another organ, or to all other cell types combined. In embodiments, the megakaryocyte-derived extracellular vesicles home in vivo to a lymphatic cell with about a 3-fold greater specificity than to another cell type, or to another organ, or to all other cell types combined. In embodiments, the megakaryocyte-derived extracellular vesicles home in vivo to a lymphatic cell with about a 4-fold greater specificity than to another cell type, or to another organ, or to all other cell types combined. In embodiments, the megakaryocyte-derived extracellular vesicles home in vivo to a lymphatic cell with about a 5-fold greater specificity than to another cell type, or to another organ, or to all other cell types combined. In embodiments, the megakaryocyte-derived extracellular vesicles home in vivo to a lymphatic cell with about a 6-fold greater specificity than to another cell type, or to another organ, or to all other cell types combined. In embodiments, the megakaryocyte-derived extracellular vesicles home in vivo to a lymphatic cell with about a 7-fold greater specificity than to another cell type, or to another organ, or to all other cell types combined. In embodiments, the megakaryocyte-derived extracellular vesicles home in vivo to a lymphatic cell with about a 8-fold greater specificity than to another cell type, or to another organ, or to all other cell types combined. In embodiments, the megakaryocyte-derived extracellular vesicles home in vivo to a lymphatic cell with about a 9-fold greater specificity than to another cell type, or to another organ, or to all other cell types combined. In embodiments, the megakaryocyte-derived extracellular vesicles home in vivo to a lymphatic cell with about a 10-fold greater specificity than to another cell type, or to another organ, or to all other cell types combined.

In embodiments, the megakaryocyte-derived extracellular vesicles are suitable for homing to a regulatory T cell in vivo. In embodiments, the megakaryocyte-derived extracellular vesicles are suitable for homing to a regulatory T cell in vitro. In embodiments, the megakaryocyte-derived extracellular vesicles home in vivo to a regulatory T cell with about a 2-fold greater specificity than to another cell type, or to another organ, or to all other cell types combined. In embodiments, the megakaryocyte-derived extracellular vesicles home in vivo to a regulatory T cell with about a 3-fold greater specificity than to another cell type, or to another organ, or to all other cell types combined. In embodiments, the megakaryocyte-derived extracellular vesicles home in vivo to a regulatory T cell with about a 4-fold greater specificity than to another cell type, or to another organ, or to all other cell types combined. In embodiments, the megakaryocyte-derived extracellular vesicles home in vivo to a regulatory T cell with about a 5-fold greater specificity than to another cell type, or to another organ, or to all other cell types combined. In embodiments, the megakaryocyte-derived extracellular vesicles home in vivo to a regulatory T cell with about a 6-fold greater specificity than to another cell type, or to another organ, or to all other cell types combined. In embodiments, the megakaryocyte-derived extracellular vesicles home in vivo to a regulatory T cell with about a 7-fold greater specificity than to another cell type, or to another organ, or to all other cell types combined. In embodiments, the megakaryocyte-derived extracellular vesicles home in vivo to a regulatory T cell with about a 8-fold greater specificity than to another cell type, or to another organ, or to all other cell types combined. In embodiments, the megakaryocyte-derived extracellular vesicles home in vivo to a regulatory T cell with about a 9-fold greater specificity than to another cell type, or to another organ, or to all other cell types combined. In embodiments, the megakaryocyte-derived extracellular vesicles home in vivo to a regulatory T cell with about a 10-fold greater specificity than to another cell type, or to another organ, or to all other cell types combined.

In embodiments, the megakaryocyte-derived extracellular vesicles are suitable for homing to a liver cell in vivo. In embodiments, the megakaryocyte-derived extracellular vesicles are suitable for homing to a liver cell ex vivo. In embodiments, the megakaryocyte-derived extracellular vesicles are suitable for homing to a liver cell in vitro. In embodiments, the megakaryocyte-derived extracellular vesicles home in vivo to a liver cell with about a 2-fold greater specificity than to another cell type, or to another organ, or to all other cell types combined. In embodiments, the megakaryocyte-derived extracellular vesicles home in vivo to a liver cell with about a 3-fold greater specificity than to another cell type, or to another organ, or to all other cell types combined. In embodiments, the megakaryocyte-derived extracellular vesicles home in vivo to a liver cell with about a 4-fold greater specificity than to another cell type, or to another organ, or to all other cell types combined. In embodiments, the megakaryocyte-derived extracellular vesicles home in vivo to a liver cell with about a 5-fold greater specificity than to another cell type, or to another organ, or to all other cell types combined. In embodiments, the megakaryocyte-derived extracellular vesicles home in vivo to a liver cell with about a 6-fold greater specificity than to another cell type, or to another organ, or to all other cell types combined. In embodiments, the megakaryocyte-derived extracellular vesicles home in vivo to a liver cell with about a 7-fold greater specificity than to another cell type, or to another organ, or to all other cell types combined. In embodiments, the megakaryocyte-derived extracellular vesicles home in vivo to a liver cell with about a 8-fold greater specificity than to another cell type, or to another organ, or to all other cell types combined. In embodiments, the megakaryocyte-derived extracellular vesicles home in vivo to a liver cell with about a 9-fold greater specificity than to another cell type, or to another organ, or to all other cell types combined. In embodiments, the megakaryocyte-derived extracellular vesicles home in vivo to a liver cell with about a 10-fold greater specificity than to another cell type, or to another organ, or to all other cell types combined.

In embodiments, the megakaryocyte-derived extracellular vesicles are suitable for homing to a spleen cell in vivo. In embodiments, the megakaryocyte-derived extracellular vesicles are suitable for homing to a spleen cell ex vivo. In embodiments, the megakaryocyte-derived extracellular vesicles are suitable for homing to a spleen cell in vitro. In embodiments, the megakaryocyte-derived extracellular vesicles home in vivo to a spleen cell with about a 2-fold greater specificity than to another cell type, or to another organ, or to all other cell types combined. In embodiments, the megakaryocyte-derived extracellular vesicles home in vivo to a spleen cell with about a 3-fold greater specificity than to another cell type, or to another organ, or to all other cell types combined. In embodiments, the megakaryocyte-derived extracellular vesicles home in vivo to a spleen cell with about a 4-fold greater specificity than to another cell type, or to another organ, or to all other cell types combined. In embodiments, the megakaryocyte-derived extracellular vesicles home in vivo to a spleen cell with about a 5-fold greater specificity than to another cell type, or to another organ, or to all other cell types combined. In embodiments, the megakaryocyte-derived extracellular vesicles home in vivo to a spleen cell with about a 6-fold greater specificity than to another cell type, or to another organ, or to all other cell types combined. In embodiments, the megakaryocyte-derived extracellular vesicles home in vivo to a spleen cell with about a 7-fold greater specificity than to another cell type, or to another organ, or to all other cell types combined. In embodiments, the megakaryocyte-derived extracellular vesicles home in vivo to a spleen cell with about a 8-fold greater specificity than to another cell type, or to another organ, or to all other cell types combined. In embodiments, the megakaryocyte-derived extracellular vesicles home in vivo to a spleen cell with about a 9-fold greater specificity than to another cell type, or to another organ, or to all other cell types combined. In embodiments, the megakaryocyte-derived extracellular vesicles home in vivo to a spleen cell with about a 10-fold greater specificity than to another cell type, or to another organ, or to all other cell types combined.

In embodiments, the present methods for transferring a deliverable therapeutic agent comprise: (a) obtaining an megakaryocyte-derived extracellular vesicle; (b) incubating the megakaryocyte-derived extracellular vesicle with a therapeutic agent to allow the therapeutic agent to populate the lumen of the megakaryocyte-derived extracellular vesicle and yield a deliverable therapeutic agent; and (c) administering the deliverable therapeutic agent to a patient or contacting the deliverable therapeutic agent with a biological cell in vitro and administering the contacted biological cell to a patient.

In embodiments, the present methods for transferring a deliverable therapeutic agent comprise: (a) obtaining an megakaryocyte-derived extracellular vesicle; (b) incubating the megakaryocyte-derived extracellular vesicle with a therapeutic agent to allow the therapeutic agent to associate with the surface of the megakaryocyte-derived extracellular vesicle and yield a deliverable therapeutic agent; and (c) administering the deliverable therapeutic agent to a patient or contacting the deliverable therapeutic agent with a biological cell in vitro and administering the contacted biological cell to a patient.

In one aspect, the disclosure provides ex vivo methods for transferring a deliverable therapeutic agent. In embodiments, the method comprises: (a) obtaining an megakaryocyte-derived extracellular vesicle; (b) incubating the megakaryocyte-derived extracellular vesicle with a therapeutic agent to allow the therapeutic agent to populate the lumen of the megakaryocyte-derived extracellular vesicle and yield a deliverable therapeutic agent; (c) obtaining a biological cell from a patient; and (d) contacting the deliverable therapeutic agent with the biological cell in vitro and administering the contacted biological cell to the patient.

In embodiments, the method comprises: (a) obtaining an megakaryocyte-derived extracellular vesicle; (b) incubating the megakaryocyte-derived extracellular vesicle with a therapeutic agent to allow the therapeutic agent to associate with the surface of the megakaryocyte-derived extracellular vesicle and yield a deliverable therapeutic agent; (c) obtaining a biological cell from a patient; and (d) contacting the deliverable therapeutic agent with the biological cell in vitro and administering the contacted biological cell to the patient.

In embodiments, the contacting of the deliverable therapeutic agent with the biological cell comprises co-culturing the deliverable therapeutic agent with the biological cell to provide a transfer of the cargo from the deliverable therapeutic agent to the biological cell.

In embodiments, the megakaryocyte-derived extracellular vesicles bind to a cell surface receptor on a cell of the patient. In embodiments, the megakaryocyte-derived extracellular vesicles bind to a cell surface receptor on the contacted biological cell of step (c). In embodiments, the biological cell is one or more of a cancer cell, a tumor cell, a cell infected by a virus, an epithelial cell, an endothelial cell, a nerve cell, a muscle cell, a connective tissue cell, a healthy cell, a diseased cell, a differentiated cell, and a pluripotent cell.

In embodiments, the megakaryocyte-derived extracellular vesicles fuse with the extracellular membrane of a cell of the patient. In embodiments, the megakaryocyte-derived extracellular vesicles fuse with the extracellular membrane of the biological cells of step (c). In embodiments, the biological cell is one or more of a cancer cell, a tumor cell, a cell infected by a virus, an epithelial cell, an endothelial cell, a nerve cell, a muscle cell, a connective tissue cell, a healthy cell, a diseased cell, a differentiated cell, and a pluripotent cell.

In embodiments, the megakaryocyte-derived extracellular vesicles are endocytosed by a cell of the patient. In embodiments, the megakaryocyte-derived extracellular vesicles are endocytosed by the biological cells of step (c). In embodiments, the biological cell is one or more of a cancer cell, a tumor cell, a cell infected by a virus, an epithelial cell, an endothelial cell, a nerve cell, a muscle cell, a connective tissue cell, a healthy cell, a diseased cell, a differentiated cell, and a pluripotent cell.

### Methods of Producing Megakaryocyte-Derived Extracellular Vesicles

In embodiments, a cell culture process is adapted to produce allogeneic megakaryocyte-derived extracellular vesicles from primary human peripheral blood CD34+ HSCs. In embodiments, the megakaryocyte-derived extracellular vesicles are produced by a method comprising obtaining primary human peripheral blood CD34+ HSCs sourced from a commercial supplier and transitioning from a stem cell maintenance medium to an HSC expansion medium. In embodiments, the megakaryocyte-derived extracellular vesicles are produced by a method comprising obtaining primary human cord blood CD34+ HSCs. In embodiments, the megakaryocyte-derived extracellular vesicles are produced by a method comprising obtaining primary human bone marrow CD34+ HSCs. In embodiments, the method further involves placing HSC cultures in a megakaryocyte differentiation medium and collecting megakaryocyte-derived extracellular vesicles from culture supernatant. Accordingly, in embodiments, the present megakaryocyte-derived extracellular vesicles are produced from starting CD34+ HSCs. In embodiments, the presently manufactured megakaryocyte-derived extracellular vesicles are engineered and comprise unique biomarkers.

In embodiments, the megakaryocyte differentiation is confirmed by biomarker expression and/or presence of one or more of CD41, CD61, CD42b, megakaryocyte-specific cytoskeletal proteins β1-tubulin, alpha granule components (e.g. platelet factor 4 and von Willebrand Factor), secretory granules, and ultrastructural characteristics (e.g. invaginated membrane system, dense tubular system, multivesicular bodies).

In embodiments, the megakaryocytes yield between about 500 to about 1500 megakaryocyte-derived extracellular vesicles/cell, which are between about 100 and about 600 nm in diameter (average about 200 nm), DNA−, and CD41+. In embodiments, the megakaryocyte-derived extracellular vesicles are further isolated/concentrated by tangential flow filtration and packaged at targeted concentrations of about 1.5×10⁸ megakaryocyte-derived extracellular vesicles/mL. In embodiments, the megakaryocyte-derived extracellular vesicles exhibit robust expression and/or presence of megakaryocyte and platelet-specific biomarkers, RNA, and cytosolic proteins.

In embodiments, nanoparticle analysis, electron microscopy, flow cytometry, and/or western blots are used to confirm biomarker expression and/or presence and composition of megakaryocyte-derived extracellular vesicles.

In embodiments, the megakaryocyte-derived extracellular vesicles are isolated from megakaryocytes, which are generated in the absence of added erythropoietin. In embodiments, the megakaryocyte-derived extracellular vesicles are isolated from megakaryocytes which are generated in the presence of added thrombopoietin.

In embodiments, the megakaryocyte-derived extracellular vesicles are isolated from the source cell, such as a megakaryocyte, using a method which is substantially free of the external application of biomechanical stress (e.g. to the source cell). Non-limiting examples of methods of isolation that are substantially free of the external application of biomechanical stress include tangential flow filtration and differential centrifugation.

In embodiments, the presently manufactured megakaryocyte-derived extracellular vesicles are substantially free of nucleic acids. In embodiments, the manufactured megakaryocyte-derived extracellular vesicles are substantially free of autologous nucleic acids. In embodiments, the manufactured megakaryocyte-derived extracellular vesicles are substantially free of RNA. In embodiments, the manufactured megakaryocyte-derived extracellular vesicles comprise nucleic acids. In embodiments, the manufactured megakaryocyte-derived extracellular vesicles comprise autologous nucleic acids. In embodiments, the megakaryocyte-derived extracellular vesicles comprise autologous RNA. Non-limiting examples of RNA include rRNA, siRNA, microRNA, regulating RNA, and/or non-coding and coding RNA. In embodiments, the megakaryocyte-derived extracellular vesicles are substantially free of RNA from the cell from which the vesicles are derived. In non-limiting examples, the megakaryocyte-derived extracellular vesicles do not contain RNA due to the method of preparing the vesicles and/or due to the use of RNase to remove native RNAs.

In embodiments, the presently manufactured megakaryocyte-derived extracellular vesicles are substantially free of autologous DNA. In embodiments, the presently megakaryocyte-derived extracellular vesicles are substantially free of DNA from the cell from which the vesicles are derived. In non-limiting examples, the megakaryocyte-derived extracellular vesicles do not contain DNA due to the method of preparing the vesicles and/or due to the use of DNase to remove native DNAs. In embodiments, the megakaryocyte-derived extracellular vesicles are substantially free of one or more of: (a) megakaryocytes, (b) megakaryocyte-derived platelets, and (c) extracellular vesicles derived from platelets.

In embodiments, frozen granulocyte colony-stimulating factor (G-CSF) mobilized human peripheral blood CD34+ cells are obtained and cultured to megakaryocytes before subsequently enriching CD41+ cells (megakaryocytes) prior to culturing, and then measuring the CD41 expression and/or presence and concentration of megakaryocyte-derived extracellular vesicles in the cell culture by flow cytometer or nanoparticle analysis. In embodiments, the megakaryocyte-derived extracellular vesicles are generated by a series of centrifugations, e.g. at escalating speeds/force. In embodiments, the megakaryocyte-derived extracellular vesicles are generated by: (a) removing cells from culture medium at, e.g., about 150×g centrifugation for, e.g., about 10 min; (b) removing platelet-like particles (PLPs) and cell debris by centrifugation at, e.g., about 1000×g for, e.g., about 10 min; and (c) enriching the megakaryocyte-derived extracellular vesicles from the supernatant by ultracentrifugation at, e.g., about 25,000 rpm (38000×g) for, e.g., about 1 hour at, e.g., about 4° C.

In embodiments, a multi-phase culture process with differing pH and pO₂ or pCO₂ and different cytokine cocktails is used to greatly increase megakaryocyte production.

In embodiments, the megakaryocytes are generated by: (a) culturing CD34+ HSCs with a molecular signal/factor/cytokine cocktail that promotes megakaryocyte progenitor production; and (b) shifting cells to different conditions to expand mature megakaryocytes from progenitors. In embodiments, commercial media is used. In embodiments, serum-free media is used. In embodiments, pH is shifted to increase megakaryocyte production. In embodiments, percent CO₂ is shifted to increase megakaryocyte production. In embodiments, the identity of the molecular signals/factors/cytokines is altered to increase megakaryocyte production. In embodiments, the molecular signal/factor/cytokine cocktail contains one or more of TPO, GM-CSF, IL-3, IL-6, IL-11, SCF, FIt3L, IL-9, and the like.

In embodiments, the present production methods further involve the step of characterizing the resultant megakaryocyte-derived extracellular vesicles for one or more proteins (e.g. at least 3, or at least 5, or at least 10, or at least 25, or at least 50, or at least 100, or at least 150, or at least 175) and/or one or more nucleic acids (e.g. at least 3, or at least 5, or at least 10, or at least 25, or at least 50, or at least 100, or at least 150, or at least 175) encoding one or more proteins (e.g. at least 3, or at least 5, or at least 10, or at least 25, or at least 50, or at least 100, or at least 150, or at least 175) selected from H4C16, H2AC17, H3C13, RPL9P9, PHB1, SETSIP, H2AZ1, XP32, COPS9, SEC61A2, MARS1, MRE11, ATP5F1C, H4C1, H2AC20, H3C1, H2BC12, FCSK, CAVIN2, YARS1, H3C15, MACROH2A1, H2AX, H3-2, SELENOF, MPIG6B, SEPTIN7, AARS1, H1-5, H1-2, H2AZ2, GARS1, LARS1, THPO, RACK1, H3-3A, CCDC191, H2BC19P, SEPTIN2, EPRS1, TARS1, ATP5F1B, ERBIN, DARS1, WARS1, VARS1, NIBAN2, SEPTIN6, NARS1, RARS1, SEPTIN11, SEPTIN5, SARS1, NIBAN1, SNRPGP15, PIP4P2, CYRIB, CARMIL1, KARS1, IARS1, SEPTIN9, H1-4, ARHGAP45, H1-0, CARS1, GCN1, FADS2, TBC1D13, GET3, RO60, LAMTOR5, ELOC, H2AC14, SCARF1, RNF24, GCSAML, NRDC, ECPAS, MACROH2A2, ATP5F1A, DMTN, TANGO2, CSF2RB, WASHC5, KCNA3, QARS1, MINDY1, PTPA, EXOC3L2, PRUNE1, PLPBP, THUMPD1, WASHC4, NECTIN2, GFUS, ADGRE2, AKAP8L, FAM234A, ADSS2, ANKRD13D, KCT2, NT5C3A, PIP4P1, CCDC9, ELOB, HPGDS, MEAK7, TOMM70, SMIM1, HARS1, ATP5PD, OGA, CHSY1, SQLE, SUSD6, IGKV1-27, PEDS1-UBE2V1, CEP44, MYG1, NRROS, IL21R, GRK2, MCEMP1, ELAPOR2, SDAD1, CERT1, UBE2F, CALHM5, H1-10, EIPR1, PBDC1, ARMH3, VIPAS39, MESD, PROSER2, RABL6, FYB1, C17orf49, RMDN3, KYAT3, TTMP, ERO1A, CD244, CZIB, PLPP3, BABAM2, H1-3, NAXE, ENSA, PALS2, GUCY1B1, UMAD1, MIX23, PRXL2B, SNU13, RTRAF, KXD1, VSIR, EPOR, and MARCHF2.

In embodiments, the present production methods further involve the step of characterizing the resultant megakaryocyte-derived extracellular vesicles for one or more proteins (e.g. at least 3, or at least 5, or at least 10, or at least 25, or at least 50, or at least 100, or at least 150, or at least 175) and/or one or more nucleic acids (e.g. at least 3, or at least 5, or at least 10, or at least 25, or at least 50, or at least 100, or 20) at least 150, or at least 175) encoding one or more proteins (e.g. at least 3, or at least 5, or at least 10, or at least 25, or at least 50, or at least 100, or at least 150, or at least 175) selected from H4C1, H2AC20, H3C1, H2BC12, FCSK, CAVIN2, YARS1, H3C15, MACROH2A1, H2AX, H3-2, SELENOF, MPIG6B, SEPTIN7, AARS1, H1-5, H1-2, H2AZ2, GARS1, LARS1, THPO, RACK1, H3-3A, CCDC191, H2BC19P, SEPTIN2, EPRS1, TARS1, ATP5F1B, ERBIN, DARS1, WARS1, VARS1, NIBAN2, SEPTIN6, NARS1, RARS1, SEPTIN11, SEPTIN5, SARS1, NIBAN1, SNRPGP15, PIP4P2, CYRIB, CARMIL1, KARS1, IARS1, SEPTIN9, H1-4, ARHGAP45, H1-0, CARS1, GCN1, FADS2, TBC1D13, GET3, RO60, LAMTOR5, ELOC, H2AC14, SCARF1, RNF24, GCSAML, NRDC, ECPAS, MACROH2A2, ATP5F1A, DMTN, TANGO2, CSF2RB, WASHC5, KCNA3, QARS1, MINDY1, PTPA, EXOC3L2, PRUNE1, PLPBP, THUMPD1, WASHC4, NECTIN2, GFUS, ADGRE2, AKAP8L, FAM234A, ADSS2, ANKRD13D, KCT2, NT5C3A, PIP4P1, CCDC9, ELOB, HPGDS, MEAK7, TOMM70, SMIM1, HARS1, ATP5PD, OGA, CHSY1, SQLE, SUSD6, IGKV1-27, PEDS1-UBE2V1, CEP44, MYG1, NRROS, IL21R, GRK2, MCEMP1, ELAPOR2, SDAD1, CERT1, UBE2F, CALHM5, H1-10, EIPR1, PBDC1, ARMH3, VIPAS39, MESD, PROSER2, RABL6, FYB1, C17orf49, RMDN3, KYAT3, TTMP, ERO1A, CD244, CZIB, PLPP3, BABAM2, H1-3, NAXE, ENSA, PALS2, GUCY1B1, UMAD1, MIX23, PRXL2B, SNU13, RTRAF, KXD1, VSIR, EPOR, and MARCHF2.

In embodiments, the present production methods further involve the step of characterizing the resultant megakaryocyte-derived extracellular vesicles for one or more proteins (e.g. at least 3, or at least 5, or at least 10, or at least 25, or at least 50, or at least 100, or at least 150, or at least 175) and/or one or more nucleic acids (e.g. at least 3, or at least 5, or at least 10, or at least 25, or at least 50, or at least 100, or at least 150, or at least 175) encoding one or more proteins (e.g. at least 3, or at least 5, or at least 10, or at least 25, or at least 50, or at least 100, or at least 150, or at least 175) selected from CAVIN2, MPIG6B, ERBIN, ELOC, CSF2RB, KCNA3, NECTIN2, IL21R, MCEMP1, PROSER2, FYB1, CD244, and EPOR.

In embodiments, about 0% to about 5%, about 0% to about 10%, about 15% to about 90%, about 30% to about 80%, or about 50% to about 70% of the megakaryocyte-derived extracellular vesicles in the population comprise one or more proteins (e.g. at least 3, or at least 5, or at least 10, or at least 25, or at least 50, or at least 100, or at least 150, or at least 175) encoding one or more proteins (e.g. at least 3, or at least 5, or at least 10, or at least 25, or at least 50, or at least 100, or at least 150, or at least 175) and/or one or more nucleic acids (e.g. at least 3, or at least 5, or at least 10, or at least 25, or at least 50, or at least 100, or at least 150, or at least 175) encoding one or more proteins (e.g. at least 3, or at least 5, or at least 10, or at least 25, or at least 50, or at least 100, or at least 150, or at least 175) selected from H4C16, H2AC17, H3C13, RPL9P9, PHB1, SETSIP, H2AZ1, XP32, COPS9, SEC61A2, MARS1, MRE11, ATP5F1C, H4C1, H2AC20, H3C1, H2BC12, FCSK, CAVIN2, YARS1, H3C15, MACROH2A1, H2AX, H3-2, SELENOF, MPIG6B, SEPTIN7, AARS1, H1-5, H1-2, H2AZ2, GARS1, LARS1, THPO, RACK1, H3-3A, CCDC191, H2BC19P, SEPTIN2, EPRS1, TARS1, ATP5F1B, ERBIN, DARS1, WARS1, VARS1, NIBAN2, SEPTIN6, NARS1, RARS1, SEPTIN11, SEPTIN5, SARS1, NIBAN1, SNRPGP15, PIP4P2, CYRIB, CARMIL1, KARS1, IARS1, SEPTIN9, H1-4, ARHGAP45, H1-0, CARS1, GCN1, FADS2, TBC1D13, GET3, RO60, LAMTOR5, ELOC, H2AC14, SCARF1, RNF24, GCSAML, NRDC, ECPAS, MACROH2A2, ATP5F1A, DMTN, TANGO2, CSF2RB, WASHC5, KCNA3, QARS1, MINDY1, PTPA, EXOC3L2, PRUNE1, PLPBP, THUMPD1, WASHC4, NECTIN2, GFUS, ADGRE2, AKAP8L, FAM234A, ADSS2, ANKRD13D, KCT2, NT5C3A, PIP4P1, CCDC9, ELOB, HPGDS, MEAK7, TOMM70, SMIM1, HARS1, ATP5PD, OGA, CHSY1, SQLE, SUSD6, IGKV1-27, PEDS1-UBE2V1, CEP44, MYG1, NRROS, IL21R, GRK2, MCEMP1, ELAPOR2, SDAD1, CERT1, UBE2F, CALHM5, H1-10, EIPR1, PBDC1, ARMH3, VIPAS39, MESD, PROSER2, RABL6, FYB1, C17orf49, RMDN3, KYAT3, TTMP, ERO1A, CD244, CZIB, PLPP3, BABAM2, H1-3, NAXE, ENSA, PALS2, GUCY1B1, UMAD1, MIX23, PRXL2B, SNU13, RTRAF, KXD1, VSIR, EPOR, and MARCHF2.

In embodiments, the present production methods further involve the step of characterizing the resultant megakaryocyte-derived extracellular vesicles for one or more nucleic acids (e.g. at least 3, or at least 5, or at least 10, or at least 25, or at least 50, or at least 100, or at least 150, or at least 175, or at least 500, or at least 1000, or at least 1500, or at least 2000, or at least 2500) listed in Table 6 (provided elsewhere herein).

In embodiments, about 0% to about 5%, about 0% to about 10%, about 15% to about 90%, about 30% to about 80%, or about 50% to about 70% of the megakaryocyte-derived extracellular vesicles in the population comprise one or more nucleic acids and/or one or more proteins encoded by one or more nucleic acids listed in Table 6.

In embodiments, the present production methods further involve the step of characterizing the resultant megakaryocyte-derived extracellular vesicles for one or more of CD54, CD18, CD43, CD11b, CD62P, CD41, CD61, CD21, CD51, CLEC-2, LAMP-1 (CD107a), CD63, CD42b, CD9, CD31, CD47, CD147, CD32a, and GPVI. e.g., without limitation by nanoparticle analysis, electron microscopy, flow cytometry, and/or western blot analysis. In embodiments, the present production methods further involve the step of characterizing the resultant megakaryocyte-derived extracellular vesicles for phosphatidylserine, e.g., without limitation by testing for Annexin V, e.g., without limitation by nanoparticle analysis, electron microscopy, flow cytometry, and/or western blot analysis.

In embodiments, the megakaryocyte-derived extracellular vesicles are generated from mature megakaryocytes. In embodiments, the megakaryocyte-derived extracellular vesicles are generated from immature megakaryocytes.

In embodiments, methods to generate megakaryocyte-derived extracellular vesicles are standardized to enable large-scale production.

In embodiments, the present methods to generate megakaryocyte-derived extracellular vesicles inter-batch/donor variability is of less than about 20%, or less than about 15%, or less than about 10%, or less than about 5%. In embodiments, methods to generate megakaryocyte-derived extracellular vesicles are developed such that inter-batch/donor variability is less than 12.5%. In embodiments, methods to generate megakaryocyte-derived extracellular vesicles are developed such that inter-batch/donor variability is less than 10%. In embodiments, methods to generate megakaryocyte-derived extracellular vesicles are developed such that inter-batch/donor variability is less than 7.5%. In embodiments, methods to generate megakaryocyte-derived extracellular vesicles are developed such that inter-batch/donor variability is less than 5%. In embodiments, methods to generate megakaryocyte-derived extracellular vesicles are developed such that inter-batch/donor variability is less than 2.5%.

In embodiments, the population comprises about 1×10⁷ or more, about 1.5×10⁷ or more, about 5×10⁷ or more, 1×10⁸ or more, about 1.5×10⁸ or more, about 5×10⁸ or more, about 1×10⁹ or more, about 5×10⁹ or more, about 1×10¹⁰ or more, or about 1×10¹⁰ or more megakaryocyte-derived extracellular vesicles.

In embodiments, the megakaryocyte-derived extracellular vesicles are isolated as a population. In embodiments, the population of megakaryocyte-derived extracellular vesicles is substantially homogenous.

In embodiments, substantially all of the megakaryocyte-derived extracellular vesicles in the population comprise CD54. In embodiments, about 0% to about 5%, about 0% to about 10%, about 15% to about 90%, about 30% to about 80%, or about 50% to about 70% of the megakaryocyte-derived extracellular vesicles in the population comprise CD54. In embodiments, greater than about 20%, greater than about 30%, greater than about 40%, greater than about 50%, greater than about 60%, greater than about 70%, greater than about 80%, greater than about 90%, or greater than about 95%, or greater than about 99% of the megakaryocyte-derived extracellular vesicles in the population comprise CD54. In embodiments, less than about 30%, less than about 25%, less than about 20%, less than about 15%, less than about 10%, less than about 5%, or less than about 1% of the megakaryocyte-derived extracellular vesicles in the population comprise CD54. In embodiments, all of the megakaryocyte-derived extracellular vesicles in the population are free of, or substantially free of CD54.

In embodiments, substantially all of the megakaryocyte-derived extracellular vesicles in the population comprise CD18. In embodiments, about 0% to about 5%, about 0% to about 10%, about 15% to about 90%, about 30% to about 80%, or about 50% to about 70% of the megakaryocyte-derived extracellular vesicles in the population comprise CD18. In embodiments, greater than about 20%, greater than about 30%, greater than about 40%, greater than about 50%, greater than about 60%, greater than about 70%, greater than about 80%, greater than about 90%, or greater than about 95%, or greater than about 99% of the megakaryocyte-derived extracellular vesicles in the population comprise CD18. In embodiments, less than about 30%, less than about 25%, less than about 20%, less than about 15%, less than about 10%, less than about 5%, or less than about 1% of the megakaryocyte-derived extracellular vesicles in the population comprise CD18. In embodiments, all of the megakaryocyte-derived extracellular vesicles in the population are free of, or substantially free of CD18.

In embodiments, substantially all of the megakaryocyte-derived extracellular vesicles in the population comprise CD43. In embodiments, about 1% to about 30%, about 1% to about 25%, about 1% to about 20%, or about 1% to about 15%, about 0% to about 10% or about 0% to about 10% of the megakaryocyte-derived extracellular vesicles in the population comprise CD43. In embodiments, less than about 30%, less than about 25%, less than about 20%, less than about 15%, less than about 10%, less than about 5%, or less than about 1% of the megakaryocyte-derived extracellular vesicles in the population comprise CD43. In embodiments, all of the megakaryocyte-derived extracellular vesicles in the population are free of, or substantially free of CD43.

In embodiments, substantially all of the megakaryocyte-derived extracellular vesicles in the population comprise CD11b. In embodiments, about 0% to about 5%, about 0% to about 10%, about 1% to about 50%, about 5% to about 40%, or about 10% to about 35% of the megakaryocyte-derived extracellular vesicles in the population comprise CD11b. In embodiments, less than about 50%, less than about 45%, less than about 40%, less than about 35%, less than about 30%, less than about 25%, less than about 20%, less than about 15%, less than about 10%, or less than about 5% of the megakaryocyte-derived extracellular vesicles in the population comprise CD11b. In embodiments, all of the megakaryocyte-derived extracellular vesicles in the population are free of, or substantially free of CD11b.

In embodiments, substantially all of the megakaryocyte-derived extracellular vesicles in the population comprise CD62P. In embodiments, about 0% to about 40%, about 0% to about 30%, about 0% to about 20%, about 0% to about 10%, or about 0% to about 5%, of the megakaryocyte-derived extracellular vesicles in the population comprise CD62P. In embodiments, less than about 40%, less than about 30%, less than about 20%, less than about 10% of the megakaryocyte-derived extracellular vesicles in the population comprise CD62P. In embodiments, all of the megakaryocyte-derived extracellular vesicles in the population are free of, or substantially free of CD62P.

In embodiments, substantially all of the megakaryocyte-derived extracellular vesicles in the population comprise CD41. In embodiments, about 15% to about 90%, about 30% to about 80%, or about 50% to about 70% of the megakaryocyte-derived extracellular vesicles in the population comprise CD41. In embodiments, greater than about 20%, greater than about 30%, greater than about 40%, greater than about 50%, greater than about 60%, greater than about 70%, greater than about 80%, greater than about 90%, or greater than about 95%, or greater than about 99% of the megakaryocyte-derived extracellular vesicles in the population comprise CD41.

In embodiments, substantially all of the megakaryocyte-derived extracellular vesicles in the population comprise CD61. In embodiments, about 40% to about 100%, about 60% to about 100%, or about 85% to about 10% of the megakaryocyte-derived extracellular vesicles in the population comprise CD61. In embodiments, greater than about 40%, greater than about 50%, greater than about 60%, greater than about 70%, greater than about 80%, greater than about 85%, greater than about 90%, or greater than about 95%, or greater than about 99% of the megakaryocyte-derived extracellular vesicles in the population comprise CD61.

In embodiments, substantially all of the megakaryocyte-derived extracellular vesicles in the population comprise CD21. In embodiments, about 0% to about 10%, about 0% to about 5%, about 15% to about 90%, about 30% to about 80%, or about 50% to about 70% of the megakaryocyte-derived extracellular vesicles in the population comprise CD21. In embodiments, greater than about 20%, greater than about 30%, greater than about 40%, greater than about 50%, greater than about 60%, greater than about 70%, greater than about 80%, greater than about 90%, or greater than about 95%, or greater than about 99% of the megakaryocyte-derived extracellular vesicles in the population comprise CD21. In embodiments, less than about 50%, less than about 45%, less than about 40%, less than about 35%, less than about 30%, less than about 25%, less than about 20%, less than about 15%, less than about 10%, or less than about 5% of the megakaryocyte-derived extracellular vesicles in the population comprise CD21. In embodiments, all of the megakaryocyte-derived extracellular vesicles in the population are free of, or substantially free of CD21.

In embodiments, substantially all of the megakaryocyte-derived extracellular vesicles in the population comprise CD51. In embodiments, about 0% to about 10%, about 0% to about 5%, about 15% to about 90%, about 30% to about 80%, or about 50% to about 70% of the megakaryocyte-derived extracellular vesicles in the population comprise CD51. In embodiments, greater than about 20%, greater than about 30%, greater than about 40%, greater than about 50%, greater than about 60%, greater than about 70%, greater than about 80%, greater than about 90%, or greater than about 95%, or greater than about 99% of the megakaryocyte-derived extracellular vesicles in the population comprise CD51. In embodiments, less than about 50%, less than about 45%, less than about 40%, less than about 35%, less than about 30%, less than about 25%, less than about 20%, less than about 15%, less than about 10%, or less than about 5% of the megakaryocyte-derived extracellular vesicles in the population comprise CD51. In embodiments, all of the megakaryocyte-derived extracellular vesicles in the population are free of, or substantially free of CD51.

In embodiments, substantially all of the megakaryocyte-derived extracellular vesicles in the population comprise CLEC-2. In embodiments, about 0% to about 10%, about 0% to about 5%, or about 0% to about 12% of the megakaryocyte-derived extracellular vesicles in the population comprise CLEC-2. In embodiments, less than about 10%, less than about 5%, or less than about 2% of the megakaryocyte-derived extracellular vesicles in the population comprise CLEC-2. In embodiments, all of the megakaryocyte-derived extracellular vesicles in the population are free of, or substantially free of CLEC-2.

In embodiments, substantially all of the megakaryocyte-derived extracellular vesicles in the population comprise LAMP-1 (CD107a). In embodiments, about 0% to about 20%, about 1% to about 15%, about 2% to about 10%, about 0% to about 5%, or about 0% to about 5% of the megakaryocyte-derived extracellular vesicles in the population comprise LAMP-1 (CD107a). In embodiments, less than about 20%, less than about 15%, less than about 10%, or less than about 5% of the megakaryocyte-derived extracellular vesicles in the population comprise LAMP-1 (CD107a). In embodiments, all of the megakaryocyte-derived extracellular vesicles in the population are free of, or substantially free of LAMP-1 (CD107a).

In embodiments, less than about 20%, less than about 15%, less than about 10%, or less than about 5% of a population of CD41+ megakaryocyte-derived extracellular vesicles comprise LAMP-1 (CD107a).

In embodiments, the megakaryocyte-derived extracellular vesicles in the population are substantially free of DRAQ5. In embodiments, about 0% to about 20%, about 0% to about 15%, about 0% to about 10%, or about 0% to about 5% of the megakaryocyte-derived extracellular vesicles in the population comprise DRAQ5. In embodiments, less than about 20%, less than about 15%, less than about 10%, or less than about 5% of the megakaryocyte-derived extracellular vesicles in the population comprise DRAQ5.

In embodiments, substantially all of the megakaryocyte-derived extracellular vesicles in the population comprise CD63. In embodiments, about 1% to about 20%, about 1% to about 15%, or about 1% to about 10% of the megakaryocyte-derived extracellular vesicles in the population comprise CD63. In embodiments, less than about 20%, less than about 15%, less than about 10%, less than about 5%, or less than about 1% of the megakaryocyte-derived extracellular vesicles in the population comprise CD63. In embodiments, all of the megakaryocyte-derived extracellular vesicles in the population are free of, or substantially free of CD63.

In embodiments, substantially all of the megakaryocyte-derived extracellular vesicles in the population comprise CD42b. In embodiments, about 0% to about 20%, about 0% to about 15%, about 0% to about 10%, or about 0% to about 5% of the megakaryocyte-derived extracellular vesicles in the population comprise CD42b. In embodiments, less than about 20%, less than about 15%, less than about 10%, less than about 5%, or less than about 1% of the megakaryocyte-derived extracellular vesicles in the population comprise CD42b. In embodiments, all of the megakaryocyte-derived extracellular vesicles in the population are free of, or substantially free of CD42b

In embodiments, substantially all of the megakaryocyte-derived extracellular vesicles in the population comprise CD9. In embodiments, about 40% to about 100%, about 50% to about 80%, or about 60% to about 70% of the megakaryocyte-derived extracellular vesicles in the population comprise CD9. In embodiments, greater than about 30%, greater than about 40%, greater than about 50%, greater than about 60%, greater than about 70%, greater than about 80%, greater than about 90%, or greater than about 95%, or greater than about 99% of the megakaryocyte-derived extracellular vesicles in the population comprise CD9.

In embodiments, substantially all of the megakaryocyte-derived extracellular vesicles in the population comprise CD31. In embodiments, about 1% to about 30%, about 1% to about 25%, about 1% to about 20%, or about 1% to about 15% of the megakaryocyte-derived extracellular vesicles in the population comprise CD31. In embodiments, less than about 30%, less than about 25%, less than about 20%, less than about 15%, less than about 10%, less than about 5%, or less than about 1% of the megakaryocyte-derived extracellular vesicles in the population comprise CD31. In embodiments, all of the megakaryocyte-derived extracellular vesicles in the population are free of, or substantially free of CD31.

In embodiments, substantially all of the megakaryocyte-derived extracellular vesicles in the population comprise CD47. In embodiments, about 1% to about 40%, about 1% to about 35%, about 1% to about 20%, about 20% to about 30%, about 30% to about 40%, or about 1% to about 15% of the megakaryocyte-derived extracellular vesicles in the population comprise CD47. In embodiments, less than about 40%, less than about 30%, less than about 25%, less than about 20%, less than about 15%, less than about 10%, less than about 5%, or less than about 1% of the megakaryocyte-derived extracellular vesicles in the population comprise CD47. In embodiments, all of the megakaryocyte-derived extracellular vesicles in the population are free of, or substantially free of CD47.

In embodiments, substantially all of the megakaryocyte-derived extracellular vesicles in the population comprise CD147. In embodiments, about 1% to about 30%, about 1% to about 25%, about 1% to about 20%, about 20% to about 30%, or about 1% to about 15% of the megakaryocyte-derived extracellular vesicles in the population comprise CD147. In embodiments, less than about 30%, less than about 25%, less than about 20%, less than about 15%, less than about 10%, less than about 5%, or less than about 1% of the megakaryocyte-derived extracellular vesicles in the population comprise CD147. In embodiments, all of the megakaryocyte-derived extracellular vesicles in the population are free of, or substantially free of CD147.

In embodiments, substantially all of the megakaryocyte-derived extracellular vesicles in the population comprise CD32a. In embodiments, about 0% to about 20%, about 1% to about 15%, or about 1% to about 10% of the megakaryocyte-derived extracellular vesicles in the population comprise CD32a. In embodiments, less than about 20%, less than about 15%, less than about 10%, less than about 5%, or less than about 1% of the megakaryocyte-derived extracellular vesicles in the population comprise CD32a. In embodiments, all of the megakaryocyte-derived extracellular vesicles in the population are free of, or substantially free of CD32a.

In embodiments, substantially all of the megakaryocyte-derived extracellular vesicles in the population comprise GVPI. In embodiments, about 0% to about 5%, about 0% to about 10%, about 0% to about 30%, about 0% to about 15%, or about 0% to about 10% of the megakaryocyte-derived extracellular vesicles in the population comprise GVPI. In embodiments, less than about 30%, less than about 25%, less than about 20%, less than about 15%, less than about 10%, less than about 5%, or less than about 1% of the megakaryocyte-derived extracellular vesicles in the population comprise GVPI. In embodiments, all of the megakaryocyte-derived extracellular vesicles in the population are free of, or substantially free of GVPI.

In embodiments, substantially all of the megakaryocyte-derived extracellular vesicles in the population comprise phosphatidylserine. In embodiments, about 15% to about 90%, about 30% to about 80%, or about 50% to about 70% of the megakaryocyte-derived extracellular vesicles in the population comprise phosphatidylserine. In embodiments, greater than about 20%, greater than about 30%, greater than about 40%, greater than about 50%, greater than about 60%, greater than about 70%, greater than about 80%, greater than about 90%, or greater than about 95%, or greater than about 99% of the megakaryocyte-derived extracellular vesicles in the population comprise phosphatidylserine. In embodiments, less than about 30%, less than about 25%, less than about 20%, less than about 15%, less than about 10%, less than about 5%, or less than about 1% of the megakaryocyte-derived extracellular vesicles in the population comprise GVPI. In embodiments, all of the megakaryocyte-derived extracellular vesicles in the population are free of, or substantially free of phosphatidylserine.

In embodiments, the megakaryocyte-derived extracellular vesicles are generated by: (a) obtaining a human pluripotent stem cell being a primary CD34+ HSC sourced from peripheral blood or cord blood; (b) differentiating the human pluripotent stem cell to a megakaryocyte in the absence of added EPO and in the presence of added TPO; and (c) isolating the megakaryocyte-derived extracellular vesicles from the megakaryocytes.

In embodiments, the method is an in vivo method. In embodiments, the method is an ex vivo method.

In embodiments, the CD34+ HSC sourced from peripheral blood are multipotent stem cells derived from volunteers whose stem cells are mobilized into the bloodstream by administration of a mobilization agent such as granulocyte colony stimulating factor (G-CSF) and granulocyte-macrophage colony stimulating factor (GM-CSF).

In embodiments, the cord blood comprises multipotent stem cells derived from blood that remains in the placenta and the attached umbilical cord after childbirth.

In embodiments, the megakaryocyte-derived extracellular vesicles are autologous with the patient. In embodiments, human pluripotent stem cells are extracted from the patient and used to generate megakaryocytes, from which megakaryocyte-derived extracellular vesicles comprising a cargo of choice are generated and then administered to the patient. In embodiments, differentiated cells are extracted from the patient and used to generate iPSCs, which in turn are used to generate megakaryocytes, from which megakaryocyte-derived extracellular vesicles comprising a cargo of choice are generated and then administered to the patient.

In embodiments, the megakaryocyte-derived extracellular vesicles are allogeneic with the patient. In embodiments, human pluripotent stem cells are extracted from a human subject who is not the patient and used to generate megakaryocytes, from which megakaryocyte-derived extracellular vesicles comprising a cargo of choice are generated and then administered to the patient. In embodiments, differentiated cells are extracted from a human subject who is not the patient and used to generate iPSCs, which in turn are used to generate megakaryocytes, from which megakaryocyte-derived extracellular vesicles comprising a cargo of choice are generated and then administered to the patient.

In embodiments, the megakaryocyte-derived extracellular vesicles are heterologous with the patient. In embodiments, pluripotent stem cells are extracted from a non-human subject and used to generate megakaryocytes, from which megakaryocyte-derived extracellular vesicles comprising a cargo of choice are generated and then administered to the patient. In embodiments, differentiated cells are extracted from a non-human subject and used to generate iPSCs, which in turn are used to generate megakaryocytes, from which megakaryocyte-derived extracellular vesicles comprising a cargo of choice are generated and then administered to the patient.

In embodiments, the incubating comprises one or more of sonication, saponin permeabilization, mechanical vibration, hypotonic dialysis, extrusion through porous membranes, cholesterol conjugation, application of electric current and combinations thereof. In embodiments, the incubating comprises one or more of electroporating, transforming, transfecting, and microinjecting.

In embodiments, the method further comprises (d) contacting the megakaryocyte-derived extracellular vesicles with radiation. In embodiments, the radiation is gamma radiation. In embodiments, the gamma radiation is at an amount greater than 12kGy, kGy, or 50kGy. In embodiments, the gamma radiation is at an amount between about 12kGy and 15kGy. In embodiments, the gamma radiation is at an amount between about 15kGy and 20kGy. In embodiments, the gamma radiation is at an amount between about 20kGy and 25kGy. In embodiments, the gamma radiation is at an amount between about 25kGy and 30kGy. In embodiments, the gamma radiation is at an amount between about 30kGy and 35kGy. In embodiments, the gamma radiation is at an amount between about 35kGy and 40kGy. In embodiments, the gamma radiation is at an amount between about 40kGy and 45kGy. In embodiments, the gamma radiation is at an amount between about 45kGy and 50kGy. In embodiments, the gamma radiation is at an amount between about 50kGy and 55kGy. In embodiments, the gamma radiation is at an amount between about 55kGy and 60kGy.

In embodiments, the method is substantially serum free. In embodiments, the method is greater than 60% serum free. In embodiments, the method is greater than 70% serum free. In embodiments, the method is greater than 80% serum free. In embodiments, the method is greater than 90% serum free.

In various embodiments, the compositions comprise substantially purified megakaryocyte-derived extracellular vesicles. In embodiments, substantially purified is synonymous with biologically pure. In embodiments, the substantially purified megakaryocyte-derived extracellular vesicles are largely free to varying degrees from components which normally accompany it as found in its native state. “Isolate” denotes a degree of separation from original source or surroundings. In embodiments, the substantially purified megakaryocyte-derived extracellular vesicles are sufficiently free of other materials such that any impurities do not materially affect the biological properties of the megakaryocyte-derived extracellular vesicles or cause other adverse consequences. In embodiments, the substantially purified megakaryocyte-derived extracellular vesicles are sufficiently free of cellular material, viral material, or culture medium that may be needed for production. Purity and homogeneity are typically determined using biochemical techniques known in the art. In embodiments, the megakaryocyte-derived extracellular vesicles are purified using size exclusion filtration. In embodiments, the filter has a pore size of about 650 nm. In embodiments, the megakaryocyte-derived extracellular vesicles are purified using size exclusion filtration. In embodiments, the filter has a pore size ranging from about 50 nm to about 600 nm. In embodiments, the filter has a pore size of at least 50 nm. In embodiments, the filter has a pore size of about 600 nm.

### Cargo of Megakaryocyte-Derived Extracellular Vesicles

Megakaryocyte-derived extracellular vesicles may contain diverse cargo such as mRNAs, microRNAs, and cytokines. Megakaryocyte-derived extracellular vesicles are able to transfer their cargo to alter the function of target cells. They exert their influence on the target cells through surface receptor signaling, plasma membrane fusion, and internalization. By loading megakaryocytes or megakaryocyte-derived extracellular vesicles with biologic or therapeutic cargo, megakaryocyte-derived extracellular vesicles can be further used as delivery vehicles to achieve a targeted therapeutic effect. Until now, small RNAs (siRNA and miRNA), small linear DNA, and plasmid DNA have all been successfully loaded into megakaryocyte-derived extracellular vesicles for a variety of delivery applications. Megakaryocyte-derived extracellular vesicles targeting is defined by their complement of surface proteins and can be further engineered to express or remove specific biomarkers of interest to refine biodistribution and cell-cell recognition. For instance, the present megakaryocyte-derived extracellular vesicles, with their unique biomarker profiles, are particularly suited for delivery of payloads, e.g. therapies.

In embodiments, the megakaryocyte-derived extracellular vesicles are suitable for loading with cargo into the lumen. In embodiments, the cargo is selected from one or more of a RNA, DNA, protein, carbohydrate, lipid, biomolecule, and small molecule. In embodiments, the cargo is a biologically produced component. In embodiments, the cargo is a synthetically produced component. In embodiments, the cargo is pre-loaded into megakaryocyte-derived extracellular vesicles. In embodiments, a biological component is overexpressed in megakaryocytes so that generated megakaryocyte-derived extracellular vesicles comprise the biological component. In embodiments, the cargo is post-loaded into megakaryocyte-derived extracellular vesicles. In embodiments, purified megakaryocyte-derived extracellular vesicles are mixed with cargo to generate cargo-loaded megakaryocyte-derived extracellular vesicles. In embodiments, the cargo is hydrophobic. In embodiments, the cargo is hydrophilic. In embodiments, the cargo is integrated into the lipid bilayer of the megakaryocyte-derived extracellular vesicles. In embodiments, the cargo is located in the lumen of the megakaryocyte-derived extracellular vesicles.

In embodiments, in addition to or as an alternative to the cargo located in the lumen of the megakaryocyte-derived extracellular vesicles, the cargo is associated with the megakaryocyte-derived extracellular vesicles. In embodiments, the cargo is associated with the surface and/or the exterior of the megakaryocyte-derived extracellular vesicles. Non-limiting examples of cargo associated with the megakaryocyte-derived extracellular vesicles includes cargo that is covalently conjugated to the surface of the vesicle or cargo that is associated with the surface via electrostatic interactions. As would be understood by one of ordinary skill in the art, cargo associated with the megakaryocyte-derived extracellular vesicles can still be transported even when not loaded into the lumen of the vesicle.

In embodiments, the cargo is loaded into the megakaryocyte-derived extracellular vesicle using an active loading strategy, which is physically-induced and/or chemically-induced. In embodiments, the active loading strategy is physically-induced. In embodiments, the physically-induced active loading strategy comprises the mechanical or physical disruption of the megakaryocyte-derived extracellular vesicle lipid bilayer through external forces, such as electroporation, sonication, freeze-thaw cycling, and extrusion. In embodiments, the electroporation involves the use of an electric field to induce spontaneous pore formation in the megakaryocyte-derived extracellular vesicle lipid bilayer, wherein the presence of the electric field disrupts the lipid bilayer, while removal of the field enables closure of pores and reformation of the lipid layer after the cargo has been taken up by the megakaryocyte-derived extracellular vesicle. In embodiments, the sonication involves ultrasound energy applied through a sonicator probe that decreases the rigidity of the megakaryocyte-derived extracellular vesicle lipid bilayer, enabling cargo diffusion. In embodiments, the freeze-thaw cycling uses thermal energy to facilitate megakaryocyte-derived extracellular vesicle cargo loading. In embodiments, extrusion is performed following established protocols for formation of synthetic liposomes, wherein megakaryocyte-derived extracellular vesicles are mixed with free cargo and passed through membranes containing nanoscale pores, wherein the sheer force disrupts the lipid bilayer, allowing exogenous cargo to enter megakaryocyte-derived extracellular vesicles.

In embodiments, the active loading strategy is chemically-induced. In embodiments, the chemically-induced active loading strategy comprises the use of chemical agents, such as saponin or transfection reagents, to bypass the megakaryocyte-derived extracellular vesicle lipid bilayer. In embodiments, the chemical agent is a detergent, such as saponin. In embodiments, the saponin is used to selectively remove cholesterol from the megakaryocyte-derived extracellular vesicle lipid bilayer, opening pores in the lipid bilayer. In embodiments, the chemical agent is a transfection agent. In embodiments, the transfection agent is used to deliver nucleic acids into the megakaryocyte-derived extracellular vesicle by exploiting cationic substances that promote interactions with the lipid bilayer and subsequent internalization. In embodiments, the transfection agent is lipofectamine and/or a lipid-based agent.

In embodiments, the loading ratio of a nucleic acid (i.e. copies of nucleic acid per vesicle) into megakaryocyte-derived extracellular vesicles of the disclosure ranges from about 1 to about 1000, about 1 to about 500, about 1 to about 100, about 10 to about 1000, about 100 to about 1000, about 500 to about 1000, about 100 to about 500,000, about 1000 to about 300,000, about 100,000 to about 300,000, about 1000, to about 10,000, or about 1000 to about 5000. In embodiments, the nucleic acid is DNA. In embodiments, the nucleic acid is plasmid DNA.

In embodiments, the loading efficiency for loading cargo, such as a nucleic acid, into megakaryocyte-derived extracellular vesicles of the disclosure ranges from about 1% to about 99%, about 10% to about 90%, about 30% to about 70%, about 40% to about 60%, about 40% to about 50%, or about 50% to about 60%. In embodiments, the cargo is a nucleic acid. In embodiments, the nucleic acid is DNA. In embodiments, the nucleic acid is plasmid DNA. In embodiments, loading efficiency is calculated using the following equation:

$$Loading efficiency ( \% ) = cargo + MV \# / Total MV \#$$

In embodiments, the surface of megakaryocyte-derived extracellular vesicles is modified to impact biodistribution and targeting capabilities of megakaryocyte-derived extracellular vesicles. In embodiments, surface ligands are added to megakaryocyte-derived extracellular vesicles through genetic engineering. In embodiments, the megakaryocyte-derived extracellular vesicles are generated that express fusion proteins in their lipid bilayers. In embodiments, the endogenous proteins in megakaryocyte-derived extracellular vesicle lipid bilayers are fused with targeting ligands through cell engineering.

In embodiments, the cargo is one or more therapeutic agents. In embodiments, the therapeutic agent is a nucleic acid therapeutic agent. In embodiments, the nucleic acid therapeutic agent encodes a functional protein.

In embodiments, the nucleic acid therapeutic agent is selected from one or more non-autologous and/or recombinant nucleic acid constructs selected from mRNA, tRNA, rRNA, siRNA, microRNA, regulating RNA, non-coding and coding RNA, linear DNA, DNA fragments, or DNA plasmids. In embodiments, the nucleic acid therapeutic agent is selected from one or more of mRNA, miRNA, siRNA, and snoRNA.

In embodiments, the nucleic acid therapeutic agent encodes a wild type gene, which is defective in the patient. In embodiments, the nucleic acid therapeutic agent is mRNA, and optionally: is in vitro transcribed or synthetic and/or comprises one or more non-canonical nucleotides, optionally selected from pseudouridine and 5-methoxyuridine.

In embodiments, the one or more non-canonical nucleotides are selected from 2-thiouridine, 5-azauridine, pseudouridine, 4-thiouridine, 5-methyluridine, 5-methylpseudouridine, 5-aminouridine, 5-aminopseudouridine, 5-hydroxyuridine, 5-hydroxypseudouridine, 5-methoxyuridine, 5-methoxypseudouridine, 5-ethoxyuridine, 5-ethoxypseudouridine, 5-hydroxymethyluridine, 5-hydroxymethylpseudouridine, 5-carboxyuridine, 5-carboxypseudouridine, 5-formyluridine, 5-formylpseudouridine, 5-methyl-5-azauridine, 5-amino-5-azauridine, 5-hydroxy-5-azauridine, 5-methylpseudouridine, 5-aminopseudouridine, 5-hydroxypseudouridine, 4-thio-5-azauridine, 4-thiopseudouridine, 4-thio-5-methyluridine, 4-thio-5-aminouridine, 4-thio-5-hydroxyuridine, 4-thio-5-methyl-5-azauridine, 4-thio-5-amino-5-azauridine, 4-thio-5-hydroxy-5-azauridine, 4-thio-5-methylpseudouridine, 4-thio-5-aminopseudouridine, 4-thio-5-hydroxypseudouridine, 2-thiocytidine, 5-azacytidine, pseudoisocytidine, N4-methylcytidine, N4-aminocytidine, N4-hydroxycytidine, 5-methylcytidine, 5-aminocytidine, 5-hydroxycytidine, 5-methoxycytidine, 5-ethoxycytidine, 5-hydroxymethylcytidine, 5-carboxycytidine, 5-formylcytydine, 5-methyl-5-azacytidine, 5-amino-5-azacytidine, 5-hydroxy-5-azacytidine, 5-methylpseudoisocytidine, 5-aminopseudoisocytidine, 5-hydroxypseudoisocytidine, N4-methyl-5-azacytidine, N4-methylpseudoisocytidine, 2-thio-5-azacytidine, 2-thiopseudoisocytidine, 2-thio-N4-methylcytidine, 2-thio-N4-aminocytidine, 2-thio-N4-hydroxycytidine, 2-thio-5-methylcytidine, 2-thio-5-aminocytidine, 2-thio-5-hydroxycytidine, 2-thio-5-methyl-5-azacytidine, 2-thio-5-amino-5-azacytidine, 2-thio-5-hydroxy-5-azacytidine, 2-thio-5-methylpseudoisocytidine, 2-thio-5-aminopseudoisocytidine, 2-thio-5-hydroxypseudoisocytidine, 2-thio-N4-methyl-5-azacytidine, 2-thio-N4-methylpseudoisocytidine, N4-methyl-5-methylcytidine, N4-methyl-5-aminocytidine, N4-methyl-5-hydroxycytidine, N4-methyl-5-methyl-5-azacytidine, N4-methyl-5-amino-5-azacytidine, N4-methyl-5-hydroxy-5-azacytidine, N4-methyl-5-methylpseudoisocytidine, N4-methyl-5-aminopseudoisocytidine, N4-methyl-5-hydroxypseudoisocytidine, N4-amino-5-azacytidine, N4-aminopseudoisocytidine, N4-amino-5-methylcytidine, N4-amino-5-aminocytidine, N4-amino-5-hydroxycytidine, N4-amino-5-methyl-5-azacytidine, N4-amino-5-amino-5-azacytidine, N4-amino-5-hydroxy-5-azacytidine, N4-amino-5-methylpseudoisocytidine, N4-amino-5-aminopseudoisocytidine, N4-amino-5-hydroxypseudoisocytidine, N4-hydroxy-5-azacytidine, N4-hydroxypseudoisocytidine, N4-hydroxy-5-methylcytidine, N4-hydroxy-5-aminocytidine, N4-hydroxy-5-hydroxycytidine, N4-hydroxy-5-methyl-5-azacytidine, N4-hydroxy-5-amino-5-azacytidine, N4-hydroxy-5-hydroxy-5-azacytidine, N4-hydroxy-5-methylpseudoisocytidine, N4-hydroxy-5-aminopseudoisocytidine, N4-hydroxy-5-hydroxypseudoisocytidine, 2-thio-N4-methyl-5-methylcytidine, 2-thio-N4-methyl-5-aminocytidine, 2-thio-N4-methyl-5-hydroxycytidine, 2-thio-N4-methyl-5-methyl-5-azacytidine, 2-thio-N4-methyl-5-amino-5-azacytidine, 2-thio-N4-methyl-5-hydroxy-5-azacytidine, 2-thio-N4-methyl-5-methylpseudoisocytidine, 2-thio-N4-methyl-5-aminopseudoisocytidine, 2-thio-N4-methyl-5-hydroxypseudoisocytidine, 2-thio-N4-amino-5-azacytidine, 2-thio-N4-aminopseudoisocytidine, 2-thio-N4-amino-5-methylcytidine, 2-thio-N4-amino-5-aminocytidine, 2-thio-N4-amino-5-hydroxycytidine, 2-thio-N4-amino-5-methyl-5-azacytidine, 2-thio-N4-amino-5-amino-5-azacytidine, 2-thio-N4-amino-5-hydroxy-5-azacytidine, 2-thio-N4-amino-5-methylpseudoisocytidine, 2-thio-N4-amino-5-aminopseudoisocytidine, 2-thio-N4-amino-5-hydroxypseudoisocytidine, 2-thio-N4-hydroxy-5-azacytidine, 2-thio-N4-hydroxypseudoisocytidine, 2-thio-N4-hydroxy-5-methylcytidine, N4-hydroxy-5-aminocytidine, 2-thio-N4-hydroxy-5-hydroxycytidine, 2-thio-N4-hydroxy-5-methyl-5-azacytidine, 2-thio-N4-hydroxy-5-amino-5-azacytidine, 2-thio-N4-hydroxy-5-hydroxy-5-azacytidine, 2-thio-N4-hydroxy-5-methylpseudoisocytidine, 2-thio-N4-hydroxy-5-aminopseudoisocytidine, 2-thio-N4-hydroxy-5-hydroxypseudoisocytidine, N6-methyladenosine, N6-aminoadenosine, N6-hydroxyadenosine, 7-deazaadenosine, 8-azaadenosine, N6-methyl-7-deazaadenosine, N6-methyl-8-azaadenosine, 7-deaza-8-azaadenosine, N6-methyl-7-deaza-8-azaadenosine, N6-amino-7-deazaadenosine, N6-amino-8-azaadenosine, N6-amino-7-deaza-8-azaadenosine, N6-hydroxyadenosine, N6-hydroxy-7-deazaadenosine, N6-hydroxy-8-azaadenosine, N6-hydroxy-7-deaza-8-azaadenosine, 6-thioguanosine, 7-deazaguanosine, 8-azaguanosine, 6-thio-7-deazaguanosine, 6-thio-8-azaguanosine, 7-deaza-8-azaguanosine, and 6-thio-7-deaza-8-azaguanosine.

In embodiments, the present methods comprise gene-editing and/or gene correction. In embodiments, the present methods encompass synthetic RNA-based gene-editing and/or gene correction, e.g. with RNA comprising non-canonical nucleotides, e.g. RNA encoding one or more of a nuclease, a transcription activator-like effector nuclease (TALEN), a zinc-finger nuclease, a meganuclease, a nickase, a clustered regularly interspaced short palindromic repeat (CRISPR)-associated protein a DNA-repair protein, a DNA-modification protein, a base-modification protein, a DNA methyltransferase, a protein that causes DNA demethylation, an enzyme for which DNA is a substrate or a natural or engineered variant, family-member, orthologue, fragment or fusion construct thereof. In embodiments, the efficiency of the gene-editing and/or gene correction is high, for example, higher than DNA-based gene editing and/or gene correction. In embodiments, the present methods of gene-editing and/or gene correction are efficient enough for in vivo application. In embodiments, the present methods of gene-editing and/or gene correction are efficient enough to not require cellular selection (e.g. selection of cells that have been edited). In embodiments, the efficiency of gene-editing of the present methods is about 1%, or about 2%, or about 3%, or about 4%, or about 5%, or about 6%, or about 7%, or about 8%, or about 9%, or about 10%, or about 20%, or about 30%, or about 40%, or about 50%, or about 60%, or about 70%, or about 80%, or about 90%, or about 100%. In embodiments, the efficiency of gene-correction of the present methods is about 1%, or about 2%, or about 3%, or about 4%, or about 5%, or about 6%, or about 7%, or about 8%, or about 9%, or about 10%, or about 20%, or about 30%, or about 40%, or about 50%, or about 60%, or about 70%, or about 80%, or about 90%, or about 100% In embodiments, the present methods comprise high-efficiency gene-editing proteins comprising engineered nuclease cleavage or DNA-modification domains. In embodiments, the methods comprise high-fidelity gene-editing proteins comprising engineered nuclease cleavage or DNA-modification domains. In embodiments, the high-efficiency gene-editing proteins comprising engineered DNA-binding domains. In embodiments, the high-fidelity gene-editing proteins comprising engineered DNA-binding domains. In embodiments, the methods comprise gene-editing proteins comprising engineered repeat sequences. In embodiments, the methods comprise gene-editing proteins comprising one or more CRISPR associated family members. In embodiments, the methods comprise altering the DNA sequence of a cell by transfecting the cell with or inducing the cell to express a gene-editing protein. In embodiments, the methods comprise altering the DNA sequence of a cell that is present in an in vitro culture. In embodiments, the methods comprise altering the DNA sequence of a cell that is present in vivo.

In embodiments, the methods comprise one or more steroids and/or one or more antioxidants in the transfection medium can increase in vivo transfection efficiency, in vivo reprogramming efficiency, and in vivo gene-editing efficiency. In embodiments, the methods comprise contacting a cell or patient with a glucocorticoid, such as hydrocortisone, prednisone, prednisolone, methylprednisolone, dexamethasone or betamethasone. In embodiments, the methods comprise inducing a cell to express a protein of interest by contacting a cell with a medium containing a steroid and contacting the cell with one or more nucleic acid molecules. In embodiments, the nucleic acid molecule comprises synthetic RNA. In embodiments, the steroid is hydrocortisone. In embodiments, the hydrocortisone is present in the medium at a concentration of between about 0.1 uM and about 10 uM, or about 1 uM. In embodiments, the methods comprise inducing a cell in vivo to express a protein of interest by contacting the cell with a medium containing an antioxidant and contacting the cell with one or more nucleic acid molecules. In embodiments, the antioxidant is ascorbic acid or ascorbic-acid-2-phosphate. In embodiments, the ascorbic acid or ascorbic-acid-2-phosphate is present in the medium at a concentration of between about 0.5 mg/L and about 500 mg/L, including about 50 mg/L. In embodiments, the methods comprise reprogramming and/or gene-editing a cell in vivo by contacting the cell with a medium containing a steroid and/or an antioxidant and contacting the cell with one or more nucleic acid molecules, wherein the one or more nucleic acid molecules encodes one or more reprogramming and/or gene-editing proteins. In embodiments, the cell is present in an organism, and the steroid and/or antioxidant are delivered to the organism.

In embodiments, the nucleic acid therapeutic agent encodes a gene-editing protein and/or associated elements for gene-editing functionality. In embodiments, the gene-editing protein is selected from a zinc finger (ZF), transcription activator-like effector (TALE), meganuclease, and clustered regularly interspaced short palindromic repeat (CRISPR)-associated protein. In embodiments, the CRISPR-associated protein is selected from Cas9, CasX, CasY, Cpf1, and gRNA complexes thereof. In embodiments, the CRISPR-associated protein is selected from Cas9, xCas9, Cas12a (Cpf1), Cas13a, Cas14, CasX, CasY, a Class 1 Cas protein, a Class 2 Cas protein, MAD7, and gRNA complexes thereof.

In embodiments, the therapeutic agent is a biologic therapeutic agent. In embodiments, the biologic therapeutic agent is a protein. In embodiments, the biologic therapeutic agent is an interferon, a monoclonal antibody, and/or an interleukin. In embodiments, the biologic therapeutic agent is used to effect immunotherapy selected from one or more of specific active immunotherapy, nonspecific active immunotherapy, passive immunotherapy, and cytotoxic therapy.

In embodiments, the biologic therapeutic agent is a recombinant protein.

In embodiments, the biologic therapeutic agent is a virus.

In embodiments, the biologic therapeutic agent is one of an antibody or an antibody fragment, fusion protein, gene-editing protein, cytokine, antigen, and peptide.

In embodiments, the therapeutic agent is a small molecule therapeutic agent. In embodiments, the small molecule therapeutic agent is one or more of a drug, inhibitor, or cofactor. In embodiments, the drug for use in cancer therapy. In embodiments, the inhibitor is one or more of a kinase inhibitor, proteasome inhibitor, and inhibitor targeting apoptosis.

In embodiments, the therapeutic agent is a vaccine and/or an immunogenic antigen.

### Methods of Treatment Using Megakaryocyte-Derived Extracellular Vesicles

In various embodiments, the compositions and methods disclosed herein may be utilized for drug delivery and treatment of one or more genetic disorders.

### Infectious Disease

Infectious diseases are disorders that are caused by pathogenic microorganisms, such as bacteria, viruses, fungi, or parasites. Zoonotic diseases are infectious diseases of animals that can cause disease when transmitted to humans.

In another aspect, the present invention relates to a method for treating or preventing an infectious disease, comprising administering an effective amount of a composition disclosed herein.

In another aspect, the present invention relates to a method for treating or preventing an infectious disease, comprising administering an effective amount of a composition comprising a cell, which is contacted with a composition disclosed herein in vitro.

In another aspect, the present invention relates to a method for treating or preventing an infectious disease, comprising administering an effective amount of a composition disclosed herein, wherein the composition comprises megakaryocyte-derived extracellular vesicles, which comprise cargo. In embodiments, the megakaryocyte-derived extracellular vesicles comprise a lipid bilayer membrane surrounding a lumen and derived from a human pluripotent stem cell, wherein the megakaryocyte-derived extracellular vesicle lumen comprises the cargo. In embodiments, in addition to or as an alternative to the cargo located in the lumen of the megakaryocyte-derived extracellular vesicle, the cargo is associated with the surface of the vesicle. In embodiments, the cargo is selected from one or more of a RNA, DNA, protein, carbohydrate, lipid, biomolecule, and small molecule. In embodiments, the cargo is one or more therapeutic agents.

In embodiments, the megakaryocyte-derived extracellular vesicles of the present compositions and methods are used to treat an infection caused by a virus (a viral infection) in a patient, wherein the viral infection is selected from one or more of: (a) the common cold, which mainly occurs due to rhinovirus, coronavirus, and adenovirus; (b) encephalitis and meningitis, resulting from enteroviruses and the herpes simplex virus (HSV), as well as West Nile Virus; (c) warts and skin infections, for which HPV and HSV are responsible; (d) gastroenteritis, caused by norovirus; (e) Zika; (f) AIDS/HIV; (g) Hepatitis; (h) polio; (i) influenza, including H1N1 swine flu; (j) Dengue fever; and (k) Ebola.

In embodiments, the megakaryocyte-derived extracellular vesicles are used to treat an infection caused by a bacterium (a bacterial infection) in a patient, wherein the bacterial infection is selected from one or more of: cholera, diphtheria, dysentery, bubonic plague, tuberculosis, typhoid, typhus, bacterial meningitis, otitis media, pneumonia, upper respiratory tract infection, gastritis, food poisoning, eye infection, sinusitis, urinary tract infection, skin infection, and sexually transmitted infection. In embodiments, the megakaryocyte-derived extracellular vesicles are used to treat an infection caused by a fungus (a fungal infection) in a patient, wherein the fungal infection is selected from one or more of: valley fever (coccidioidomycosis), histoplasmosis, candidiasis, athlete's foot, ringworm, eye infection, and skin infection. In embodiments, the megakaryocyte-derived extracellular vesicles are used to treat an infection caused by a parasite (a parasitic infection) in a patient, wherein the parasitic infection is selected from one or more of: malaria, sleeping sickness, amebiasis, trypanosomiasis, pediculosis, Chagas disease, cyclosporiasis, tapeworm infection, echinococcosis, foodborne disease, giardiasis, keratitis, leishmaniasis, onchocerciasis, trichinosis, waterborne disease, and zoonotic disease.

In embodiments, the megakaryocyte-derived extracellular vesicles are used to treat one or more symptoms associated with a coronavirus infection.

Coronaviruses (CoVs) are members of the family Coronaviridae, including betacoronavirus and alphacoronavirus-respiratory pathogens that have relatively recently become known to invade humans. The Coronaviridae family includes such betacoronavirus as Severe acute respiratory syndrome coronavirus 2 (SARS-COV-2), SARS-COV, Middle East Respiratory Syndrome-Corona Virus (MERS-COV), HCoV-HKU1, and HCoV-OC43. Alphacoronavirus includes, e.g., HCoV-NL63 and HCoV-229E. In embodiments, the present invention relates to the therapeutic use of the present megakaryocyte-derived extracellular vesicles for the treatment of one or more symptoms of infection with any of Severe acute respiratory syndrome coronavirus 2 (SARS-COV-2), SARS-COV, Middle East Respiratory Syndrome-Corona Virus (MERS-COV), HCoV-HKU1, and HCoV-OC43. Alphacoronavirus includes, e.g., HCoV-NL63 and HCoV-229E.

Without wishing to be bound by theory, coronaviruses invade cells through utilization of their “spike” surface glycoprotein that is responsible for viral recognition of Angiotensin Converting Enzyme 2 (ACE2), a transmembrane receptor on mammalian hosts that facilitate viral entrance into host cells. (Zhou et al., A pneumonia outbreak associated with a new coronavirus of probable bat origin, Nature 2020).

Symptoms associated with coronavirus infections include, but are not limited to, fever, tiredness, dry cough, aches and pains, shortness of breath and other breathing difficulties, diarrhea, upper respiratory symptoms (e.g. sneezing, runny nose, nasal congestion, cough, sore throat), and/or pneumonia. In embodiments, the present compositions and methods are useful in treating or mitigating any of these symptoms.

In embodiments, the present invention relates to the therapeutic use of the present megakaryocyte-derived extracellular vesicles for the treatment of one or more symptoms of infection with SARS-COV-2, including Coronavirus infection 2019 (COVID-19), caused by SARS-COV-2 (e.g., 2019-nCOV).

In embodiments, the infectious disease is a coronavirus infection. In embodiments, the coronavirus infection is infection by a betacoronavirus or an alphacoronavirus, optionally wherein the betacoronavirus is selected from a SARS-COV-2, SARS-COV, MERS-COV, HCoV-HKU1, and HCoV-OC43 or the alphacoronavirus is selected from a HCoV-NL63 and HCoV-229E. In embodiments, the coronavirus infection is infection by SARS-COV-2. In embodiments, the infectious disease is COVID-19.

In embodiments, the infectious disease is an influenza infection, optionally selected from Type A, Type B, Type C, and Type D influenza.

In embodiments, the infectious disease is a retroviral infection, optionally selected from human immune deficiency (HIV) and simian immune deficiency (SIV).

In embodiments, the composition comprises megakaryocyte-derived extracellular vesicles, which comprise a nucleic acid molecule encoding a vaccine protein and/or an immunogenic antigen. In embodiments, the composition comprises megakaryocyte-derived extracellular vesicles, which comprise a nucleic acid molecule encoding a protein related to infectivity.

In embodiments, the vaccine protein is a betacoronavirus protein or an alphacoronavirus protein, optionally wherein the betacoronavirus protein is selected from a SARS-COV-2, SARS-COV, MERS-COV, HCoV-HKU1, and HCoV-OC43 protein, or an antigenic fragment thereof or the alphacoronavirus protein is selected from a HCoV-NL63 and HCoV-229E protein, or an antigenic fragment thereof.

In embodiments, the SARS-COV-2 protein is the spike surface glycoprotein, membrane glycoprotein M, envelope protein E, and nucleocapsid phosphoprotein, or an antigenic fragment thereof. In embodiments, the spike surface glycoprotein is the S1 or S2 subunit, or an antigenic fragment thereof.

In embodiments, the nucleic acid molecule encoding a protein related to infectivity is mRNA, and the mRNA is optionally in vitro transcribed or synthetic. In embodiments, the mRNA comprises one or more non-canonical nucleotides, optionally selected from pseudouridine and 5-methoxyuridine.

In embodiments, the mRNA encodes SARS-COV-2 spike surface glycoprotein, membrane glycoprotein M, envelope protein E, and nucleocapsid phosphoprotein, or an antigenic fragment thereof.

In embodiments, the composition comprises megakaryocyte-derived extracellular vesicles, which comprise a nucleic acid encoding a protein having reduced C—C chemokine receptor type 5 (CCR5) and C—X—C chemokine receptor type 4 (CXCR4) activity. In embodiments, the composition comprises megakaryocyte-derived extracellular vesicles, which comprise a nucleic acid molecule encoding a mutant CCR5 or CXCR4.

In embodiments, the composition comprises megakaryocyte-derived extracellular vesicles, which comprise a nucleic acid molecule encoding a gene-editing protein that is capable of reducing C—C chemokine receptor type 5 (CCR5) and C—X—C chemokine receptor type 4 (CXCR4) activity. In embodiments, the nucleic acid molecule encoding a gene-editing protein reduces the chemokine receptor type 5 (CCR5) and C—X—C chemokine receptor type 4 (CXCR4) activity by between about 10% to about 20%. In embodiments, the nucleic acid molecule encoding a gene-editing protein reduces the chemokine receptor type 5 (CCR5) and C—X—C chemokine receptor type 4 (CXCR4) activity by between about 20% to about 30%. In embodiments, the nucleic acid molecule encoding a gene-editing protein reduces the chemokine receptor type 5 (CCR5) and C—X—C chemokine receptor type 4 (CXCR4) activity by between about 30% to about 40%. In embodiments, the nucleic acid molecule encoding a gene-editing protein reduces the chemokine receptor type 5 (CCR5) and C—X—C chemokine receptor type 4 (CXCR4) activity by between about 40% to about 50%. In embodiments, the nucleic acid molecule encoding a gene-editing protein reduces the chemokine receptor type 5 (CCR5) and C—X—C chemokine receptor type 4 (CXCR4) activity by between about 50% to about 60%. In embodiments, the nucleic acid molecule encoding a gene-editing protein reduces the chemokine receptor type 5 (CCR5) and C—X—C chemokine receptor type 4 (CXCR4) activity by between about 60% to about 70%. In embodiments, the nucleic acid molecule encoding a gene-editing protein reduces the chemokine receptor type 5 (CCR5) and C—X—C chemokine receptor type 4 (CXCR4) activity by between about 70% to about 80%.

### Thrombocytopenias/Anemias

In various embodiments, the present invention relates to a method for treating a disease or disorder characterized by abnormal numbers or functionality of a blood cell. Such disease or disorder is, in embodiments, a genetic disease or disorder.

In various embodiments, the present invention relates to a method for treating a disease or disorder of hematopoiesis.

Thrombocytopenias relates to a serum platelet count of less than 150,000/μL. Thrombocytopenias can be stratified into mild, moderate, and severe (corresponding to platelet counts of 75,000-150,000/μL, 50,000-75,000/μL, and less than 50,000/μL, respectively).

In an aspect, the present invention relates to a method for treating a thrombocytopenia, comprising administering an effective amount of a composition disclosed herein, wherein the composition comprises megakaryocyte-derived extracellular vesicles, which comprise cargo. In embodiments, the megakaryocyte-derived extracellular vesicles comprise a lipid bilayer membrane surrounding a lumen and derived from a human pluripotent stem cell, wherein the megakaryocyte-derived extracellular vesicle lumen comprises the cargo. In embodiments, in addition to or as an alternative to the cargo located in the lumen of the megakaryocyte-derived extracellular vesicle, the cargo is associated with the surface of the vesicle. In embodiments, the cargo is selected from one or more of a RNA, DNA, protein, carbohydrate, lipid, biomolecule, and small molecule. In embodiments, the cargo is one or more therapeutic agents.

In an aspect, the present invention relates to a method for treating a thrombocytopenia, comprising administering an effective amount of a composition disclosed herein, wherein the composition comprises megakaryocyte-derived extracellular vesicles, which comprise a nucleic acid encoding a functional thrombocytopenia-related gene, or a protein product thereof, or a nucleic acid encoding a gene-editing protein capable of creating a functional thrombocytopenia-related gene, or a protein product thereof.

In another aspect, the present invention relates to a method for treating a thrombocytopenia, comprising administering an effective amount of a composition comprising a cell which is contacted with a composition disclosed herein in vitro, wherein the composition comprises megakaryocyte-derived extracellular vesicles which comprise a nucleic acid encoding a functional thrombocytopenia-related gene, or a protein product thereof, or a nucleic acid encoding a gene-editing protein capable of creating a functional thrombocytopenia-related gene, or a protein product thereof.

In embodiments, the megakaryocyte-derived extracellular vesicles of the present compositions and methods are used to treat patients having mild thrombocytopenia. In embodiments, the megakaryocyte-derived extracellular vesicles of the present compositions and methods are used to treat patients having moderate thrombocytopenia. In embodiments, the megakaryocyte-derived extracellular vesicles of the present compositions and methods are used to treat patients having severe thrombocytopenia.

In embodiments, a patient having thrombocytopenia is administered a treatment comprising megakaryocyte-derived extracellular vesicles in combination with a treatment selected from one or more of (a) platelet transfusion and (b) administration of a TPO receptor agonist.

In embodiments, the thrombocytopenia is selected from congenital amegaryocytic thrombocytopenia (CAMT), thrombocytopenia with absent radii, radio ulnar synostosis with congenital thrombocytopenia, X-linked macrothrombocytopenia with thalassemia, GB11b-related thrombocytopenia, X-Linked Thrombocytopenia/Wiskott-Aldrich syndrome, Von Willebrand diseases Type 2B, platelet-type Von Willebrand disease, CYCS-Related thrombocytopenia, immune thrombocytopenia (idiopathic thrombocytopeni purpura), and myeloablation/chemotherapy induced thrombocytopenia.

In embodiments, the thrombocytopenia is CAMT.

In embodiments, the method promotes megakaryopoeisis in the patient.

In embodiments, the method causes an increase in platelet counts in the patient.

In embodiments, the increase in platelet counts is greater than about 100×10⁷ platelets/L, or greater than about 100×10⁸ platelets/L, or greater than about 100×10⁹ platelets/L, or greater than about 110×10⁹ platelets/L, or greater than about 120×10⁹ platelets/L, or greater than about 130×10⁹ platelets/L, or greater than about 140×10⁹ platelets/L, or greater than about 150×10⁹ platelets/L.

In embodiments, the method reduces the likelihood of the patient developing aplastic anemia and/or leukemia.

In embodiments, the method obviates the need for hematopoietic stem cell (HSC) transplantation.

In embodiments, the patient has advanced liver disease. In embodiments, the patient with advanced liver disease has increased concentration of von Willebrand factor as compared to a human without advanced liver disease. In embodiments, the patient with advanced liver disease has decreased concentrations of anticoagulant factors, such as antithrombin and protein C, and/or elevated levels of procoagulant factor VIII.

In embodiments, the patient is an infant.

In embodiments, the method provides a functional thrombopoietin (TPO) receptor in the patient.

In embodiments, the gene is a functional c-Mpl gene or encodes a gene-editing protein that is capable of forming a functional c-Mpl gene.

In embodiments, the disease or disorder is characterized by abnormal (e.g. reduced relative to an undiseased state) blood cell functionality. For instance, in embodiments, the present disease or disorder may not be characterized by a reduction in blood cells numbers but activity (e.g. due to a misfunctional protein).

### Hemoglobinopathies

Hemoglobinopathies are among the most common inherited diseases around the world. In embodiments, the megakaryocyte-derived extracellular vesicles of the present methods and compositions are used to treat a hemoglobinopathy in a patient. In embodiments, the hemoglobinopathy falls into the group of (a) thalassemia syndromes or (b) structural hemoglobin (Hb) variants (abnormal hemoglobins). In embodiments, the thalassemia syndrome is α-thalassemia or β-thalassemia. In embodiments, the structural hemoglobin variant is an Hb variant. In embodiments, the structural hemoglobin variant is HbS, HbE or HbC. In embodiments, the megakaryocyte-derived extracellular vesicles are used to treat one or more of the clinical manifestations of hemoglobinopathies selected from mild hypochromic anemia, moderate hematological disease, and severe, lifelong, transfusion-dependent anemia with multiorgan involvement.

In embodiments, the hemoglobinopathy is sickle cell disease (SCD). Sickle cell disease (SCD) encompasses a group of hematologic disorders caused by a single nucleotide-single gene mutation transposition from a normal adenine to thymine in one or both alleles in the chromosome 11 in the SNP rs334. The transposition of thymine instead of adenine causes the transcription of an abnormal hemoglobin (e.g. HbS) that causes intermittent or permanent episodes of ischemia and/or infarction. Sickle hemoglobin changes the anatomy and elastic properties of normal hemoglobin and make red blood cells contained in sickled hemoglobin more viscous with less capacity to transport and deliver oxygen and nutrients to distal organs and tissues. In embodiments, the present compositions and methods treat heterozygous sickled hemoglobin (a.k.a. sickle cell anemia), in which both alleles are affected with a translocation of thymine (T) instead of adenine (A) in SNP rs334. In embodiments, the present compositions and methods treat heterozygous sickled hemoglobin, in which one allele is affected (A/T).

In another aspect, the present invention relates to a method for treating a hemoglobinopathy, comprising administering an effective amount of a composition disclosed herein, wherein the composition comprises megakaryocyte-derived extracellular vesicles, which comprise cargo. In embodiments, the megakaryocyte-derived extracellular vesicles comprise a lipid bilayer membrane surrounding a lumen and derived from a human pluripotent stem cell, wherein the megakaryocyte-derived extracellular vesicle lumen comprises the cargo. In embodiments, in addition to or as an alternative to the cargo located in the lumen of the megakaryocyte-derived extracellular vesicle, the cargo is associated with the surface of the vesicle. In embodiments, the cargo is selected from one or more of a RNA, DNA, protein, carbohydrate, lipid, biomolecule, and small molecule. In embodiments, the cargo is one or more therapeutic agents.

In an aspect, the present invention relates to a method for treating a hemoglobinopathy, comprising administering an effective amount of a composition disclosed herein, wherein the composition comprises megakaryocyte-derived extracellular vesicles which comprise a nucleic acid encoding a functional hemoglobinopathy-related gene, or a protein product thereof, or a nucleic acid encoding a gene-editing protein capable of creating a functional hemoglobinopathy-related gene, or a protein product thereof.

In another aspect, the present invention relates to a method for treating a hemoglobinopathy, comprising administering an effective amount of a composition comprising a cell which is contacted with a composition disclosed herein in vitro, wherein the composition comprises megakaryocyte-derived extracellular vesicles which comprise a nucleic acid encoding a functional hemoglobinopathy-related gene, or a protein product thereof, or a nucleic acid encoding a gene-editing protein capable of creating a functional hemoglobinopathy-related gene, or a protein product thereof.

In embodiments, treatment with megakaryocyte-derived extracellular vesicles is combined with one or more of (a) stem cell transplantation; (b) periodic blood transfusions for life, combined with iron chelation; and (c) drugs, including analgesics, antibiotics, ACE inhibitors, and hydroxyurea. In embodiments, treatment with megakaryocyte-derived extracellular vesicles is combined with diagnostic testing. In embodiments, the diagnostic testing is selected from one or more of: (a) iron deficiency test; (b) red blood cell count; (c) DNA test; and (d) hemoglobin test.

In embodiments, the megakaryocyte-derived extracellular vesicles are used to treat a thalassemic hemoglobin synthesis disorder. In embodiments, the megakaryocyte-derived extracellular vesicles are used to treat a patient with abnormal hemoglobins. Sickle cell disease includes all manifestations of abnormal HbS levels, particularly HbS of greater than 50%.

In embodiments, the hemoglobinopathy is sickle cell disease. In embodiments, the hemoglobinopathy is β-thalassemia.

In embodiments, the method reduces or prevents one or more of red cell distortion, hemolytic anemia, microvascular obstruction, and ischemic tissue damage.

In embodiments, the functional hemoglobinopathy-related gene is a gene encoding a portion of hemoglobin. In embodiments, the functional hemoglobinopathy-related gene is a gene encoding one of the globin chains of hemoglobin.

In embodiments, the functional hemoglobinopathy-related gene restores hemoglobin solubility, stability, and/or oxygen affinity to undiseased levels. In embodiments, the functional hemoglobinopathy-related gene restores hemoglobin solubility, stability, and/or oxygen affinity to between about 40% and about 50% of undiseased levels. In embodiments, the functional hemoglobinopathy-related gene restores hemoglobin solubility, stability, and/or oxygen affinity to about 50% and about 60% of undiseased levels. In embodiments, the functional hemoglobinopathy-related gene restores hemoglobin solubility, stability, and/or oxygen affinity to about 60% and about 70% of undiseased levels. In embodiments, the functional hemoglobinopathy-related gene restores hemoglobin solubility, stability, and/or oxygen affinity to about 70% and about 80% of undiseased levels. In embodiments, the functional hemoglobinopathy-related gene restores hemoglobin solubility, stability, and/or oxygen affinity to about 80% and about 90% of undiseased levels. In embodiments, the functional hemoglobinopathy-related gene restores hemoglobin solubility, stability, and/or oxygen affinity to about 90% and about 100% of undiseased levels. In embodiments, the functional hemoglobinopathy-related gene improves hemoglobin solubility, stability, and/or oxygen affinity compared to undiseased levels. In embodiments, the functional hemoglobinopathy-related gene increases hemoglobin solubility, stability, and/or oxygen affinity. In embodiments, the functional hemoglobinopathy-related gene increases hemoglobin solubility, stability, and/or oxygen affinity compared to undiseased levels.

In embodiments, the functional hemoglobinopathy-related gene restores hemoglobin quantity to undiseased levels. In embodiments, the functional hemoglobinopathy-related gene restores hemoglobin quantity to between about 40% and about 50% of undiseased levels. In embodiments, the functional hemoglobinopathy-related gene restores hemoglobin quantity to about 50% and about 60% of undiseased levels. In embodiments, the functional hemoglobinopathy-related gene restores hemoglobin quantity to about 60% and about 70% of undiseased levels. In embodiments, the functional hemoglobinopathy-related gene restores hemoglobin quantity to about 70% and about 80% of undiseased levels. In embodiments, the functional hemoglobinopathy-related gene restores hemoglobin quantity to about 80% and about 90% of undiseased levels. In embodiments, the functional hemoglobinopathy-related gene restores hemoglobin quantity to about 90% and about 100% of undiseased levels. In embodiments, the functional hemoglobinopathy-related gene improves hemoglobin quantity compared to undiseased levels. In embodiments, the functional hemoglobinopathy-related gene increases hemoglobin quantity. In embodiments, the functional hemoglobinopathy-related gene increases hemoglobin quantity compared to undiseased levels

In embodiments, the functional hemoglobinopathy-related gene prevents or reduces RBC sickling.

In embodiments, the functional hemoglobinopathy-related gene prevents or reduces sickle hemoglobin polymerization.

In embodiments, the functional hemoglobinopathy-related gene is beta globin (HBB). In embodiments, the gene encodes a gene-editing protein that is capable of forming a functional beta globin (HBB) gene.

### Pharmaceutical Compositions

Therapeutic treatments comprise the use of one or more routes of administration and of one or more formulations that are designed to achieve a therapeutic effect at an effective dose, while minimizing toxicity to the patient to which treatment is administered.

In embodiments, the effective dose is an amount that substantially avoids cell toxicity in vivo. In various embodiments, the effective dose is an amount that substantially avoids an immune reaction in a human patient. For example, the immune reaction may be an immune response mediated by the innate immune system. Immune response can be monitored using markers known in the art (e.g. cytokines, interferons, TLRs). In embodiments, the effective dose obviates the need for treatment of the human patient with immune suppressants agents used to moderate the residual toxicity.

Upon formulation, solutions may be administered in a manner compatible with the dosage formulation and in such amount as is therapeutically effective, as described herein. The formulations may easily be administered in a variety of dosage forms such as injectable solutions and the like. For parenteral administration in an aqueous solution, for example, the solution generally is suitably buffered and the liquid diluent first rendered isotonic with, for example, sufficient saline or glucose. Such aqueous solutions may be used, for example, for intravenous, intramuscular, subcutaneous and intraperitoneal administration. Preferably, sterile aqueous media are employed as is known to those of skill in the art.

Pharmaceutical preparations may additionally comprise delivery reagents (a.k.a. “transfection reagents”, a.k.a. “vehicles”, a.k.a. “delivery vehicles”) and/or excipients. Pharmaceutically acceptable delivery reagents, excipients, and methods of preparation and use thereof, including methods for preparing and administering pharmaceutical preparations to patients are well known in the art, and are set forth in numerous publications, including, for example, in US Patent Appl. Pub. No. US 2008/0213377, the entirety of which is incorporated herein by reference. In aspects, the present invention relates to a pharmaceutical composition comprising a composition disclosed herein and a pharmaceutically acceptable excipient or carrier.

For example, the present compositions can be in the form of pharmaceutically acceptable salts. Such salts include those listed in, for example, J. Pharma. Sci. 66, 2-19 (1977) and The Handbook of Pharmaceutical Salts; Properties, Selection, and Use. P. H. Stahl and C. G. Wermuth (eds.), Verlag, Zurich (Switzerland) 2002, which are hereby incorporated by reference in their entirety. Non-limiting examples of pharmaceutically acceptable salts include: sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, pamoate, phenylacetate, trifluoroacetate, acrylate, chlorobenzoate, dinitrobenzoate, hydroxybenzoate, methoxybenzoate, methylbenzoate, o-acetoxybenzoate, naphthalene-2-benzoate, isobutyrate, phenylbutyrate, α-hydroxybutyrate, butyne-1,4-dicarboxylate, hexyne-1,4-dicarboxylate, caprate, caprylate, cinnamate, glycollate, heptanoate, hippurate, malate, hydroxymaleate, malonate, mandelate, mesylate, nicotinate, phthalate, teraphthalate, propiolate, propionate, phenylpropionate, sebacate, suberate, p-bromobenzenesulfonate, chlorobenzenesulfonate, ethylsulfonate, 2-hydroxyethylsulfonate, methylsulfonate, naphthalene-1-sulfonate, naphthalene-2-sulfonate, naphthalene-1,5-sulfonate, xylenesulfonate, tartarate salts, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or tri-alkylamines, dicyclohexylamine; tributyl amine; pyridine; N-methyl, N-ethylamine; diethylamine; triethylamine; mono-, bis-, or tris-(2-OH-lower alkylamines), such as mono-; bis-, or tris-(2-hydroxyethyl)amin e, 2-hydroxy-tert-butylamine, or tris-(hydroxymethyl)methy lamine, N,N-di-lower alkyl-N-(hydroxyl-lower alkyl)-amines, such as N, N-dimethyl-N-(2-hydroxyethyl)amin e or tri-(2-hydroxyethyl)amin e; N-methyl-D-glucamine; and amino acids such as arginine, lysine, and the like.

The present pharmaceutical compositions can comprise excipients, including liquids such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In embodiments, the pharmaceutically acceptable excipients are sterile when administered to a patient.

Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any agent described herein, if desired, can also comprise minor amounts of wetting or emulsifying agents, or pH buffering agents.

In embodiments, the composition is formulated for one or more of topical, intrathecal, intra-lesional, intra-coronary, intravenous (IV), intra-articular, intramuscular, intra-nasal, and intra-endobronchial administration and administration via intrapancreatic endovascular injection, intra-nucleus pulposus, lumbar puncture, intra-myocardium, transendocardium, intra-fistula tract, intermedullary space, intra-nasal, and intradural space injection.

In embodiments, the composition is formulated for infusion. In embodiments, the composition is formulated for infusion, wherein the composition is delivered to the bloodstream of a patient through a needle in a vein of the patient through a peripheral line, a central line, a tunneled line, an implantable port, and/or a catheter. In embodiments, the patient may also receive supportive medications or treatments, such as hydration, by infusion. In embodiments, the composition is formulated for intravenous infusion. In embodiments, the infusion is continuous infusion, secondary intravenous therapy (IV), and/or IV push. In embodiments, the infusion of the composition may be administered through the use of equipment selected from one or more of an infusion pump, hypodermic needle, drip chamber, peripheral cannula, and pressure bag.

In embodiments, the composition is introduced into or onto the skin, for instance. intraepidermally, intradermally or subcutaneously, in the form of a cosmeceutical (see, e.g., Epstein, H., Clin. Dermatol. 27 (5): 453-460 (2009)). In embodiments, the composition is in the form of a cream, lotion, ointment, gel, spray, solution and the like. In embodiments, the composition further includes a penetration enhancer such as, but not limited to, surfactants, fatty acids, bile salts, chelating agents, non-chelating non-surfactants, and the like. In embodiments, the composition may also include a fragrance, a colorant, a sunscreen, an antibacterial and/or a moisturizer.

In order that the invention disclosed herein may be more efficiently understood, examples are provided below. It should be understood that these examples are for illustrative purposes only and are not to be construed as limiting the invention in any manner.

### EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples, therefore, specifically point out the preferred embodiments of the present invention, and are not to be construed as limiting in any way the remainder of the disclosure.

The materials and methods employed in the experiments disclosed herein are now described.

### Materials and Methods

MKEVs were concentrated using 100 KDa spin filters, isolated by size exclusion chromatography, and processed for proteomic and transcriptomic analyses.

For proteomic analyses, extracellular vesicle proteins were precipitated by incubation in acetone followed by centrifugation and resuspension in ammonium bicarbonate buffer supplemented by urea. Protein pellet was resuspended for trypsin/Lys-C digestion and peptides were extracted on C18 column. Peptide samples were analyzed using LC-MS/MS on a timsTOF Pro mass spectrometer (Bruker Daltonics, USA). Data was acquired using data-dependent auto-MS/MS with a 100-1700 m/z mass range, with PASEF enabled with a number of PASEF scans set at 10 (1.27 seconds duty cycle) and a dynamic exclusion of 0.4 minute, m/z dependent isolation window and collision energy of 42.0 eV. The target intensity was set to 20,000, with an intensity threshold of 2,500. For data processing, initial searches were performed with Preview™ version 4.0.12 (Protein Metrics, USA) against the Homo sapiens reference proteome (Uniprot, 78 120 proteins and 20 600 genes as of Mar. 7, 2021) to measure mass errors, digestion specificity and modifications for subsequent full search by Byonic™ version 4.0.12 (Protein Metrics, USA). The Byonic™ search strategy was set by Preview™ on the same protein database with the following parameters: mass tolerance of 20 ppm for the precursor ions and 40 ppm for the fragment ions, a false discovery rate (FDR) of <1% as estimated using concatenated forward-reverse database search at the peptide-spectrum match (PSM) level, semi-specific N-ragged trypsin cleavage with a maximum of two missed cleaveage and static cysteine carbamidomethylation (+57.021464 Da). The following variable modifications were specified: methionine mono/dioxidation (+15.994915/+31.989829 Da), dethiomethylation of methionine (−48.003371 Da), deamidation of asparagine (+0.984016 Da), N-terminal glutamine/glutamate conversion to pyro-Glu (−17.026549/−18.010565 Da), N-terminal ammonia-loss (−17.026549 Da), carbamylation of lysine and arginine in addition to peptide N-terminus (+43.005814 Da) and N-terminal peptide acetylation (+42.010565 Da). Byonic output files corresponding to six replicates were merged with Batcher™ version 4.0 (Protein Metrics, USA). Decoys protein IDs and common contaminants were filtered out of the final dataset (e.g., trypsin, serum albumin, human keratins, etc). Only proteins with identification p-value ≤0.001 (i.e. log 10 of the protein p-value ≥3) were reported.

For transcriptomic analyses, RNA was extracted and transcriptomic analyses were performed with 40 ng of total RNA for each library using the NEBNext ultrall directional RNA library prep kit for illumina. Specifically, the NEBNext Ultra II directional RNA library prep kit for Illumina (New Englands Biolabs Inc., Ipswich, MA, USA) was used to prepare total RNA sequencing libraries, according to manufacturer's instruction. Fragmented RNA was used as a template for cDNA synthesis by reverse transcriptase with random primers. cDNA was converted to double-stranded DNA and end-repaired. Ligation of adaptors was followed by a purification step with AxyPrep Mag PCR Clean-up kit (Axygen, Big Flats, NY, USA), by an excision of the strands containing the dUTPs and finally, by a PCR enrichment step of 8 cycles to incorporate specific indexed adapters for the multiplexing. The quality of final amplified libraries was examined with a DNA screentape D1000 on a TapeStation 2200 (Agilent Technologies, Santa Clara, USA) and the quantification was done on the QBit 3.0 fluorometer (ThermoFisher Scientific, Canada). Total RNA-seq libraries with unique index were pooled together in equimolar ratio and sequenced for paired-end 100 pb sequencing on a NovaSeq 6000 flowcell S1 at the Next-Generation Sequencing Platform, Genomics Center, CHU de Québec-Université Laval Research Center, Québec City, Canada. The average insert size for the libraries was 240 bp. The mean coverage/sample was 75M of paired-end reads. Sequencing quality was ensured for all 100-bp reads obtained from the sequencer using fastqc (0.11.2) (at world wide web at bioinformatics.babraham.ac.uk/projects/fastqc/). Only reads longer than 30 bp after trimming were kept and aligned to the human genome transcriptome (Homo_sapiens.GRCh38.cdna.all.fa) (release-104; at Hypertext Transfer Protocol Secure ftp.ensembl.org/pub/release-104/fasta/homo_sapiens/cdna/). Transcript abundances were obtained using transcriptome indexing and the pseudo-alignment procedure from Kallisto (Bray et al. 2016).

Data analysis and visualization of LC-MS/MS and bulk RNA-seq data was conducted using R (version 4.0.3). LC-MS/MS: Identified and quantified protein groups with fewer than 3 unique peptides were excluded from the downstream analysis. The acquired presently disclosed MkEV proteome was compared against the ExoCarta and VesiclePedia databases (Keerthikumar et al. J Mol Biol. 2016 Feb. 22; 428 (4): 688-692.; Pathan et al. Nucleic Acids Res. 2019 Jan. 8; 47 (D1): D516-D519.) In addition, the STRM MKEV proteome was compared against a collection of previously published and newly generated plasma EV proteomes of healthy donors (Karimi et al. Cell Mol Life Sci. 2018; 75 (15): 2873-2886; Muraoka et al. iScience. 2022; 25 (4): 104012., Keerthikumar et al. J Mol Biol. 2016 Feb. 22; 428 (4): 688-692.; Pathan et al. Nucleic Acids Res. 2019 Jan. 8; 47 (D1): D516-D519.). In brief, identified proteins from human exosome/EV samples were extracted from ExoCarta and VesiclePedia. The disclosed MkEV proteins not contained within ExoCarta/VesiclePedia were identified as previously uncharacterized, MkEV-specific proteins. Surface and membrane proteins were defined as previously described (Bausch-Fluck et al, PLOS One. 2015 Apr. 20; 10 (3): e0121314; Thul et al, Science. 2017 May 26; 356 (6340): eaal3321.). Kyoto Encyclopedia of Genes and Genomes (KEGG) pathway analysis (Kanehisa and Goto, Nucleic Acids Res. 2000 Jan. 1; 28 (1): 27-30.) was performed using Limma (version 3.44.3). Protein-protein interaction networks were constructed using the STRING database (Szklarcyk et al. 2021) and visualized using Cytoscape (version 3.8.2). A physical interaction network and a functional/physical STRING network (database and experimental evidence only) were computed using an interaction confidence score cutoff of 0.4. The proteomes of STRM MKEV batches were inspected for reproducibility, requiring separate normalization of each sample. Protein intensities were sample loading normalized, using the total sum of protein intensities in each given sample. RNA-seq: Raw counts were normalized to gene length and sequencing depth by computing transcript per million (TPM) values. Protein coding genes were extracted, and lowly expressed genes were excluded from the analysis (cutoff: <1 TPM for each technical replicate). The derived set of identified protein coding transcripts was compared against mRNA evidence from human exosome/EV samples, contained within ExoCarta and VesiclePedia. Genes previously not identified in either database were classified as unique MkEV transcripts. KEGG pathway analysis was performed as previously described.

Confocal microscopy was performed to determine the internalization of MkEV-delivered cargo by primary HSPCs. First, bone marrow was harvested from wild type mice and subjected to red cell lysis using NH₄Cl (STEMCELL Technologies) and lineage depletion (STEMCELL Technologies). HSPCs were isolated by fluorescence-activated cell sorting (FACS) and defined as Lineage depleted CD150+CD48-cells. MkEVs were loaded with GFP-tagged Cas9 (Sigma) and guide RNA (Sigma), pre-formed to RNP. HSPCs were cocultured with loaded MkEVs for 18 h. Following the incubation period, unfixed cells were transferred onto a microscope slide and imaged using a Zeiss 780 confocal microscope and a x63 objective. Zen Black Software was utilised for image capture and processing.

Co-cultures were conducted using primary wild type whole bone marrow cells post red cell lysis using NH₄Cl (STEMCELL Technologies). MkEVs were either loaded with GFP-tagged Cas9 or labelled with DID Vybrant dye (Invitrogen). DID labelling was performed according to manufacturer's instructions, followed by 2× wash steps. Loaded or labelled MkEVs were co-cultured with 1e6 whole bone marrow cells for 24 h. Co-cultures were subsequently analysed by flow cytometry (LSR Fortessa X-20, BD Bioscience) using the following antibodies: CD3, CD11b, CD19, GR-1, TER119, CD45R/B220, 7AAD, cKit, Sca-1. Flow cytometry data analysis was conducted using FlowJo (version 10.8.1, BD Bioscience).

### Example 1: Megakaryocyte-Derived Extracellular Vesicles (MkEVs) Contain Unique Protein Markers

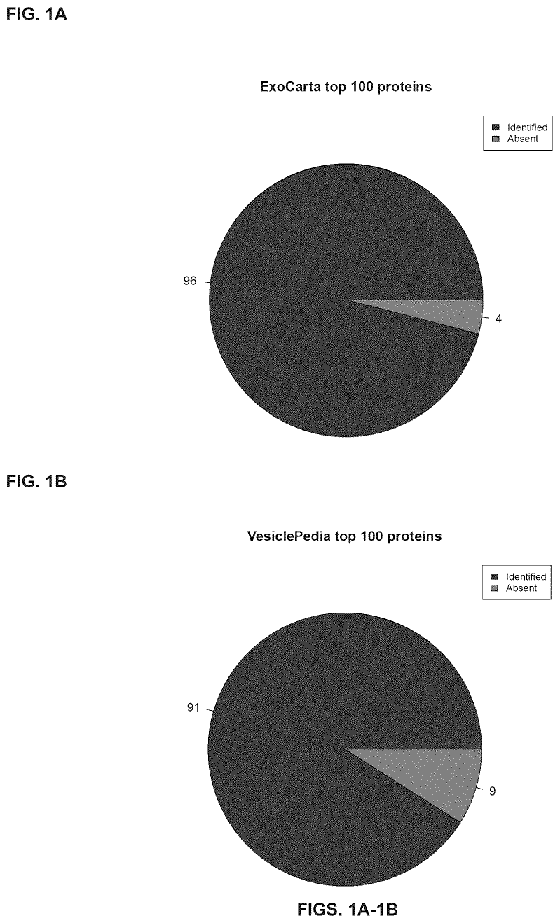

As shown in FIGS. 1A-1B, an overlap of top 100 exosome marker proteins was identified in the database ExoCarta (Keerthikumar et al. J Mol Biol. 2016; 428 (4): 688-692) (at world wide web at exocarta.org) with the disclosed MkEV proteome. 96 of these proteins were identified in the disclosed MkEV proteome, while 4 were not identified (FIG. 1A). As shown in FIG. 1B, of the top 100 EV marker proteins as defined by the VesiclePedia database (Pathan et al. Nucleic Acids Res. 2019 Jan. 8; 47: D516-D519) (at world wide web at microvesicles.org), 91 of these proteins were identified within the present MkEV proteome, while 9 were not. Collectively, the presently disclosed MkEVs contain unique as well as common extracellular vesicle and exosome markers.

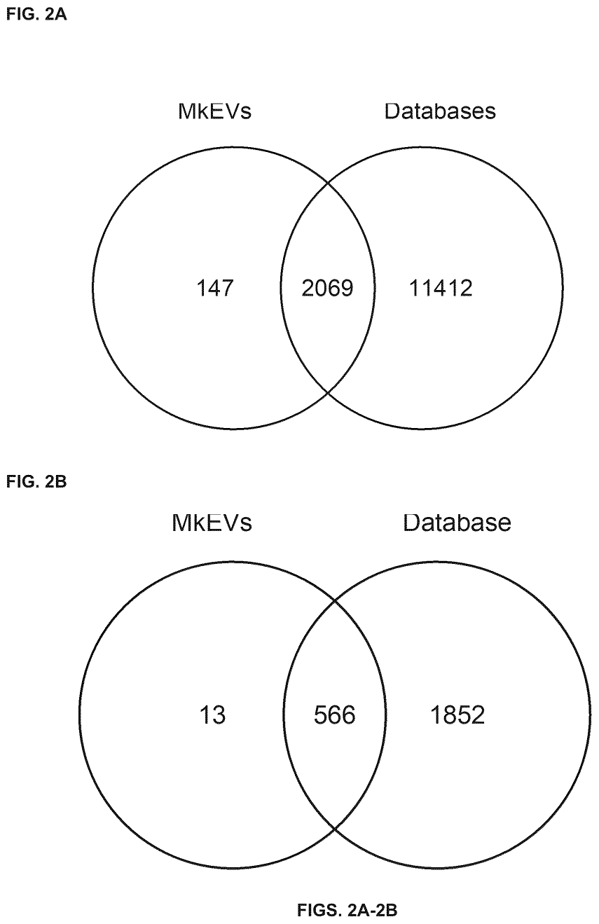

As shown in the Venn diagrams of FIGS. 2A-2B, 147 proteins that are unique to disclosed MKEVs (FIG. 2A). Overlap of cell surface proteins on the presently disclosed MkEVs and surface proteins captured by the VesiclePedia database showed that 13 cell surface and membrane proteins are unique to disclosed MkEVs (FIG. 2B). Surface and membrane proteins were defined according to Bausch-Fluck et al. (Bausch-Fluck et al. PLOS One. 10: e0121314) and the Human Protein Atlas (Thul P. J., Akesson L., Science. 2017. 365 (6340)).

Table 1 below provides a full list of the proteins unique to the presently disclosed MkEVs, previously not identified in extracellular vesicles and exosomes as reported in high and low thruput screens, captured in the curated VesiclePedia and ExoCarta databases.

TABLE 1
Unique MkEVs proteins

| Accession | Gene | Total. Intensity |
|---|---|---|
| P62805 | H4C1 | 3.80E+08 |
| Q16777 | H2AC20 | 2.90E+08 |
| P68431 | H3C1 | 2.60E+08 |
| O60814 | H2BC12 | 2.40E+08 |
| Q8N0W3-2 | FCSK | 5.50E+07 |
| O95810 | CAVIN2 | 4.20E+07 |
| P54577 | YARS1 | 4.10E+07 |
| Q71DI3 | H3C15 | 2.30E+07 |
| O75367 | MACROH2A1 | 2.30E+07 |
| P16104 | H2AX | 1.80E+07 |
| Q5TEC6 | H3-2 | 1.60E+07 |
| O60613 | SELENOF | 1.50E+07 |
| O95866-2 | MPIG6B | 1.40E+07 |
| Q16181-2 | SEPTIN7 | 1.40E+07 |
| P49588 | AARS1 | 1.30E+07 |
| P16401 | H1-5 | 1.30E+07 |
| P16403 | H1-2 | 1.30E+07 |
| Q71UI9 | H2AZ2 | 1.30E+07 |
| P41250 | GARS1 | 1.20E+07 |
| Q9P2J5 | LARS1 | 1.00E+07 |
| A0A3B3ITS0 | THPO | 1.00E+07 |
| P63244 | RACK1 | 1.00E+07 |
| P84243 | H3-3A | 1.00E+07 |
| Q8NCU4 | CCDC191 | 1.00E+07 |
| Q6DRA6 | H2BC19P | 1.00E+07 |
| Q15019 | SEPTIN2 | 9.80E+06 |
| P07814 | EPRS1 | 9.30E+06 |
| P26639 | TARS1 | 8.20E+06 |
| P06576 | ATP5F1B | 8.10E+06 |
| Q96RT1-3 | ERBIN | 8.10E+06 |
| P14868 | DARS1 | 7.90E+06 |
| P23381 | WARS1 | 7.30E+06 |
| P26640 | VARS1 | 7.00E+06 |
| Q96TA1 | NIBAN2 | 6.50E+06 |
| Q14141 | SEPTIN6 | 6.30E+06 |
| O43776 | NARS1 | 6.20E+06 |
| P54136 | RARS1 | 5.70E+06 |
| Q9NVA2 | SEPTIN11 | 4.80E+06 |
| Q99719 | SEPTIN5 | 3.90E+06 |
| Q5T5C7 | SARS1 | 3.80E+06 |
| Q9BZQ8 | NIBAN1 | 3.70E+06 |
| A8MWD9 | SNRPGP15 | 3.60E+06 |
| Q8N4L2 | PIP4P2 | 3.60E+06 |
| Q9NUQ9 | CYRIB | 3.50E+06 |
| Q5VZK9-2 | CARMIL1 | 3.50E+06 |
| Q15046 | KARS1 | 3.30E+06 |
| P41252 | IARS1 | 3.00E+06 |
| Q9UHD8 | SEPTIN9 | 2.80E+06 |
| P10412 | H1-4 | 2.70E+06 |
| Q92619-2 | ARHGAP45 | 2.60E+06 |
| P07305 | H1-0 | 2.60E+06 |
| P49589-3 | CARS1 | 2.50E+06 |
| Q92616 | GCN1 | 2.40E+06 |
| O95864 | FADS2 | 2.40E+06 |
| Q9NVG8 | TBC1D13 | 2.40E+06 |
| O43681 | GET3 | 2.40E+06 |
| P10155 | RO60 | 2.30E+06 |
| O43504 | LAMTOR5 | 2.30E+06 |
| Q15369 | ELOC | 2.30E+06 |
| Q99878 | H2AC14 | 2.30E+06 |
| Q14162 | SCARF1 | 2.20E+06 |
| Q9Y225 | RNF24 | 2.10E+06 |
| Q5JQS6 | GCSAML | 2.10E+06 |
| O43847-2 | NRDC | 2.10E+06 |
| Q5VYK3 | ECPAS | 2.00E+06 |
| Q9P0M6 | MACROH2A2 | 2.00E+06 |
| P25705 | ATP5F1A | 1.90E+06 |
| Q08495-2 | DMTN | 1.90E+06 |
| Q6ICL3 | TANGO2 | 1.80E+06 |
| P32927 | CSF2RB | 1.80E+06 |
| Q12768 | WASHC5 | 1.70E+06 |
| P22001 | KCNA3 | 1.70E+06 |
| P47897 | QARS1 | 1.60E+06 |
| Q8N5J2 | MINDY1 | 1.60E+06 |
| Q15257-2 | PTPA | 1.60E+06 |
| A0A1C7CYX0 | EXOC3L2 | 1.60E+06 |
| Q86TP1 | PRUNE1 | 1.60E+06 |
| O94903 | PLPBP | 1.50E+06 |
| Q9NXG2 | THUMPD1 | 1.40E+06 |
| Q2M389 | WASHC4 | 1.40E+06 |
| Q92692 | NECTIN2 | 1.30E+06 |
| Q13630 | GFUS | 1.30E+06 |
| M0R0K5 | ADGRE2 | 1.30E+06 |
| Q9ULX6-2 | AKAP8L | 1.20E+06 |
| Q9H0X4 | FAM234A | 1.20E+06 |
| P30520 | ADSS2 | 1.20E+06 |
| Q6ZTN6-3 | ANKRD13D | 1.20E+06 |
| Q8NC54 | KCT2 | 1.10E+06 |
| Q9H0P0-1 | NT5C3A | 1.10E+06 |
| Q86T03 | PIP4P1 | 1.10E+06 |
| A0A2R8Y4Z8 | CCDC9 | 1.10E+06 |
| Q15370 | ELOB | 1.00E+06 |
| O60760 | HPGDS | 1.00E+06 |
| Q6P9B6 | MEAK7 | 9.80E+05 |
| O94826 | TOMM70 | 9.70E+05 |
| B2RUZ4 | SMIM1 | 9.70E+05 |
| P12081-3 | HARS1 | 9.70E+05 |
| O75947 | ATP5PD | 9.60E+05 |
| O60502-4 | OGA | 9.40E+05 |
| Q86X52 | CHSY1 | 9.20E+05 |
| Q14534 | SQLE | 9.10E+05 |
| Q92537 | SUSD6 | 9.10E+05 |
| A0A075B6S5 | IGKV1-27 | 8.70E+05 |
| I3L0A0 | PEDS1-UBE2V1 | 8.40E+05 |
| Q9C0F1-2 | CEP44 | 8.40E+05 |
| Q9HB07 | MYG1 | 8.10E+05 |
| Q86YC3 | NRROS | 8.00E+05 |
| Q9HBE5 | IL21R | 7.90E+05 |
| P25098 | GRK2 | 7.90E+05 |
| Q8IX19 | MCEMP1 | 7.60E+05 |
| C9JA41 | ELAPOR2 | 7.50E+05 |
| Q9NVU7 | SDAD1 | 7.40E+05 |
| Q9Y5P4-3 | CERT1 | 7.20E+05 |
| Q969M7-6 | UBE2F | 7.10E+05 |
| Q8N5C1 | CALHM5 | 7.00E+05 |
| Q92522 | H1-10 | 7.00E+05 |
| A8MUM1 | EIPR1 | 6.60E+05 |
| Q9BVG4 | PBDC1 | 6.50E+05 |
| Q5T2E6 | ARMH3 | 6.30E+05 |
| Q9H9C1 | VIPAS39 | 6.10E+05 |
| Q14696 | MESD | 6.10E+05 |
| Q86WR7 | PROSER2 | 6.00E+05 |
| Q3YEC7 | RABL6 | 5.90E+05 |
| O15117 | FYB1 | 5.90E+05 |
| F8W038 | C17orf49 | 5.90E+05 |
| Q96TC7 | RMDN3 | 5.80E+05 |
| Q6YP21 | KYAT3 | 4.90E+05 |
| Q5BVD1 | TTMP | 4.70E+05 |
| Q96HE7 | ERO1A | 4.60E+05 |
| Q9BZW8 | CD244 | 4.40E+05 |
| Q9NWV4 | CZIB | 4.40E+05 |
| O14495 | PLPP3 | 4.30E+05 |
| Q9NXR7 | BABAM2 | 4.30E+05 |
| P16402 | H1-3 | 4.00E+05 |
| Q8NCW5 | NAXE | 3.90E+05 |
| Q5T5H1 | ENSA | 3.50E+05 |
| Q9NZW5 | PALS2 | 3.50E+05 |
| Q02153 | GUCY1B1 | 3.30E+05 |
| C9J7I0 | UMAD1 | 3.20E+05 |
| Q4VC31 | MIX23 | 3.00E+05 |
| Q8TBF2-6 | PRXL2B | 3.00E+05 |
| A0A3B3IUA2 | SNU13 | 2.60E+05 |
| Q9Y224 | RTRAF | 2.60E+05 |
| M0QY77 | KXD1 | 2.30E+05 |
| Q9H7M9 | VSIR | 1.80E+05 |
| P19235 | EPOR | 1.20E+05 |
| Q9P0N8 | MARCHF2 | 1.00E+05 |

The analysis of the presently disclosed MkEVs-unique proteins identified some that are specific for cell surface and membrane.

Table 2 shows a list of the surface or membrane proteins identified that are unique to the disclosed MkEVs when compared to the documented (VesiclePedia and ExoCarta) proteomes of extracellular vesicles and exosomes.

TABLE 2
Unique Surface and/or Membrane MkEV proteins

| Accession | Gene | Total. Intensity |
|---|---|---|
| O95810 | CAVIN2 | 4.20E+07 |
| O95866-2 | MPIG6B | 1.40E+07 |
| Q96RT1-3 | ERBIN | 8.10E+06 |
| Q15369 | ELOC | 2.30E+06 |
| P32927 | CSF2RB | 1.80E+06 |
| P22001 | KCNA3 | 1.70E+06 |
| Q92692 | NECTIN2 | 1.30E+06 |
| Q9HBE5 | IL21R | 7.90E+05 |
| Q8IX19 | MCEMP1 | 7.60E+05 |
| Q86WR7 | PROSER2 | 6.00E+05 |
| O15117 | FYB1 | 5.90E+05 |
| Q9BZW8 | CD244 | 4.40E+05 |
| P19235 | EPOR | 1.20E+05 |

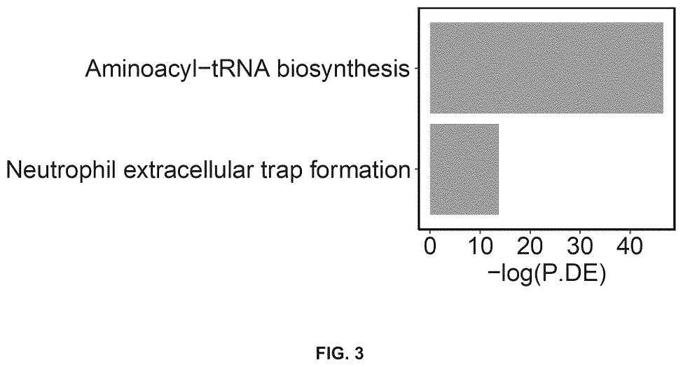

A list of significantly enriched KEGG pathways of unique disclosed MkEV proteins (p-value <0.05) is shown in FIG. 3 and includes pathways involved in aminoacyl-tRNA biosynthesis and neutrophil extracellular trap formation.

Table 3 below provides a list of proteins unique to the disclosed MkEVs that are associated with selected KEGG pathways.

TABLE 3
Identified proteins for each KEGG pathway

| | Aminoacyl-tRNA | Neutrophil Extracellular |
|---|---|---|
| | biosynthesis | Trap Formation |
| KEGG pathway ID | HSA00970 | HSA04613 |
| MkEV Protein name | YARS1 | H4C1 |
| | AARS1 | H2AC20 |
| | GARS1 | H3C1 |
| | LARS1 | H2BC12 |
| | EPRS1 | H3C15 |
| | TARS1 | MACROH2A1 |
| | DARS1 | H2AX |
| | WARS1 | H2AZ2 |
| | VARS1 | H3-3A |
| | NARS1 | H2AC14 |
| | RARS1 | MACROH2A2 |
| | SARS1 | |
| | KARS1 | |
| | IARS1 | |
| | CARS1 | |
| | QARS1 | |
| | HARS1 | |

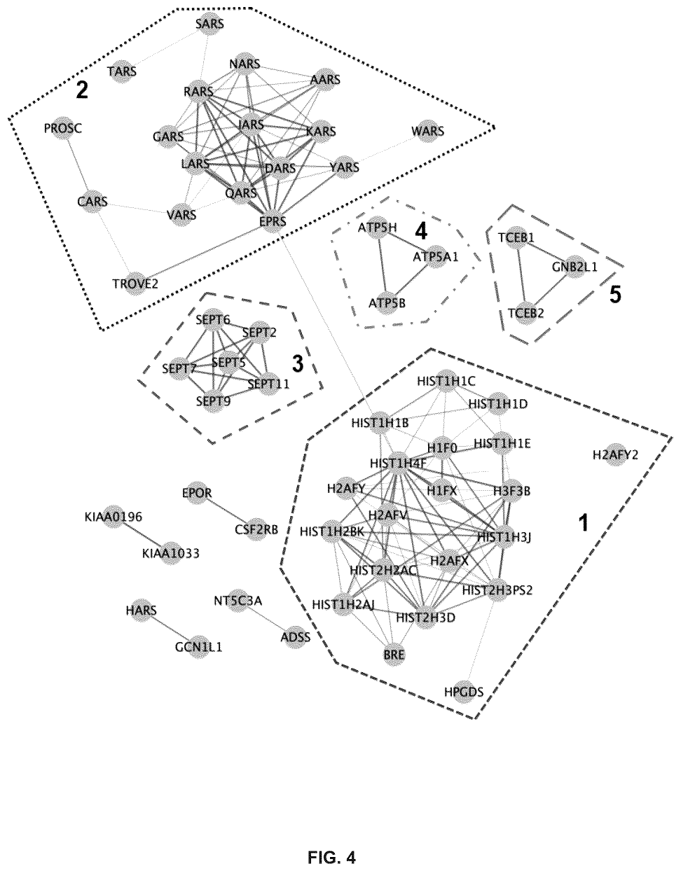

As shown in FIG. 4, there is a direct protein-protein interaction network of unique the disclosed MkEV proteins. The network was constructed to include curated direct protein-protein interactions (interaction score >0.4). Edge intensity depicts the associated interaction score (confidence of interaction). Clusters with >3 nodes were highlighted and numbered in descending order. Non-interacting nodes were excluded. Number 1 represents a histone cluster, 2 represents the tRNA aminoacylation for protein translation cluster, 3 represents the cluster of SEPTIN proteins, 4 represents a mitochondrial-related cluster, and 5 represents cellular biosynthesis processes (transcription and translation).

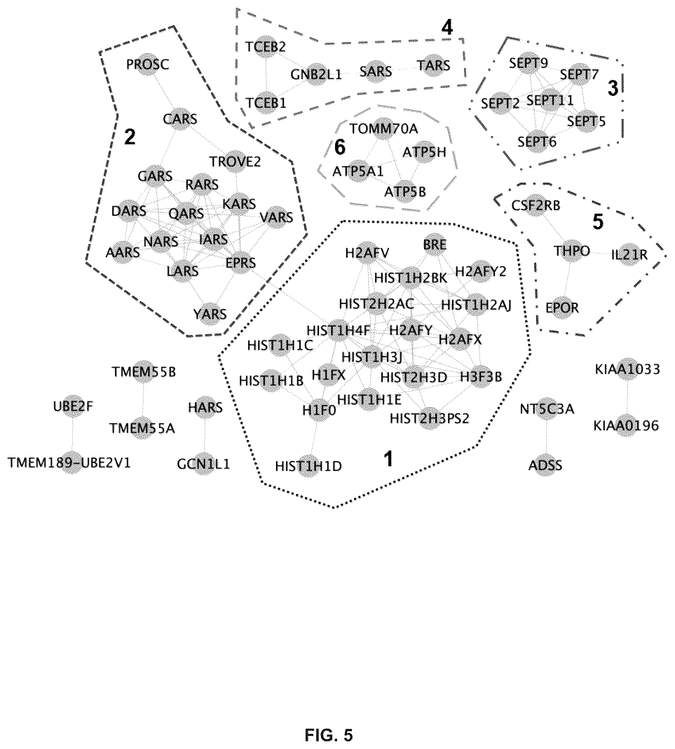

More specifically, a functional and physical protein-protein interaction network was identified for the unique MkEV proteins of the present invention (FIG. 5).

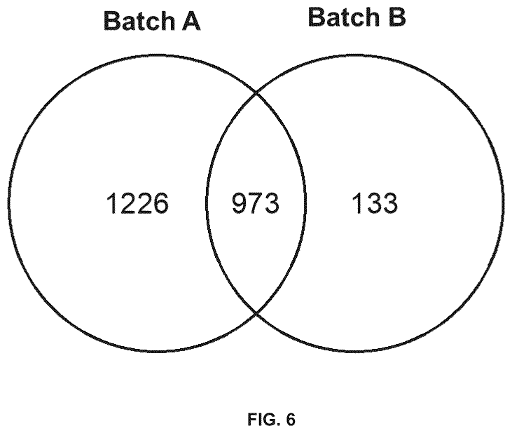

To determine the reproducibility of MkEVs, a second batch was subjected to proteomic screening and compared to the previous dataset, revealing an 88% overlap in protein IDs between both batches (FIG. 6).

Amongst the 133 newly identified MkEV proteins, 13 were not previously captured by comprehensive EV proteome databases (VesiclePedia and ExoCarta). The resulting set of unique MkEV proteins are listed in Table 4.

TABLE 4
Combined set of unique MkEV proteins (2 batches)

| Accession | Gene |
|---|---|
| P62805 | H4C16 |
| P0C0S8 | H2AC17 |
| Q71DI3 | H3C13 |
| P32969 | RPL9P9 |
| P35232 | PHB1 |
| P0DME0 | SETSIP |
| P0C0S5 | H2AZ1 |
| Q5T750 | XP32 |
| Q8WXC6 | COPS9 |
| Q9H9S3 | SEC61A2 |
| P56192 | MARS1 |
| P49959 | MRE11 |
| P36542 | ATP5F1C |
| P62805 | H4C1 |
| Q16777 | H2AC20 |
| P68431 | H3C1 |
| O60814 | H2BC12 |
| Q8N0W3-2 | FCSK |
| O95810 | CAVIN2 |
| P54577 | YARS1 |
| Q71DI3 | H3C15 |
| O75367 | MACROH2A1 |
| P16104 | H2AX |
| Q5TEC6 | H3-2 |
| O60613 | SELENOF |
| Q16181-2 | SEPTIN7 |
| P49588 | AARS1 |
| P16401 | H1-5 |
| P16403 | H1-2 |
| Q71UI9 | H2AZ2 |
| P41250 | GARS1 |
| Q9P2J5 | LARS1 |
| A0A3B3ITS0 | THPO |
| P63244 | RACK1 |
| P84243 | H3-3A |
| Q8NCU4 | CCDC191 |
| Q6DRA6 | H2BC19P |
| Q15019 | SEPTIN2 |
| P07814 | EPRS1 |
| P26639 | TARS1 |
| P06576 | ATP5F1B |
| Q96RT1-3 | ERBIN |
| P14868 | DARS1 |
| P23381 | WARS1 |
| P26640 | VARS1 |
| Q96TA1 | NIBAN2 |
| Q14141 | SEPTIN6 |
| O43776 | NARS1 |
| P54136 | RARS1 |
| Q9NVA2 | SEPTIN11 |
| Q99719 | SEPTIN5 |
| Q5T5C7 | SARS1 |
| Q9BZQ8 | NIBAN1 |
| A8MWD9 | SNRPGP15 |
| Q8N4L2 | PIP4P2 |
| Q9NUQ9 | CYRIB |
| Q5VZK9-2 | CARMIL1 |
| Q15046 | KARS1 |
| P41252 | IARS1 |
| Q9UHD8 | SEPTIN9 |
| P10412 | H1-4 |
| Q92619-2 | ARHGAP45 |
| P07305 | H1-0 |
| P49589-3 | CARS1 |
| Q92616 | GCN1 |
| O95864 | FADS2 |
| Q9NVG8 | TBC1D13 |
| O43681 | GET3 |
| P10155 | RO60 |
| O43504 | LAMTOR5 |
| Q15369 | ELOC |
| Q99878 | H2AC14 |
| Q14162 | SCARF1 |
| Q9Y225 | RNF24 |
| Q5JQS6 | GCSAML |
| O43847-2 | NRDC |
| Q5VYK3 | ECPAS |
| Q9P0M6 | MACROH2A2 |
| P25705 | ATP5F1A |
| Q08495-2 | DMTN |
| Q6ICL3 | TANGO2 |
| P32927 | CSF2RB |
| Q12768 | WASHC5 |
| P22001 | KCNA3 |
| P47897 | QARS1 |
| O95866 | MPIG6B |
| Q8N5J2 | MINDY1 |
| Q15257-2 | PTPA |
| A0A1C7CYX0 | EXOC3L2 |
| Q86TP1 | PRUNE1 |
| O94903 | PLPBP |
| Q9NXG2 | THUMPD1 |
| Q2M389 | WASHC4 |
| Q92692 | NECTIN2 |
| Q13630 | GFUS |
| M0R0K5 | ADGRE2 |
| Q9ULX6-2 | AKAP8L |
| Q9H0X4 | FAM234A |
| P30520 | ADSS2 |
| Q6ZTN6-3 | ANKRD13D |
| Q8NC54 | KCT2 |
| Q9H0P0-1 | NT5C3A |
| Q86T03 | PIP4P1 |
| A0A2R8Y4Z8 | CCDC9 |
| Q15370 | ELOB |
| O60760 | HPGDS |
| Q6P9B6 | MEAK7 |
| O94826 | TOMM70 |
| B2RUZ4 | SMIM1 |
| P12081-3 | HARS1 |
| O75947 | ATP5PD |
| O60502-4 | OGA |
| Q86X52 | CHSY1 |
| Q14534 | SQLE |
| Q92537 | SUSD6 |
| A0A075B6S5 | IGKV1-27 |
| I3L0A0 | PEDS1-UBE2V1 |
| Q9C0F1-2 | CEP44 |
| Q9HB07 | MYG1 |
| Q86YC3 | NRROS |
| Q9HBE5 | IL21R |
| P25098 | GRK2 |
| Q8IX19 | MCEMP1 |
| C9JA41 | ELAPOR2 |
| Q9NVU7 | SDAD1 |
| Q9Y5P4-3 | CERT1 |
| Q969M7-6 | UBE2F |
| Q8N5C1 | CALHM5 |
| Q92522 | H1-10 |
| A8MUM1 | EIPR1 |
| Q9BVG4 | PBDC1 |
| Q5T2E6 | ARMH3 |
| Q9H9C1 | VIPAS39 |
| Q14696 | MESD |
| Q86WR7 | PROSER2 |
| Q3YEC7 | RABL6 |
| O15117 | FYB1 |
| F8W038 | C17orf49 |
| Q96TC7 | RMDN3 |
| Q6YP21 | KYAT3 |
| Q5BVD1 | TTMP |
| Q96HE7 | ERO1A |
| Q9BZW8 | CD244 |
| Q9NWV4 | CZIB |
| O14495 | PLPP3 |
| Q9NXR7 | BABAM2 |
| P16402 | H1-3 |
| Q8NCW5 | NAXE |
| Q5T5H1 | ENSA |
| Q9NZW5 | PALS2 |
| Q02153 | GUCY1B1 |
| C9J7I0 | UMAD1 |
| Q4VC31 | MIX23 |
| Q8TBF2-6 | PRXL2B |
| A0A3B3IUA2 | SNU13 |
| Q9Y224 | RTRAF |
| M0QY77 | KXD1 |
| Q9H7M9 | VSIR |
| P19235 | EPOR |
| Q9P0N8 | MARCHF2 |

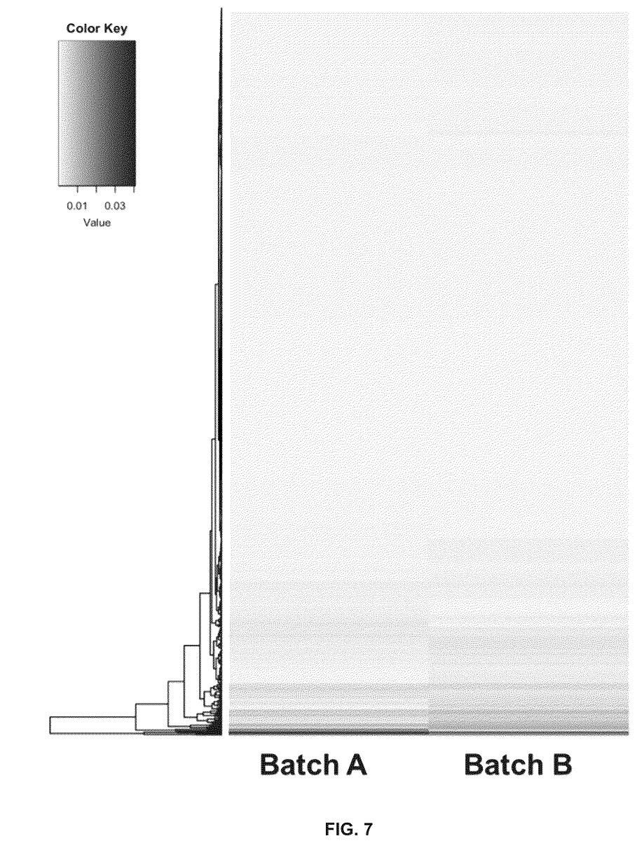

Protein intensities of the 973 overlapping proteins were sample loading (SL) normalized. Hierarchical clustering of normalized protein intensities was performed for both MkEV batches to outline inter-batch variation of protein abundances. The majority of both low and highly abundant proteins showed strong reproducibility between batches (FIG. 7).

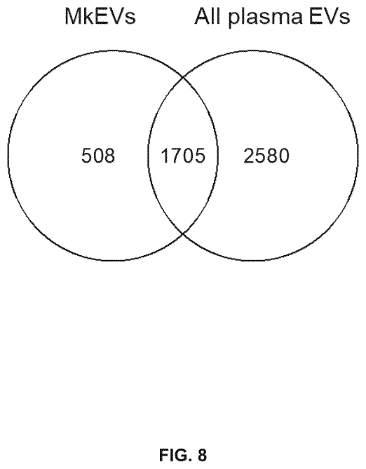

To determine the molecular overlap between MkEVs and naturally occurring plasma EVs from healthy donors, a comprehensive plasma EV reference proteome was generated (described in Materials and Methods). The subsequent comparison with the MkEV proteome (all unique proteins from two batches) revealed 508 MkEV-specific proteins (FIG. 8).

The MkEV-specific surface and/or membrane proteins are listed in Table 5.

TABLE 5
Surface and/or membrane proteins specific for MkEVss.

| Accession | Gene |
|---|---|
| Q9UKV3-5 | ACIN1 |
| P55263 | ADK |
| Q16186 | ADRM1 |
| Q06481 | APLP2 |
| P84077 | ARF1 |
| O43852 | CALU |
| Q13555-6 | CAMK2G |
| Q5ZPR3 | CD276 |
| Q00535 | CDK5 |
| Q9Y281 | CFL2 |
| Q9NPF2 | CHST11 |
| Q9NRB3 | CHST12 |
| H3BNK7 | CLN3 |
| Q9H0A8 | COMMD4 |
| O75629 | CREG1 |
| P46108 | CRK |
| C9JP16 | CRTAP |
| P32927 | CSF2RB |
| Q8WXX5 | DNAJC9 |
| P49770 | EIF2B2 |
| Q9BXX0 | EMILIN2 |
| P19235 | EPOR |
| Q9Y5Y0 | FLVCR1 |
| Q9NWU2 | GID8 |
| P06280 | GLA |
| P16278 | GLB1 |
| Q7LGA3 | HS2ST1 |
| Q9HBE5 | IL21R |
| Q9Y257 | KCNK6 |
| Q99538 | LGMN |
| P38571 | LIPA |
| Q9Y561 | LRP12 |
| Q8WXG6-4 | MADD |
| O00754 | MAN2B1 |
| P50579 | METAP2 |
| Q86UW6 | N4BP2 |
| P30419 | NMT1 |
| O00567 | NOP56 |
| Q9NPF4 | OSGEP |
| Q9Y6V0 | PCLO |
| Q9NRX4 | PHPT1 |
| Q5SGD2 | PPM1L |
| P50897 | PPT1 |
| P13727 | PRG2 |
| Q86WR7 | PROSER2 |
| Q86XS8 | RNF130 |
| C9IZU8 | RPE |
| P07093-2 | SERPINE2 |
| P50454 | SERPINH1 |
| Q9C0K1 | SLC39A8 |
| Q9Y6M7-13 | SLC4A7 |
| O43291 | SPINT2 |
| Q9Y2Z0 | SUGT1 |
| P62837 | UBE2D2 |
| Q8TAA9 | VANGL1 |
| P12956 | XRCC6 |
| P67809 | YBX1 |
| O15231 | ZNF185 |

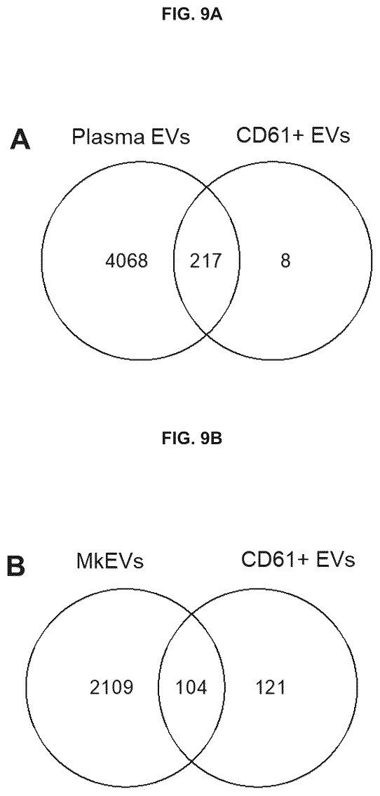

The proteome of CD61+ EVs, isolated from healthy donors, strongly overlapped with naturally occurring plasma EVs, with 96% of CD61+ EV proteins being identified in naturally occurring plasma EVs (FIG. 9A). In contrast, the overlap of the MkEV proteome with CD61+ EVs was limited to 46% (FIG. 9B).

### Example 2: Megakaryocyte-Derived Extracellular Vesicles (MkEVs) Contain Unique Gene Markers

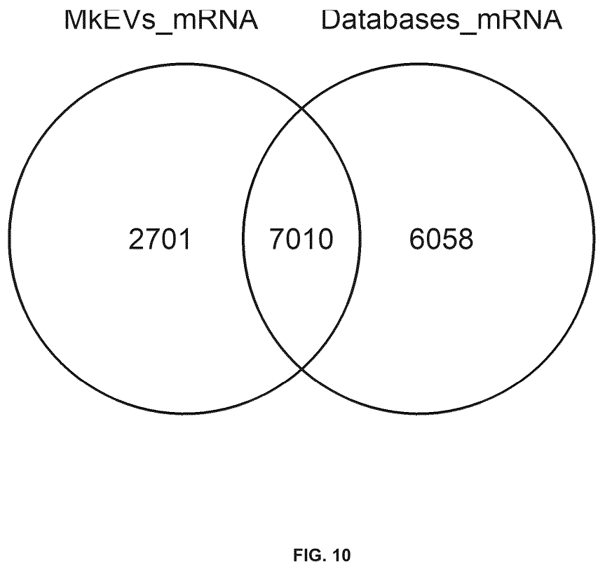

The presently disclosed MkEVs include unique protein coding transcripts. As shown in the Venn diagram of FIG. 10, an overlap exists between MkEV protein coding genes (RNA-seq) and transcripts identified across the VesiclePedia database. This diagram also revealed 2701 mRNA transcripts that are unique to the presently disclosed MKEVs.

Table 6 below provides the detailed list of these unique transcripts.

TABLE 6
Unique MkEV genes (RNA-seq)
Unique MkEV genes (RNA-seq)

STPG1, CFTR, RAD52, TMEM176A, CASP10, CAMKK1, VPS50, SLC4A1, MCUB, REXO5,
PRKAR2B, MPO, ITGAL, ABCC8, KDM7A, FARP2, MARK4, DNAH9, GAS7, MATK, CRAMP1,
CDKL5, PLEKHG6, PGLYRP1, MMP25, IL32, IYD, TRAF3IP3, PHF7, STAB1, FUZ, SLC6A13,
BTK, HFE, FMO1, MRC2, MKS1, ANOS1, MBTD1, PPP5C, CD22, LTF, ALOX5, EHD3, NUB1,
RGPD5, CNTN1, VSIG2, MARCO, PRDM11, MRE11, CYP3A43, CPS1, RTF2, NLRP2,
SLC45A4, GRAMD1B, TYMP, BTN3A1, POU2F2, ANK1, SARS1, LRRC7, SLC4A7, DAPK2,
ADSS2, STAP1, SLC18A1, CASR, MSR1, MTREX, RIPOR1, SPDL1, SLC66A1, RIPOR3,
ADAM28, JADE2, LCP2, GPM6B, XK, ARHGAP6, HDAC9, CELF2, ADAMTS6, PER3,
TNFRSF9, LTBP1, FOXP3, MCUR1, PTGER3, TNIP3, SIKE1, FNIP2, FSTL4, METTL24,
MCF2L2, KCNQ1, LY75, CHRDL2, FAM168A, KMT2C, ITIH1, TRAF1, TRAF3IP2, SERPINB3,
CDK14, NDC1, UNKL, GATB, PSD, YBX3, GNA15, QSER1, MPC1, BCAT1, GUCY1B1, DGAT2,
APPBP2, SEZ6, SLC6A16, BICRA, INTS13, TMEM161A, SNCAIP, FAR2, CHI3L2, CALCRL,
TAF11, ANKRD44, KARS1, ZC3H15, PACC1, SNAP91, COL17A1, TBC1D1, TIE1, KDM4A,
SLC9A3, CD84, SPI1, MPPED2, ACSM2B, ZFAT, DDX3Y, CACNB1, ZFY, NAV3, ATP11A,
ABCC9, PLA2G10, HDHD5, UFD1, LRP6, SPTB, DAPP1, TMEM260, FRMPD1, CFAP20,
RAD18, ATP2B1, HLTF, ADGRL1, SPP2, LNX1, UBE2D1, RHOBTB1, FCGR2B, EVC, SIDT1,
IRAG1, MOV10L1, SELENOO, CLCN4, DHRS9, PTGS2, FRY, ATP2A3, HACD3, MYDGF, TXK,
SEMA3C, VPS9D1, SEC31B, ARHGAP15, BAZ2A, SLC46A1, MBNL3, CTTNBP2, RARB,
PPP1R12B, APBB1IP, JADE1, UBE2A, N4BP2, ITM2A, NRDC, PKD2L2, CDH17, FKBP7, LXN,
OPHN1, CARMIL1, PTPRH, CRYBG3, CPB2, CFHR2, MAGI3, CXCL2, MEF2C, PTPRC, DELE1,
SLC13A1, FYB1, MPP4, EPB41L3, ERC1, TRPM3, DOP1A, TUT7, ZNF264, HAL, FAM234B,
SLCO1A2, WBP11, NKAIN1, AK6, PILRA, WNT11, USP40, CEACAM6, ERO1B, HSD17B14,
PGS1, CETP, LPCAT2, TXNDC16, RTRAF, NID2, CASS4, AAMDC, GP6, TMEM40, KIF9,
ARHGAP28, SIGLEC1, KIZ, GCN1, BRAP, HEPH, CDIP1, RCOR1, IRAK3, LYZ, CERS4, AARS1,
PITPNM2, RBM27, SLC26A4, MAP3K20, MYO15A, CCDC80, CMA1, JPH4, TRPM7, CAPN3,
AGO1, ADA2, VNN3, OR1I1, GABRP, FMO2, SUCO, PTGS1, SH2D3C, TPSD1, SMIM24,
CRISP3, PALMD, WASHC2A, STX1B, HSD3B7, ATP5F1D, MARCHF2, CECR2, GGT5, CYTH4,
ESS2, GRK3, PIK3IP1, PATZ1, CRYBB1, SNU13, SEPTIN3, DMC1, RTCB, HMGXB4, TOM1,
NEFH, CABP7, GRAP2, NCF4, CSF2RB, CHADL, ZC3H7B, CDKL1, DDHD1, CNIH1, TMED8,
RIN3, SUSD6, SLC10A1, SLC8A3, GSKIP, PPP4R3A, PRORP, GINS1, CD40, SGK2, SLA2,
RAB5IF, NFATC2, NELFCD, PRELID3B, MRGBP, GID8, ANGPT4, SNPH, SIRPB1, PLCB4, HCK,
TLDC2, MROH8, RPRD1B, BPI, EPPIN, MAP1LC3A, TNNC2, APMAP, CELF4, POLI, TLR8,
SYP, FMR1, EEA1, JADE3, PCYT1B, CD40LG, ZDHHC15, TAF7L, PBDC1, RUBCNL, HTR2A,
CDADC1, FLT1, VWA8, OLFM4, MT4, TSNAXIP1, CCL22, USB1, SMPD3, ESRP2, WFDC1,
SEC14L5, ABCC1, CIAO3, CAPN15, EARS2, ELOB, XYLT1, VPS35L, KNOP1, AQP9, IQCH,
ACSBG1, CD276, ZNF106, BMF, DMXL2, SLC30A4, EYA1, TRPA1, CPQ, BPNT2, TRPS1,
ANXA13, RETN, TLE6, CCDC61, RASAL3, SLC1A6, CLC, EBI3, YJU2, APLP1, DMAC2,
CEACAM4, ICAM4, NOP53, NKG7, ICAM5, CD33, CRX, CCDC114, RAB3D, CAPS, FAM83E,
KLF1, MAST1, KCNN1, UPK1A, ETV2, HPN, SCN1B, CFAP69, DNAJC2, PIK3CG, ITGB8,
GSDME, TFEC, H2AZ2, RNF32, CPED1, MINDY4, PTPRZ1, HYAL4, USP42, ZNF862, SFRP4,
POU6F2, TMEM176B, MYL7, GALNTL5, CNTNAP3, PRUNE2, TRIM14, OGN, ENG, RIC1,
KCNT1, MPDZ, GLIS3, ABHD17B, GATA3, DNMBP, CXCL12, VSIR, CCSER2, STN1, TASOR2,
SHOC2, DNAJC12, ASPA, SEPTIN4, SLC6A4, MED31, C17orf75, CCL8, PEX12, ABI3,
PTGES3L-AARSD1, ALOX12, TMEM33, SLAIN2, CRACD, PF4V1, GAB1, GALNT7, CLNK,
NSD2, BST1, RAPGEF2, LRP2BP, KLF3, ZBTB16, ZPR1, TECTA, SC5D, JHY, VWASA, KMT5B,
MS4A6A, HPX, FOLR3, SLC15A3, C11orf21, SOX6, POU2AF1, CD69, CLEC2B, BIN2, TNS2,
SLC6A12, GPN3, AKAP3, GSG1, OGFOD2, CLEC4A, C2CD5, COL12A1, FRK, MAK, SMIM8,
RIPOR2, OPRM1, RHAG, PHACTR1, CILK1, MDN1, FBXL4, VNN1, ADGRG6, BICRAL,
DNPH1, IMPG1, TENT5A, ERBIN, TENT4A, CERT1, PDE8B, HAVCR1, ITK, GABRG2,
POLR3G, CDH6, NPR3, TARS1, ST8SIA4, FGF1, NR3C1, TRAPPC13, C9, SEC24A, RARS1,
MACROH2A1, SELENOK, GNAT1, HHLA2, MORC1, CFAP92, NEK11, PLSCR4, HEMK1,
PEX5L, PLCH1, NEK4, MOB1A, PIKFYVE, SF3B6, CYTIP, CLIP4, PAPOLG, IL1R2, IL1RL1,
RMDN2, DARS1, CEP104, OPRD1, EDEM3, ASH1L, C1orf21, NCF2, PRDM2, RO60, NR5A2,
ADPRS, WARS2, NID1, MYCL, SLAMF1, CD48, PADI2, GBP3, RAB29, P3H1, PTCH2, FASLG,
TNFSF4, RSRP1, KMT2A, TREH, MMP8, KLF7, TTR, CA14, IRAG2, ADGB, ALDH8A1, TCF21,
SLC16A7, RXYLT1, RARRES1, EEF2KMT, LDAH, FGF23, ABITRAM, PTPA, NR4A3, CSF3R,
YLPM1, BBOF1, NEK9, ABCD4, NRDE2, SUPT7L, ATAD2B, FKBP1B, CNRIP1, SLF2,
DENND10, KCNIP2, CFAP58, C10orf95, TEK, INSL6, CD274, PLEKHG1, MYCT1, PLXDC2,
MTERF2, CHRNA2, EPX, TMEM131L, TENT4B, IAPP, ZNF549, ZSCAN18, RNF2, DPPA4,
NAA50, KIF18A, GJB6, ADGRB2, FABP3, CCRL2, ZNF639, ZSCAN20, CSMD2, LRIF1, POLK,
CD244, LY9, HS3ST2, SHLD2, PRXL2A, LRRC39, SLC66A2, SEPTIN7, NT5C3A, SRGN,
HVCN1, SPRYD7, ITIH5, NCKAP1L, NR4A1, EEF1AKMT3, USP45, TNFAIP6, G0S2, C4BPB,
RAB38, AGO2, FAM124B, ENKD1, CTCFL, SEMG2, ATP5F1E, CDH26, ZBP1, IL9R, POF1B,
CEACAM8, GRM4, H1-3, H1-1, H2BC11, TREM1, KCNK17, RREB1, CXCL6, EREG, SSUH2,
HROB, SEPTIN6, AMELX, DMAC2L, GRK4, FNDC11, IL1B, CD93, PAX1, MGME1, OTOR,
SIRPD, AGO3, PRRG2, SCAF1, PRR12, PCNX4, PZP, ARMCX1, TMEM35A, ZC4H2, INTS11,
RGS13, MASP1, TSPAN8, AUNIP, ADGRE2, OR7C1, F2RL3, METTL25, GNGT1, FGL2,
STEAP4, KDR, IGLL1, C22orf23, POT1, LSM8, STRIP2, IFT22, LRRC4, IRF5, LRRC17,
PDE11A, ARHGAP22, WDFY4, TMOD2, TTBK2, CCDC32, ICE2, ALDH1A2, VPS13C, KCNC1,
PIMREG, ATP1B2, AP1M2, SIGLEC9, RIPK3, HEATR5A, QRICH2, ARHGEF6, SGO1, PGAP6,
ABHD17A, LBP, CBFA2T3, ERMARD, GALNT8, NECTIN2, XPO7, COLGALT1, QRSL1,
MTRES1, AFDN, IQCN, SULT4A1, SAG, HELZ2, LSP1, TNNT3, HBZ, METTL26, ZC3H4,
ATP5IF1, THEMIS2, PPIL4, ULBP3, LATS1, AAR2, ZNF141, GSE1, ADGRE3, RBSN, GALNT15,
KIF3A, G6PC, CA6, ZNF304, SELENOS, PODNL1, TRIM22, TIMM10B, CLUH, DDC, H3-3B,
RIDA, VPS13B, MATN2, MTSS2, KHDC4, TESMIN, ZSWIM3, OSER1, DMGDH, PATJ,
FBXO44, CPLANE2, PDE6A, PIK3C2B, SPART, GPALPP1, STARD13, SLF1, CEP20, SLC2A11,
C1QTNF6, ATP13A3, CA1, LYVE1, SARAF, ERG28, DGLUCY, ADAMDEC1, KATNBL1,
GNAT2, VAV3, CHIA, PTPN22, LAMTOR5, CMPK2, IAH1, FST, NARS1, RELCH, FBH1,
ECHDC3, HRH4, RERG, KLRD1, SPX, LRP4, RTL8C, RAB33A, STK26, TUT4, C5AR2, PDGFRA,
GGACT, SLC37A2, APC, CEMIP2, HAVCR2, OASL, CCDC146, CD36, UGT2B28, MDFIC,
ANKRD6, LCA5, AMHR2, TESPA1, AGAP2, FAIM2, AFG1L, TEC, MDM2, PCNX2, RGS8,
RNASEL, NIBAN1, RC3H1, PAX3, DOCK10, SLC19A3, ANKRD36, PLXNC1, WASHC4, LRCH1,
EDNRB, RCBTB2, LCP1, RAPGEF5, NUP42, AOAH, HUS1, CIDEB, ZFHX2, TM6SF1,
MYCBPAP, LIMD2, MARCHF7, SCN7A, EPRS1, HLX, GAD2, ECPAS, NIBAN2, TLR4, ALDOB,
GARNL3, GABBR2, HEMGN, NR5A1, TRMO, ENPP2, IL33, DMAC1, FOXP4, ZSCAN9,
CAPN11, TLR2, ANKRD42, LRRC32, TMPRSS4, TRPC6, C11orf1, TMPRSS13, CASP1, SQOR,
KNL1, RMDN3, DUOX1, SLC28A2, SEMA6D, SPTBN5, IFI44L, SELENOI, SLC3A1, ATRAID,
CYP2C9, MFSD13A, ENTPD1, PLCE1, FAM149B1, CARF, SLC49A4, APH1B, PPCDC,
SEMA7A, FAM13A, HERC3, ZGRF1, RASGEF1B, MMRN1, SEPTIN11, CNOT6L, EGF, FBN2,
GUCD1, ALG10, PRICKLE1, AMIGO2, COL2A1, PPFIA2, MARCHF9, TPH2, TMEM19, RITA1,
NUP58, CCDC65, RDH16, GPR84, N4BP2L1, BRCA2, MYG1, SRRM4, ABHD13, TTC6, ESR2,
STON2, EFCAB11, GPR65, PTGR2, WARS1, SLC12A6, ZSCAN29, FGF7, TLNRD1, CYP1A1,
ZNF710, CRTC3, EFL1, RUSF1, IGSF6, MYLK3, CPNE2, MEAK7, RPL3L, ZSCAN32, GID4,
ZNF287, KSR1, UTP4, VPS53, ABCA8, C17orf64, BCAS3, ADCYAP1, RMC1, CCDC40, TTYH2,
SECTM1, CEP131, ZNF750, WDR45B, RNF165, ELAC1, MBD1, TNFRSF11A, P3H4,
CACNA1A, MISP3, TPGS1, VAV1, MVB12A, TMEM91, ZFP14, SIRT3, TRPM2, DOP1B,
ADAMTS10, MYO1F, ERVK3-1, NLRP12, TIMM29, SIGLEC10, SLC2A5, EVA1B, STRIP1,
CD53, MPC2, TBX19, XCL1, NR1I3, HMCN1, ZNF687, ANP32E, MINDY1, ANXA9, SEMA6C,
KCNH1, S100A8, CEP170, SDE2, REN, HNRNPLL, EXOC6B, ST6GAL2, LNPK, TMEFF2,
UNC80, KANSL1L, RETREG2, CNOT9, CNTN4, GADL1, ITGA9, STAC, NXPE3, RUBCN, AMT,
CENPC, SLC10A4, PLAC8, SNCA, PYURF, SPATA5, FABP2, MARCHF1, RNF175, PDGFC,
ROPN1L, MARCHF6, ADAMTS16, OTULINL, HAPLN1, BHMT, PPIP5K2, TNFAIP8, LECT2,
GABRB2, PLA2G7, MMUT, DOK3, ABT1, SCML4, ARMT1, CREB5, NIPSNAP2, TMEM140,
RAB19, LANCL3, DIPK2B, ZNF182, KRBOX4, GPR174, ZNF711, NXF3, RADX, IGSF1,
CSGALNACT1, DOK2, PLPBP, SNTG1, RGS20, PLPP5, NSD3, ADHFE1, ATP6V0D2,
MFSD14B, AOPEP, NR6A1, WDR31, GBGT1, PAXX, DPH7, USP6NL, MINDY3, ST8SIA6,
FAM13C, NRBF2, CDHR1, RGR, LRMDA, LRRC27, CNNM2, ADAM12, SERPING1, HIKESHI,
CCDC82, ZC3H12C, DRD2, SLX4IP, GRIK4, TKFC, TMC2, INCENP, MS4A3, CPSF7, MS4A2,
CNBD2, LSM14B, FERMT3, VPS51, TLCD3B, CNKSR2, CLEC1A, MPP7, FCGR1A, CDH8,
TMEM218, MIA2, ADRA2A, GPM6A, WDR17, CD226, RGS18, ATPSCKMT, NKAPD1,
ABCB9, RILPL2, NOCT, ENKUR, GPR158, TMEM263, IPMK, PLBD2, SLC2A13, DIP2C,
MIPOL1, VIPAS39, QTRT2, AKR1C2, ADAM8, KIN, KCNJ1, CENPU, ZNF385D, GABRA2,
TMEM267, KCNE4, AP1S3, GPATCH11, OBI1, ARL14EP, EPG5, ATP5F1A, PDK1, GUCY1A2,
XRCC4, ZNF776, OLAH, PLEKHH2, SPEF2, NADK2, WDR78, HNRNPDL, ZNF117, PLOD2,
NRSN1, CPB1, ACMSD, PTPRR, CD96, ADGRF1, CYRIB, FAM81B, UBALD1, SPACA7,
TMPRSS11D, SPHKAP, ZNF599, LGI4, GRAP, CABYR, IMPACT, ANKH, TOMM70, ABI3BP,
ANGPT1, ABCA9, ABCA10, ABCA5, MIA3, DIPK1A, ATP5MC3, ELOC, LY96, PSMA8,
TMSB4Y, JAM2, ATP5PF, SLFN13, PIEZO2, SEPTIN14, AK9, PIP4P2, HSPA13, SAMSN1,
GRAMD2B, ZCCHC10, NIFK, LRGUK, MIER3, TTN, VSIG4, FLACC1, C2CD6, ELMO1, SAXO1,
PXYLP1, SLA, CLIC2, AFF2, KIF5A, CARNMT1, MIDEAS, C8orf37, CFAP161, ADAMTSL3,
ART3, ANKRD9, ATP5MPL, MTERF3, PPP2R2B, SAMD8, UNC5D, GLYATL2, TBC1D31,
NTAQ1, FRRS1, MFSD14A, SASS6, B3GNT7, MPV17L, BRPF1, ATP2B2, SLC6A1, RBPMS,
KLHL40, NRG1, ODR4, FCSK, CACNA1D, KIT, ZNF19, KCNJ15, TSPAN18, LCA5L,
PALM2AKAP2, DGKI, TMEM268, SVOPL, SLC34A2, CABP1, PRXL2B, MEGF11, ANKRD61,
BABAM2, CFAP251, UBXN11, GALNT14, PRXL2C, TENT5B, H2BC5, CATIP, CNOT11, CD1C,
NCF1, DYNC1I1, ALAS2, PPP1R15B, PKD1L1, SLAMF8, ITLN2, DMTN, TOMM40L, CCAR2,
CFAP298, PAXBP1, C1QC, ATP5MC1, CIART, GOLGA6A, M1AP, DRC7, ZDHHC1, ZFYVE28,
CARMIL2, PSKH1, ZNF221, CCDC28B, UBASH3A, GATD3A, CFAP410, ZNF66, SPATC1L,
S100B, ZNF208, JAML, S100A1, ZBTB7B, IL6R, MYL3, LRRC71, PWWP3A, CELF5, NPHS1,
ZNF382, SIGLEC11, MPP3, FAM171A2, FMNL3, BCL6B, ASGR2, TNFSF13, CCDC42,
TBC1D24, NEU3, CYB561A3, PPP1R32, LBHD1, PDZK1IP1, TAL1, COA7, CZIB, PLPP3,
MATN1, TSSK3, MYSM1, ADGRL4, LRRIQ3, WDR63, KCNT2, NLRP3, SLAMF6, FCGR3B,
AXDND1, KIF26B, KLHDC8A, REL, MEMO1, TYW5, MAIP1, SLC66A3, KCNJ3, DUSP19,
CFAP36, WDCP, H3-3A, COQ8A, XIRP2, CTSS, BNIPL, TNFAIP8L2, TMOD4, DHX57,
S100A12, ABRAXAS1, GPR155, PBXIP1, INAVA, NAXE, SLC22A15, SLC15A2, PROK2,
TMEM169, NEK10, CIP2A, FBLN2, SGO2, SERPINI1, SPTA1, MNDA, PPM1L, NEPRO,
SPICE1, CCDC191, CADPS, WDFY3, COX18, PRICKLE2, ADAMTS9, SMIM14, CFAP20DC,
CXCL5, PF4, CPA3, CCR1, ELP6, FBXO40, LIPH, RPL39L, SLC51A, RNF168, EXO5, EMCN,
SLC9B1, CAMP, INTU, ABHD18, DUSP7, ETNPPL, MAP9, GUCY1A1, CEP44, HPGD,
C4orf45, TMEM144, NPY1R, ICE1, HHIP, TMEM184C, PRMT9, TMEM161B, ZNF474,
LMBRD2, ADGRV1, F2RL2, SPINK1, CDC20B, ARSK, TIGD6, PRIMPOL, ERAP1, CFAP97,
SEPTIN8, CGAS, SAMD3, IL31RA, ANKRD55, RAET1E, BMT2, KIF6, ELAPOR2, IQUB,
TAGAP, ADCY1, PHKG1, KCNV1, CSMD3, SPIDR, DNAAF5, GPR146, UNCX, VIRMA,
TMEM67, MYORG, CCDC171, ABCA1, C8orf34, KDM1B, HGSNAT, RASEF, CYBB,
METTL27, CARD19, ATP7A, ZNF367, AQP7, INTS6L, MARCHF8, DDIAS, EML5, AKR1E2,
FAAH2, ATP5F1C, FAM204A, CLEC1B, ENTR1, AK8, SPACA9, DIPK1B, JCAD, PIP4P1,
NSMF, C12orf50, CCDC186, TRUB1, CYP2C19, C10orf82, HSPA12A, TTC7B, IFI27, CLMN,
CACNB2, HACD1, SMCO4, CEP295, AMOTL1, LIPC, TCP11L2, GPR176, CMTM5, GLB1L3,
AVPR1A, C16orf71, BORCS7, ATP9B, GARRE1, TRIM66, DENND2B, CDYL2, PRTG,
C16orf46, HDGFL3, CCDC68, CLEC4E, CLEC4D, CIAO2B, CFAP52, BEX3, GOLM2, BMERB1,
MARF1, LDHAL6A, PCLAF, LDHD, CHP2, NEMP1, C15orf48, MS4A7, MS4A14, EPB42,
MEI1, B4GALNT2, SAMD14, PIP5KL1, SLC27A4, LOXHD1, DPEP2, DUS2, ENGASE,
RBFOX3, ATP5MG, OR51I1, FN3K, GNG8, LPO, JSRP1, NIBAN3, SPATA33, KMT2D,
ZNF528, PROSER3, LAIR1, TMIGD2, HDGFL2, SLC43A2, KRT1, CD300C, MRPL58,
TMEM88, NLRC3, BEST1, SPINDOC, ATG16L2, TRANK1, ENTPD3, NAALADL1, CCDC88B,
PTGDR, COA6, H1-4, DEGS2, SCN11A, SEPTIN2, ING5, RHOH, CAVIN2, MTCL1, CHRM1,
GFRA2, ADAM9, IL7R, LRP1B, TET2, PPIP5K1, ZNF507, NSG1, SIGLEC7, GSG1L, MN1,
NPIPB12, GCSAML, NPIPB3, CXCL11, ADRB2, SHE, P2RY12, OR5AU1, RNASE2, RSPH10B2,
PTAFR, CXCL8, HTRA4, GPR183, ZEB2, DTWD2, RAMAC, PROKR1, RGPD8, HEXD, CENPX,
TMEM266, CSGALNACT2, ROBO1, P2RY1, AHSP, ITGAM, ZNF764, TMEM154, SIMC1,
GYPA, ADPRM, FAXDC2, ELP5, TCAF2, ZNF804A, RIOX1, PYM1, MZB1, SLC23A1, EMB,
DLGAP1, ATF7, POLH, HTRA3, CEL, RNF139, TANC2, OR1M1, S1PR1, PKIA, FPR2, FPR1,
OR2M4, LRG1, SOSTDC1, FAM98B, ZNF440, DSEL, COL24A1, LPAR3, PTGER4, OR2AT4,
NMUR1, PTCRA, PAH, GATM, IFNB1, MRM3, FBXW10, DRC3, ZNF554, USP19, SLFN12,
FRMD3, AZU1, TPSAB1, CLEC7A, NEGR1, MACROD2, COPRS, OR5A1, CLEC12A, OR5A2,
CFAP53, PRSS27, RSPH9, COPS9, TRIM40, CTSW, PDE3A, RASGRP1, KLHL6, RND1,
AGAP5, WASHC2C, FUT10, OR4D9, ZNF596, EFCAB12, PSME3IP1, CBWD1, RAB37,
SNX32, RARG, CES3, PHF8, ADH6, ARPP21, TADA2B, GRK2, HECTD4, HPSE, MRPL57,
CYSLTR1, PARP15, ABLIM3, MFSD4B, SYT12, ZBTB21, PPP1R3B, OR10K1, NDNF, IHO1,
MICOS10, EHBP1L1, THAP2, ZNRD2, TNFRSF10C, CEP83, SULT1B1, UGT2A1, TRAPPC3L,
APOBEC4, BCLAF3, CD7, ATP5MD, MARCHF3, NRROS, FBXO45, C9orf131, CD34, TLR6,
TRMT10C, SELP, SNX31, ZNF80, SLC16A13, STARD6, CNTNAP2, GCSAM, MYO1H, MSL2,
ZNF497, OR7D4, B4GAT1, FAM241A, WDR49, PCP2, ADGRE1, DENND6A, CATSPERD,
P2RY14, GPR171, FBXW8, CA5A, TVP23C, NPPA, CENPS, PHYKPL, APOF, ARL10, PCSK1,
LPL, LRRC25, TSGA10IP, LIPT2, KCNE3, ERCC4, MTLN, EIF4E1B, ZNF169, GAPT, CALCB,
ZDHHC21, TSPEAR, ENTHD1, LRRTM4, SMIM19, RTTN, OR4K17, ZNF135, SYNE3, PLAAT3,
USF3, RMDN1, OR4F17, RBIS, NCKAP5, LRRC37A3, ANKLE2, ACER2, PHETA2, FAM9B,
NUDT4B, OR2T12, GATD1, TRIM72, KCNA3, BEND2, SAMD9L, MIEF2, NAP1L5, OR2T8,
CAVIN1, RABEP2, SAMD12, CD163, C18orf32, HASPIN, PGBD5, CRACR2B, NECTIN3,
TMEM94, SOX12, ZNF619, AP3S1, DPY19L2, ADAMTSL1, CALHM5, ZNF518B, ZNF366,
POGLUT3, SPAG11A, GEN1, ZNF354B, KCNG2, KLHL11, CD28, DYNAP, THBD, GP5,
EFCAB13, MSC, DPY19L3, ZNF852, CYBC1, ZNF552, SELENOW, SNX18, EXOC3L1, IGSF22,
PER1, TCAIM, PXT1, MKRN3, ZBTB18, CIITA, OR6C4, FCER1A, PCED1B, AKAP5, GIPC3,
ZNF154, NRXN1, ZNF771, C3orf80, WFDC11, SEPTIN1, TDRP, MYLPF, TIGD2, HCLS1,
MTURN, ARHGAP45, ZNF571, MIGA1, KCNE1, H2BC4, TMEM64, CXCR2, CMTR2, ST20,
OR52E4, BBS12, ZFP82, LSMEM1, SLC25A42, EHMT1, F2R, H2AW, TMEM102, NME9,
OGFOD3, WASHC1, ZNF678, P2RY13, ZFP41, ZNF875, DIPK2A, MACIR, OR5AS1, SLC9A9,
RELL1, COA4, TMEM259, GET1, ERCC6L2, P2RY8, NLRP10, SPACA6, ZSCAN22, KIAA2012,
PGBD4, FES, SATB1, EPGN, NDN, PPP1R27, BRICD5, RGS6, SLC35D3, PAPPA, ADGRG3,
SRPRA, ZNF320, SPNS2, MCEMP1, CSMD1, PTGDR2, GABRR3, MICA, CCDC60, PLGLB1,
CCBE1, SELENOF, ZNF623, C19orf71, GPR132, MX2, TENT5C, PRR14L, H3C13, HMCES,
RP1L1, ZNF530, RFLNB, NOG, MACC1, ACSM2A, CHEK2, TUBA8, CCR4, BTBD9, ANKRD45,
UTY, SDR42E2, KMT5A, CLDN5, OR1D2, GOLGA6L4, CMSS1, PCDH9, H2AC20, H2AC21,
SRPK3, H1-5, CSF1, MAML2, BPIFC, PROS1, STING1, PDE4B, KRBA2, SEPTIN9, H2BC21,
SEPTIN5, APOBR, SMIM10, SATL1, H1-10, RBM43, CHRM5, TMEM106A, NELFA,
SLC24A3, EFCAB10, ADSS1, H2BC13, PRSS57, ZNF445, TCEAL9, RAB11B, PRPF39, ZNF74,
UBALD2, KLHL33, ZBTB37, NUPR2, IL3RA, ARL15, PRKN, TEDC1, SP140L, TARS3,
METTL7A, ZSWIM9, GPRIN3, STAC3, PRKG1, DLK1, TLCD2, LSAMP, INKA1, PCGF3, LMLN,
SHC4, SYN3, MOSMO, ADARB2, C11orf87, IFIT1, IKZF1, DNAH14, EVI2B, NPIPB4, ZNF829,
TMPRSS11B, POMK, C16orf54, KLHDC8B, ZNF267, ZNF566, CD300LF, AGBL4, ARGFX,
LRRC70, ZBTB6, KIF18B, ZNRF1, BPIFB3, MTARC1, BLOC1S4, ZNF749, MRTFB, ZNF555,
TMEM212, GOLGA8R, CCDC30, ZNF501, GNG2, SEPTIN10, SMYD4, SMIM29, PDE2A,
PRG2, CYP27C1, OR10H1, SPIN2B, ZSCAN30, TRABD2A, PPARA, KRTAP13-4, EMID1, RHD,
ENTPD5, AL162231.1, TSPYL4, SESTD1, FAM9C, MAGI2, H1-6, GJA4, IFT140, AGMO,
TLR5, TET3, VMAC, DNAJB13, GABRD, FANCA, FANCM, ZNF70, TMEM220, H1-2, OR6C75,
CCDC157, CLEC17A, PLA2G2C, OR7D2, TPRG1, ZNF490, CLCN1, TMPPE, ZC3H6,
LAMTOR4, POTEE, ZNF793, AGAP4, PEAK3, GPR21, TCTEX1D4, SELL, BLOC1S5, H2AX,
CCDC9B, RTL6, DPYD, RHCE, C2orf80, SMIM15, FSIP2, SNTN, CALHM6, TMC3, FBF1,
MSL1, INSYN2A, HACD4, UTS2B, NELFB, ZNF292, ADAT2, APOD, H1-0, BLOC1S3,
FAM47E, JPT1, NUGGC, GJB5, SLC35E2B, NEMP2, IL1RAP, MYBPC1, ZNF676, ZNF699,
CCDC189, OR8A1, KIF19, SRGAP3, SULT1C3, OR2C3, ZNF107, ZNF471, IARS1, SRGAP2B,
PRTN3, CRYBA4, NPIPB15, ZNF605, C20orf96, ESRRG, BORCS6, MME, CACNA1H, HBG2,
LAMA2, MRTFA, SLC22A25, SDHAF3, RABL6, HSH2D, NF1, DAPK1, H2AC13, OR52K1,
H2AC11, CHRNG, MVB12B, ILRUN, POTEI, H2AC7, H2BU1, WDR5B, EOLA2, H4C3,
ZSCAN26, ZNF772, PCNX3, ADAM32, LRRC8B, ABCB8, H3C12, NOL4L, FITM2, FBXL22,
DACT3, H3C4, VEPH1, GYPE, OR6Y1, ELANE, ZNF624, KCNMB2, CPLANE1, ZNF841,
EOLA1, SERPINB2, DPP4, DNAH10, KRTAP26-1, ZNF460, CFAP43, LHFPL5, TXNRD3,
STPG3, MAP1LC3C, EME2, ZNF81, ZNF780A, SGTB, ZNF790, H2BC12, ZNF677, ERO1A,
ZNF347, FCHSD1, ZNF71, KEL, ZNF667, MARCHF5, NPIPB13, ZBTB14, ADH4, OR2L3,
RPS6KL1, GET3, OGA, TCAF1, OR14L1P, ZNF568, ZNF43, C20orf203, CTNND1, RYR2, TAT,
TAFA2, TUSC1, EIF1AY, MT-ND6, MT-CO2, TOGARAM1, MT-CYB, LDB1, F5, PLXNB3, MT-
ND2, SYCP1, RCSD1, MT-ND5, BRINP2, MT-CO1, LRRC10, SUCNR1, SELENOM, MT-ND3,
SELENOT, TOX, MT-ND4, MT-ND1, CIPC, MT-ATP6, SPOUT1, MT-CO3, SGMS1, C6orf47,
OR2L2, CHML, STUM, FCGR3A, H3C14, H2AC18, H3C15, HSD3B1, SAMD13, NEU4, CHIC1,
NHSL2, AGAP9, MACO1, PTPN20, OXLD1, NBDY, PJVK, SMIM5, NELFE, LY6G6F, AIF1,
ZNF805, KRTAP5-11, ASPDH, OR14J1, SPDYC, MALRD1, MROH6, IGFL4, SPINK9, UQCC3,
FBXO48, CD177, ZNF783, PKHD1L1, TMEM231, ACCSL, TRIQK, ZBTB10, TMEM170B,
ADGRG1, SAMD9, EXOC3L4, TP53TG3D, TMEM256, DENND6B, MFSD2B, HTN3, DPF3,
ETFRF1, CYS1, PLPP6, OLIG2, NYNRIN, GOLGA8O, ANKUB1, B3GALT4, HLA-DRA, SNHG32,
RAB12, CD200R1L, LNP1, COLQ, SELENOH, RNF208, MT-ND4L, DENND1B, CEP43,
TRIM59, OR2F1, GSTM2, ERV3-1, GBP7, RBMXL1, STIMATE, TREX1, UGT2B11, ZNF888,
LTB4R, HBG1, ITGA1, NUDT19, ZNF726, GANC, ZNF891, SMIM30, C19orf38, BBIP1,
FAM187A, PLIN5, SEC14L6, NUTM2D, B3GNT10, ZSWIM8, TOMM6, MS4A4E, TBC1D26,
UBXN2B, GOLGA8B, RPL17-C18orf32, SLC35E2A, IGSF23, CDC14C, CEBPZOS, LINGO3,
PLXNA4, ZNF880, BTBD19, TRIM49D1, ZNF844, TINCR, CPTP, HLA-DPA1, PRR29,
SMIM13, FAM236D, Z82206.1, MCRIP1, HLA-DQA1, LY6G6E, TMEM185B, C13orf42,
AKR1B15, MRLN, OR2M3, MT-ATP8, ORM2, FAM174C, NUTM2E, FNDC10, ARL17B,
PGA4, ORM1, PATL2, PET100, IQCF3, OR5H6, ANKRD18B, HLA-DPB1, STMND1,
CFAP97D1, TSPAN19, PRH1, SMIM26, AJM1, LYRM9, NPIPA9, USP17L11, NPIPB7,
AC092835.1, H2BC15, ZNF879, PINLYP, NPIPB2, IQCM, BRD3OS, SMIM1, HGH1,
SMIM27, ANKRD34C, KIAA0040, SLFN14, C3orf84, C4orf51, ZNF737, LRRC37A2, OR2W3,
DEFA3, TEX35, PLCXD2, TDGF1, AC244197.3, UGT1A1, HYPK, ARPIN, SERPINB10,
CCDC169, STRC, NAA80, ZNF487, LEFTY1, TMEM35B, UGT1A7, CCDC13, H2AJ, INSL3,
APELA, PCP4L1, USP17L22, LGALS16, TERB1, ARPIN-AP3S2, TRMT9B, ZNF718, GYPB,
AC068580.4, SELENOP, ZNF345, ALG1L2, MSANTD3-TMEFF1, C8orf88, CCDC71L,
TMEM200B, SIGLEC14, AP002495.1, SIGLEC12, FPGT, IGLL5, HTD2, NOX5, HCAR3,
AC005833.1, MTRNR2L8, AL117348.2, COA8, TRAPPC2B, SMIM3, ZNF225, AP002748.5,
AP003419.1, SMIM10L1, MTRNR2L1, HP, MGAM, RPL36A-HNRNPH2, INAFM1, TEN1,
CLEC5A, AP001781.2, TRIM34, AC007040.2, AL355916.3, SMIM6, INAFM2, ST20-MTHFS,
AC090527.3, FRRS1L, AL049634.2, AC026464.4, AC140504.1, CLEC19A, ZNF865,
PECAM1, GOLGA6L7, BOLA2-SMG1P6, GFY, LINC00514, AC233723.1, CCNQ, MYZAP,
DPEP2NB, LINC00672, RHEX, GJA5, AC134669.1, ANXA8, FXYD1, AC006538.2, PTGES3L,
AC012309.1, UPK3BL1, FDX2, EDDM13, AC004076.1, SMIM17, AC018755.2,
AC006486.1, AC010522.1, MTRNR2L12, ZNF587B, SLC25A53, NCBP2AS2, HSPE1-MOB4,
RPS10-NUDT3, H4C14, NBPF19, CCL5, MILR1, H2AC19, KMT2B, CD24, GRIN2B, NBPF26,
H4C12, H2BC14, FCAR, SMN2, H2BC8, AC244489.1, USP27X, H3C8, LILRA2, CASTOR2,
LILRA5, MYOM2, NATD1, CCL3, H2BC6, TBC1D3L, LILRB2, RCC1L, H4C6, H2BC17, H3C6,
BDP1, H2AC12, PPP4R3B, H4C13, H2AC15, CCL4L2, H3C11, FCGBP, MUC20, FP565260.1,
H2BC9, H3C1, AC091959.3, TMEM251, AC139677.1, SPATA31A7, MAPT, H4C9, PIK3R6,
AC011043.1, H2AC14, H2BC3, AC245049.1, H2AC16, H4C5, H2AC8, MMEL1, H4C4,
H2BC7, H3C7, ZNF8, CARS1, LENG9, H2AC4, H2BC10, NAIP, C11orf98, MRM1, H4C1,
H2AC17, IQCA1L, BX004987.1, H4C2, H3C10, TSTD3, OR2L8, AC013394.1, OR51F1,
GATD3B, FP565260.6, CCDC163, PAGR1, ZNF546, AC243733.1, HNRNPCL4, NSMCE3,
AL157392.5, AC058822.1, LBHD2, C13orf46, AC001226.2, TEX54, FAM236C, MYOCOS,
VSIG10L2, AC007326.4, AC270107.1, AC270107.2, PLCL2, AC270107.3, AC270107.5,
AC270107.6, AC270107.7, AC025287.4, AC015802.6, AL022318.4, SCYGR4, EEF1AKMT4,
CCDC39, AP000812.5, AC270301.3, AC104389.6, BX255925.3, UPK3BL2, AC093512.2,
AC022506.1, DEFA1B, AC026316.4, AC090517.4, ABCF2-H2BE1, FO393400.1,
AC093827.5, USP17L7, AC097636.1, AC024598.1, AC072022.2, AL135905.2,
NOTCH2NLB, SPEGNB, AC008770.4, DERPC, NOTCH2NLC, AP001453.4, H3C2, H3C3,
AL445524.2, AC275546.1, MG910336.1, AC118282.7, AC270106.1, AL035699.1,
ELOA3D, AC106873.8, AL049569.3, and AC084756.2

A KEGG pathway analysis revealed a list of the genes unique to the disclosed MKEVs that are associated with selected pathways. These pathway associated genes include H3-3A, H4C12, H2AC18, H2AC13, H2AC19, H3-3B, H2BC12, H4C3, H3C10, H2AC14, H2BC4, H2AC20, H3C13, H4C14, H3C2, H2AC8, H2BC3, H3C7, H2AC17, H3C4, H2BC11, SELP, H3C11, H4C2, H2AC15, H2AC12, H3C1, H4C4, H2AC4, H4C13, H2BC5, H2AC7, H2AC16, H2BC17, H2AJ, H3C3, H2BC10, H2AC21, H3C14, H3C15, H2AC11, H3C12, FPR1, H2BC13, H4C1, MACROH2A1, H2BC14, H2AX, H2BC6, NCF4, H4C6, H2BC15, H2BC21, H2BC7, H2BC8, CYBB, FCGR1A, H2AZ2, H4C5, AQP9, H2BC9, CLCN4, FPR2, H4C9, HDAC9, ITGAL, FCGR3B, ITGAM, H3C8, TLR2, NCF1, NCF2, H3C6, CAMP, MPO, ELANE, FCGR3A, AZU1, H2AW, SIGLEC9, CLEC7A, PLCB4, CASP1, H2BU1, TLR4, TLR8, CD36, IL9R, FCGR1A, CSF3R, KIT, GP5, CSF1, ITGAM, CD33, IL1B, IL7R, IL6R, HLA-DPB1, CD24, CD22, GYPA, MME, ITGA1, CD1C, IL3RA, HLA-DPA1, IL1R2, CD34, HLA-DRA, HLA-DQA1, CD7, CD226, SELP, PECAM1, VSIR, PTPRC, NRXN1, NECTIN2, ITGAL, ITGAM, SELL, CD28, HLA-DPB1, CNTNAP2, CLDN5, JAM2, CD40LG, LRRC4, CD274, CD40, CD276, CD22, ITGA9, NECTIN3, NEGR1, HLA-DPA1, CNTN1, ITGB8, CD34, HLA-DRA, SIGLEC1, HLA-DQA1, CFTR, ABCD4, ABCA5, ABCA9, ABCC1, ABCB8, ABCA8, ABCB9, ABCC9, ABCA1, ABCA10, ABCC8, F2R, LCP2, FYB1, FPR1, F2RL3, P2RY1, RAPGEF2, KIT, EGF, CSF1, ANGPT1, PDGFC, ITGAL, VAV1, VAV3, PDGFRA, ITGAM, RAPGEF5, MAGI2, FARP2, CTNND1, FGF23, FLT1, FGF1, DRD2, AFDN, PLCB4, APBB1IP, PLCE1, MAGI3, KDR, LPAR3, GRIN2B, ADCY1, FGF7, ANGPT4, TEK, PF4, CCL5, CXCL2, CXCL8, ELMO1, GRK2, GNG8, CCR4, PF4V1, VAV1, VAV3, PIK3R6, PIK3CG, SHC4, GRK3, NCF1, HCK, GRK4, ITK, GNG2, CXCR2, CCR1, CXCL5, CCL3, PLCB4, CXCL6, CCL22, CCL8, CCL4L2, CXCL12, GNGT1, CXCL11, ADCY1, XCL1, TUBA8, NCF4, CD36, CYBB, FCGR1A, CTSS, FCGR3B, PIKFYVE, ITGAM, TLR2, NCF1, NCF2, HLA-DPB1, MPO, EEA1, MSR1, FCGR3A, FCAR, DYNC111, CLEC7A, FCGR2B, HLA-DPA1, ATP6VOD2, MRC2, TLR4, TLR6, HLA-DRA, MARCO, HLA-DQA1, RARS1, DARS1, KARS1, SARS1, AARS1, TARS1, NARS1, WARS1, GATB, CARS1, EPRS1, TARS3, EARS2, IARS1, QRSL1, and WARS2.

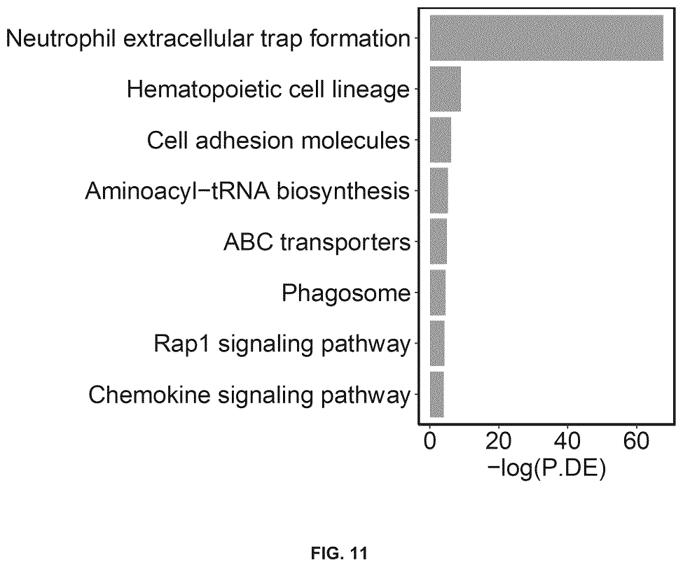

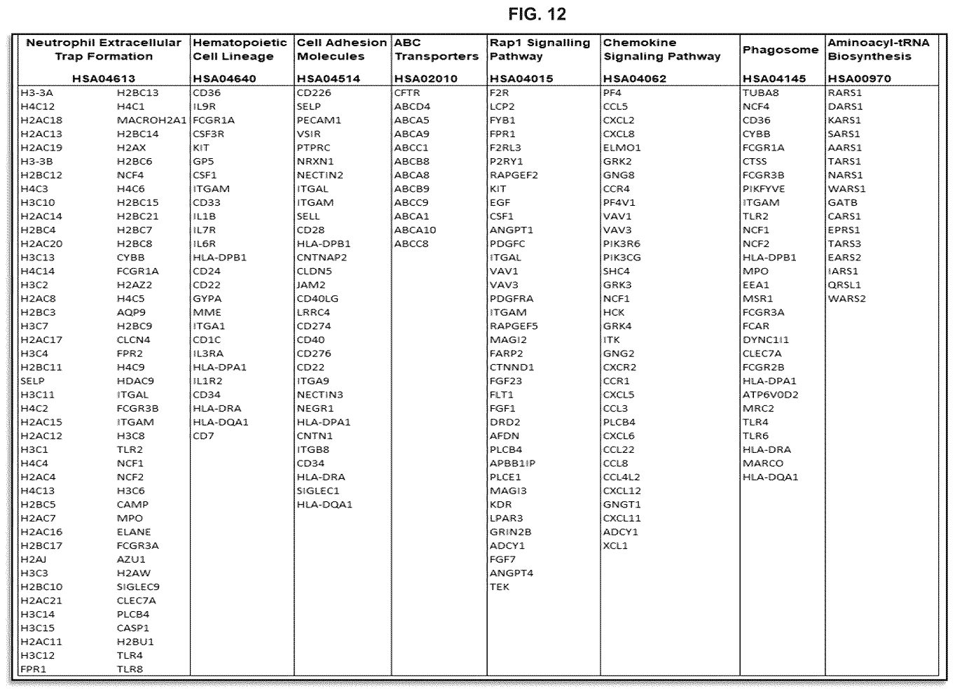

Some the KEGG relevant pathways include, for instance, neutrophil extracellular trap formation, hematopoietic cell lineage, cell adhesion, ATP-binding cassette (ABC) transport, chemokine signaling pathway, phagocytosis, and aminoacyl-tRNA biosynthesis (See details in FIG. 11 and FIG. 12 and Table 7 below).

TABLE 7
Relevant KEGG pathways and their related MkEVs genes

| KEGG | | |
|---|---|---|
| pathway | Neutrophil Extracellular | Hematopoietic Cell |
| KEGG | Trap Formation | Lineage |
| pathway ID | HSA04613 | HSA04640 |
| | H3-3A, H4C12, H2AC18, | CD36, IL9R, FCGR1A, CSF3R, |
| | H2AC13, H2AC19, H3-3B, | KIT, GP5, CSF1, ITGAM, |
| | H2BC12, H4C3, H3C10, | CD33, IL1B, IL7R, IL6R, HLA- |
| | H2AC14, H2BC4, H2AC20, | DPB1, CD24, CD22, GYPA, |
| | H3C13, H4C14, H3C2, H2AC8, | MME, ITGA1, CD1C, IL3RA, |
| | H2BC3, H3C7, H2AC17, H3C4, | HLA-DPA1, IL1R2, CD34, |
| | H2BC11, SELP, H3C11, H4C2, | HLA-DRA, HLA-DQA1, and |
| | H2AC15, H2AC12, H3C1, H4C4, | CD7 |
| | H2AC4, H4C13, H2BC5, H2AC7, | |
| | H2AC16, H2BC17, H2AJ, H3C3, | |
| | H2BC10, H2AC21, H3C14, | |
| | H3C15, H2AC11, H3C12, FPR1, | |
| | H2BC13, H4C1, MACROH2A1, | |
| | H2BC14, H2AX, H2BC6, NCF4, | |
| | H4C6, H2BC15, H2BC21, | |
| | H2BC7, H2BC8, CYBB, FCGR1A, | |
| | H2AZ2, H4C5, AQP9, H2BC9, | |
| | CLCN4, FPR2, H4C9, HDAC9, | |
| | ITGAL, FCGR3B, ITGAM, H3C8, | |
| | TLR2, NCF1, NCF2, H3C6, | |
| | CAMP, MPO, ELANE, FCGR3A, | |
| | AZU1, H2AW, SIGLEC9, CLEC7A, | |
| | PLCB4, CASP1, H2BU1, TLR4, | |
| | and TLR8 | |
| KEGG | | |
| pathway | | |
| KEGG | ABC Transporters | Rap1 Signaling Pathway |
| pathway ID | HSA02010 | HSA04015 |
| | CFTR, ABCD4, ABCA5, ABCA9, | F2R, LCP2, FYB1, FPR1, |
| | ABCC1, ABCB8, ABCA8, ABCB9, | F2RL3, P2RY1, RAPGEF2, KIT, |
| | ABCC9, ABCA1, ABCA10, and | EGF, CSF1, ANGPT1, PDGFC, |
| | ABCC8 | ITGAL, VAV1, VAV3, |
| | | PDGFRA, ITGAM, RAPGEF5, |
| | | MAGI2, FARP2, CTNND1, |
| | | FGF23, FLT1, FGF1, DRD2, |
| | | AFDN, PLCB4, APBB1IP, |
| | | PLCE1, MAGI3, KDR, LPAR3, |
| | | GRIN2B, ADCY1, FGF7, |
| | | ANGPT4, and TEK |
| KEGG | | |
| pathway | | Aminoacyl-tRNA |
| KEGG | Phagosome | Biosynthesis |
| pathway ID | HSA04145 | HSA00970 |
| | TUBA8, NCF4, CD36, CYBB, | RARS1, DARS1, KARS1, |
| | FCGR1A, CTSS, FCGR3B, | SARS1, AARS1, TARS1, |
| | PIKFYVE, ITGAM, TLR2, NCF1, | NARS1, WARS1, GATB, |
| | NCF2, HLA-DPB1, MPO, EEA1, | CARS1, EPRS1, TARS3, |
| | MSR1, FCGR3A, FCAR, DYNC1I1, | EARS2, IARS1, QRSL1, and |
| | CLEC7A, FCGR2B, HLA-DPA1, | WARS2 |
| | ATP6V0D2, MRC2, TLR4, TLR6, | |
| | HLA-DRA, MARCO, HLA-DQA1 | |
| KEGG | | |
| pathway | | Chemokine Signaling |
| KEGG | Cell Adhesion Molecules | Pathway |
| pathway ID | HSA04514 | HSA04062 |
| | CD226, SELP, PECAM1, VSIR, | PF4, CCL5, CXCL2, CXCL8, |
| | PTPRC, NRXN1, NECTIN2, | ELMO1, GRK2, GNG8, CCR4, |
| | ITGAL, ITGAM, SELL, CD28, HLA- | PF4V1, VAV1, VAV3, PIK3R6, |
| | DPB1, CNTNAP2, CLDN5, JAM2, | PIK3CG, SHC4, GRK3, NCF1, |
| | CD40LG, LRRC4, CD274, CD40, | HCK, GRK4, ITK, GNG2, |
| | CD276, CD22, ITGA9, NECTIN3, | CXCR2, CCR1, CXCL5, CCL3, |
| | NEGR1, HLA-DPA1, CNTN1, | PLCB4, CXCL6, CCL22, CCL8, |
| | ITGB8, CD34, HLA-DRA, | CCL4L2, CXCL12, GNGT1, |
| | SIGLEC1, and HLA-DQA1. | CXCL11, ADCY1, and XCL1 |

### Example 3: Biodistribution Data Related to MKEVs

This example describes data related to biodistribution data of MkEVs, including delivery of cargo-loaded MKEVs, targeting of MkEVs ex vivo, and in vivo MkEV biodistribution data.

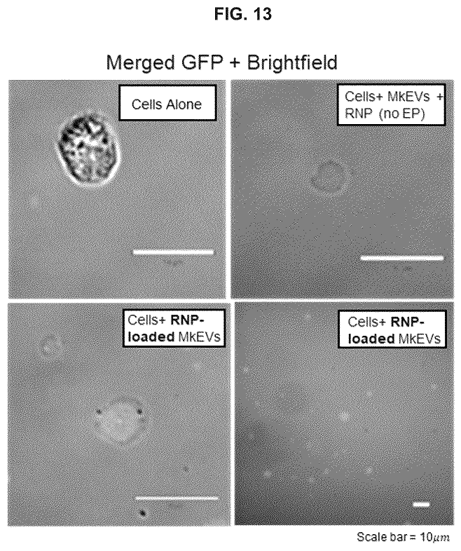

Confocal microscopy images of HSPCs (Lineage depleted CD150+ CD48− murine bone marrow cells) cocultured with MkEVs loaded with GFP-tagged Cas9 ribonucleoprotein (RNP) were obtained (FIG. 13). Cells cocultured with the cargo-loaded MkEVs showed GFP-positive cells, indicating cellular uptake of the GFP-tagged Cas9 loaded MkEVs. In contrast, control samples including cells alone and cells cocultured with MkEVs that were mock loaded with RNP (no electroporation (no EP)) showed no GFP positivity. These data indicate successful delivery of RNP cargo-loaded MkEVs into HSPCs.

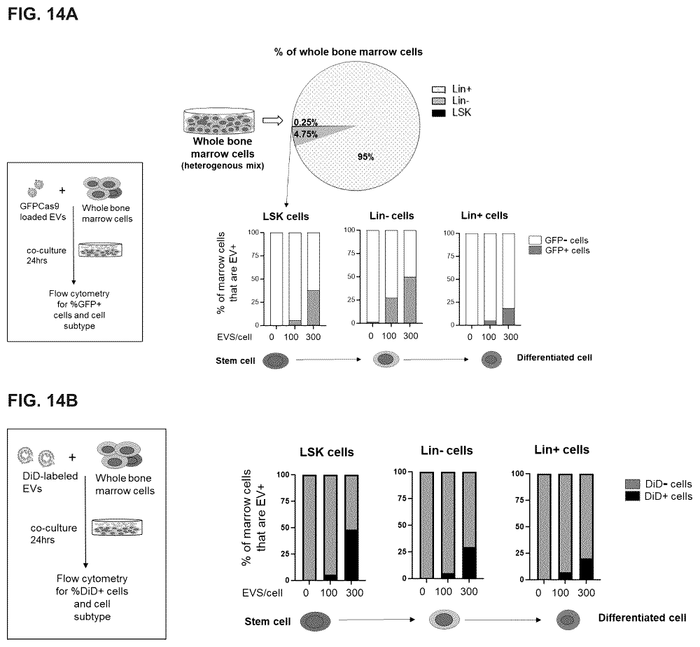

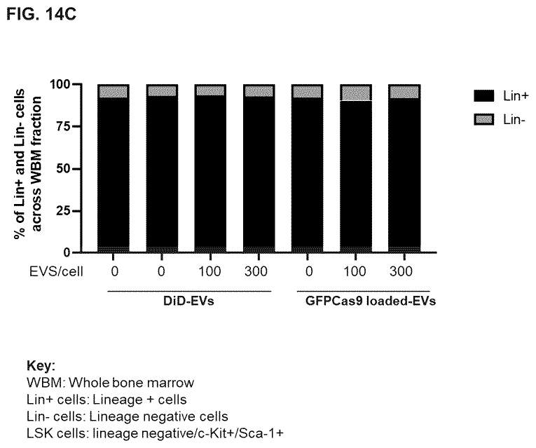

As shown in FIGS. 14A-14C, MkEVs preferentially target hematopoietic stem and progenitor cells ex vivo. MkEVs that were loaded with either a GFP-tagged Cas9 protein (FIG. 14A) or labelled with a lipophilic fluorescent dye, DiD (FIG. 14B). Loaded and DiD-labeled MkEVs were then cocultured with primary whole bone marrow derived from wild type mice. Following 24-hours in co-culture, cells were analyzed by flow cytometry for the % of cells that were GFP+ or DiD+ (i.e., MkEV+). The percent of cells that were GFP+ or DID+, indicating cell uptake/association with MkEVs was quantified by flow cytometry. In addition, the percent of Lineage positive (Lin+), Lineage negative (Lin−), and Lineage negative/c-Kit+/Sca-1+ (LSK) cells were simultaneously determined using fluorescently labelled antibodies against Lineage positive markers, Sca-1, and c-Kit cell surface proteins. The percentage of each subtype of cells in the heterogenous whole bone marrow population is shown in FIG. 14A. For cells cocultured with GFP-tagged Cas9 loaded MkEVs (as shown by the bar graphs in FIG. 14A), despite the vast majority (95%) of the cells in culture being Lin+ cells (differentiated cells), only up to 23% of these cells were positive for MkEVs. In contrast, while <5% were the hematopoietic stem and progenitor cells (Lin-cells), almost 50% of these cells were positive for MkEVs at the 300EVs per cell dose. Finally, for the rarest and most pluripotent hematopoietic stem cells evaluated in these cultures, the LSK cells, making up only 0.25% of the population, almost 40% of this population were positive for MKEVs. These data indicate the preferential ex vivo targeting of bone marrow-derived hematopoietic stem and progenitor cells. Similarly, as shown in FIG. 14B, for whole bone marrow cells cocultured with DiD-labeled MkEVs; only 20% of Lin+ cells were positive for MkEVs. In contrast, 30% of the rarer population of Lin-cells were positive for MkEVs. Finally, for the rarest and most pluripotent hematopoietic stem cells evaluated in these cultures (LSKs), up to 48% of this population were positive for MKEVs. There were no significant changes in the percentage of total Lin+ and Lin-cells in the whole bone marrow cultures across all the conditions of MkEV co-culture when compared to controls (FIG. 14C), indicating lack of toxicity.

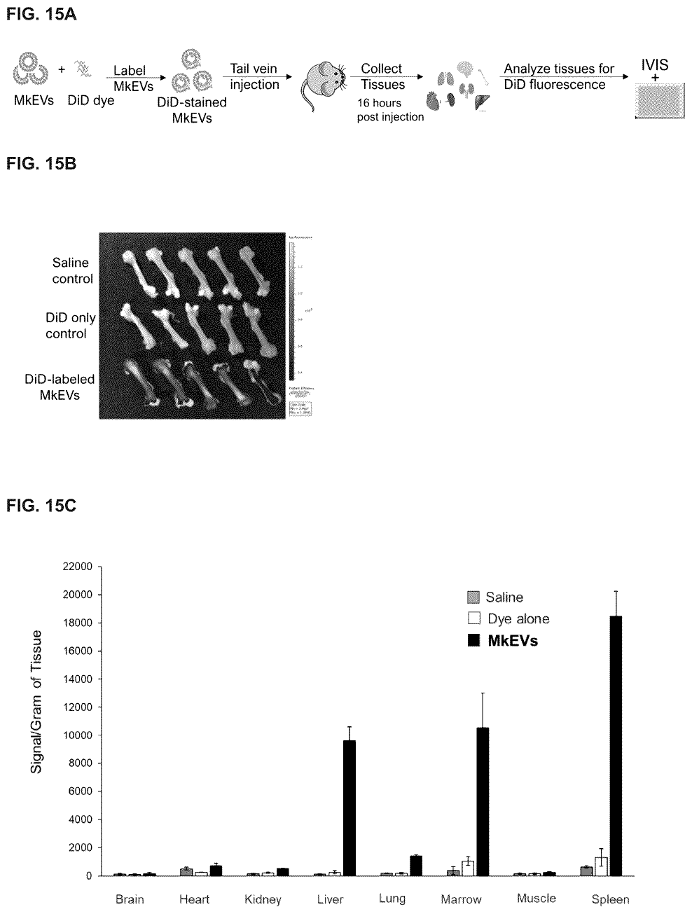

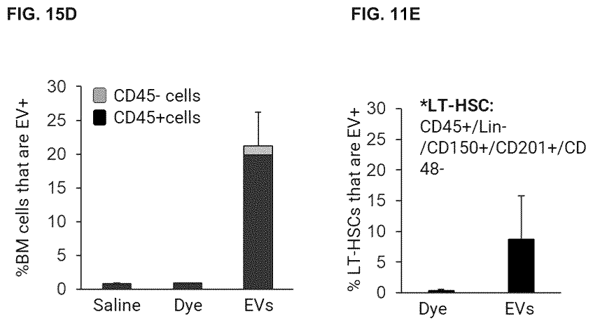

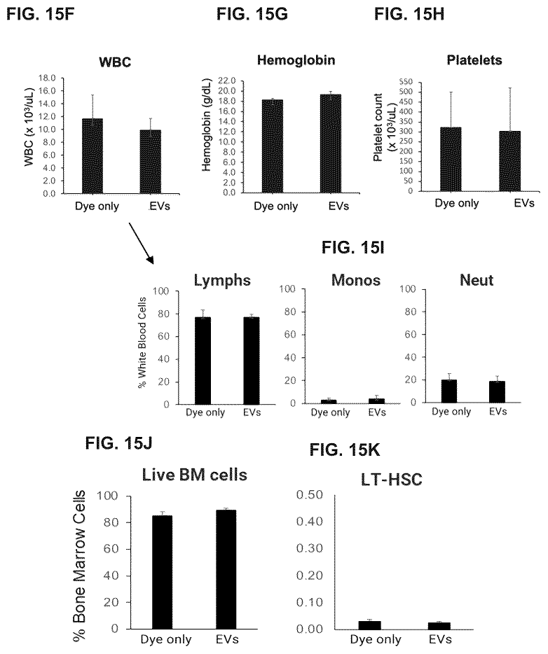

As shown in FIGS. 15A-15K, in vivo biodistribution of fluorescently-labeled MkEVs was examined following in vivo delivery to wild type mice. The experimental design is shown in FIG. 15A (n=3-5 mice/group). Fluorescently-labeled MkEVs were injected intravenously via tail vein into wild type mice, and tissues were harvested and analyzed for fluorescence 16 hours post injection. FIG. 15B shows fluorescent signal detected by IVIS in femurs dissected from mice. N=5 mice/group. Fluorescence in each homogenized tissue, as assayed by plate reader and normalized by tissue weight (FIG. 15C). As shown in FIG. 15C, there was significant in vivo MkEV targeting to the bone marrow following injection. FIGS. 15D and 15E show graphs of experimental data of bone marrow cells stained using antibodies against CD45, Lineage markers, CD150, CD201, and CD48 are analyzed by flow cytometry to determine the % of hematopoietic (CD45+ cells; FIG. 15D) and % of very primitive long-term hematopoietic stem cells (CD45+/Lin−/CD150+/CD201+/CD48-cells; FIG. 15E) that were positive for MkEVs. MkEVs preferentially target the hematopoietic cells within the bone marrow (FIG. 15D) and within that compartment, are targeting the very rare (<0.03% of marrow cells) long-term hematopoietic stem cells (FIG. 15E). There was no evidence of toxicity with regards to the hematopoietic compartment as indicated by lack of change in the peripheral white blood cell count (FIG. 15F), hemoglobin (FIG. 15G), platelet count (FIG. 15H), WBC differential (FIG. 15I), or % of CD45+ and CD45-cells (FIG. 15J), or % of the long-term hematopoietic stem cells in the marrow (FIG. 15K) 16 hours following MkEV injection.

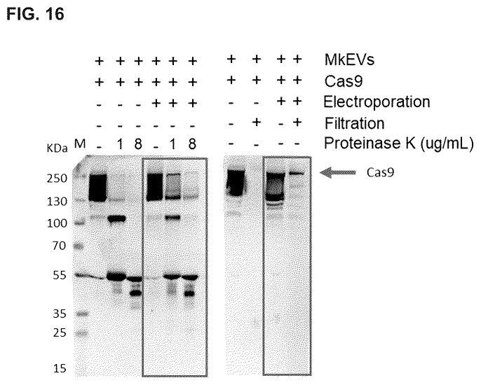

As shown in FIG. 16, Cas9 was loaded into MkEVs. MkEVs were electroporated with Cas9, and either treated with proteinase K to remove any un-internalized cargo (free cargo and vesicle surface-associated cargo), or treated with filtration to remove any free cargo, and then analyzed by western blotting for quantification of Cas9. Controls included MkEVs plus Cas9 without electroporation±Proteinase K±filtration. Cas9 was present in electroporated MkEVs, but not in control un-electroporated MkEVs, following filtration, indicating the successful vesicle association and/or internalization of protein cargo. Similarly, Cas9 was present in electroporated MkEVs, but not in control un-electroporated MkEVs following proteinase K digestion indicating both successful internalization and protection of loaded protein cargo following electroporation.

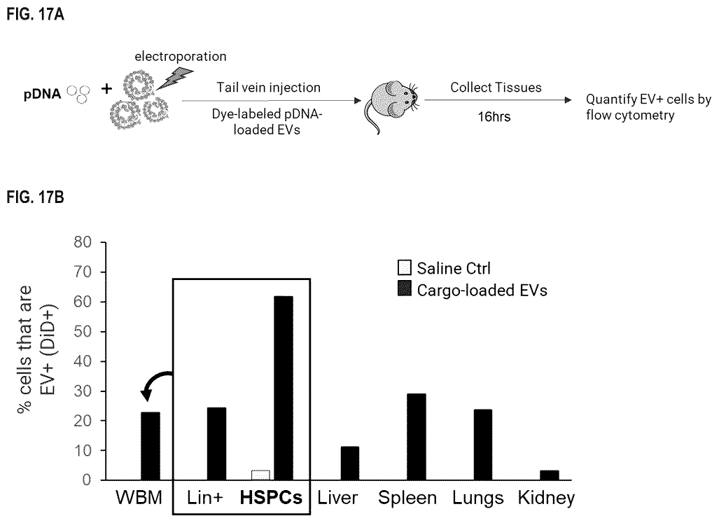

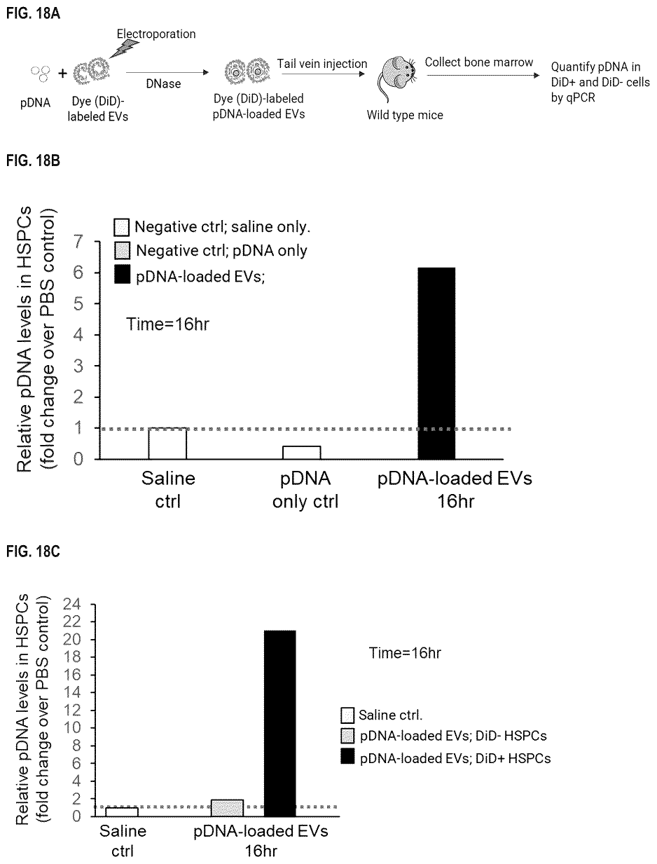

Gene therapy assets can be delivered to bone marrow cells following in vivo administration. MkEVs were labeled with DiD, loaded with a CMV promoter driven GFP expressing pDNA by electroporation (200V, 6 pulses), and injected into immunocompetent wild type mice via tail vein injection. Tissues were isolated 16 hours post injection and analyzed for EV association by flow cytometry. Vehicle alone injected (Saline Ctrl) served as a negative control (FIG. 17A). As shown in FIG. 17B, cargo-loaded EVs targeted bone marrow following intravenous injection and showed preferential uptake in hematopoietic stem and progenitor cells within the bone marrow compartment. Finally, in order to evaluate pDNA cargo delivery to HSPCs (Lineage-/c-Kit+/Sca-1+ cells) within the bone marrow following intravenous administration, MkEVs were loaded with pDNA cargo (CMV promoter-driven GFP) by electroporation (200V, 6 pulses) and injected into immunocompetent wild type mice via tail vein injection. HSPCs were isolated 16 hours post intravenous injection and pDNA within the cells was quantified by qPCR (FIG. 18A). As shown in FIG. 18B, pDNA was successfully delivered to HSPCs in mice receiving pDNA-loaded MkEVs, but not in control mice injected with saline or pDNA alone. Furthermore in a separate experiment, as shown in FIG. 18C, pDNA cargo was recovered from those HSPCs that were positive for EVs (DiD+) following intravenous injection. In contrast, minimal to no pDNA was recovered from the HSPCs that did not take up EVs (DiD−) following intravenous injection. These data demonstrate that the MPN assets can be loaded into MkEVs and successfully delivered to hematopoietic cells within the bone marrow following in vivo delivery.

### EQUIVALENTS

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present invention is not entitled to antedate such publication by virtue of prior invention.

As used herein, all headings are simply for organization and are not intended to limit the disclosure in any manner. The content of any individual section may be equally applicable to all sections.

### REFERENCES

1. Bray, Nicolas L., Harold Pimentel, Pall Melsted, and Lior Pachter. 2016. “Near-Optimal Probabilistic RNA-Seq Quantification.” Nature Biotechnology 34 (5): 525-27.
2. Bausch-Fluck D, Hofmann A, Bock T, Frei A P, Cerciello F, Jacobs A, Moest H, Omasits U, Gundry R L, Yoon C, Schiess R, Schmidt A, Mirkowska P, Härtlová A, Van Eyk J E, Bourquin J P, Aebersold R, Boheler K R, Zandstra P, Wollscheid B. A mass spectrometric-derived cell surface protein atlas. PLOS One. 2015 Apr. 20; 10 (3): e0121314. doi: 10.1371/journal.pone.0121314. PMID: 25894527; PMCID: PMC4404347.
3. Thul P J, Åkesson L, Wiking M, Mahdessian D, Geladaki A, Ait Blal H, Alm T, Asplund A, Björk L, Breckels L M, Bäckström A, Danielsson F, Fagerberg L, Fall J, Gatto L, Gnann C, Hober S, Hjelmare M, Johansson F, Lee S, Lindskog C, Mulder J, Mulvey C M, Nilsson P, Oksvold P, Rockberg J, Schutten R, Schwenk J M, Sivertsson Å, Sjöstedt E, Skogs M, Stadler C, Sullivan D P, Tegel H, Winsnes C, Zhang C, Zwahlen M, Mardinoglu A, Pontén F, von Feilitzen K, Lilley K S, Uhlén M, Lundberg E. A subcellular map of the human proteome. Science. 2017 May 26; 356 (6340): eaal3321. doi: 10.1126/science.aal3321. Epub 2017 May 11. PMID: 28495876.
4. Kanehisa M, Goto S. KEGG: kyoto encyclopedia of genes and genomes. Nucleic Acids Res. 2000 Jan. 1; 28 (1): 27-30. doi: 10.1093/nar/28.1.27. PMID: 10592173; PMCID: PMC102409.
5. Keerthikumar S, Chisanga D, Ariyaratne D, Al Saffar H, Anand S, Zhao K, Samuel M, Pathan M, Jois M, Chilamkurti N, Gangoda L, Mathivanan S. ExoCarta: A Web-Based Compendium of Exosomal Cargo. J Mol Biol. 2016 Feb. 22; 428 (4): 688-692. doi: 10.1016/j.jmb.2015.09.019. Epub 2015 Oct. 3. PMID: 26434508; PMCID: PMC4783248.
6. Pathan M, Fonseka P, Chitti S V, Kang T, Sanwlani R, Van Deun J, Hendrix A, Mathivanan S. Vesiclepedia 2019: a compendium of RNA, proteins, lipids and metabolites in extracellular vesicles. Nucleic Acids Res. 2019 Jan. 8; 47 (D1): D516-D519. doi: 10.1093/nar/gkyl029. PMID: 30395310; PMCID: PMC6323905.
7. Szklarczyk D, Gable A L, Nastou K C, Lyon D, Kirsch R, Pyysalo S, Doncheva N T, Legeay M, Fang T, Bork P, Jensen L J, von Mering C. The STRING database in 2021: customizable protein-protein networks, and functional characterization of user-uploaded gene/measurement sets. Nucleic Acids Res. 2021 Jan. 8; 49 (D1): D605-D612. doi: 10.1093/nar/gkaa1074. Erratum in: Nucleic Acids Res. 2021 Oct. 11; 49 (18): 10800. PMID: 33237311; PMCID: PMC7779004.

What is claimed is:

1. A composition comprising:
a plurality of substantially purified megakaryocyte-derived extracellular vesicles derived from a human pluripotent stem cell, wherein the composition comprises one or more proteins that are unique to or preferentially present in the megakaryocyte-derived extracellular vesicles as compared to other types of extracellular vesicles.

2. A composition comprising:
a plurality of substantially purified megakaryocyte-derived extracellular vesicles derived from a human pluripotent stem cell, wherein the composition comprises one or more nucleic acids encoding one or more proteins that are unique to or preferentially present in the megakaryocyte-derived extracellular vesicles as compared to other types of extracellular vesicles.

3. The composition of claim 1 or 2, wherein the one or more proteins (e.g. at least 3, or at least 5, or at least 10, or at least 25, or at least 50, or at least 100, or at least 150, or at least 175) or the one or more nucleic acids (e.g. at least 3, or at least 5, or at least, or at least 25, or at least 50, or at least 100, or at least 150, or at least 175) encoding one or more proteins (e.g. at least 3, or at least 5, or at least 10, or at least 25, or at least 50, or at least 100, or at least 150, or at least 175) are involved in aminoacyl-tRNA biosynthesis and optionally are selected from YARS1, AARS1, GARS1, LARS1, EPRS1, TARS1, DARS1, WARS1, VARS1, NARS1, RARS1, SARS1, KARS1, IARS1, CARS1, QARS, and HARS1.

4. The composition of claim 1 or 2, wherein the one or more proteins (e.g. at least 3, or at least 5, or at least 10, or at least 25, or at least 50, or at least 100, or at least 150, or at least 175) or the one or more nucleic acids (e.g. at least 3, or at least 5, or at least, or at least 25, or at least 50, or at least 100, or at least 150, or at least 175) encoding one or more proteins (e.g. at least 3, or at least 5, or at least 10, or at least 25, or at least 50, or at least 100, or at least 150, or at least 175) are involved in neutrophil extracellular trap formation, and optionally are selected from H4C1, H2AC20, H3C1, H2BC12, H3C15, MACROH2A1, H2AX, H2AZ2, H3-3A, H2AC14, and MACROH2A2.

5. The composition of claim 1 or 2, wherein the one or more proteins (e.g. at least 3, or at least 5, or at least 10, or at least 25, or at least 50, or at least 100, or at least 150, or at least 175) or the one or more nucleic acids (e.g. at least 3, or at least 5, or at least, or at least 25, or at least 50, or at least 100, or at least 150, or at least 175) encoding one or more proteins (e.g. at least 3, or at least 5, or at least 10, or at least 25, or at least 50, or at least 100, or at least 150, or at least 175) are selected from H4C16, H2AC17, H3C13, RPL9P9, PHB1, SETSIP, H2AZ1, XP32, COPS9, SEC61A2, MARS1, MRE11, ATP5F1C, H4C1, H2AC20, H3C1, H2BC12, FCSK, CAVIN2, YARS1, H3C15, MACROH2A1, H2AX, H3-2, SELENOF, MPIG6B, SEPTIN7, AARS1, H1-5, H1-2, H2AZ2, GARS1, LARS1, THPO, RACK1, H3-3A, CCDC191, H2BC19P, SEPTIN2, EPRS1, TARS1, ATP5F1B, ERBIN, DARS1, WARS1, VARS1, NIBAN2, SEPTIN6, NARS1, RARS1, SEPTIN11, SEPTIN5, SARS1, NIBAN1, SNRPGP15, PIP4P2, CYRIB, CARMIL1, KARS1, IARS1, SEPTIN9, H1-4, ARHGAP45, H1-0, CARS1, GCN1, FADS2, TBC1D13, GET3, RO60, LAMTOR5, ELOC, H2AC14, SCARF1, RNF24, GCSAML, NRDC, ECPAS, MACROH2A2, ATP5F1A, DMTN, TANGO2, CSF2RB, WASHC5, KCNA3, QARS1, MINDY1, PTPA, EXOC3L2, PRUNE1, PLPBP, THUMPD1, WASHC4, NECTIN2, GFUS, ADGRE2, AKAP8L, FAM234A, ADSS2, ANKRD13D, KCT2, NT5C3A, PIP4P1, CCDC9, ELOB, HPGDS, MEAK7, TOMM70, SMIM1, HARS1, ATP5PD, OGA, CHSY1, SQLE, SUSD6, IGKV1-27, PEDS1-UBE2V1, CEP44, MYG1, NRROS, IL21R, GRK2, MCEMP1, ELAPOR2, SDAD1, CERT1, UBE2F, CALHM5, H1-10, EIPR1, PBDC1, ARMH3, VIPAS39, MESD, PROSER2, RABL6, FYB1, C17orf49, RMDN3, KYAT3, TTMP, ERO1A, CD244, CZIB, PLPP3, BABAM2, H1-3, NAXE, ENSA, PALS2, GUCY1B1, UMAD1, MIX23, PRXL2B, SNU13, RTRAF, KXD1, VSIR, EPOR, and MARCHF2.

6. A composition comprising:
a plurality of substantially purified megakaryocyte-derived extracellular vesicles derived from a human pluripotent stem cell, wherein the composition comprises a lipid bilayer membrane surrounding a lumen and, wherein:
the megakaryocyte-derived extracellular vesicle lumen comprises one or more megakaryocyte-derived nucleic acid molecules selected from mRNA, tRNA, rRNA, siRNA, microRNA, regulating RNA, and non-coding and coding RNA and the lipid bilayer membrane comprises one or more proteins associated with or embedded within, wherein the one or more proteins are unique to or preferentially present in the megakaryocyte-derived extracellular vesicles as compared to other types of extracellular vesicles.

7. A composition comprising:
a plurality of substantially purified megakaryocyte-derived extracellular vesicles derived from a human pluripotent stem cell, wherein the composition comprises a lipid bilayer membrane surrounding a lumen and, wherein:
the megakaryocyte-derived extracellular vesicle lumen comprises one or more megakaryocyte-derived nucleic acid molecules selected from mRNA, tRNA, rRNA, siRNA, microRNA, regulating RNA, and non-coding and coding RNA and
the lipid bilayer membrane comprises one or more nucleic acids, wherein the one or more nucleic acids encode one or more proteins that are associated with or embedded within the lipid bilayer membrane and are unique to or preferentially present in the megakaryocyte-derived extracellular vesicles as compared to other types of extracellular vesicles.

8. The composition of claim 6 or 7, wherein the lipid bilayer membrane comprises the one or more proteins (e.g. at least 3, or at least 5, or at least 10) or the one or more nucleic acids (e.g. at least 3, or at least 5, or at least 10) encoding one or more proteins (e.g. at least 3, or at least 5, or at least 10) are selected from CAVIN2, MPIG6B, ERBIN, ELOC, CSF2RB, KCNA3, NECTIN2, IL21R, MCEMP1, PROSER2, FYB1, CD244, and EPOR.

9. The composition of claim 8, wherein more than about 1%, or more than about 5%, or more than about 10%, or more than about 15%, or more than about 20%, or more than about 25%, or more than about 30%, or more than about 35%, or more than about 40%, or more than about 45%, or more than about 50%, or more than about 55%, or more than about 60%, or more than about 65%, or more than about 70%, or more than about 75%, or more than about 80%, or more than about 85%, or more than about 90%, or more than about 95% of the megakaryocyte-derived extracellular vesicles comprise a lipid bilayer membrane comprising the one or more proteins, or the one or more nucleic acids encoding one or more proteins.

10. The composition of claim 8, wherein less than about 95%, or less than about 90%, or less than about 85%, or less than about 80%, or less than about 75%, or less than about 70%, or less than about 65%, or less than about 60%, or less than about 55%, or less than about 50%, or less than about 45%, or less than about 40%, or less than about 35%, or less than about 30%, or less than about 25%, or less than about 20%, or less than about 15%, or less than about 10%, or less than about 5%, or less than about 1% of the megakaryocyte-derived extracellular vesicles comprise a lipid bilayer membrane comprising the one or more proteins, or the one or more nucleic acids encoding one or more proteins.

11. A composition comprising:
a plurality of substantially purified megakaryocyte-derived extracellular vesicles derived from a human pluripotent stem cell, wherein the composition comprises one or more nucleic acids that are unique to or preferentially present in the megakaryocyte-derived extracellular vesicles as compared to other types of extracellular vesicles.

12. A composition comprising:
a plurality of substantially purified megakaryocyte-derived extracellular vesicles derived from a human pluripotent stem cell, wherein the composition comprises one or more proteins encoded by one or more nucleic acids that are unique to or preferentially present in the megakaryocyte-derived extracellular vesicles as compared to other types of extracellular vesicles.

13. The composition of claim 11 or 12, wherein the one or more nucleic acids (e.g. at least 3, or at least 5, or at least 10, or at least 25, or at least 50, or at least 100, or at least 150, or at least 175, or at least 500, or at least 1000, or at least 1500, or at least 2000, or at least 2500) are listed in Table 6.

14. The composition of claim 11 or 12, wherein the one or more nucleic acids (e.g. at least 3, or at least 5, or at least 10, or at least 25, or at least 50, or at least 100, or at least 150, or at least 175), or the one or more proteins (e.g. at least 3, or at least 5, or at least 10, or at least 25, or at least 50, or at least 100, or at least 150, or at least 175) encoded by one or more nucleic acids (e.g. at least 3, or at least 5, or at least 10, or at least 25, or at least 50, or at least 100, or at least 150, or at least 175) are involved in neutrophil extracellular trap formation, and optionally are selected from H3-3A, H4C12, H2AC18, H2AC13, H2AC19, H3-3B, H2BC12, H4C3, H3C10, H2AC14, H2BC4, H2AC20, H3C13, H4C14, H3C2, H2AC8, H2BC3, H3C7, H2AC17, H3C4, H2BC11, SELP, H3C11, H4C2, H2AC15, H2AC12, H3C1, H4C4, H2AC4, H4C13, H2BC5, H2AC7, H2AC16, H2BC17, H2AJ, H3C3, H2BC10, H2AC21, H3C14, H3C15, H2AC11, H3C12, FPR1, H2BC13, H4C1, MACROH2A1, H2BC14, H2AX, H2BC6, NCF4, H4C6, H2BC15, H2BC21, H2BC7, H2BC8, CYBB, FCGR1A, H2AZ2, H4C5, AQP9, H2BC9, CLCN4, FPR2, H4C9, HDAC9, ITGAL, FCGR3B, ITGAM, H3C8, TLR2, NCF1, NCF2, H3C6, CAMP, MPO, ELANE, FCGR3A, AZU1, H2AW, SIGLEC9, CLEC7A, PLCB4, CASP1, H2BU1, TLR4, and TLR8.

15. The composition of claim 11 or 12, wherein the one or more nucleic acids (e.g. at least 3, or at least 5, or at least 10, or at least 25, or at least 50, or at least 100, or at least 150, or at least 175), or the one or more proteins (e.g. at least 3, or at least 5, or at least 10, or at least 25, or at least 50, or at least 100, or at least 150, or at least 175) encoded by one or more nucleic acids (e.g. at least 3, or at least 5, or at least 10, or at least 25, or at least 50, or at least 100, or at least 150, or at least 175) are involved in hematopoietic cell lineage, and optionally are selected from CD36, IL9R, FCGR1A, CSF3R, KIT, GP5, CSF1, ITGAM, CD33, IL1B, IL7R, IL6R, HLA-DPB1, CD24, CD22, GYPA, MME, ITGA1, CD1C, IL3RA, HLA-DPA1, IL1R2, CD34, HLA-DRA, HLA-DQA1, and CD7.

16. The composition of claim 11 or 12, wherein the one or more nucleic acids (e.g. at least 3, or at least 5, or at least 10, or at least 25, or at least 50, or at least 100, or at least 150, or at least 175), or the one or more proteins (e.g. at least 3, or at least 5, or at least 10, or at least 25, or at least 50, or at least 100, or at least 150, or at least 175) encoded by one or more nucleic acids (e.g. at least 3, or at least 5, or at least 10, or at least 25, or at least 50, or at least 100, or at least 150, or at least 175) are involved in cell adhesion, and optionally selected from CD226, SELP, PECAM1, VSIR, PTPRC, NRXN1, NECTIN2, ITGAL, ITGAM, SELL, CD28, HLA-DPB1, CNTNAP2, CLDN5, JAM2, CD40LG, LRRC4, CD274, CD40, CD276, CD22, ITGA9, NECTIN3, NEGR1, HLA-DPA1, CNTN1, ITGB8, CD34, HLA-DRA, SIGLEC1, and HLA-DQA1.

17. The composition of claim 11 or 12, wherein the one or more nucleic acids (e.g. at least 3, or at least 5, or at least 10, or at least 25, or at least 50, or at least 100, or at least 150, or at least 175), or the one or more proteins (e.g. at least 3, or at least 5, or at least 10, or at least 25, or at least 50, or at least 100, or at least 150, or at least 175) encoded by one or more nucleic acids (e.g. at least 3, or at least 5, or at least 10, or at least 25, or at least 50, or at least 100, or at least 150, or at least 175) are involved in ATP-binding cassette (ABC) transport, and optionally are selected from CFTR, ABCD4, ABCA5, ABCA9, ABCC1, ABCB8, ABCA8, ABCB9, ABCC9, ABCA1, ABCA10, and ABCC8.

18. The composition of claim 11 or 12, wherein the one or more nucleic acids (e.g. at least 3, or at least 5, or at least 10, or at least 25, or at least 50, or at least 100, or at least 150, or at least 175), or the one or more proteins (e.g. at least 3, or at least 5, or at least 10, or at least 25, or at least 50, or at least 100, or at least 150, or at least 175) encoded by one or more nucleic acids (e.g. at least 3, or at least 5, or at least 10, or at least 25, or at least 50, or at least 100, or at least 150, or at least 175) are involved in Rap1 signaling pathway, and optionally are selected from F2R, LCP2, FYB1, FPR1, F2RL3, P2RY1, RAPGEF2, KIT, EGF, CSF1, ANGPT1, PDGFC, ITGAL, VAV1, VAV3, PDGFRA, ITGAM, RAPGEF5, MAGI2, FARP2, CTNND1, FGF23, FLT1, FGF1, DRD2, AFDN, PLCB4, APBB1IP, PLCE1, MAGI3, KDR, LPAR3, GRIN2B, ADCY1, FGF7, ANGPT4, and TEK.

19. The composition of claim 11 or 12, wherein the one or more nucleic acids (e.g. at least 3, or at least 5, or at least 10, or at least 25, or at least 50, or at least 100, or at least 150, or at least 175), or the one or more proteins (e.g. at least 3, or at least 5, or at least 10, or at least 25, or at least 50, or at least 100, or at least 150, or at least 175) encoded by one or more nucleic acids (e.g. at least 3, or at least 5, or at least 10, or at least 25, or at least 50, or at least 100, or at least 150, or at least 175) are involved in chemokine signaling pathway, and optionally are selected from PF4, CCL5, CXCL2, CXCL8, ELMO1, GRK2, GNG8, CCR4, PF4V1, VAV1, VAV3, PIK3R6, PIK3CG, SHC4, GRK3, NCF1, HCK, GRK4, ITK, GNG2, CXCR2, CCR1, CXCL5, CCL3, PLCB4, CXCL6, CCL22, CCL8, CCL4L2, CXCL12, GNGT1, CXCL11, ADCY1, and XCL1.

20. The composition of claim 11 or 12, wherein the one or more nucleic acids (e.g. at least 3, or at least 5, or at least 10, or at least 25, or at least 50, or at least 100, or at least 150, or at least 175), or the one or more proteins (e.g. at least 3, or at least 5, or at least 10, or at least 25, or at least 50, or at least 100, or at least 150, or at least 175) encoded by one or more nucleic acids (e.g. at least 3, or at least 5, or at least 10, or at least 25, or at least 50, or at least 100, or at least 150, or at least 175) are involved in phagocytosis, and optionally are selected from TUBA8, NCF4, CD36, CYBB, FCGR1A, CTSS, FCGR3B, PIKFYVE, ITGAM, TLR2, NCF1, NCF2, HLA-DPB1, MPO, EEA1, MSR1, FCGR3A, FCAR, DYNC111, CLEC7A, FCGR2B, HLA-DPA1, ATP6VOD2, MRC2, TLR4, TLR6, HLA-DRA, MARCO, and HLA-DQA1.

21. The composition of claim 11 or 12, wherein the one or more nucleic acids (e.g. at least 3, or at least 5, or at least 10, or at least 25, or at least 50, or at least 100, or at least 150, or at least 175), or the one or more proteins (e.g. at least 3, or at least 5, or at least 10, or at least 25, or at least 50, or at least 100, or at least 150, or at least 175) encoded by one or more nucleic acids (e.g. at least 3, or at least 5, or at least 10, or at least 25, or at least 50, or at least 100, or at least 150, or at least 175) are involved in aminoacyl-tRNA biosynthesis, and optionally are selected from RARS1, DARS1, KARS1, SARS1, AARS1, TARS1, NARS1, WARS1, GATB, CARS1, EPRS1, TARS3, EARS2, IARS1, QRSL1, and WARS2.

22. The composition of claim 11 or 12, wherein the one or more nucleic acids (e.g. at least 3, or at least 5, or at least 10, or at least 25, or at least 50, or at least 100, or at least 150, or at least 175), or the one or more proteins (e.g. at least 3, or at least 5, or at least 10, or at least 25, or at least 50, or at least 100, or at least 150, or at least 175) encoded by one or more nucleic acids (e.g. at least 3, or at least 5, or at least 10, or at least 25, or at least 50, or at least 100, or at least 150, or at least 175) are selected from H4C16, H2AC17, H3C13, RPL9P9, PHB1, SETSIP, H2AZ1, XP32, COPS9, SEC61A2, MARS1, MRE11, ATP5F1C, H4C1, H2AC20, H3C1, H2BC12, FCSK, CAVIN2, YARS1, H3C15, MACROH2A1, H2AX, H3-2, SELENOF, MPIG6B, SEPTIN7, AARS1, H1-5, H1-2, H2AZ2, GARS1, LARS1, THPO, RACK1, H3-3A, CCDC191, H2BC19P, SEPTIN2, EPRS1, TARS1, ATP5F1B, ERBIN, DARS1, WARS1, VARS1, NIBAN2, SEPTIN6, NARS1, RARS1, SEPTIN11, SEPTIN5, SARS1, NIBAN1, SNRPGP15, PIP4P2, CYRIB, CARMIL1, KARS1, IARS1, SEPTIN9, H1-4, ARHGAP45, H1-0, CARS1, GCN1, FADS2, TBC1D13, GET3, RO60, LAMTOR5, ELOC, H2AC14, SCARF1, RNF24, GCSAML, NRDC, ECPAS, MACROH2A2, ATP5F1A, DMTN, TANGO2, CSF2RB, WASHC5, KCNA3, QARS1, MINDY1, PTPA, EXOC3L2, PRUNE1, PLPBP, THUMPD1, WASHC4, NECTIN2, GFUS, ADGRE2, AKAP8L, FAM234A, ADSS2, ANKRD13D, KCT2, NT5C3A, PIP4P1, CCDC9, ELOB, HPGDS, MEAK7, TOMM70, SMIM1, HARS1, ATP5PD, OGA, CHSY1, SQLE, SUSD6, IGKV1-27, PEDS1-UBE2V1, CEP44, MYG1, NRROS, IL21R, GRK2, MCEMP1, ELAPOR2, SDAD1, CERT1, UBE2F, CALHM5, H1-10, EIPR1, PBDC1, ARMH3, VIPAS39, MESD, PROSER2, RABL6, FYB1, C17orf49, RMDN3, KYAT3, TTMP, ERO1A, CD244, CZIB, PLPP3, BABAM2, H1-3, NAXE, ENSA, PALS2, GUCY1B1, UMAD1, MIX23, PRXL2B, SNU13, RTRAF, KXD1, VSIR, EPOR, and MARCHF2.

23. The composition of any one of claims 11-22, wherein more than about 1%, or more than about 5%, or more than about 10%, or more than about 15%, or more than about 20%, or more than about 25%, or more than about 30%, or more than about 35%, or more than about 40%, or more than about 45%, or more than about 50%, or more than about 55%, or more than about 60%, or more than about 65%, or more than about 70%, or more than about 75%, or more than about 80%, or more than about 85%, or more than about 90%, or more than about 95% of the megakaryocyte-derived extracellular vesicles comprise the one or more nucleic acids or the one or more proteins encoded by the one or more nucleic acids.

24. The composition of any one of claims 11-22, wherein less than about 95%, or less than about 90%, or less than about 85%, or less than about 80%, or less than about 75%, or less than about 70%, or less than about 65%, or less than about 60%, or less than about 55%, or less than about 50%, or less than about 45%, or less than about 40%, or less than about 35%, or less than about 30%, or less than about 25%, or less than about 20%, or less than about 15%, or less than about 10%, or less than about 5%, or less than about 1% of the megakaryocyte-derived extracellular vesicles comprise the one or more nucleic acids or the one or more proteins encoded by the one or more nucleic acids.

25. The composition of any one of claims 1-24, wherein the megakaryocyte-derived extracellular vesicles are substantially of a diameter in the range between about 100 nm to about 600 nm.

26. The composition of any one of claims 1-25, wherein the megakaryocyte-derived extracellular vesicles are substantially of a diameter in the range between about 30 nm to about 100 nm.

27. The composition of any one of claims 1-26, wherein the megakaryocyte-derived extracellular vesicles are substantially of a diameter in the range between about 100 nm to about 300 nm.

28. The composition of any one of claims 1-27, wherein about 90% or more, or about 95% or more, or about 97% or more, or about 99% or more of the megakaryocyte-derived extracellular vesicles are of a diameter of between about 100 nm and about 600 nm.

29. The composition of any one of claims 1-27, wherein about 90% or more, or about 95% or more, or about 97% or more, or about 99% or more of the megakaryocyte-derived extracellular vesicles are of a diameter of between about 100 nm and about 300 nm.

30. The composition of any one of claims 1-29, wherein the megakaryocyte-derived extracellular vesicles are substantially free of autologous DNA.

31. The composition of any one of claims 1-29, wherein the megakaryocyte-derived extracellular vesicles are substantially free of:
(a) megakaryocytes, and/or
(b) platelets.

32. The composition of any one of claims 1-31, wherein the megakaryocyte-derived extracellular vesicles are suitable for homing to a hematopoietic stem cell in vivo and/or in vitro.

33. The composition of any one of claims 1-31, wherein the megakaryocyte-derived extracellular vesicles are suitable for homing to bone marrow in vivo and/or in vitro.

34. The composition of claim 33, wherein the megakaryocyte-derived extracellular vesicles are suitable for homing to a lymphatic cell in vivo and/or in vitro.

35. The composition of claim 34, wherein the megakaryocyte-derived extracellular vesicles are suitable for homing to a regulatory T cell in vivo and/or in vitro.

36. The composition of any one of claims 1-35, wherein the megakaryocyte-derived extracellular vesicles are suitable for loading with cargo into the lumen and/or loading with cargo associated with the surface of the megakaryocyte-derived extracellular vesicles.

37. The composition of claim 36, wherein the cargo is one or more therapeutic agents.

38. The composition of claim 37, wherein the therapeutic agent is a nucleic acid therapeutic agent.

39. The composition of claim 38, wherein the nucleic acid therapeutic agent is selected from one or more non-autologous and/or recombinant nucleic acid constructs selected from mRNA, tRNA, rRNA, siRNA, microRNA, regulating RNA, non-coding and coding RNA, linear DNA, DNA fragments, or DNA plasmids.

40. The composition of claim 39, wherein the nucleic acid therapeutic agent is mRNA, and optionally: is in vitro transcribed or synthetic and/or comprises one or more non-canonical nucleotides, optionally selected from pseudouridine and 5-methoxyuridine.

41. The composition of claim 40, wherein the nucleic acid therapeutic agent encodes a functional protein.

42. The composition of claim 40, wherein the nucleic acid therapeutic agent encodes a gene-editing protein and/or associated elements for gene-editing functionality.

43. The composition of claim 42, wherein the gene-editing protein is selected from a zinc finger (ZF), transcription activator-like effector (TALE), meganuclease, and clustered regularly interspaced short palindromic repeat (CRISPR)-associated protein.

44. The composition of claim 43, wherein the CRISPR-associated protein is selected from Cas9, CasX, CasY, Cpf1, and gRNA complexes thereof.

45. The composition of claim 37, wherein the therapeutic agent is a biologic therapeutic agent.

46. The composition of claim 45, wherein the biologic therapeutic agent is a protein.

47. The composition of claim 46, wherein the biologic therapeutic agent is a recombinant protein.

48. The composition of claim 46 or 47, wherein the biologic therapeutic agent is one of an antibody or an antibody fragment, fusion protein, gene-editing protein, cytokine, antigen, and peptide.

49. The composition of claim 37, wherein the therapeutic agent is a small molecule therapeutic agent.

50. The composition of any one of claims 37-49, wherein the therapeutic agent is a vaccine and/or an immunogenic antigen.

51. The composition of any one of claims 1-50, wherein the human pluripotent stem cell is a primary CD34+ hematopoietic stem cell.

52. The composition of claim 51, wherein the primary CD34+ hematopoietic stem cell is sourced from peripheral blood or cord blood.

53. The composition of claim 52, wherein the peripheral blood is granulocyte colony-stimulating factor-mobilized adult peripheral blood (mPB).

54. The composition of any one of claims 1-53, wherein the human pluripotent stem cell is an embryonic stem cell (ESC).

55. The composition of any one of claims 1-54, wherein the human pluripotent stem cell is an induced pluripotent stem cell (IPS).

56. The composition of any one of claims 1-55, wherein the megakaryocyte-derived extracellular vesicles are isolated from megakaryocytes, which are generated in the absence of added erythropoietin.

57. The composition of any one of claims 1-56, wherein the megakaryocyte-derived extracellular vesicles are isolated from megakaryocytes, which are generated in the presence of added thrombopoietin.

58. A pharmaceutical composition comprising the composition of any one of claims 1-57 and a pharmaceutically acceptable excipient or carrier.

59. A method for transferring a deliverable therapeutic agent, comprising:
(a) obtaining the megakaryocyte-derived extracellular vesicles of any one of claims 1-57;
(b) incubating the megakaryocyte-derived extracellular vesicle with a therapeutic agent to allow the therapeutic agent to populate the lumen of the megakaryocyte-derived extracellular vesicle and/or associate with the surface of the megakaryocyte-derived extracellular vesicle and yield a deliverable therapeutic agent; and
(c) administering the deliverable therapeutic agent to a patient or contacting the deliverable therapeutic agent with a biological cell in vitro and administering the contacted biological cell to a patient.

60. The method of claim 59, wherein the method is an in vivo method.

61. The method of claim 59, wherein the method is an ex vivo method.

62. The method of claim 61, wherein the method further comprises obtaining a biological cell from a patient.

63. The method of claim 61 or 62, wherein the contacting of the deliverable therapeutic agent with the biological cell comprises co-culturing the deliverable therapeutic agent with the biological cell.

64. The method of any one of claims 59-63, wherein the megakaryocyte-derived extracellular vesicles are autologous with the patient.

65. The method of any one of claims 59-63, wherein the megakaryocyte-derived extracellular vesicles are allogeneic with the patient.

66. The method of any one of claims 59-63, wherein the megakaryocyte-derived extracellular vesicles are heterologous with the patient.

67. The method of any one of claims 59-66, wherein the therapeutic agent is a nucleic acid therapeutic agent.

68. The method of claim 67, wherein the nucleic acid therapeutic agent is selected from one or more non-autologous and/or recombinant nucleic acid constructs selected from mRNA, RNA, rRNA, siRNA, microRNA, regulating RNA, non-coding and coding RNA, linear DNA, DNA fragments, or DNA plasmids.

69. The method of claim 67 or 68, wherein the nucleic acid therapeutic agent encodes a wild type gene which is defective in the patient.

70. The method of any one of claims 67-69, wherein the nucleic acid therapeutic agent encodes a gene-editing protein and/or associated elements for gene-editing functionality.

71. The method of claim 70, wherein the gene-editing protein is selected from a zinc finger (ZF), transcription activator-like effector (TALE), meganuclease, and clustered regularly interspaced short palindromic repeat (CRISPR)-associated protein.

72. The method of claim 71, wherein the CRISPR-associated protein is selected from Cas9, CasX, CasY, Cpf1, and gRNA complexes thereof.

73. The method of any one of claims 59-66, wherein the therapeutic agent is a biologic therapeutic agent, optionally a virus.

74. The method of claim 73, wherein the biologic therapeutic agent is a protein.

75. The method of claim 74, wherein the biologic therapeutic agent is a recombinant protein.

76. The method of claim 74 or 75, wherein the therapeutic agent is one of an antibody or an antibody fragment, fusion protein, gene-editing protein, cytokine, antigen, and peptide.

77. The method of any one of claims 59-66, wherein the therapeutic agent is a small molecule therapeutic agent.

78. The method of any one of claims 59-67, wherein the incubating comprises one or more of sonication, saponin permeabilization, mechanical vibration, hypotonic dialysis, extrusion through porous membranes, cholesterol conjugation, application of electric current and combinations thereof.

79. The method of any one of claims 59-68, wherein the incubating comprises one or more of electroporating, transforming, transfecting, and microinjecting.

80. The method of any one of claims 59-79, wherein the megakaryocyte-derived extracellular vesicles bind to a cell surface receptor on a cell of the patient.

81. The method of any one of claims 59-80, wherein the megakaryocyte-derived extracellular vesicles bind to a cell surface receptor on the contacted biological cell of step (c).

82. The method of any one of claims 59-81, wherein the megakaryocyte-derived extracellular vesicles fuse with the extracellular membrane of a cell of the patient.

83. The method of any one of claims 59-82, wherein the megakaryocyte-derived extracellular vesicles fuse with the extracellular membrane of the biological cells of step (c).

84. The method of any one of claims 59-81, wherein the megakaryocyte-derived extracellular vesicles are endocytosed by a cell of the patient.

85. The method of any one of claims 59-82, wherein the megakaryocyte-derived extracellular vesicles are endocytosed by the biological cells of step (c).

86. A method of generating the megakaryocyte-derived extracellular vesicles of any one of claims 1-57, comprising:
(a) obtaining a human pluripotent stem cell, the human pluripotent stem cell being a primary CD34+ hematopoietic stem cell sourced from peripheral blood or cord blood;
(b) differentiating the human pluripotent stem cell to a megakaryocyte in the absence of added erythropoietin and in the presence of added thrombopoietin; and
(c) isolating megakaryocyte-derived extracellular vesicles from the megakaryocytes.

87. The method of claim 86, wherein the method further comprises (d) contacting the megakaryocyte-derived extracellular vesicles with radiation.

88. The method of claim 87, wherein the radiation is gamma radiation.

89. The method of claim 88, wherein the gamma radiation is at an amount greater than about 12kGy, or about 25kGy, or about 50kGy.

90. The method of any one of claims 86-89, wherein the method is substantially serum free.

91. A method for treating or preventing an infectious disease, comprising administering an effective amount of a composition of any one of claims 1-58.

92. A method for treating or preventing an infectious disease, comprising administering an effective amount of a composition comprising a cell which is contacted with a composition of any one of claims 1-58 in vitro.

93. The method of claim 91 or 92, wherein the composition comprises megakaryocyte-derived extracellular vesicles, which comprise (i) a nucleic acid molecule encoding a vaccine protein and/or an immunogenic antigen or (ii) a vaccine protein and/or an immunogenic antigen.

94. The method of claim 91 or 92, wherein the composition comprises megakaryocyte-derived extracellular vesicles which comprise (i) a nucleic acid molecule encoding a protein related to infectivity or (ii) a protein related to infectivity.

95. The method of any one of claims 91-94, wherein the infectious disease is a coronavirus infection.

96. The method of claim 95, wherein the coronavirus infection is infection by a betacoronavirus or an alphacoronavirus, optionally wherein the betacoronavirus is selected from a SARS-COV-2, SARS-COV, MERS-COV, HCOV-HKU1, and HCoV-OC43 or the alphacoronavirus is selected from a HCoV-NL63 and HCoV-229E.

97. The method of claim 96, wherein the coronavirus infection is infection by SARS-CoV-2.

98. The method of claim 97, wherein the infectious disease is COVID-19.

99. The method any one of claims 95-98, wherein the vaccine protein is a betacoronavirus protein or an alphacoronavirus protein, optionally wherein the betacoronavirus protein is selected from a SARS-COV-2, SARS-COV, MERS-COV, HCoV-HKU1, and HCoV-OC43 protein, or an antigenic fragment thereof or the alphacoronavirus protein is selected from a HCoV-NL63 and HCoV-229E protein, or an antigenic fragment thereof.

100. The method of claim 99, wherein the SARS-COV-2 protein is the spike surface glycoprotein, membrane glycoprotein M, envelope protein E, and nucleocapsid phosphoprotein, or an antigenic fragment thereof.

101. The method of claim 100, wherein the spike surface glycoprotein is the S1 or S2 subunit, or an antigenic fragment thereof.

102. The method of any one of claims 94-98, wherein the nucleic acid molecule encoding a protein related to infectivity is mRNA, and the mRNA is optionally in vitro transcribed or synthetic.

103. The method of claim 102 wherein the mRNA encodes SARS-COV-2 spike surface glycoprotein, membrane glycoprotein M, envelope protein E, and nucleocapsid phosphoprotein, or an antigenic fragment thereof.

104. The method of claim 102 or 103, wherein the mRNA comprises one or more non-canonical nucleotides, optionally selected from pseudouridine and 5-methoxyuridine.

105. The method of claim 91-94, wherein the infectious disease is an influenza infection, optionally selected from Type A, Type B, Type C, and Type D influenza.

106. The method of claim 91-94, wherein the infectious disease is a retroviral infection, optionally selected from human immune deficiency (HIV) and simian immune deficiency (SIV).

107. The method of claim 106, wherein the composition comprises megakaryocyte-derived extracellular vesicles which comprise a nucleic acid encoding a protein having reduced C—C chemokine receptor type 5 (CCR5) and C—X—C chemokine receptor type 4 (CXCR4) activity.

108. The method of claim 106 or 107, wherein the composition comprises megakaryocyte-derived extracellular vesicles, which comprise a nucleic acid molecule encoding a mutant CCR5 or CXCR4.

109. The method of claim 108, wherein the composition comprises megakaryocyte-derived extracellular vesicles which comprise a nucleic acid molecule encoding a gene-editing protein that is capable of reducing C—C chemokine receptor type 5 (CCR5) and C—X—C chemokine receptor type 4 (CXCR4) activity.

110. The method of claim 109, wherein the gene-editing protein is selected from a zinc finger (ZF), transcription activator-like effector (TALE), meganuclease, and clustered regularly interspaced short palindromic repeat (CRISPR)-associated protein.

111. The method of claim 110, wherein the CRISPR-associated protein is selected from Cas9, CasX, CasY, Cpf1, and gRNA complexes thereof.

112. A method for treating a thrombocytopenia, comprising administering an effective amount of a composition of any one of claims 1-58, wherein the composition comprises megakaryocyte-derived extracellular vesicles which comprise a nucleic acid encoding a functional thrombocytopenia-related gene, or a protein product thereof, or a nucleic acid encoding a gene-editing protein capable of creating a functional thrombocytopenia-related gene, or a protein product thereof.

113. A method for treating a thrombocytopenia, comprising administering an effective amount of a composition comprising a cell which is contacted with composition of any one of claims 1-58 in vitro, wherein the composition and/or pharmaceutical composition comprises megakaryocyte-derived extracellular vesicles which comprise a nucleic acid encoding a functional thrombocytopenia-related gene, or a protein product thereof, or a nucleic acid encoding a gene-editing protein capable of creating a functional thrombocytopenia-related gene, or a protein product thereof.

114. The method of claim 112 or 113, wherein the thrombocytopenia is selected from congenital amegaryocytic thrombocytopenia (CAMT), thrombocytopenia with absent radii, radio ulnar synostosis with congenital thrombocytopenia, X-linked macrothrombocytopenia with thalassemia, GB11b-related thrombocytopenia, X-Linked Thrombocytopenia/Wiskott-Aldrich syndrome, Von Willebrand diseases Type 2B, platelet-type Von Willebrand disease, CYCS-Related thrombocytopenia, immune thrombocytopenia (idiopathic thrombocytopenia purpura), and myeloablation/chemotherapy induced thrombocytopenia.

115. The method of claim 114, wherein the thrombocytopenia is CAMT.

116. The method of claim 115, wherein the method provides a functional thrombopoietin (TPO) receptor in the patient.

117. The method of claim 115 or 116, wherein the gene is a functional c-Mpl gene or encodes a gene-editing protein that is capable of forming a functional c-Mpl gene.

118. The method of claim 117, wherein the gene-editing protein is selected from a zinc finger (ZF), transcription activator-like effector (TALE), meganuclease, and clustered regularly interspaced short palindromic repeat (CRISPR)-associated protein.

119. The method of claim 118, wherein the CRISPR-associated protein is selected from Cas9, CasX, CasY, Cpf1, and gRNA complexes thereof.

120. The method of any one of claims 112-119, wherein the method promotes megakaryopoeisis in the patient.

121. The method of any one of claims 118-120, wherein the method causes an increase in platelet counts in the patient.

122. The method of claim 121, wherein the increase in platelet counts is greater than about 100×10⁹ platelets/L, or greater than about 110×10⁹ platelets/L, or greater than about 120×10⁹ platelets/L, or greater than about 130×10⁹ platelets/L, or greater than about 140×10⁹ platelets/L, or greater than about 150×10⁹ platelets/L.

123. The method of any one of claims 112-122, wherein the method reduces the likelihood of the patient developing aplastic anemia and/or leukemia.

124. The method of any one of claims 112-123, wherein the method obviates the need for hematopoietic stem cell (HSC) transplantation.

125. The method of any one of claims 112-124, wherein the patient is an infant.

126. A method for treating a hemoglobinopathy, comprising administering an effective amount of a composition of any one of claims 1-58, wherein the composition comprises megakaryocyte-derived extracellular vesicles which comprise a nucleic acid encoding a functional hemoglobinopathy-related gene, or a protein product thereof, or a nucleic acid encoding a gene-editing protein capable of creating a functional hemoglobinopathy-related gene, or a protein product thereof.

127. A method for treating a hemoglobinopathy, comprising administering an effective amount of a composition comprising a cell which is contacted with composition of any one of claims 1-58 in vitro, wherein the composition comprises megakaryocyte-derived extracellular vesicles which comprise a nucleic acid encoding a functional hemoglobinopathy-related gene, or a protein product thereof, or a nucleic acid encoding a gene-editing protein capable of creating a functional hemoglobinopathy-related gene, or a protein product thereof.

128. The method of claim 126 or 127, wherein the functional hemoglobinopathy-related gene is a gene encoding a portion of hemoglobin.

129. The method of any one of claims 126-128, wherein the functional hemoglobinopathy-related gene is a gene encoding one of the globin chains of hemoglobin.

130. The method of any one of claims 126-129, wherein the functional hemoglobinopathy-related gene restores hemoglobin solubility, stability, and/or oxygen affinity to undiseased levels.

131. The method of any one of claims 126-130, wherein the functional hemoglobinopathy-related gene restores hemoglobin quantity to undiseased levels.

132. The method of claim 126-131, wherein the functional hemoglobinopathy-related gene is beta globin (HBB).

133. The method of claim 126 or 127, wherein the gene encodes a gene-editing protein that is capable of forming a functional beta globin (HBB) gene.

134. The method of claim 133, wherein the gene-editing protein is selected from a zinc finger (ZF), transcription activator-like effector (TALE), meganuclease, and clustered regularly interspaced short palindromic repeat (CRISPR)-associated protein.

135. The method of claim 134, wherein the CRISPR-associated protein is selected from Cas9, CasX, CasY, Cpf1, and gRNA complexes thereof.

136. The method of any one of claims 126-135, wherein the hemoglobinopathy is sickle cell disease.

137. The method of any one of claims 126-136, wherein the hemoglobinopathy is β-thalassemia.

138. The method of any one of claims 126-137, wherein the method reduces or prevents one or more of red cell distortion, hemolytic anemia, microvascular obstruction, and ischemic tissue damage.
